(19) United States
(12) Patent Application Publication
(10) Pub. No.: US 2023/0386661 A1
(43) Pub. Date: Nov. 30, 2023

(54) ANIMAL DIAGNOSTICS USING MACHINE LEARNING

(71) Applicant: MARS, INCORPORATED, McLean, VA (US)

(72) Inventors: Corryn WALLIS, Melton Mowbray, Leicestershire (GB); Lucy Jane HOLCOMBE, Melton Mowbray, Leicestershire (GB); Avika Kishorlal RUPARELL, Melton Mowbray, Leicestershire (GB)

(73) Assignee: MARS, INCORPORATED, McLean, VA (US)

(21) Appl. No.: 18/029,729

(22) PCT Filed: Oct. 4, 2021
(86) PCT No.: PCT/US2021/053411

§ 371 Date: Mar. 31, 2023

(30) Foreign Application Priority Data
Oct. 2, 2020 (GB) 2015675.8

(51) Int. Cl. G16H 50/20

(52) U.S. Cl. CPC G16H 50/20

(57) ABSTRACT

A device that is configured to obtain input data for an animal that is a member of the canid family is provided herein. The input data includes a first array having a first plurality of entries, where each entry within the first plurality of entries contains a numerical value that indicates an amount of a type of bacteria that is present within a sample from the animal. The device is further configured to input the input data for the animal into a machine learning model that is configured to receive the input data for the animal and to output an animal age value based at least in part on the input data for the animal. The animal age value identifies a predicted age for the animal. The device is further configured to obtain the animal age value from the machine learning model and to output the animal age value.

### CROSS-REFERENCE TO RELATED APPLICATIONS

This application claims priority to U.K. Patent Application No. 2015675.8, filed Oct. 2, 2020, the contents of which are incorporated herein by reference as if reproduced in its entirety.

### TECHNICAL FIELD

The present disclosure relates generally to machine learning, and more specifically to animal diagnostics using machine learning for determining an animal's age.

### BACKGROUND

The understanding of the oral microbiome and its impact on health has increased significantly in recent years. The prevalence and severity of periodontitis has been associated with pathological changes in the kidney, myocardium and liver of dogs [45, 46]. There is also some evidence of an increased likelihood of being diagnosed with endocarditis, cardiomyopathy, hepatopathy, hepatitis and chronic renal failure [47, 48]. The oral microbiome is distinct from the gut microbiome. The diversity of bacterial species found in the canid oral microbiome was initially studied by culture based methods, and more recently has been described using culture independent molecular methods, in which clear differences between the bacterial populations in the human versus canid oral microbiome were shown [7]. Further, the bacterial composition of the subgingival microbiota in the UK dog population has been described previously [20]. Given the importance of the oral microbiome to health and wellbeing, it is important to find ways to determine the status of the oral microbiome of an animal. An enhanced understanding of associations between the canid oral microbiome and oral age status is desirable.

The oral microbiome of an animal is a complex environment that comprises a large number of different types of organisms such as bacteria, bacteriophage, fungi, protozoa, etc. Each organisms type also comprises a large number of sub-classifications that describe the composition of an organism. Analyzing a large number of organisms and sub-classifications of organisms that can be present within the oral microbiome of an animal and connecting these organisms with other types of metadata for the animal is an intractable problem. Conventional computers are typically unable to solve or handle intractable problems due to their complexity and their computationally intensive nature. When a conventional computer system performs computationally intensive tasks, the number of resources (e.g., hardware processors and memory) that are consumed increases, and the number of available resources is reduced. In addition, the consumed resources are occupied for longer durations of time. This reduced supply of available resources limits the ability of the computer system to perform other tasks, limits the throughput of the computer system, and reduces the overall performance of the computer system.

### SUMMARY

The disclosed system provides several practical applications and technical advantages that overcome the previously discussed technical problems. For example, the disclosed system provides a practical application by providing the ability for a diagnostic system to efficiently analyze the oral microbiome of animals, to identify relationships between the ages of animals and different types of organisms that are present in the mouths of the animals, and to predict the age of an animal based on the analysis. This process allows the system to predict the age of an animal based on the health and physical attributes of the animal. The disclosed system employs a machine learning model that is configured to receive various types of inputs that describe the oral microbiome, the health, and/or the physical attributes of an animal and to output a predicted age for the animal based on the provided inputs. This process generally involves first training the machine learning model using samples and information that are collected for a large number of animals. During this process, the collected data is organized and formatted so that it can be easily ingested by the machine learning model using a supervised learning training process. Through the training process, the machine learning model is configured to map different types of input values to a predicted animal age. Once trained, the machine learning model can then be deployed to predict the age of an animal based on certain information about the animal. This process improves the operation of the system by offloading the complexity of analyzing the oral microbiome and other attributes of an animal to the trained machine learning model. Once the machine learning model is trained, the system is able to reduce the number of resources that are used to predict the age of an animal. Thus, the disclosed process provides a technical improvement that improves the operation of the system by improving resource utilization which in turn improves the throughput and the overall operation of the system.

In one embodiment, the diagnostic system comprises a device that is configured to obtain input data for an animal. The input data includes a first array having a first plurality of entries, where each entry within the first plurality of entries contains a numerical value that indicates an amount of a type of bacteria that is present within a sample from the animal. The device is further configured to input the input data for the animal into a machine learning model that is configured to receive the input data for the animal and to output an animal age value based at least in part on the input data for the animal. The animal age value identifies a predicted age for the animal. The device is further configured to obtain the animal age value from the machine learning model and to output the animal age value.

In certain embodiments, the input data for the animal further comprises an animal breed identifier that identifies a breed of the animal; and the machine learning model is further configured to output the animal age value based at least in part on the animal breed identifier.

In certain embodiments, the input data for the animal further comprises an animal size classification value; and the machine learning model is further configured to output the animal age value based at least in part on the animal size classification.

In certain embodiments, the input data for the animal further comprises a weight value that identifies a weight for the animal; and the machine learning model is further configured to output the animal age value based at least in part on the weight value.

In certain embodiments, the input data for the animal further comprises a gingivitis value for the animal; the gingivitis value is associated with a time to bleeding when probing a mouth of the animal; and the machine learning model is further configured to output the animal age value based at least in part on the gingivitis value.

In certain embodiments, the input data for the animal further comprises a periodontitis value for the animal; the periodontitis value is associated with an amount of periodontitis that is present in a mouth of the animal; and the machine learning model is further configured to output the animal age value based at least in part on the periodontitis value.

In certain embodiments, the input data for the animal further comprises geographical location (e.g., geolocation) information for a physical location associated with the animal; and the machine learning model is further configured to output the animal age value based at least in part on the geographical information.

The samples used in the system can comprise bacteria from a gingival area in a mouth of the animal. Alternatively, or in addition, the sample used in the system can comprises bacteria from a subgingival or supragingival area in a mouth of the animal. The samples used by the system can be collected while the animal is conscious or unconscious.

In certain embodiments, the processor is further configured to obtain training data for a second plurality of animals, wherein the training data indicates an amount of a type of bacteria that is present within a sample for each animal from among the second plurality of animals; associate the training data with animal age values, wherein associating the training data with the animal age values comprises associating each animal from among the second plurality of animals with an animal age value; and train the machine learning model using the training data that is associated with the animal age values.

In certain embodiments, the processor is further configured to associate the training data with animal size classification values before training the machine learning model; associating the training data with the animal size classification values comprises associating each animal from among the second plurality of animals with an animal size classification value.

In certain embodiments, the processor is further configured to associate the training data with animal breed identifiers before training the machine learning model; associating the training data with the animal breed identifiers comprises associating each animal from among the second plurality of animals with an animal breed identifier.

In certain embodiments, the processor is further configured to associate the training data with weight values before training the machine learning model; associating the training data with the weight values comprises associating each animal from among the second plurality of animals with a weight value.

In certain embodiments, the processor is further configured to associate the training data with gingivitis values before training the machine learning model; associating the training data with the gingivitis values comprises associating each animal from among the second plurality of animals with a gingivitis value.

In certain embodiments, the processor is further configured to associate the training data with periodontitis values before training the machine learning model; associating the training data with the periodontitis values comprises associating each animal from among the second plurality of animals with a periodontitis value.

In certain embodiments, the processor is further configured to associate the training data with geographic location information before training the machine learning model; associating the training data with the geographic location information comprises associating each animal from among the second plurality of animals with a physical location.

In certain embodiments, the sample comprises one or more bacteria selected from a group comprising denovo483, denovo7761, denovo13434, denovo11506, denovo6559, denovo11018, denovo11779, denovo5898, denovo7616, and denovo4478.

In certain embodiments, the sample comprises one or more bacteria selected from a group comprising denovo483, denovo7761, denovo5898, denovo13434, denovo248, denovo11018, denovo2415, denovo11506, denovo264, and denovo715.

The disclosed subject matter also provides for an age determination method, comprising: obtaining input data for an animal, wherein: the animal is a member of the canid family; the input data comprises a first array comprising a first plurality of entries; and each entry within the first plurality of entries comprises a numerical value that indicates an amount of a type of bacteria that is present within a sample from the animal; inputting the input data for the animal into a machine learning model, wherein the machine learning model is configured to: receive the input data for the animal; and output an animal age value based at least in part on the input data for the animal, wherein the animal age value identifies a predicted age for the animal; obtaining the animal age value from the machine learning model; and outputting the animal age value.

In certain embodiments of the method, the input data for the animal further comprises an animal breed identifier that identifies a breed of the animal; and the machine learning model is further configured to output the animal age value based at least in part on the animal breed identifier.

In certain embodiments, the input data for the animal further comprises an animal size classification value; and the machine learning model is further configured to output the animal age value based at least in part on the animal size classification.

In certain embodiments, the input data for the animal further comprises a weight value that identifies a weight for the animal; and the machine learning model is further configured to output the animal age value based at least in part on the weight value.

In certain embodiments, the input data for the animal further comprises a gingivitis value for the animal; the gingivitis value is associated with a time to bleeding when probing a mouth of the animal; and the machine learning model is further configured to output the animal age value based at least in part on the gingivitis value.

In certain embodiments, the input data for the animal further comprises a periodontitis value for the animal; the periodontitis value is associated with an amount of periodontitis that is present in a mouth of the animal; and the machine learning model is further configured to output the animal age value based at least in part on the periodontitis value.

In certain embodiments, the input data for the animal further comprises geographic location information for a physical location associated with the animal; and the machine learning model is further configured to output the animal age value based at least in part on the geographical information.

The samples used in the method can comprise bacteria from a gingival area in a mouth of the animal. Alternatively, or in addition, the sample used in the method can comprises bacteria from a subgingival or supragingival area in a mouth of the animal. The samples used by the method can be collected while the animal is conscious or unconscious.

In certain embodiments, the method further comprises obtaining training data for a second plurality of animals, wherein the training data indicates an amount of a type of bacteria that is present within a sample for each animal from among the second plurality of animals; associating the training data with animal age values, wherein associating the training data with the animal age values comprises associating each animal from among the second plurality of animals with an animal age value; and training the machine learning model using the training data that is associated with the animal age values.

In certain embodiments, the method further comprises associating the training data with animal size classification values before training the machine learning model; associating the training data with the animal size classification values comprises associating each animal from among the second plurality of animals with an animal size classification value.

In certain embodiments, the method further comprises associating the training data with animal breed identifiers before training the machine learning model; associating the training data with the animal breed identifiers comprises associating each animal from among the second plurality of animals with an animal breed identifier.

In certain embodiments, the method further comprises associating the training data with weight values before training the machine learning model; associating the training data with the weight values comprises associating each animal from among the second plurality of animals with a weight value.

In certain embodiments, the method further comprises associating the training data with gingivitis values before training the machine learning model; associating the training data with the gingivitis values comprises associating each animal from among the second plurality of animals with a gingivitis value.

In certain embodiments, the method further comprises associating the training data with periodontitis values before training the machine learning model; associating the training data with the periodontitis values comprises associating each animal from among the second plurality of animals with a periodontitis value.

In certain embodiments, the method further comprises associating the training data with geographic location information before training the machine learning model; associating the training data with the geographic location information comprises associating each animal from among the second plurality of animals with a physical location. The disclosed subject matter also provides for a computer program comprising executable instructions stored in a non-transitory computer-readable medium that when executed by a processor causes the processor to: obtain input data for an animal, wherein: the animal is a member of the canid family; the input data comprises a first array comprising a first plurality of entries; and each entry within the first plurality of entries comprises a numerical value that indicates an amount of a type of bacteria that is present within a sample from the animal; input the input data for the animal into a machine learning model, wherein the machine learning model is configured to: receive the input data for the animal; and output an animal age value based at least in part on the input data for the animal, wherein the animal age value identifies a predicted age for the animal; obtain the animal age value from the machine learning model; and output the animal age value.

In certain embodiments, the input data for the animal further comprises an animal size classification value; and the machine learning model is further configured to output the animal age value based at least in part on the animal size classification.

In certain embodiments, the input data for the animal further comprises an animal breed identifier that identifies a breed of the animal; and the machine learning model is further configured to output the animal age value based at least in part on the animal breed identifier.

In certain embodiments, the input data for the animal further comprises a weight value that identifies a weight for the animal; and the machine learning model is further configured to output the animal age value based at least in part on the weight value.

In certain embodiments, the input data for the animal further comprises a gingivitis value for the animal; the gingivitis value is associated with a time to bleeding when probing a mouth of the animal; and the machine learning model is further configured to output the animal age value based at least in part on the gingivitis value.

In certain embodiments, the input data for the animal further comprises a periodontitis value for the animal; the periodontitis value is associated with an amount of periodontitis that is present in a mouth of the animal; and the machine learning model is further configured to output the animal age value based at least in part on the periodontitis value.

In certain embodiments, the input data for the animal further comprises geographic location information for a physical location associated with the animal; and the machine learning model is further configured to output the animal age value based at least in part on the geographical information.

The samples used by the computer program product can comprise bacteria from a gingival area in a mouth of the animal. Alternatively, or in addition, the sample used in the method can comprises bacteria from a subgingival or supragingival area in a mouth of the animal. The samples used by the computer program product can be collected while the animal is conscious or unconscious.

In certain embodiments, the sample comprises one or more bacteria selected from a group comprising denovo483, denovo7761, denovo13434, denovo11506, denovo6559, denovo11018, denovo11779, denovo5898, denovo7616, and denovo4478.

In certain embodiments, the sample comprises one or more bacteria selected from a group comprising denovo483, denovo7761, denovo5898, denovo13434, denovo248, denovo11018, denovo2415, denovo11506, denovo264, and denovo715.

In certain embodiments, the computer program product further comprising instructions that when executed by the processor causes the processor to obtain training data for a second plurality of animals; the training data indicates an amount of a type of bacteria that is present within a sample for each animal from among the second plurality of animals; associate the training data with animal age values, wherein associating the training data with the animal age values comprises associating each animal from among the second plurality of animals with an animal age value; and train the machine learning model using the training data that is associated with the animal age values.

In certain embodiments, the computer program product further comprising instructions that when executed by the processor causes the processor to associate the training data with animal size classification values before training the machine learning model; associating the training data with the animal size classification values comprises associating each animal from among the second plurality of animals with an animal size classification value.

In certain embodiments, the computer program product further comprising instructions that when executed by the processor causes the processor to associate the training data with animal breed identifiers before training the machine learning model; associating the training data with the animal breed identifiers comprises associating each animal from among the second plurality of animals with an animal breed identifier.

In certain embodiments, the computer program product further comprising instructions that when executed by the processor causes the processor to associate the training data with weight values before training the machine learning model; associating the training data with the weight values comprises associating each animal from among the second plurality of animals with a weight value.

In certain embodiments, the computer program product further comprising instructions that when executed by the processor causes the processor to associate the training data with gingivitis values before training the machine learning model; associating the training data with the gingivitis values comprises associating each animal from among the second plurality of animals with a gingivitis value.

In certain embodiments, the computer program product further comprising instructions that when executed by the processor causes the processor to associate the training data with periodontitis values before training the machine learning model; associating the training data with the periodontitis values comprises associating each animal from among the second plurality of animals with a periodontitis value.

In certain embodiments, the computer program product further comprising instructions that when executed by the processor causes the processor to associate the training data with geographic location information before training the machine learning model; associating the training data with the geographic location information comprises associating each animal from among the second plurality of animals with a physical location.

In certain embodiments, the sample comprises one or more bacteria selected from a group comprising denovo483, denovo7761, denovo13434, denovo11506, denovo6559, denovo11018, denovo11779, denovo5898, denovo7616, and denovo4478.

In certain embodiments, the sample comprises one or more bacteria selected from a group comprising denovo483, denovo7761, denovo5898, denovo13434, denovo248, denovo11018, denovo2415, denovo11506, denovo264, and denovo715.

The presently disclosed subject matter also provides for a machine learning model training method, comprising: obtaining training data for a plurality of animals, wherein: the training data indicates an amount of a type of bacteria that is present within a sample for each animal from among the plurality of animals; and the plurality of animals are members of the canid family; associating the training data with animal age values, wherein associating the training data with the animal age values comprises associating each animal from among the second plurality of animals with an animal age value; and training a machine learning model using the training data that is associated with the animal age values, wherein the machine learning model is configured to: receive input data for an animal; and output an animal age value based at least in part on the input data for the animal, wherein the animal age value identifies a predicted age for the animal.

In certain embodiments, the method further comprises associating the training data with animal size classification values before training the machine learning model, wherein associating the training data with the animal size classification values comprises associating each animal from among the plurality of animals with an animal size classification value.

In certain embodiments, the method further comprises associating the training data with animal breed identifiers before training the machine learning model, wherein associating the training data with the animal breed identifiers comprises associating each animal from among the plurality of animals with an animal breed identifier.

In certain embodiments, the method further comprises associating the training data with weight values before training the machine learning model, wherein associating the training data with the weight values comprises associating each animal from among the plurality of animals with a weight value.

In certain embodiments, the method further comprises associating the training data with gingivitis values before training the machine learning model, wherein associating the training data with the gingivitis values comprises associating each animal from among the plurality of animals with a gingivitis value.

In certain embodiments, the method further comprises associating the training data with periodontitis values before training the machine learning model, wherein associating the training data with the periodontitis values comprises associating each animal from among the plurality of animals with a periodontitis value.

In certain embodiments, the method further comprises associating the training data with geographic location information before training the machine learning model, wherein associating the training data with the geographic location information comprises associating each animal from among the plurality of animals with a physical location.

In one aspect, the disclosed subject matter provides a method of determining the oral microbiome age status of a canid, comprising quantifying one or more bacterial taxa in a sample obtained from the oral cavity of the canid to determine the abundance or relative abundance of the bacterial taxa; and comparing the abundance and/or relative abundance of that bacterial taxa to the abundance or relative abundance of the same bacterial taxa in a control data set; and determining the oral microbiome age status. These methods are particularly useful for assessing a canid's health, as a discrepancy between the oral microbiome age status and the canid's actual age can be indicative of its health status. When a discrepancy is identified between the oral microbiome age status compared to its actual age, the canid's owner is notified of the discrepancy. For example, it could be undesirable for a young canid to have an oral microbiome status that is generally associated with an older canid and vice versa. In certain embodiments, the discrepancy indicates the canid's oral microbiome age status is less than the actual age of the canid or the canid's oral microbiome age status is greater than the actual age of the canid. In certain embodiments, the notification recommends an intervention comprising a diet change or increased vet care.

In certain embodiments, the method comprises quantifying one or more bacterial taxa selected from the group Peptostreptococcaceae bacterium COT-030, Helcococcus sp. COT-069, Peptostreptococcaceae bacterium COT-068, Novel Saccharibacteria (TM7) sp., Peptostreptococcaceae bacterium COT-077, Clostridiales bacterium COT-028, Proteiniphilum sp. COT-385, Spirochaeta sp. COT-314, Erysipelotrichaceae bacterium COT-302, Novel Rikenellaceae sp., and Saccharibacteria (TM7) sp. COT-308.

Further provided is a method of monitoring a canid comprising a step of determining the oral microbiome age status of a canid by the disclosed method on at least two time points, for example at least 6 months or 1 year apart. Such time points can be greater apart, including for example more than 1 year apart. This is particularly useful where a canid is receiving treatment to shift the oral microbiome as the method can monitor the progress of the therapy. It is also useful for monitoring health of the canid as a rapid shift from, for example, an adult microbiome to a senior microbiome, may be indicative of disease. This aspect, can also be used to assess whether the canid's microbiome progresses as the animal gets older. In certain aspects, the control data set comprises oral microbiome data from at least two, preferably three, preferably all four life stages of a canid selected from the list consisting of a puppy, an adult canid, a senior canid and a geriatric canid.

The methods of the disclosed subject matter include control data sets that consist of oral microbiome data taken from canids from a plurality of geographical locations or oral microbiome data taken from canids from a single geographical location, wherein optionally the canid is also from the same geographical location. In certain aspects, the control data set consists of oral microbiome data taken from canids of one breed size and the canid to be assessed is of the same breed size, optionally wherein the control data set consists of oral microbiome data taken from toy breed size and the canid to be assessed is of toy breed size, the control data set consists of oral microbiome data taken from small breed size and the canid to be assessed is of small breed size, the control data set consists of oral microbiome data taken from medium breed size and the canid to be assessed is of medium breed size, or the control data set consists of oral microbiome data taken from large breed size and the canid to be assessed is of large breed size. In various embodiments, the canid is a dog.

In certain embodiments, the method includes quantifying one or more bacterial taxa selected from specific groups of taxa specified herein and optionally the bacterial taxa has a 16S rDNA sequence with at least about 80%, about 85%, about 90%, about 95%, about 96%, about 97%, about 98%, or about 99% identical to the sequence of any one of SEQ ID Nos: 1, 3-6, 8-11, 13-30, 32-60, 62-63, 65-85, 87-88, 90-98, 100-147. In certain embodiments, the method includes quantifying one or more bacterial taxa selected from specific groups of taxa specified herein and optionally the bacterial taxa has a 16S rDNA sequence set forth in SEQ ID Nos: 1, 3-6, 8-11, 13-30, 32-60, 62-63, 65-85, 87-88, 90-98, 100-147. In one embodiment, the method includes quantifying one or more bacterial taxa selected from the group consisting of Aquaspirillum sp. FOT-079/COT-091, novel Erysipelotrichaceae sp. (OTU 11710), novel Tissierellaceae/Peptostreptococcaceae sp. (OTU 11779), Catonella sp. (COT-098/COT-158/FOT-010) and novel Alloprevotella/Prevotella sp. (OTU 11854), and optionally the bacterial taxa has a 16S rDNA sequence with at least about 80%, about 85%, about 90%, about 95%, about 96%, about 97%, about 98%, or about 99% identical to the sequence of any one of SEQ ID NOs 5, 13, 14, 15 and/or 16. In one embodiment, the method includes quantifying one or more bacterial taxa selected from the group consisting of Aquaspirillum sp. FOT-079/COT-091, novel Erysipelotrichaceae sp. (OTU 11710), novel Tissierellaceae/Peptostreptococcaceae sp. (OTU 11779), Catonella sp. (COT-098/COT-158/FOT-010) and novel Alloprevotella/Prevotella sp. (OTU 11854), and optionally the bacterial taxa has a 16S rDNA sequence set forth in SEQ ID NOs 5, 13, 14, 15 and/or 16. The method can also include quantifying one or more bacterial taxa selected from the group Blautia sp. (COT-337), Novel Bergeyella/Novel Weeksellaceae/Loacibacterium sp. COT-320 (OTU 1233), Capnocytophaga canimorsus, Prevotella sp. COT-226 and Conchiformibius steedae, and optionally, the bacterial taxa has a 16S rDNA sequence with at least about 80%, about 85%, about 90%, about 95%, about 96%, about 97%, about 98%, or about 99% identical to the sequences of at least 2, 3, 4 or all of SEQ ID NOs: 17, 18, 21, 23 and 27. In certain embodiments, the method can also include quantifying one or more bacterial taxa selected from the group Blautia sp. (COT-337), Novel Bergeyella/Novel Weeksellaceae/Loacibacterium sp. COT-320 (OTU 1233), Capnocytophaga canimorsus, Prevotella sp. COT-226 and Conchiformibius steedae, and optionally, the bacterial taxa has a 16S rDNA sequence identical to the sequences of at least 2, 3, 4 or all of SEQ ID NOs: 17, 18, 21, 23 and 27. The bacterial species can be detected or quantified by means of DNA sequencing, RNA sequencing, protein sequence homology or other biological marker indicative of the bacterial species.

The methods of the disclosed subject matter can comprise a further step of changing the composition of the oral microbiome. This can be achieved through a change in the oral care regime (such as tooth brushing and/or professional tooth cleaning), a dietary change or a functional food or supplement and/or through administration of a nutraceutical or pharmaceutical composition or of a preparation or oral chew and/or oral care solution (preferably dietary change, oral chew and/or oral care solution). Such nutraceutical, composition or preparation can contain one or more bacteria. This is particularly useful where the methods have identified an oral microbiome that has an age status that is not consistent with the canid's actual age, e.g., where the canid may therefore be less healthy in the context of the animal's actual age. This will usually be done where the oral microbiome is deemed to require or benefit from enhancement or where it has an incompatible oral microbiome age status, but can also be undertaken pre-emptively.

Also provided is a method of monitoring the microbiome age status in a canid who has undergone a change in the oral care regime (such as tooth brushing and/or professional tooth cleaning), a dietary change and/or who has received a supplement, a functional food, a nutraceutical composition, a pharmaceutical composition or a preparation, e.g., comprising bacteria that is able to change the microbiome composition, comprising determining the microbiome age status by a method according to the disclosed subject matter. Such methods allow a skilled person to determine the success of the treatment. Preferably, these methods comprise determining the microbiome age status before and after treatment as this helps to evaluate the success of the treatment.

Also provided is a method of assessing the oral microbiome age status of a canid to determine whether an intervention is required, comprising (a) quantitating one or more bacterial taxa in a sample obtained from the canid; (b) determining the abundance or relative abundance of said bacterial taxa; (c) comparing the abundance or relative abundance determined in step (b) to that of a control data set; wherein if the comparing of step (c) indicates a difference in microbiome age status to actual age of the animal, an intervention is recommended.

The samples obtained from the oral cavity of the canid can comprise oral plaque (such as subgingival or gingival margin dental plaque, supragingival dental plaque, plaque from the tongue and/or plaque from the cheeks), or saliva, wherein the control data set comprises abundance or relative abundance data of the one or more bacterial taxa found in the oral plaque (such as subgingival or gingival margin dental plaque, plaque from the tongue and/or plaque from the cheeks) of the one or more canids. Alternatively, or additionally, the sample obtained from the oral cavity of the canid can comprise subgingival or gingival margin dental plaque, supragingival dental plaque, preferably gingival margin dental plaque, supragingival dental plaque, wherein the control data set comprises abundance or relative abundance data of the one or more bacterial taxa found in the subgingival, gingival margin dental plaque or supragingival dental plaque, preferably supragingival plaque, of the one or more canids.

Certain embodiments of the present disclosure can include some, all, or none of these advantages. These advantages and other features will be more clearly understood from the following detailed description taken in conjunction with the accompanying drawings and claims.

### BRIEF DESCRIPTION OF THE DRAWINGS

For a more complete understanding of this disclosure, reference is now made to the following brief description, taken in connection with the accompanying drawings and detailed description, wherein like reference numerals represent like parts.

FIG. 1 is a schematic diagram of an embodiment of a diagnostic system configured to perform animal diagnostics using machine learning;

FIG. 2 is a flowchart of an embodiment of an age determination process for the information;

FIG. 3 is a schematic diagram of an embodiment of a hardware configuration for a network device configured to perform animal diagnostics using machine learning;

FIG. 4A is an example of principal component scores for operational taxonomic units (OTUs) based on geographical location for a canid;

FIG. 4B is an example of principal component scores for OTUs based on breed size of a canid;

FIG. 4C is an example of principal component scores for OTUs based on age of a canid;

FIG. 4D is an example of principal component scores for OTUs based on average gingivitis of a canid;

FIG. 4E is an example of principal component scores for OTUs based on percentage of healthy teeth on a canid;

FIG. 4F is an example of principal component scores for OTUs based on percentage of periodontitis teeth on a canid;

FIG. 5 is an example of odds ratios for OTUs with age effects;

FIG. 6 is an example of bacterial taxa that have significant interaction with age by location;

FIG. 7 is an example of bacterial taxa that were shown to have significantly altered relative abundance with age;

FIG. 8 is another example of bacterial taxa that were shown to have significantly altered relative abundance with age;

FIG. 9 is an example of a list of bacterial taxa that can be used for describing the biological age of the oral microbiome of canids;

FIG. 10 is an example of 16S rDNA sequences for the bacterial taxa identified in FIG. 7;

FIG. 11 is an example of a summary of the estimated mean proportions, odds ratios, 95% confidence intervals and p-values for the OTUs identified in FIG. 4B;

FIG. 12 is an example of the 16S rDNA sequences for the bacterial taxa identified in FIG. 4B;

FIG. 13A is an example of a principal component scores for an analysis based on plaque sample types;

FIG. 13B is an example of a principal component scores for an analysis based on geographic location;

FIG. 14 is an example of an average taxonomic composition per plaque location with 95% confidence intervals;

FIG. 15 is an example of Shannon diversity index with 95% confidence intervals for subgingival and gingival margin plaque samples;

FIGS. 16A and 16B are examples of discrimination of plaque sample locations by oxygen requirement status;

FIG. 17A is an example of Euler diagrams depicting OTUs with associations with proportions of healthy teeth, proportions of periodontitis teeth, or average gingivitis score;

FIG. 17B is an example of OTUs with fewer than 50% zeros;

FIG. 17C is an example of OTUs that were modeled based on presence/absence as data contained between 50% and 80% zeros;

FIG. 18A is another example of Euler diagrams depicting OTUs with significant associations with proportions of healthy teeth, proportions of periodontitis teeth, or average gingivitis score and location;

FIG. 18B is another example of OTUs with fewer than 50% zeros;

FIG. 18C is another example of OTUs that were modeled based on presence/absence as data contained between 50% and 80% zeros;

FIG. 19 is an example of OTUs for a machine learning model;

FIG. 20 is an example of the relationship between age and the relative abundance of OTU #7791 by location in the mouth;

FIG. 21 is an example of the relationship between age and the relative abundance of OTU #28682 by location in the mouth; and

FIG. 22 is an example of the relationship between age and the relative abundance of OTU #23212 by location in the mouth.

### DETAILED DESCRIPTION

General Overview

The presently disclosed subject matter is directed to the discovery that the amount (e.g., the abundance or relative abundance) of specific bacterial taxa in the canid oral microbiome changes throughout the life of a canid. By analyzing the oral microbiome in a group of canids of different ages and observing that the relative abundance of a large number of bacterial taxa positively or negatively correlates with the canid's age, the presently disclosed subject matter has demonstrated that canine oral bacteria levels can be used as a means of tracking and maintaining canine health. More specifically, information about the abundance or relative abundance of one or more of these bacterial taxa in a sample from a canid can thus be used to determine an oral microbiome age status to the canid.

The terms used in this specification generally have their ordinary meanings in the art, within the context of this description and in the specific context where each term is used. Certain terms are discussed below, or elsewhere in the specification, to provide additional guidance to the practitioner in describing the methods and compositions of the disclosed subject matter and how to make and use them.

References to a percentage sequence identity between two nucleotide sequences mean that, when aligned, that percentage of nucleotides are the same in comparing the two sequences. This alignment and the percent homology or sequence identity can be determined using any suitable software programs. For example, those described in section 7.7.18 of reference [18]. In one embodiment, an alignment is determined using the BLAST algorithm or the Smith-Waterman homology search algorithm using an affine gap search with a gap open penalty of 12 and a gap extension penalty of 2, BLOSUM matrix of 62. The Smith-Waterman homology search algorithm is disclosed in reference [19]. The alignment can be over the entire reference sequence, i.e., it can be over 100% length of the sequences disclosed herein.

As used herein, the use of the word “a” or “an” when used in conjunction with the term “comprising” in the claims and/or the specification can mean “one,” but it is also consistent with the meaning of “one or more,” “at least one,” and “one or more than one.” Still further, the terms “having,” “containing,” and “comprising” are interchangeable, and one of skill in the art is cognizant that these terms are open ended terms. Further, the term “comprising” encompasses “including” as well as “consisting,” e.g., a composition “comprising” X can consist exclusively of X or can include something additional, e.g., X+Y.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, or alternatively up to 10%, or alternatively up to 5%, and alternatively still up to 1% of a given value. Alternatively, particularly with respect to biological systems or processes, the term can mean within an order of magnitude, preferably within 5-fold, and more preferably within 2-fold, of a value. In certain embodiments, the term “about” in relation to a numerical value x is optional and means, for example, x+10%.

The term “effective treatment” or “effective amount” of a substance means the treatment or the amount of a substance that is sufficient to effect beneficial or desired results, including clinical results, and, as such, an “effective treatment” or an “effective amount” depends upon the context in which it is being applied. In the context of administering a composition (e.g., a dietary change, a functional food, a supplement, a nutraceutical composition, or a pharmaceutical composition) to change the composition of a microbiome having an unhealthy microbiome, the effective amount is an amount sufficient to bring the health status of the microbiome back to a healthy state, which is determined according to one of the methods disclosed herein. In certain embodiments, an effective treatment, as described herein, can also include administering a treatment in an amount sufficient to decrease any symptoms associated with an unhealthy microbiome. The decrease can be an about 0.01%, about 0.1%, about 1%, about 5%, about 10%, about 20%, about 30%, about 40%, about 50%, about 60%, about 70%, about 80%, about 90%, about 95%, about 98% or about 99% decrease in severity of symptoms of an unhealthy microbiome. An effective amount can be administered in one or more administrations. A likelihood of an effective treatment described herein is a probability of a treatment being effective, i.e., sufficient to alter the microbiome, or treat or ameliorate a disorder and/or inflammation, as well as decrease the symptoms.

As used herein, and as well-understood in the art, “treatment” is an approach for obtaining beneficial or desired results, including clinical results. For purposes of this subject matter, beneficial or desired clinical results include, but are not limited to, alleviation or amelioration of one or more symptoms, diminishment of extent of a disorder, stabilized (i.e., not worsening) state of a disorder, prevention of a disorder, delay or slowing of the progression of a disorder, and/or amelioration or palliation of a state of a disorder. In certain embodiments, the decrease can be an about 0.01%, about 0.1%, about 1%, about 5%, about 10%, about 20%, about 30%, about 40%, about 50%, about 60%, about 70%, about 80%, about 90%, about 95%, about 98% or about 99% decrease in severity of complications or symptoms. “Treatment” can also mean prolonging survival as compared to expected survival if not receiving treatment.

The word “substantially” does not exclude “completely” e.g. a composition which is “substantially free” from Y can be completely free from Y. Where necessary, the word “substantially” can be omitted from the definition of the present disclosure.

The term “taxa” refers to taxonomical groups, for example, kingdom, phylum, class, order, family, genus, and species. The term “abundance” can refer to an absolute amount (including presence or absence) of given bacterial taxa present within a sample. For example, an abundance can refer to the count of bacterial sequences of bacterial taxa after appropriate amplification of 16S rDNA. The term “relative abundance” can refer to a percentage composition of bacteria of particular bacterial taxa (e.g., species) relative to the total number of bacteria in the sample. It can be calculated by determining the number of sequences of given bacterial taxa divided by the total number of all bacterial sequences which is then multiplied by 100. For example, the relative abundance can refer to the amounts and relative amounts of nucleic acid present in a sample after appropriate amplification of 16S rDNA. In certain embodiments, the relative abundance can refer to a binary classification of bacteria taxa. For example, without any limitation, binary classification can include detected versus undetected taxa or presence versus absence of taxa. In certain embodiments, the relative abundance is calculated as odds ratio. As used herein, odds ratio can be a fold change, i.e., it is a measure of how much higher or lower the abundance or relative abundance is when comparing one group to another group.

As used herein, the term “biomarker” can refer to a characteristic that is objectively measured and evaluated as an indicator of physiological biological processes, pathogenic processes, or pharmacologic responses to a therapeutic intervention. In certain non-limiting embodiments, the term “biomarker” can refer to any substance, structure, or process that can be measured in the body or its products and influence or predict the incidence of outcome or disease.

As used herein, the terms “OTU” and “Operational Taxonomic Unit” refer to classified bacteria based on sequence similarity of the 16S marker gene (e.g., 16S rRNA or 16S rDNA). In certain embodiments, an OTU includes a group of bacteria whose 16S marker gene shows a sequence identity of at least about 80%. In certain embodiments, an OTU includes a group of bacteria whose 16S marker gene shows a sequence identity of at least about 97%. In certain embodiments, OTU is used to classify bacteria at the genus level.

The Canid Family

In one embodiment, the diagnostic system 100 can be used to determine the microbiome age status of an animal that is a canid. This genus comprises domestic dogs (Canis lupus familiaris), wolves, coyotes, foxes, jackals, and dingoes. For example, the subject can be a domestic dog herein referred to simply as a dog.

There are numerous different breeds of domestic dogs which show a diverse habitus. Different breeds also have different life expectancies with smaller dogs generally being expected to live longer than bigger breeds. Accordingly, different breeds are considered to be puppies, adults, seniors, or geriatric at different time points in their life. Table 1 is an example of a summary of the different life stages for a dog.

TABLE 1
An example of a summary of the different life stages for a dog

| | Puppy | Adult | Senior | Geriatric |
|---|---|---|---|---|
| Toy | Up to 7 years | 8-11 years | 12-13 years | 14+ years |
| Small | Up to 7 years | 8-11 years | 12-13 years | 14+ years |
| Medium | Up to 5 years | 6-9 years | 10-13 years | 14+ years |
| Large | Up to 5 years | 6-9 years | 10-11 years | 12+ years |

Toy breeds, extra-small breeds, and puppies can an average weight of up to about 6.5 kg (although exceptions may exist). Examples of toy breeds include, but are not limited to, Affenpinscher, Australian Silky Terrier, Bichon Frise, Bolognese, Cavalier King Charles Spaniel, Chihuahua, Chinese Crested, Coton De Tulear, English Toy Terrier, Griffon Bruxellois, Havanese, Italian Greyhound, Japanese Chin, King Charles Spaniel, Lowchen (Little Lion Dog), Maltese, Miniature Pinscher, Papillon, Pekingese, Pomeranian, Pug, Russian Toy, and Yorkshire Terrier. Small breeds are larger on average than toy breeds with an average body weight of up to about 10 kg or about 6.5 kg to about 9 kg. Examples of small breeds include, but are not limited to, French Bulldog, Beagle, Dachshund, Pembroke Welsh Corgi, Miniature Schnauzer, Cavalier King Charles Spaniel, Shih Tzu, and Boston Terrier. Medium dog breeds have an average weight of about 11 kg to about 26 kg. More specifically and/or alternatively, medium small breeds can range from about 9 kg to about 15 kg; whereas medium large breeds can range from about 15 kg to less than about 30 kg. Examples of medium dog breeds include, but are not limited to, Bulldog, Cocker Spaniel, Shetland Sheepdog, Border Collie, Basset Hound, Siberian Husky, and Dalmatian. Large breeds are those with an average body weight of at least 27 kg. Alternatively, large breeds can range from about 30 kg to less than about 40 kg. Examples of large breed dogs include, but are not limited to, Great Dane, Neapolitan mastiff, Scottish Deerhound, Dogue de Bordeaux, Newfoundland, English mastiff, Saint Bernard, Leonberger, and Irish Wolfhound. Giant breeds can have an average weight of less than about 40 kg. Cross-breeds can generally be categorized as toy, small, medium, and large dogs depending on their body weight.

System Overview

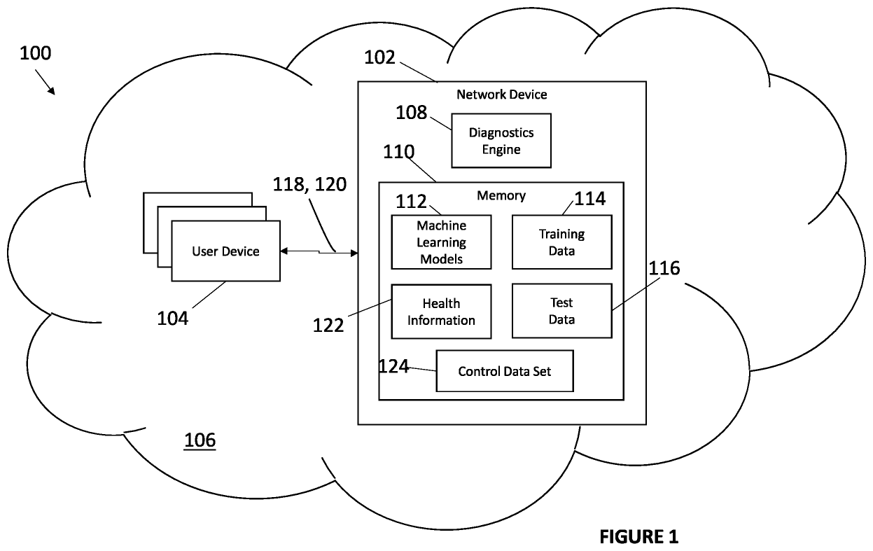

FIG. 1 is a schematic diagram of an embodiment of a diagnostic system 100 that is configured to perform animal diagnostics using machine learning. In one embodiment, the diagnostic system 100 can be configured to perform diagnostics on animals that are members of the canid family. The diagnostic system 100 is generally configured to input various types of information that are associated with the health and attributes of an animal into a machine learning model 112. The machine learning model 112 is configured to predict the age of the animal based on the provided inputs. This process allows the diagnostic system 100 to determine the age of an animal based on the physical attributes of the animal.

In one embodiment, the diagnostic system 100 comprises one or more user devices 104 and a network device 102 that are in signal communication with each other over a network 106. The network 106 can be any suitable type of wireless and/or wired network including, but not limited to, all or a portion of the Internet, an Intranet, a private network, a public network, a peer-to-peer network, the public switched telephone network, a cellular network, a local area network (LAN), a metropolitan area network (MAN), a personal area network (PAN), a wide area network (WAN), and a satellite network. The network 106 can be configured to support any suitable type of communication protocol as would be appreciated by one of ordinary skill in the art.

User Devices

Examples of user devices 104 include, but are not limited to, a computer, a laptop, a tablet, a smartphone, a smart device, an Internet-of-Things (IoT) device, a data storage device (e.g., a Universal Serial Bus (USB) drive or flash drive), or any other suitable type of device. A user device 104 is configured to provide input data 118 for an animal to the network device 102. The input data 118 can comprise information associated with bacterial taxa operational taxonomic units (OTUs), an animal breed identifier, an animal size, an animal weight, animal health information, geographical location information, or any other suitable type of information that is associated with an animal. In response to providing the input data 118 for an animal to the network device 102, the user device 104 is configured to receive an animal age value from the network device 102 and to display the animal age value to a user. For example, the user device 104 can comprise a graphical user interface (e.g., a display or a touchscreen) that allows a user to the animal age value. The user device 104 can further comprise a touchscreen, a touchpad, keys, buttons, a mouse, or any other suitable type of hardware that allows a user to provide inputs into the user device 104.

Network Device

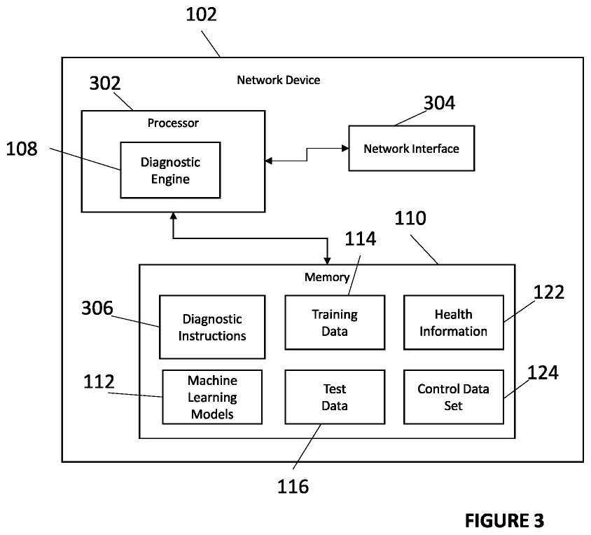

Examples of the network device 102 include, but are not limited to, a server (e.g., a cloud server), a computer, a laptop, or any other suitable type of network device. In one embodiment, the network device 102 comprises a diagnostics engine 108 and a memory 110. Additional details about the hardware configuration of the network device 102 are described in FIG. 3. The memory 110 is configured to store machine learning models 112, training data 114, test data 116, health information 122, a control data set 124, and/or any other suitable type of data.

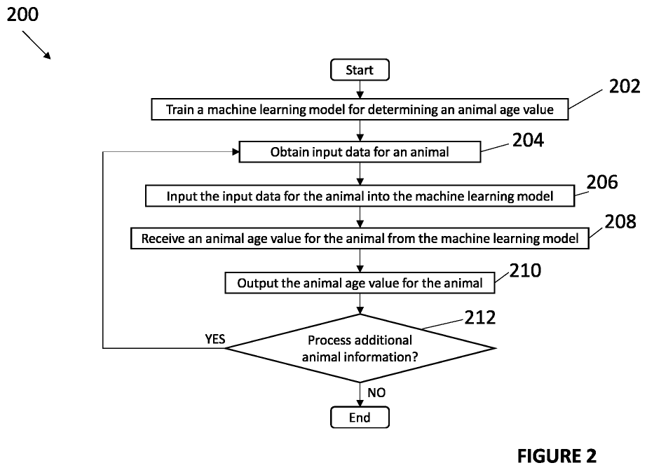

In one embodiment, the diagnostics engine 108 is generally configured to employ a machine learning model 112 to determine an animal's age based on information that is associated with an animal. An example of the diagnostics engine 108 in operation is described in more detail below in FIG. 2.

Examples of machine learning model types include, but are not limited to, a multi-layer perceptron, a recurrent neural network (RNN), an RNN long short-term memory (LSTM), a convolutional neural network (CNN), deep learning algorithms, probabilistic models, a linear regression, a non-linear regression, or any other suitable type of algorithm or model. The machine learning model 112 can be configured with any suitable type of hyperparameters or settings. Examples of hyperparameters and settings include, but are not limited to, a sensitivity level, a tolerance level, an epoch value, a number of layers (e.g., hidden layers), a number of inputs, a number of outputs, an output type, an output format, or any other suitable type or combination of settings. As an example, the machine learning model 112 can be configured with hyperparameters such as a learning rate of 0.15, a max depth of 2, and a maximum number of rounds set to 22. As another example, the machine learning model 112 can be configured with hyperparameters such as a learning rate of 0.15, a max depth of 5, and a maximum number of rounds set to 32. In other examples, the machine learning model 112 can be configured with any other suitable hyperparameters.

The machine learning model 112 is generally configured to receive input data 118 for an animal as an input and to output an animal age value 120 based on the provided input data 118. The animal age value 120 is a numeric value that corresponds with a predicted age for the animal based on the provided input data 118. In one embodiment, the machine learning model 112 is trained using supervised learning with training data 114 that comprises information associated with different animals with their corresponding labels (e.g., animal age values 120). During the training process, the machine learning model 112 determines weights and bias values that allow the machine learning model 112 to map information associated with different animals to different animal age values 120. Through this process, the machine learning model 112 is able to identify an animal age value 120 based on the provided input data 118. The diagnostics engine 108 can be configured to train the machine learning model 112 using any suitable technique as would be appreciated by one of ordinary skill in the art. For example, the machine learning model 112 can be trained using an XGBoost algorithm. In some embodiments, the machine learning model 112 can be stored and/or trained by a device that is external from the network device 102.

In some embodiments, the network device 102 may be configured to use statistical models, regression models (e.g. non-linear regression models), parametric models, or any other suitable type of model with or in place of the machine learning model 112.

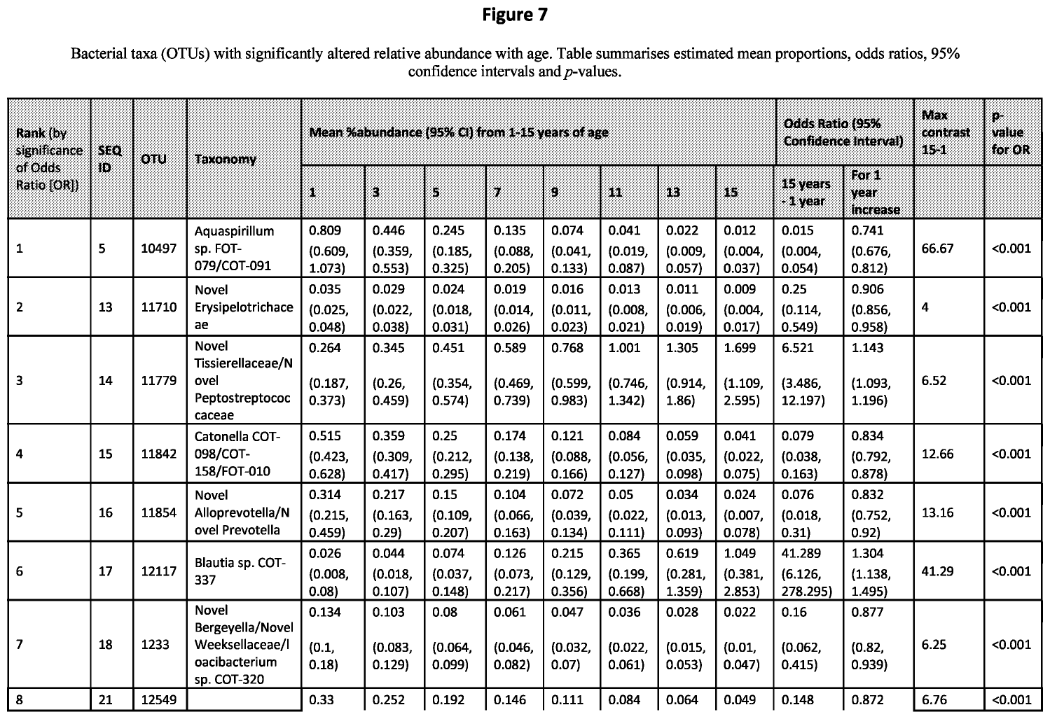

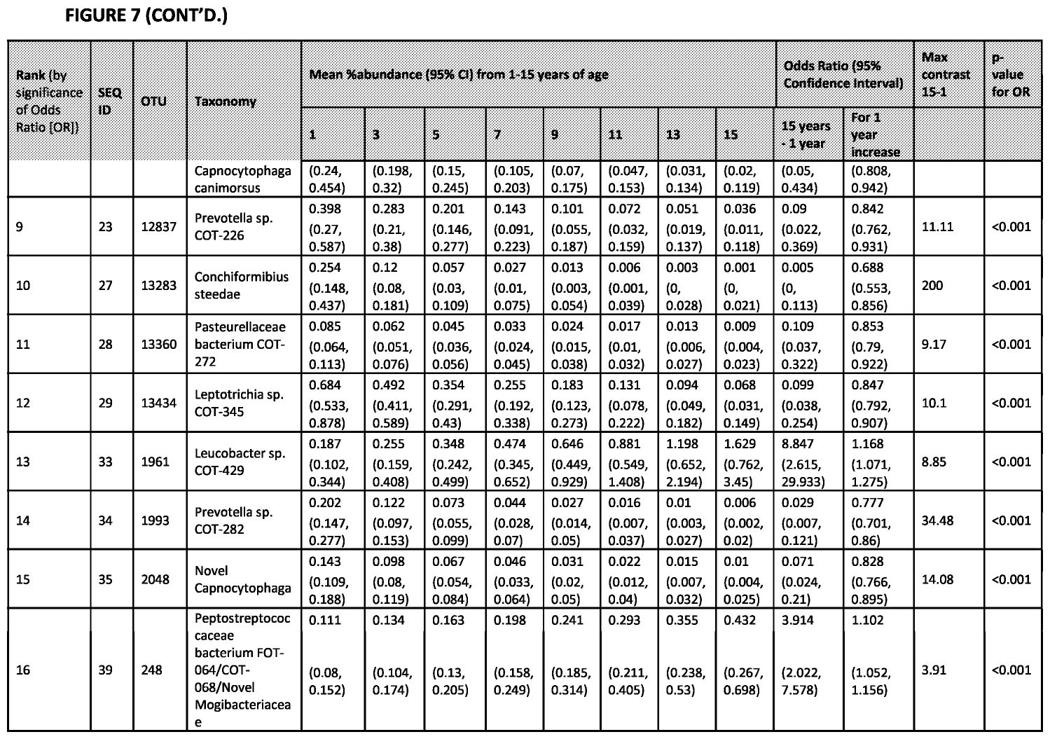

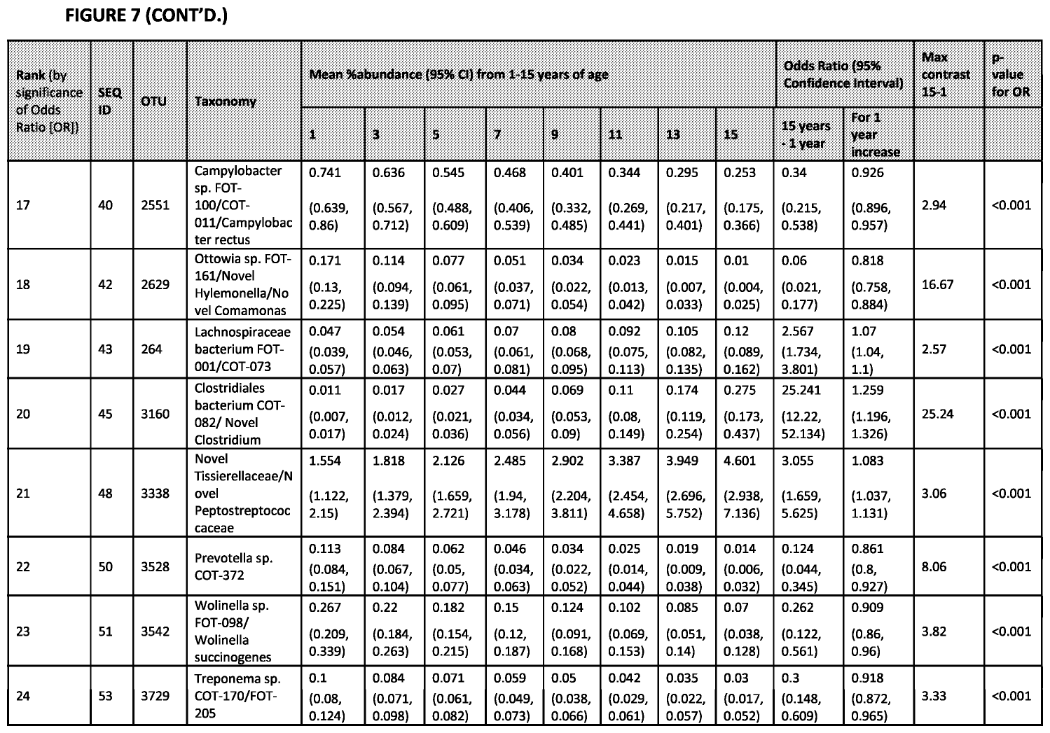

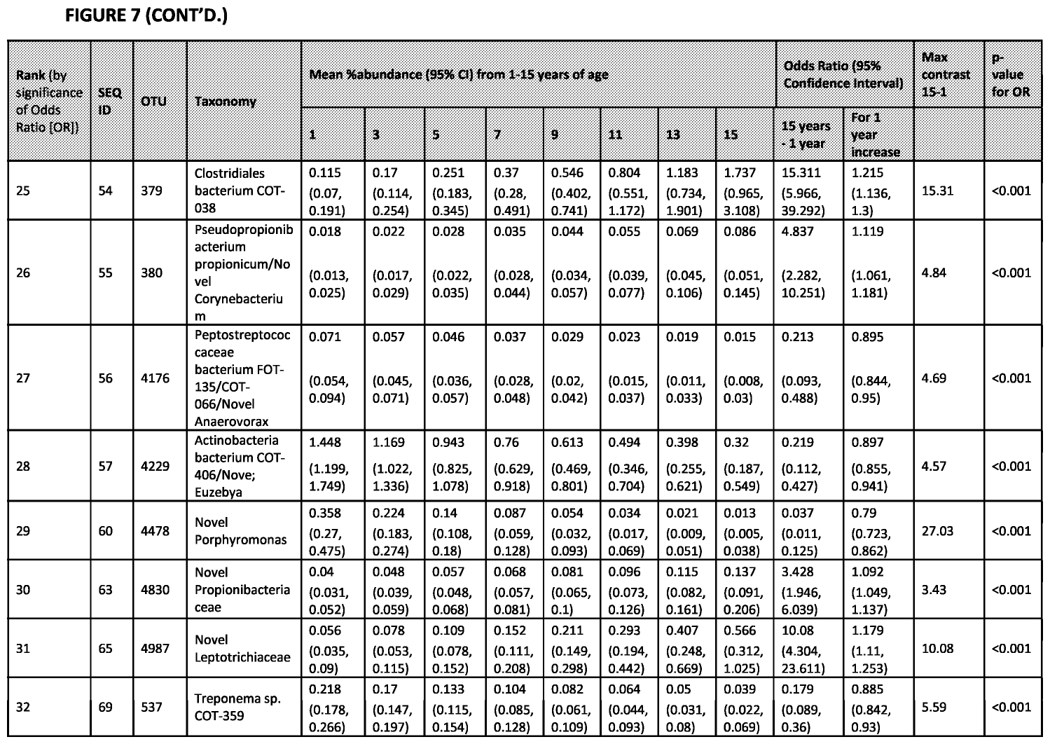

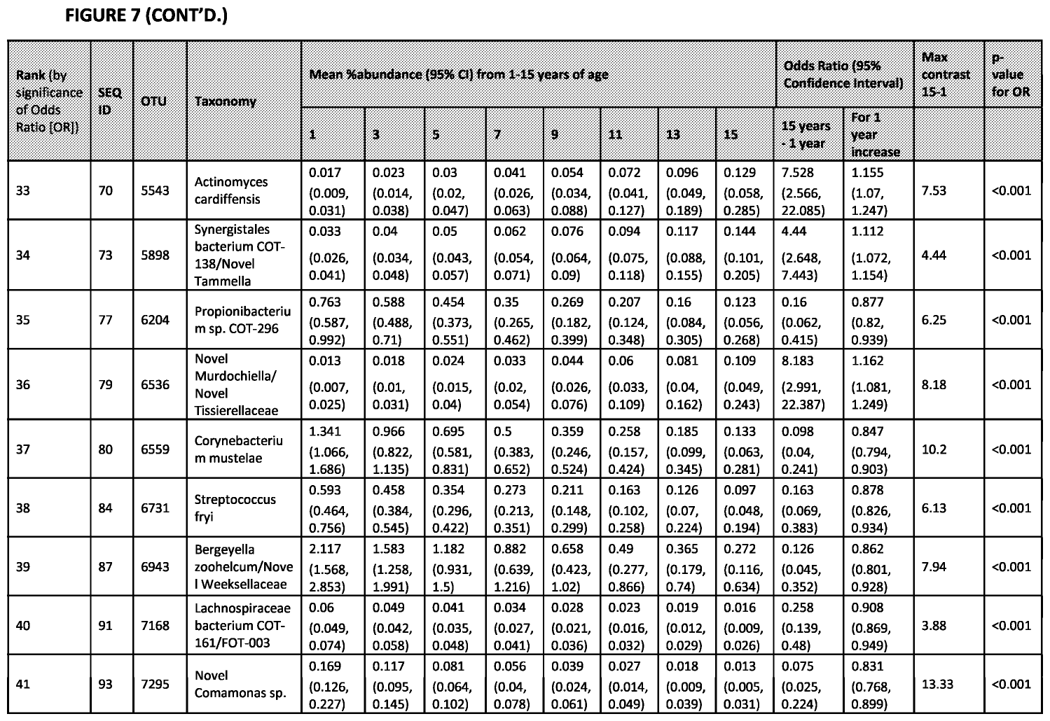

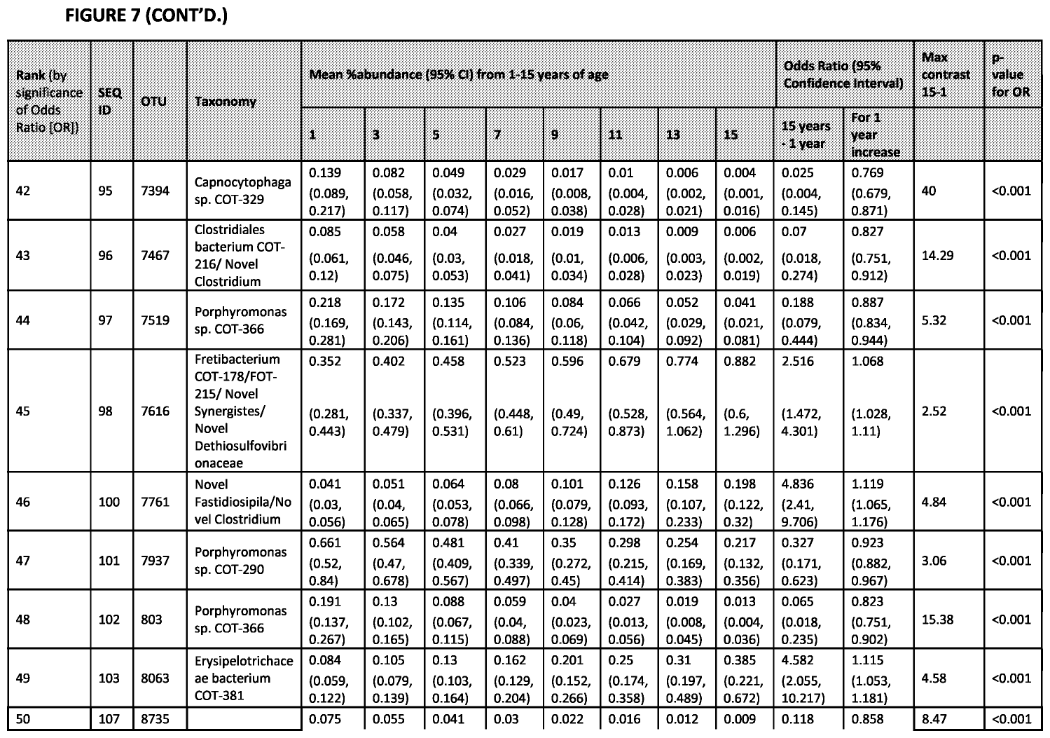

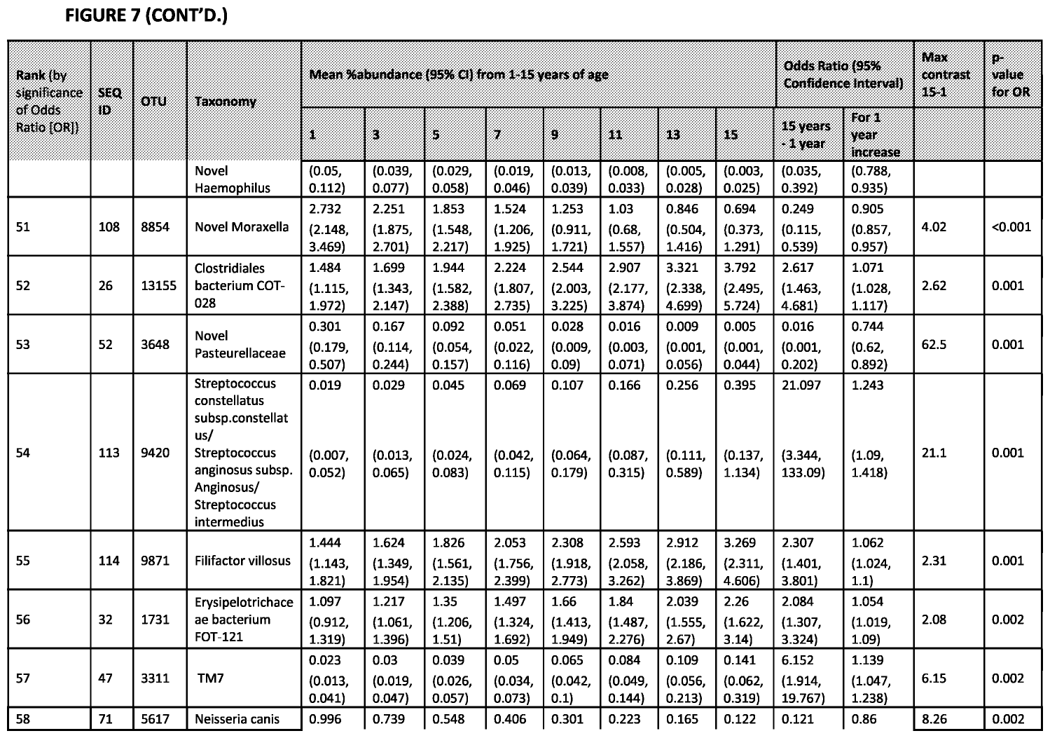

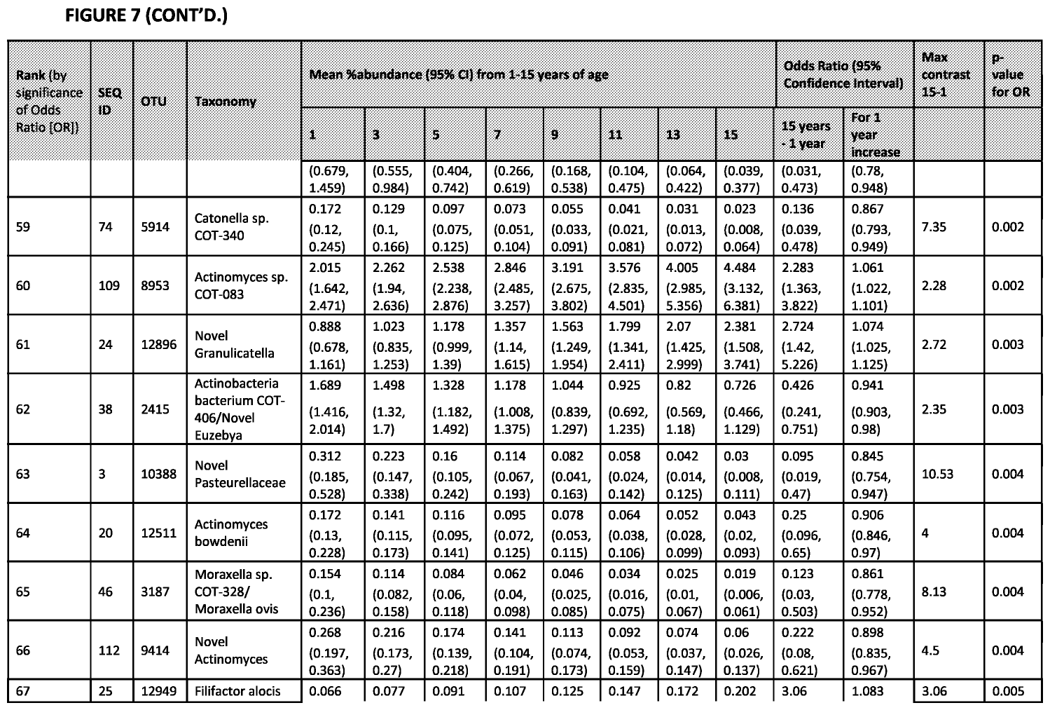

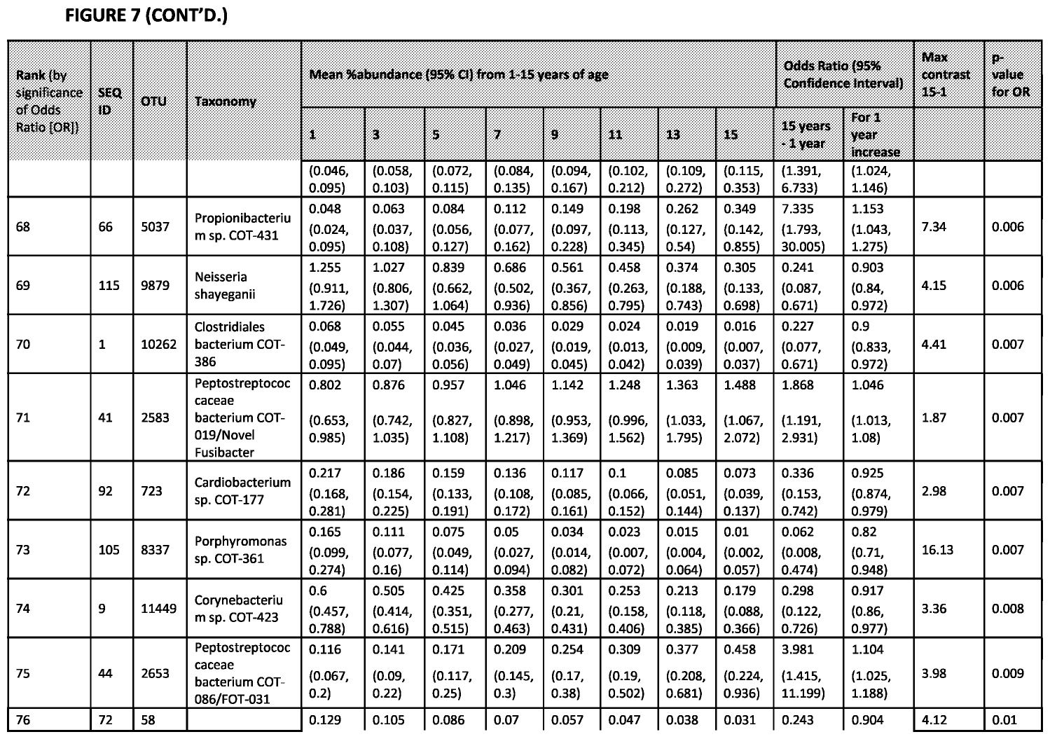

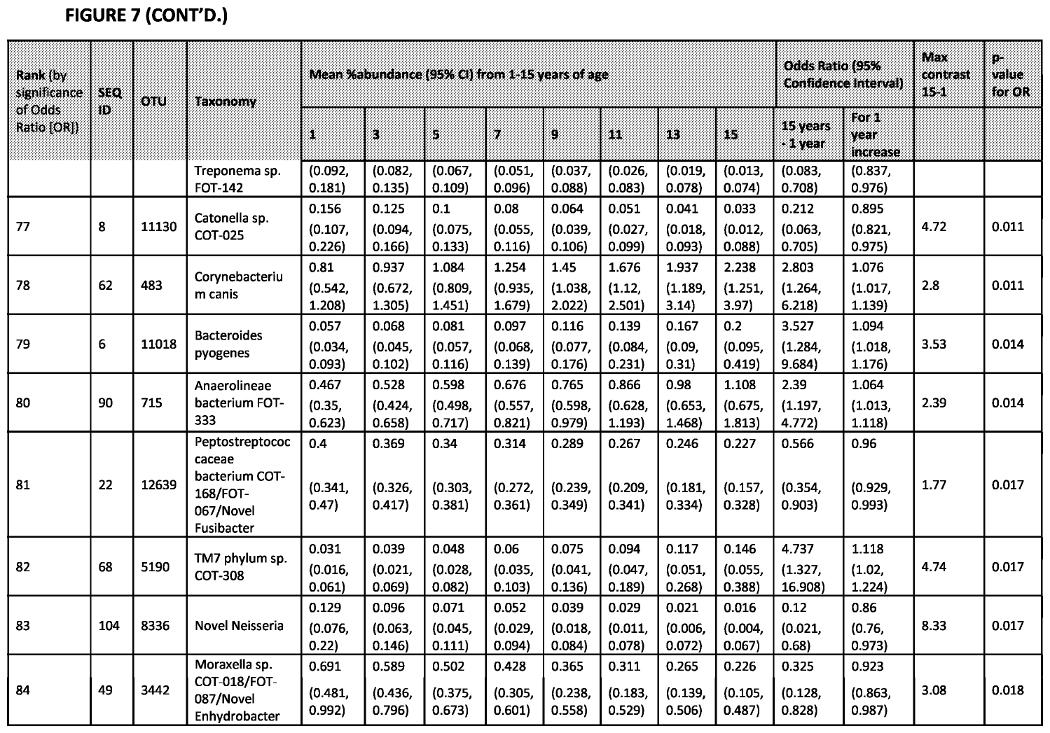

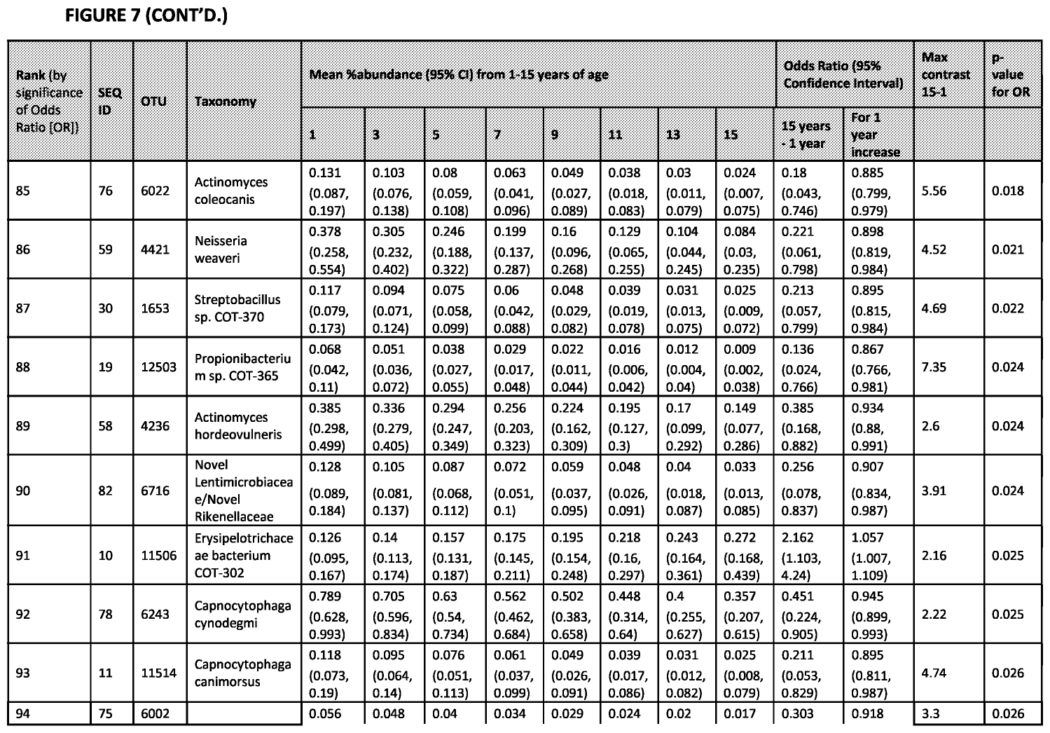

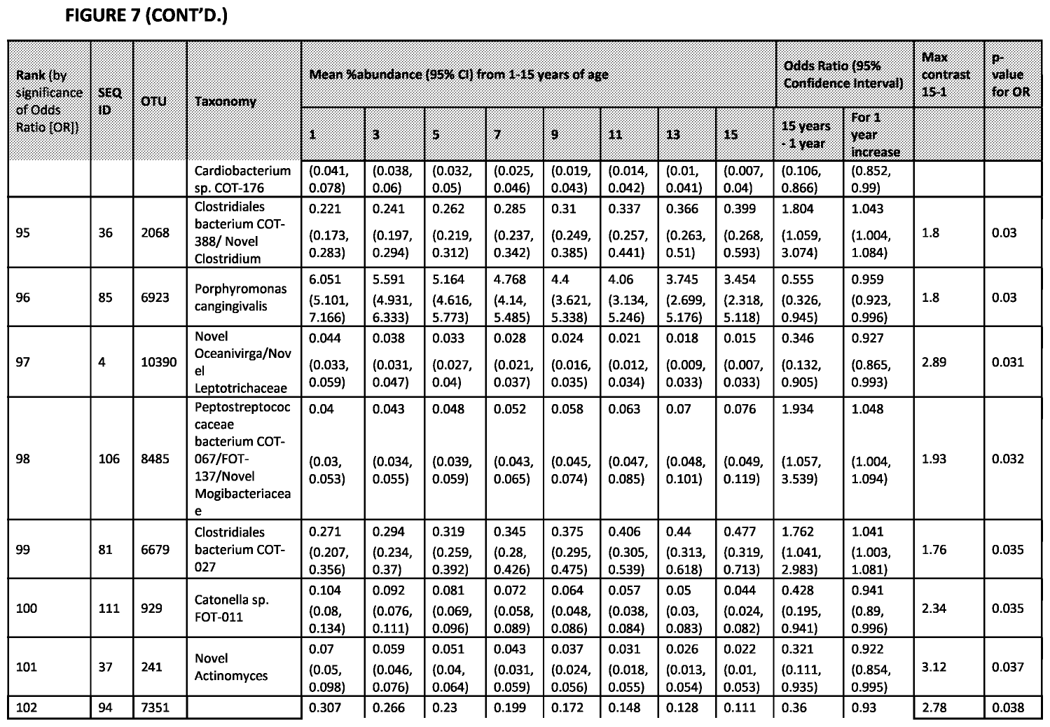

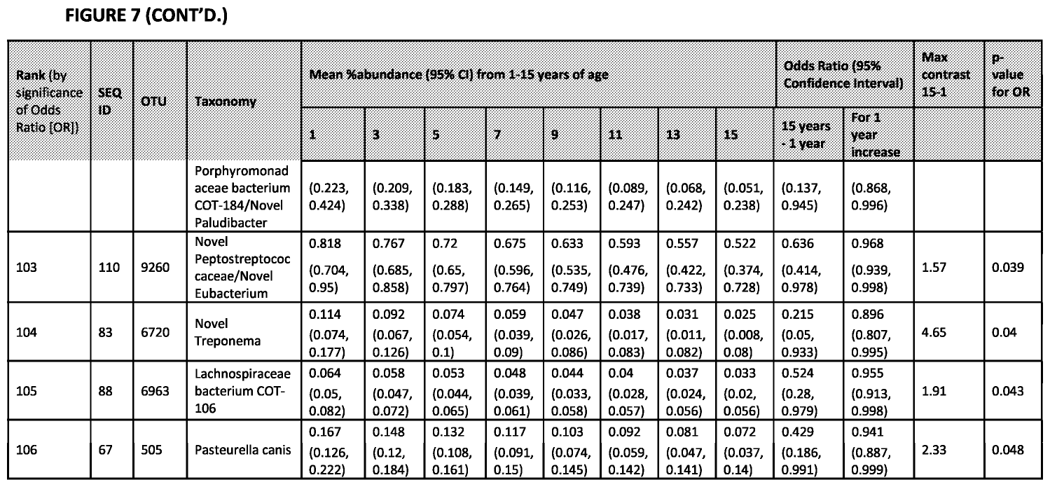

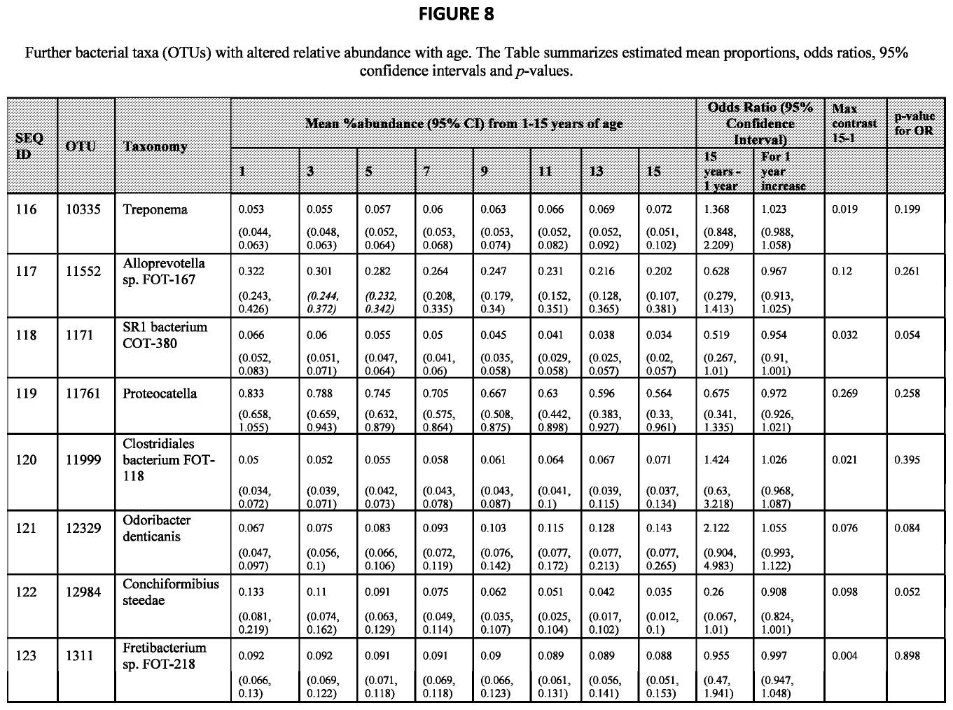

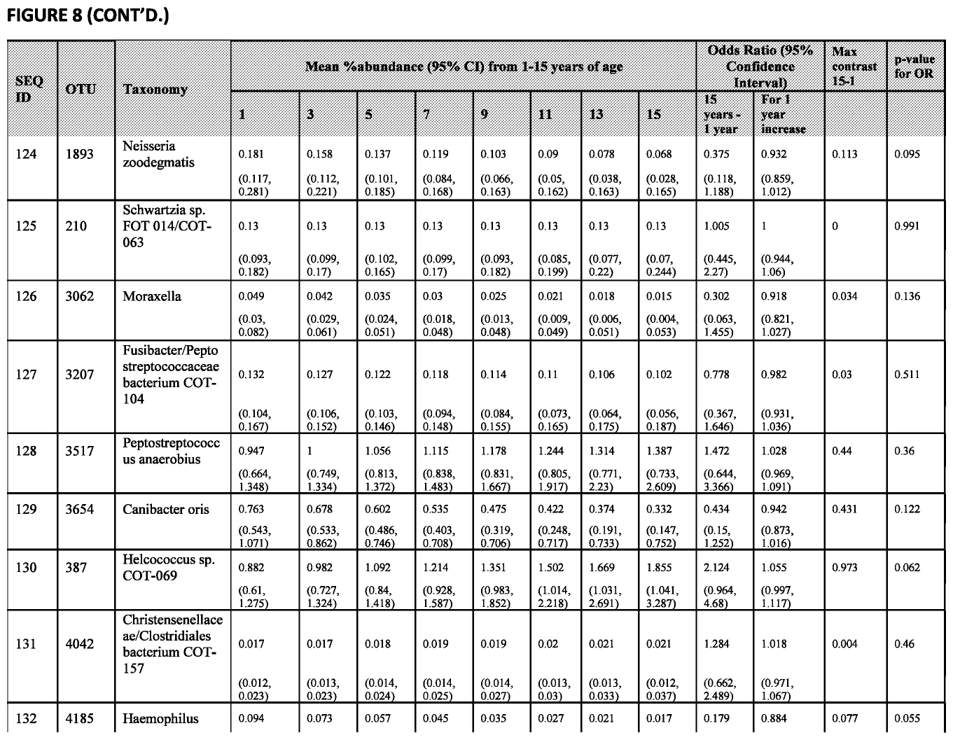

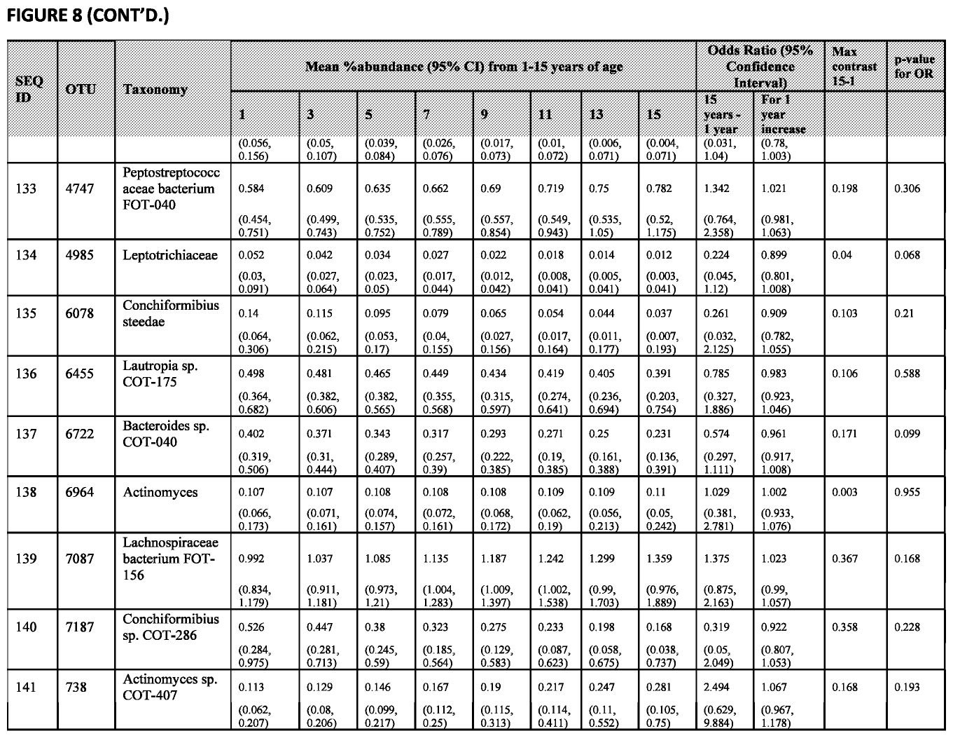

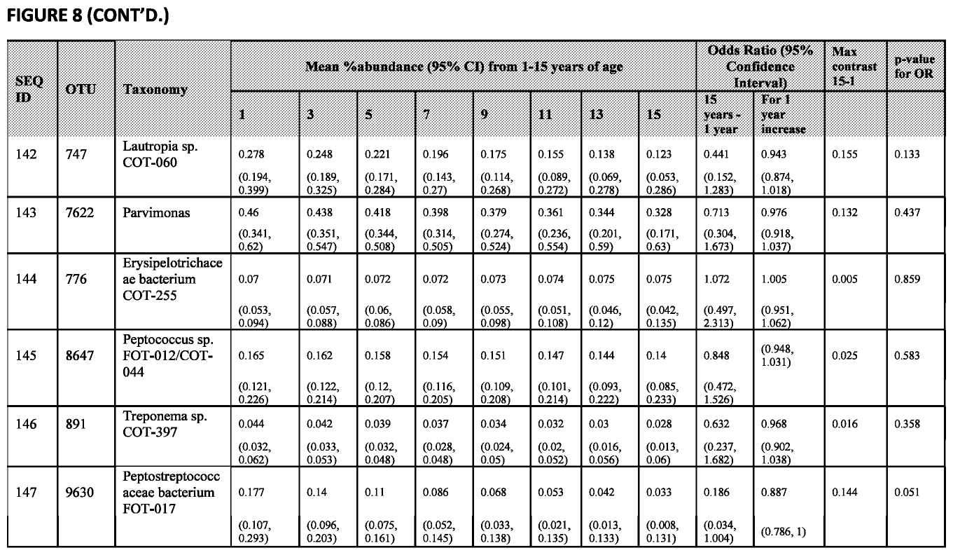

The control data set 124 can comprise information associated with bacterial taxa OTUs, an animal breed identifier, an animal size, an animal weight, animal health information, geographical location information, or any other suitable type of information that is associated with a plurality of animals. For example, the control data set 124 can comprise information about the oral microbiome of canids at different ages. Additional information about the control data set 124 is provided below. Examples of the control data set 124 are shown in FIGS. 7 and 8.

The training data 114 can comprise information associated with bacterial taxa OTUs, an animal breed identifier, an animal size, an animal weight, animal health information, geographical location information, sample location (e.g., gingival margin or subgingival), or any other suitable type of information that is associated with an animal that can be input into a machine learning model 112. For example, the training data 114 can comprise at least a portion of the control data set 124 that is collected for a plurality of animals. An example of control data 124 is shown in FIGS. 7 and 8. The test data 116 is the same data type as the training data 114. In some embodiments, the test data 116 is a subset or a portion of the training data 114. For example, twenty percent of the training data 114 can be used as test data 116. In other examples, any other suitable percent of the training data 114 can be used as test data 116.

The health information 122 comprises information that is associated with one or more animals. Examples of health information include, but are not limited to, contact information for an owner of an animal, an animal name or identifier, information associated with bacterial taxa OTUs, an animal breed identifier, DNA information, an animal size, an animal weight, animal health information, geographical location information, gingivitis information, periodontitis information, or any other suitable type of information that is associated with an animal.

Age Determination Process Using Machine Learning

FIG. 2 is a flowchart of an embodiment of an age determination process 200 for the diagnostic system 100. The diagnostic system 100 can employ process 200 to predict the age of an animal using machine learning. Process 200 allows the diagnostic system 100 to input various types of information that are associated with the health and physical attributes of an animal into a machine learning model 112 that is configured to predict the age of the animal. This process allows the diagnostic system 100 to determine the age of an animal based on the physical attributes of the animal.

At step 202, before employing the machine learning model 112 to determine an animal age value 120 for an animal, the network device 102 first trains the machine learning model 112 for determining an animal age value 120. During the training process, the machine learning model 112 determines weights and bias values that allow the machine learning model 112 to map certain types of training data 114 to different types of animal age values 120. In one embodiment, the machine learning model 112 is trained using a supervised learning training process using labeled training data 114. The supervised learning training process may comprise obtaining training data 114 for a plurality of animals, associating the training data 114 for each animal with an animal age value 120, and then training the machine learning model 112 using the training data 114 that is associated with the animal age value 120. Associating the training data 114 with the animal age values 120 links the metadata for each animal with its corresponding animal age value 120. After training, each machine learning model 112 is configured to receive bacterial taxa OTUs, an animal breed identifier, an animal size, an animal weight, animal health information, geographical location information, sample location (e.g., gingival margin, supragingival, or subgingival), or any other suitable type of information that is associated with an animal as an input and to output an animal age value 120 based on the input data 118. Through this process, each machine learning model 112 is trained to predict an animal's age (i.e., an animal age values 120) based on the input data 118. The network device 102 can be configured to train the machine learning model 112 using any suitable technique. In some embodiments, the machine learning model 112 can be trained by a third-party device (e.g., a cloud server) that is external from the network device 102. After training the machine learning model 112, the machine learning model 112 is stored in memory (e.g. memory 110). This concludes the training process for the machine learning model 112.

At step 204, the network device 102 obtains input data 118 for an animal. In one embodiment, the network device 102 can obtain the input data 118 from a user device 104. For example, the user device 104 can send or transfer the input data 118 to the network device 102 as a message or a data file. In this example, the user device 104 can send or transfer the input data 118 to the network device 102 using any suitable messaging or data transfer technique. In some embodiments, a user can directly provide the input data 118 to the network device 102. For example, the user can enter (e.g., type) the input data 118 into the network device 102 using a user interface (e.g., keyboard, mouse, and/or touch screen) on the network device 102. The input data 118 may comprise any suitable combination of bacterial taxa OTUs, an animal breed identifier, an animal size, an animal weight, animal health information, geographical location information, sample location (e.g., gingival margin, supragingival, or subgingival), or any other information associated with the animal.

In one embodiment, the input data 118 comprises an array of bacterial taxa OTU values. The bacterial taxa OTUs identify the type and/or amount of bacteria that are present within a sample that is collected from the mouth of the animal. The sample can comprise bacteria from a gingival area (e.g., near the gums), subgingival area (e.g., below the gum line) and/or supragingival (e.g., above the gum) in the mouth of the animal. Additional details for the process of collecting a sample and identifying bacterial taxa OTUs from within the sample are also provided below. Examples of bacterial taxa OTUs are described below and shown in FIGS. 7 and 8. In one embodiment, the bacterial taxa OTUs can be identified using one-hot encoding. For example, the input data 118 can comprise an array that is associated with different types of bacteria that can be present within the mouth of the animal. The array comprises a plurality of entries that are each associated with a particular type of bacteria. In this example, a value of zero for an entry indicates that the type of bacteria that is associated with the entry was not present or detected within the mouth of the animal. A value of one for an entry indicates that the type of bacteria that is associated with the entry was present or detected within the mouth of the animal. In some embodiment, an entry can comprise a numeric value that indicates the amount of the bacteria for each bacteria type that was present or detected within the mouth of the animal. In other embodiments, a bacterial taxa OTU can be a numeric value or code that uniquely identifies a bacteria type that is present in the mouth of the animal. For example, each bacteria type can be linked with a unique numerical value or code. In other embodiments, the bacterial taxa OTUs can use any other suitable type of format or data structure to identify a bacteria type that is present in the mouth of the animal. In one embodiment, the bacterial taxa OTU may comprise one or more, preferably two, bacterial taxa OTU that are selected from Table 6. In some embodiment, the bacterial taxa OTUs are preferably collected from a subgingival portion of the mouth.

In some embodiments, the bacterial taxa OTUs may be collected from an animal while it is conscious. In this case, the bacterial taxa OTUs may be collected from a gingival or supragingival area in the mouth of the animal. In some embodiments, the bacterial taxa OTUs may be collected from an animal while it is unconscious. In this case, the bacterial taxa OTUs may be collected from a gingival, subgingival, or supragingival area in the mouth of the animal.

In some embodiments, the input data 118 can further comprise an animal breed identifier that identifies a breed of the animal. Examples of animal breeds include, but are not limited to, Affenpinscher, Australian Silky Terrier, Bichon Frise, Bolognese, Cavalier King Charles Spaniel, Chihuahua, Chinese Crested, Coton De Tulear, English Toy Terrier, Griffon Bruxellois, Havanese, Italian Greyhound, Japanese Chin, King Charles Spaniel, Lowchen (Little Lion Dog), Maltese, Miniature Pinscher, Papillon, Pekingese, Pomeranian, Pug, Russian Toy, Yorkshire Terrier, French Bulldog, Beagle, Dachshund, Pembroke Welsh Corgi, Miniature Schnauzer, Cavalier King Charles Spaniel, Shih Tzu, Boston Terrier, Bulldog, Cocker Spaniel, Shetland Sheepdog, Border Collie, Basset Hound, Siberian Husky, Dalmatian, Great Dane, Neapolitan mastiff, Scottish Deerhound, Dogue de Bordeaux, Newfoundland, English mastiff, Saint Bernard, Leonberger and Irish Wolfhound, and cross-breeds. In one embodiment, the animal breed type can be identified using one-hot encoding. For example, the input data 118 can comprise an array that is associated with different breeds of the animal. The array comprises a plurality of entries that are each associated with a particular breed type. In this example, a value of zero for an entry indicates that the animal is not a member of the breed type that is associated with the entry. A value of one for an entry indicates that the animal is a member of the breed type that is associated with the entry. In other embodiments, the animal breed identifier can be a numeric value or code that uniquely identifies a breed type. For example, each breed type can be linked with a unique numerical value. In other embodiments, the animal breed identifier can use any other suitable type of format or data structure to identify a breed type for the animal.

In some embodiments, the input data 118 can further comprise an animal size classification value. The animal size classification value identifies the size of the animal based on the physical size and/or weight of the animal. Examples of animal sizes include, but are not limited to, puppy, toy breeds, extra-small breeds, small breeds, medium breeds, and large breeds. As an example, a toy breed can correspond with animals that are physically smaller than small breed animals. A small breed can correspond with animals that have an average body weight of up to ten kilograms. A medium breed can correspond with an animal that has an average body weight between eleven and twenty-six kilograms. A large breed can correspond with an animal that has an average body weight of over twenty-seven kilograms. In one embodiment, the animal size classification value can be identified using one-hot encoding. For example, the input data 118 can comprise an array that is associated with different animal size classifications. The array comprises a plurality of entries that are each associated with a particular animal size. In this example, a value of zero for an entry indicates that the animal is not a member of the animal size classification (e.g., toy breed, small breed, medium breed, or large breed) that is associated with the entry. A value of one for an entry indicates that the animal is a member of the animal size classification that is associated with the entry. In other embodiments, the animal size classification value can be a numeric value or code that uniquely identifies an animal size classification. For example, each animal size classification can be linked with a unique numerical value. In other embodiments, the animal size classification value can use any other suitable type of format or data structure to identify an animal size classification for the animal. In one embodiment, the input data 118 may comprise the animal size classification value and one or more, preferably two, bacterial taxa OTU that are selected from Table 7. In some embodiment, the bacterial taxa OTUs are preferably collected from a subgingival portion of the mouth.

In some embodiments, the input data 118 can further comprise a sample location value that identifies a location in the mouth where a sample was collected for the animal. For example, the sample location value can comprise a numeric value that corresponds with a gingival location, a subgingival location, a supragingival location, or a combination thereof.

In some embodiments, the input data 118 can further comprise a weight value that identifies a weight for the animal. For example, the weight value can comprise a numeric value that corresponds with the weight of the animal in pounds or kilograms. In other examples, the weight value can be in any other suitable of units.

In some embodiments, the input data 118 can further comprise a gingivitis value for the animal. The gingivitis value is a numeric value that is associated with a time to bleeding in the gums of the animal when probing the mouth of the animal. In some instances, the gingivitis value can be an average value that is associated with a plurality of teeth in the mouth of the animal.

In some embodiments, the input data 118 can further comprise a periodontitis value for the animal. The periodontitis value is a numeric value that is associated with the amount of periodontitis that is present in the mouth of the animal. For example, the periodontitis value can correspond with a periodontitis stage as defined by the American Veterinary Dental College (AVDC) or the number/proportion of teeth in the mouth with periodontitis.

In some embodiments, the input data 118 can further comprise geographic location information that identifies a physical location that is associated with the animal. For example, the geographic location information can identify a country or region where the animal is physically located. For instance, the geographic location information can identify a country such as China, Thailand, the United Kingdom, the United States of America, etc. In other embodiments, the input data 118 can further comprise any other suitable type or combination of information that is associated with the animal. In one embodiment, the geographic location information can be identified using one-hot encoding. For example, the input data 118 can comprise an array that is associated with the geographic location information. The array comprises a plurality of entries that are each associated with a particular country or region. In this example, a value of zero for an entry indicates that the animal is not located within a country or region that is associated with the entry. A value of one for an entry indicates that the animal is located within a country or region that is associated with the entry. In other embodiments, the geographic location information can be a numeric value or code that uniquely identifies a particular country or region. For example, each country and region can be linked with a unique numerical value. In other embodiments, the geographic location information can use any other suitable type of format or data structure to identify a physical location for the animal.

At step 206, the network device 102 inputs the input data 118 for the animal into the machine learning model 112. Here, the network device 102 inputs any suitable combination of information from the input data 118 that was obtained in step 204 into the machine learning model 112. For example, the network device 102 can input the input data 118 as a sequential or parallel combination of arrays or values into the machine learning model 112.

At step 208, the network device 102 receives an animal age value 120 for the animal from the machine learning model 112. The machine learning model 112 is configured to predict an animal age value for the animal based on the bacterial taxa OTU values, the breed of the animal, the size of the animal, the weight of the animal, the health of the animal, the gingivitis value associated with the animal, the periodontitis value associated with the animal, the geographic location information associated with the animal, or any other suitable type of information, or combination of information, thereof. In response to inputting the input data 118 in the machine learning model 112, the network device 102 receives an animal age value 120 as an output from the machine learning model 112

At step 210, the network device 102 outputs the animal age value 120. Here, the network device 102 outputs the animal age value 120 for a user to view. As an example, the network device 102 can output the animal age value 120 by displaying the animal age value 120 on a graphical user interface (e.g., a display). As another example, the network device 102 can output the animal age value 120 by writing and saving the animal age value 120 within a document of file. As another example, the network device 102 can output the animal age value 120 by sending the animal age value to a user device 104. In this example, the network device 102 can send the animal age value 120 to the user device 104 as a message, an email, a text document, a file, a link, or in any other suitable format. After receiving the animal age value 120 from the network device 102, the user device 104 can then display the animal age value 120 to a user using a graphical user interface (e.g., a display). In other examples, the network device 102 can use any other suitable technique for outputting the animal age value 120.

At step 212, the network device 102 determines whether to process additional animal information. Here, the network device 102 determines whether there is any more animal information to process for other animals. For example, a user can provide samples to the network device 102 for one or more other animals to process to determine their ages. The network device 102 determines to process additional animal information when there are one or more samples remaining to process. The network device 102 returns to step 204 in response to determining to process additional animal information. In this case, the network device 102 returns to step 204 to obtain input data 118 for another animal and to repeat the process of using the machine learning model 112 to determine the age of the animal based on the new input data 118. Otherwise, the network device 102 terminates process 200. In this case, the network device 102 determines that there are no more animals to process and terminates process 200.

Hardware Configuration for the Network Device

FIG. 3 is an embodiment of a network device 102 for the diagnostic system 100. As an example, the network device 102 can be a server or a computer. The network device 102 comprises a processor 302, a memory 110, and a network interface 304. The network device 102 can be configured as shown or in any other suitable configuration.

Processor The processor 302 is a hardware device that comprises one or more processors operably coupled to the memory 110. The processor 302 is any electronic circuitry including, but not limited to, state machines, one or more CPU chips, logic units, cores (e.g., a multi-core processor), field-programmable gate array (FPGAs), application-specific integrated circuits (ASICs), or digital signal processors (DSPs). The processor 302 can be a programmable logic device, a microcontroller, a microprocessor, or any suitable combination of the preceding. The processor 302 is communicatively coupled to and in signal communication with the memory 110 and the network interface 304. The one or more processors are configured to process data and can be implemented in hardware or software. For example, the processor 302 can be 8-bit, 16-bit, 32-bit, 64-bit, or of any other suitable architecture. The processor 302 can include an arithmetic logic unit (ALU) for performing arithmetic and logic operations, processor registers that supply operands to the ALU and store the results of ALU operations, and a control unit that fetches instructions from memory and executes them by directing the coordinated operations of the ALU, registers and other components.

The one or more processors are configured to implement various instructions. For example, the one or more processors are configured to execute diagnostics instructions 306 to implement the diagnostics engine 108. In this way, processor 302 can be a special-purpose computer designed to implement the functions disclosed herein. In an embodiment, the diagnostics engine 108 is implemented using logic units, FPGAs, ASICs, DSPs, or any other suitable hardware. The diagnostics engine 108 is configured to operate as described in FIGS. 1-2. For example, the diagnostics engine 108 can be configured to perform the steps of process 200 as described in FIG. 2.

Memory

The memory 110 is a hardware device that is operable to store any of the information described above with respect to FIGS. 1-12 along with any other data, instructions, logic, rules, or code operable to implement the function(s) described herein when executed by the processor 302. The memory 110 comprises one or more disks, tape drives, or solid-state drives, and can be used as an over-flow data storage device, to store programs when such programs are selected for execution, and to store instructions and data that are read during program execution. The memory 110 can be volatile or non-volatile and can comprise a read-only memory (ROM), random-access memory (RAM), ternary content-addressable memory (TCAM), dynamic random-access memory (DRAM), and static random-access memory (SRAM).

The memory 110 is operable to store diagnostics instructions 306, machine learning models 112, training data 114, test data 116, health information 122, a control data set 124, and/or any other data or instructions. The diagnostics instructions 306 can comprise any suitable set of instructions, logic, rules, or code operable to execute the diagnostics engine 108. The machine learning models 112, the training data 114, the test data 116, the health information 122, and the control data set 124 are configured similar to the machine learning models 112, the training data 114, the test data 116, the health information 122, and the control data set 124 described in FIGS. 1-2, respectively.

Network Interface

The network interface 304 is a hardware device that is configured to enable wired and/or wireless communications. The network interface 304 is configured to communicate data between user devices 104 and other devices, systems, or domains. For example, the network interface 304 can comprise an NFC interface, a Bluetooth interface, a Zigbee interface, a Z-wave interface, a radio-frequency identification (RFID) interface, a WIFI interface, a LAN interface, a WAN interface, a PAN interface, a modem, a switch, or a router. The processor 302 is configured to send and receive data using the network interface 304. The network interface 304 can be configured to use any suitable type of communication protocol as would be appreciated by one of ordinary skill in the art.

Age Determination Process Using a Control Data Set

The processes described below can be implemented, unless otherwise indicated, using conventional chemistry, biochemistry, molecular biology, immunology, and pharmacology, within the skill of the art. Such techniques are explained fully in the literature. For example, see references [10-17].

In another embodiment, the diagnostic system 100 can be configured to determine the amount (e.g., the abundance or relative abundance) of one or more bacterial taxa OTUs in a sample from the canid and compare the determined abundance or relative abundance of that bacterial taxa in a control data set 124 to determine an oral microbiome age for the canid. In this configuration, the diagnostic system 100 identifies the oral microbiome age status of the canid and assigns it to a particular “age” or “life stage” by comparing the abundance or relative abundance of one or more bacterial taxa in the sample to the abundance or relative abundance of one or more bacterial taxa in a control data set 124.

The control data set 124 generally comprises information about the oral microbiome of canids at different ages. For example, the oral microbiome composition of several canids from a life stage group (e.g., puppy, adult, senior, or geriatric) or from a specific age group (e.g., about 1, about 3, about 5, about 7, about 9, about 11, about 13, about 15 years) can be analyzed to generate the control data set 124. The term “about” in relation to a numerical value x is optional and can refer to a range of numerical values, for example, x+10%. The oral microbiome composition can also be analyzed from the same canid at intervals within a life stage or in different life stages. Bacterial taxa which show differences in abundance or relative abundance at different age groups across the group of tested individuals can then be used as a control data set 124. An example of a control data set 124 is shown in FIGS. 7 and 8. The control data set 124 can be obtained from healthy canids where “healthy” in this context means that they do not suffer from a periodontal disease such as gingivitis or periodontitis.

The diagnostic system 100 is configured to determine the oral microbiome age status by comparing the oral microbiome (e.g., the abundance or relative abundance of one or more bacterial taxa) in the sample to the oral microbiome from animals having a known age status (control microbiome) and then determine the oral microbiome age status based on the similarity to the control data set 124. Thus, the control data set 124 can comprise typical oral microbiomes of canids at different life stages (e.g., puppy, adult, senior, or geriatric) and optionally at different ages within these life stages (e.g., one or more of about 1, about 3, about 5, about 7, about 9, about 11, about 13, about 15). These oral microbiome data can have been obtained using techniques discussed elsewhere herein.

In some embodiment, the control data set 124 comprises data from a canid at a particular life stage only. In these embodiments, the oral microbiome composition from the canid can be compared to the control data set 124. If the composition of the oral microbiome is similar to the control data set 124 then the oral microbiome composition will be deemed healthy if the control data set 124 matches the biological age of the canid from which the sample was obtained. For example, if the control data set 124 is from an adult canid and a sample from an adult canid is similar to the control data set 124, the oral microbiome composition is considered healthy. Alternatively, if the control data set 124 is from a geriatric canid and is similar to the oral microbiome composition of an adult canid, the oral microbiome composition is also considered healthy.

The analysis of the oral microbiome generally comprises determining the abundance or relative abundance of bacterial taxa. In some embodiments, one or more bacterial taxa (e.g., fewer than 5, 7, 10, 20, 30, 40, 50, 60, 70, 80, 90 or 100) are quantified. For example, one or more, or a minimum of two bacterial taxa can be quantified (e.g., 2-100, 3-90, 4-80, 5-70, 6-60, 7-50, 8-40, 9-30, 10-20). The bacterial taxa that are analyzed will generally be bacterial taxa which allows an unambiguous allocation to one (or possibly two) of the different life stages. In these embodiments, the one or more bacterial taxa that are assessed are based on a data set that shows that these are indicative of a certain life stage. Thus, in these embodiments, the quantifying of the bacterial taxa of interest and the subsequent assignment of the microbiome age status based on these data constitute correlating the bacterial taxa in the sample to a control data set 124.

FIG. 7 is an example of bacterial taxa that have significantly altered relative abundance with age. By knowing the abundance or relative abundance of the bacterial taxa in a sample, the diagnostic system 100 is able to provide high consistency in accurately assigning samples to dogs at different life stages or ages. The results can be assessed to determine whether the oral microbiome age status concurs with the canid's actual age. If not, steps to shift the oral microbiome age status can be taken, as discussed below.

A higher abundance/relative abundance of the bacterial taxa in the sample is indicative of an older oral microbiome age status, and vice versa. Canids that show a higher abundance/relative abundance of the bacterial taxa in question in the sample have an older oral microbiome age status compared to canids which have a lower relative abundance of one or more bacterial taxa. A lower abundance/relative abundance of the bacterial taxa in the sample is indicative of an older oral microbiome age status, and vice versa. Canids that show a lower abundance/relative abundance of the bacterial taxa in question in the sample have an older oral microbiome age status compared to canids which have a higher relative abundance of one or more bacterial taxa.

Detecting and Quantifying Bacterial Taxa

In one embodiment, the diagnostic system 100 is configured to detect and/or quantify one or more bacterial taxa, for example, one or more bacterial genera and/or one or more bacterial species, through the detection of gene sequences or other biomarkers. In other embodiments, the diagnostic system 100 can employ any other suitable technique for detecting and quantifying bacterial taxa within a sample. Examples of techniques for detecting and quantifying bacterial taxa include, but are not limited to, 454 pyrosequencing, mass spectrometry, polymerase chain reaction (PCR) and quantitative PCR (qPCR), 16S rDNA amplicon sequencing, shotgun sequencing, metagenome sequencing, Illumina sequencing, and nanopore DNA sequencing techniques (e.g., MinION, PacBio). For example, the bacterial taxa can be determined using qPCR amplification or sequencing of 16S rDNA. Other techniques for detecting and quantifying bacterial taxa include shotgun sequencing to determine characteristic whole genome gene sequences or spectrometry for detection of metabolites and a range of methods for biomarker detection for identification of the taxa.

In certain embodiments, the bacterial taxa can be determined by sequencing the 16S rDNA. The 16S rDNA/rRNA gene includes nine hypervariable regions of varying conservation identified as V1-V9. In certain embodiments, the bacterial taxa are determined by sequencing V1-V3 region of the 16S rDNA. For example, but without any limitation, sequencing can be performed by pyrosequencing, Sanger sequencing, Illumina sequencing, or nanopore sequencing (e.g., MinION or PacBio).

In certain embodiments, the 16S rRNA is amplified and/or sequenced using a forward and a reverse primer. In certain embodiments, the forward primer comprises or consists of the nucleotide sequence set forth in SEQ ID NO: 148. In certain embodiments, the forward primer comprises or consists of the nucleotide sequence set forth in SEQ ID NO: 149. In certain embodiments, the reverse primer comprises or consists of the nucleotide sequence set forth in SEQ ID NO: 150. SEQ ID Nos: 148-150 are provided below:
AGAGTTTGATYMTGGCTCAG (SEQ ID NO: 148)
AGGGTTCGATTCTGGCTCAG (SEQ ID NO:149)

TYACCGCGGCTGCTGG (SEQ ID NO: 150) The bacterial taxa can also be detected by other means known in the art such as, for example, RNA sequencing, protein sequence homology, or other biological markers indicative of the bacterial taxa or the function of the bacteria. In this case, the sequencing data can be used to quantitate different bacterial taxa in the sample. For example, without any limitation, the sequences can be clustered at about 98%, about 99%, or about 100% identity, and the bacterial taxa (e.g., abundant taxa representing e.g., more than about 0.001%, about 0.01%, or about 0.05% of the total sequences) can then be assessed for their relative proportions. The count for the bacterial taxa out of the total number of sequences is the relative bacterial abundance (i.e., the relative bacterial abundance cf. the total sequence population for each sample).

Suitable techniques for determining the descriptive nature of bacterial taxa and biomarkers or combinations of bacteria or biomarkers for their ability to assign a sample to a particular study group such as an age range can include, but are not limited to, logistic regression analysis, partial least squares discriminate analysis (PLSDA), and random forest analysis and other univariate and multivariate methods. The bacterial taxa or biomarkers are then ranked based on their specificity for a particular microbiome age status.

Intervention Process

In some embodiments, the diagnostic system 100 is further configured to determine whether an intervention is required. This determination process generally comprises (a) quantifying one or more bacterial taxa in a sample obtained from a canid, (b) determining the abundance or relative abundance of said bacterial taxa, and (c) comparing the abundance or relative abundance determined in step (b) to that of a control data set 124. If the comparison of step (c) indicates a difference in oral microbiome age status to the actual age of the canid, then an intervention can be recommended. The intervention is a process that changes the canid oral microbiome, as detailed below. The one or more bacterial taxa can comprise 5 or more, 10 or more, or more, 30 or more, 40 or more, or 50 or more bacterial taxa. The control data set 124 can comprise bacterial taxa data from a plurality of other canids of the same life stage as the canid tested.

In some embodiments, the diagnostic system 100 is configured to (a) quantifying one or more bacterial taxa in a sample obtained from a canid, (b) determining the abundance or relative abundance of said bacterial taxa, and (c) comparing the abundance or relative abundance determined in step (b) to that of a control data set 124. In this case, if the comparison of step (c) indicates none or a slight difference in oral microbiome age status to the actual age of the canid, then an intervention can be recommended to decrease the oral microbiome age status of the canid to the oral microbiome age status of a younger actual aged canid control data set 124.

In some embodiments, the diagnostic system 100 can recommend an intervention to decrease the oral microbiome age status of the canid regardless of its comparison to the control data set 124.

Classifying Bacterial Taxa

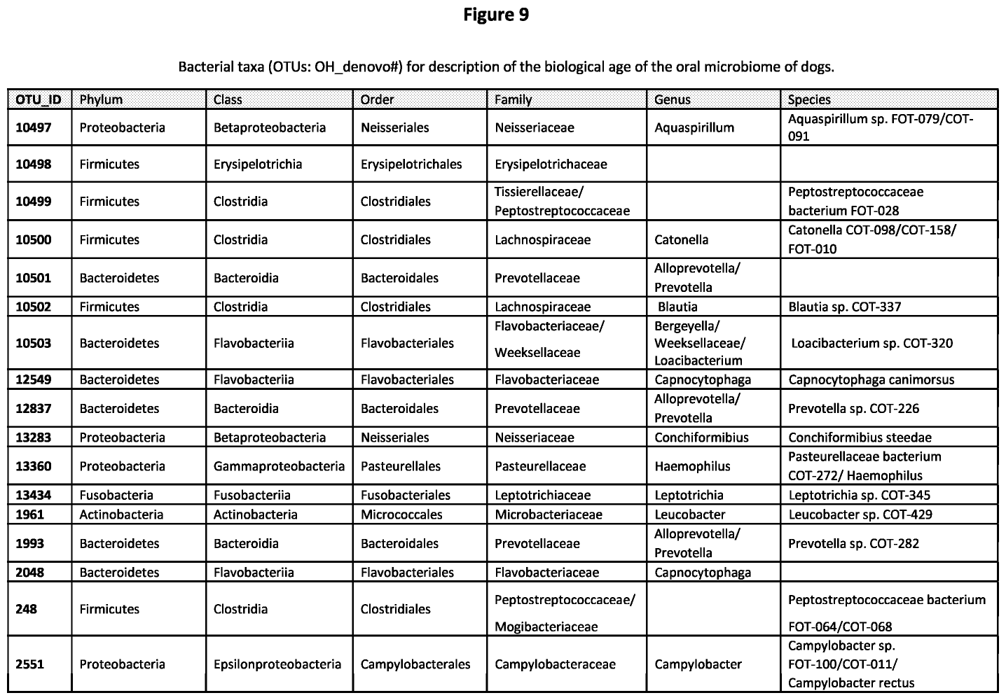

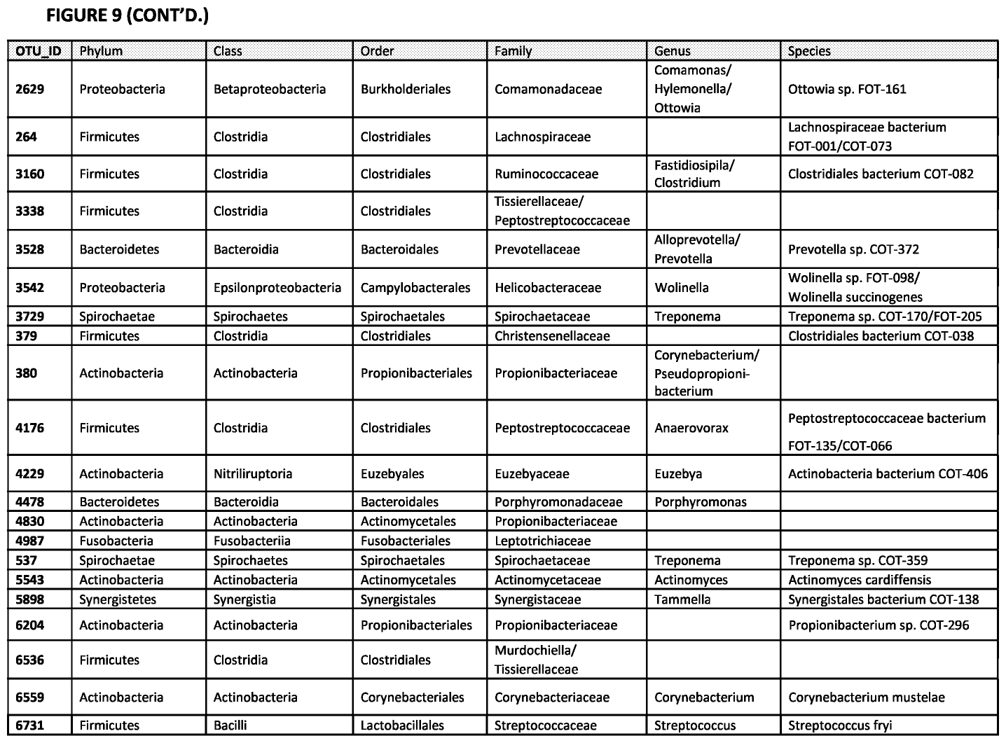

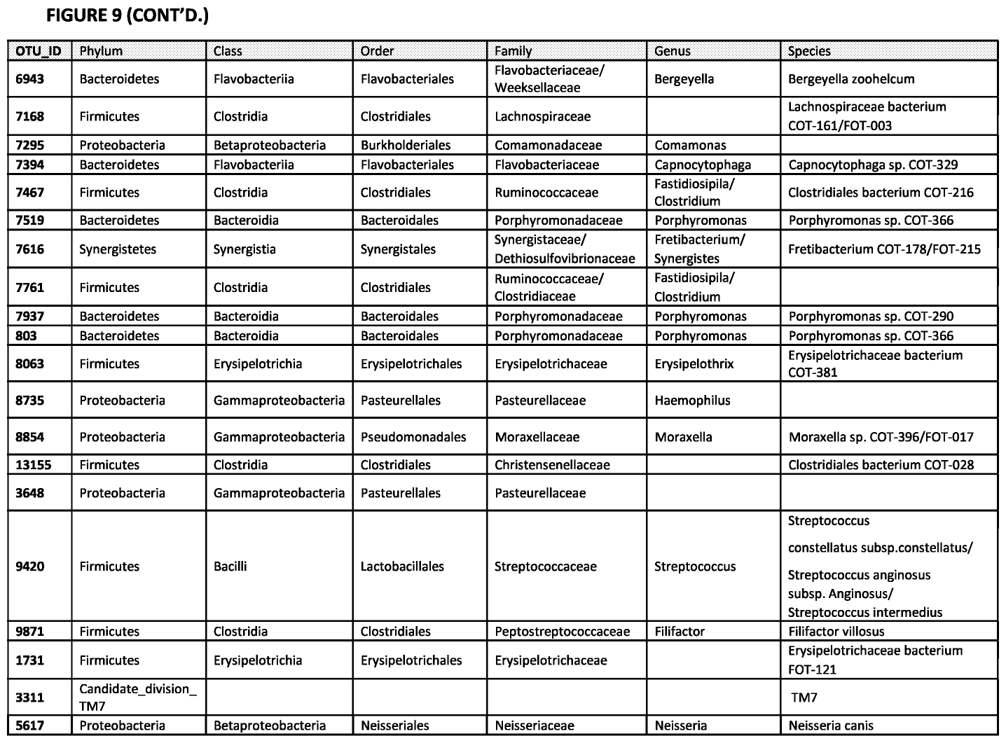

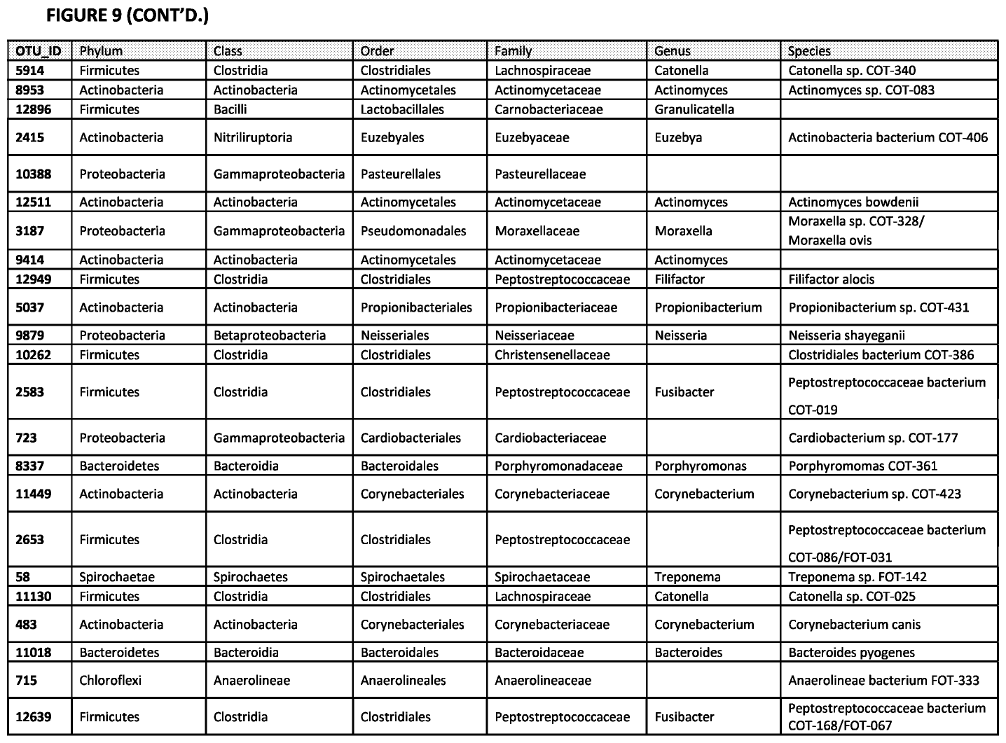

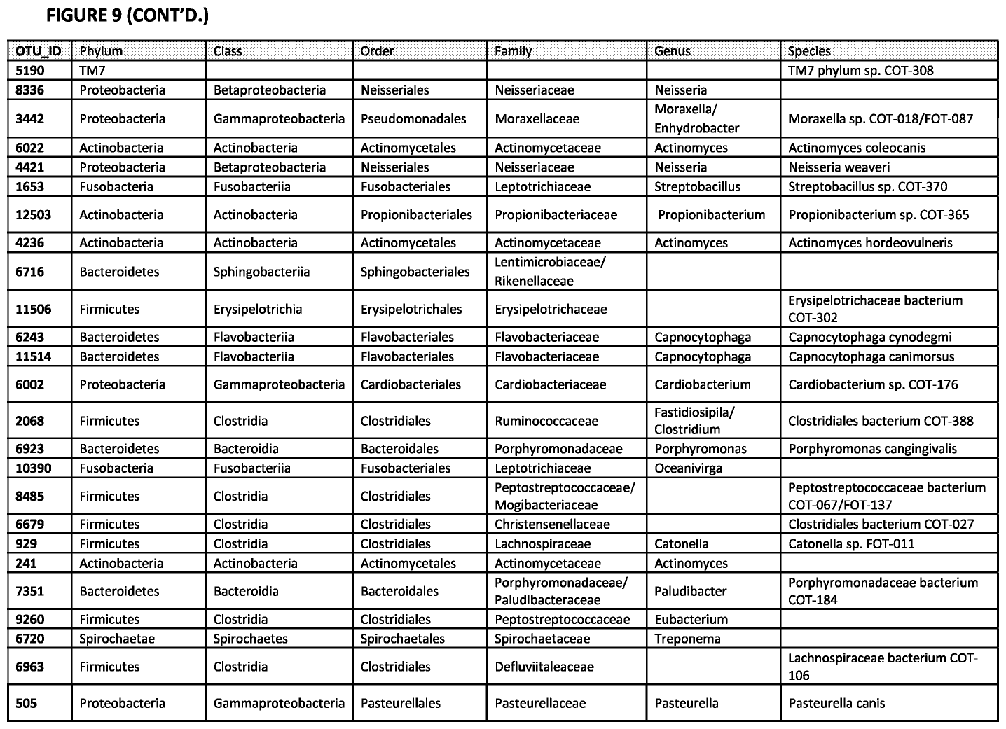

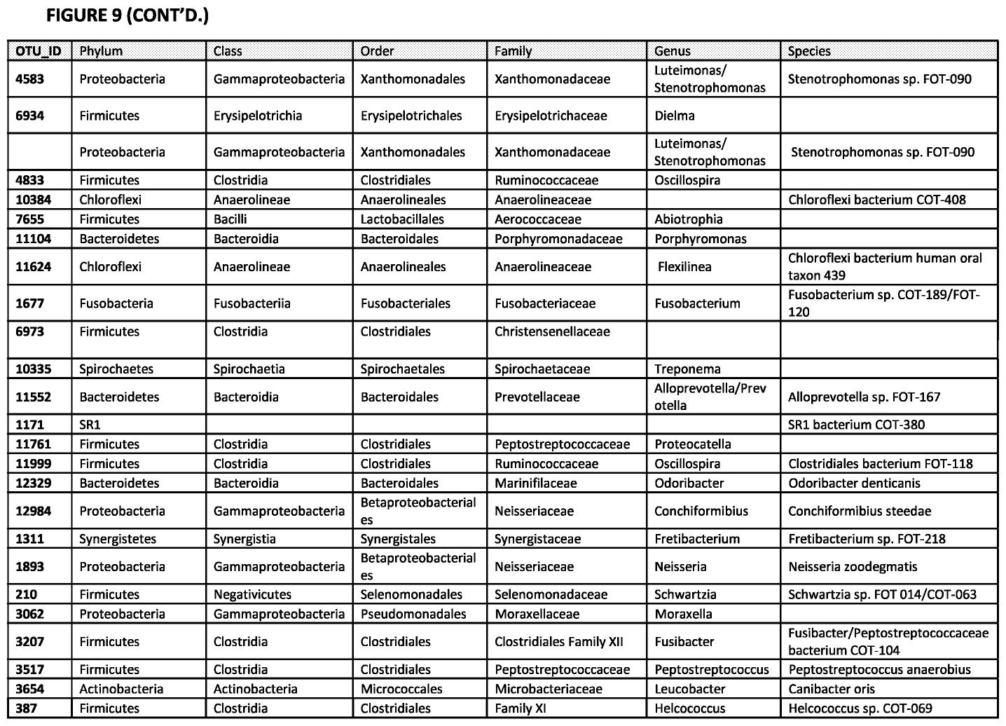

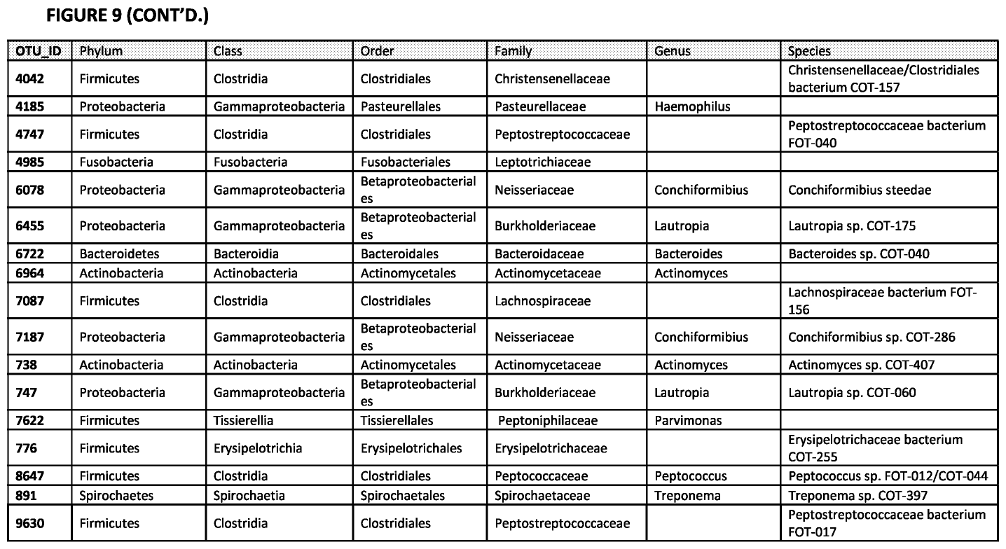

In one embodiment, analyzing the oral microbiome can comprise annotating observed taxonomic units, for example, using the basic local alignment search tool (BLAST). Depending on how precisely the alignment matches the top hit, an OTU can be allocated a suitable taxonomic assignment. As an example, if the alignment matches the top hit (e.g., top BLAST hit) with ≥98% sequence identity and ≥98% sequence coverage then a species level is assigned. If these criteria are not met, the next appropriate level of taxonomic assignment is allocated, for example, ≥94% genus, ≥92% family, ≥90% order, ≥85% class, or ≥80% phyla. This means that the various OTUs in a sample can be assigned to a species, a genus, a family, an order, a class, or a phyla. These are collectively referred to herein as “bacterial taxa”. FIG. 9 is an example of a list of bacterial taxa that were identified in the present examples as being useful for describing the biological age of the oral microbiome of canids. FIG. 9 also shows the level to which they were described. As shown in FIG. 9, in most cases the bacterial taxa will be a species, but in some cases, it will be a genus or family.

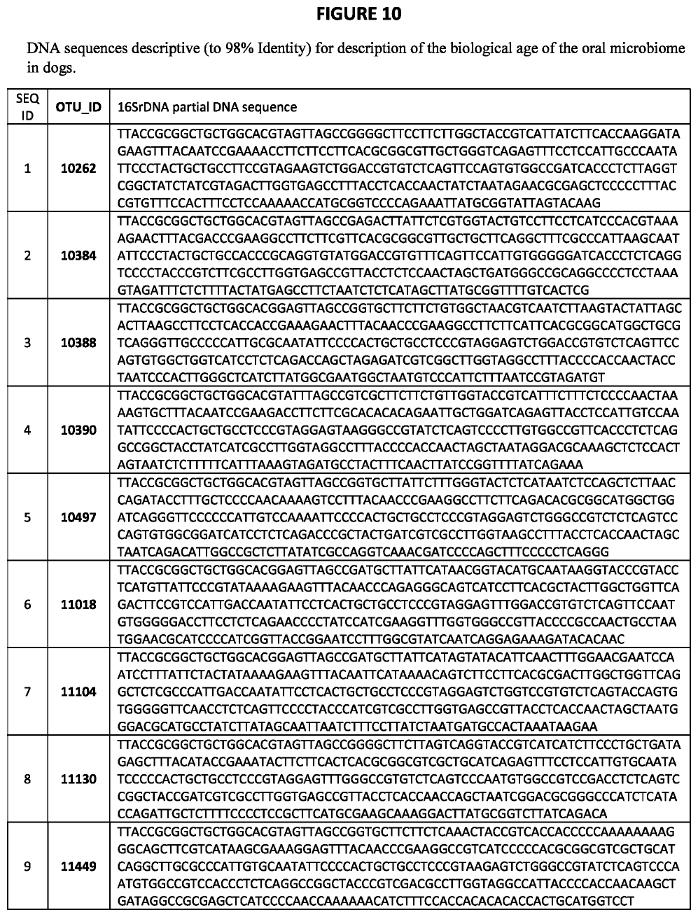

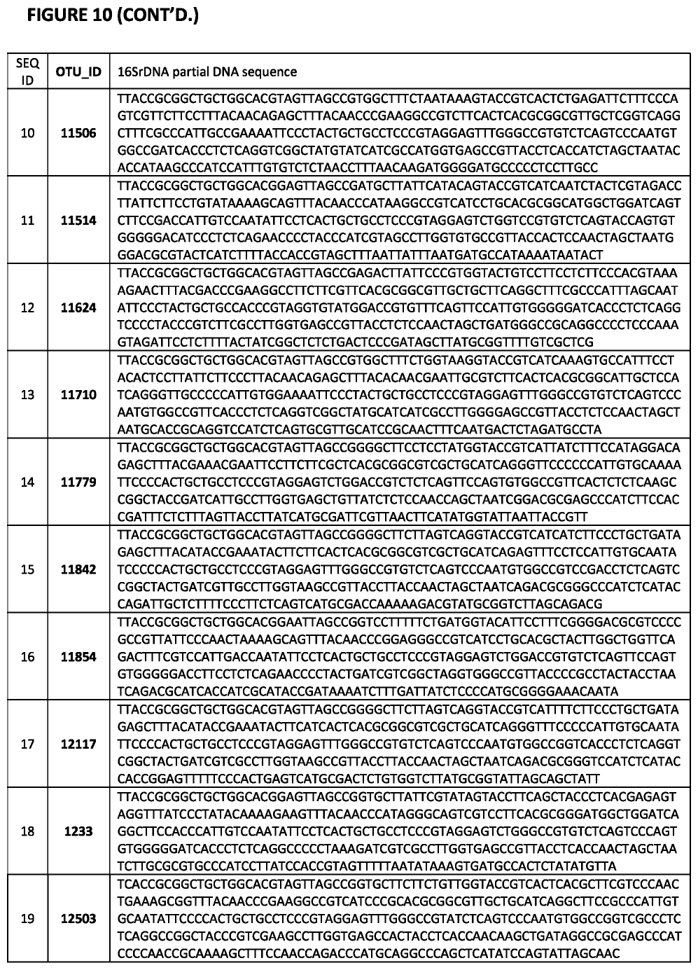

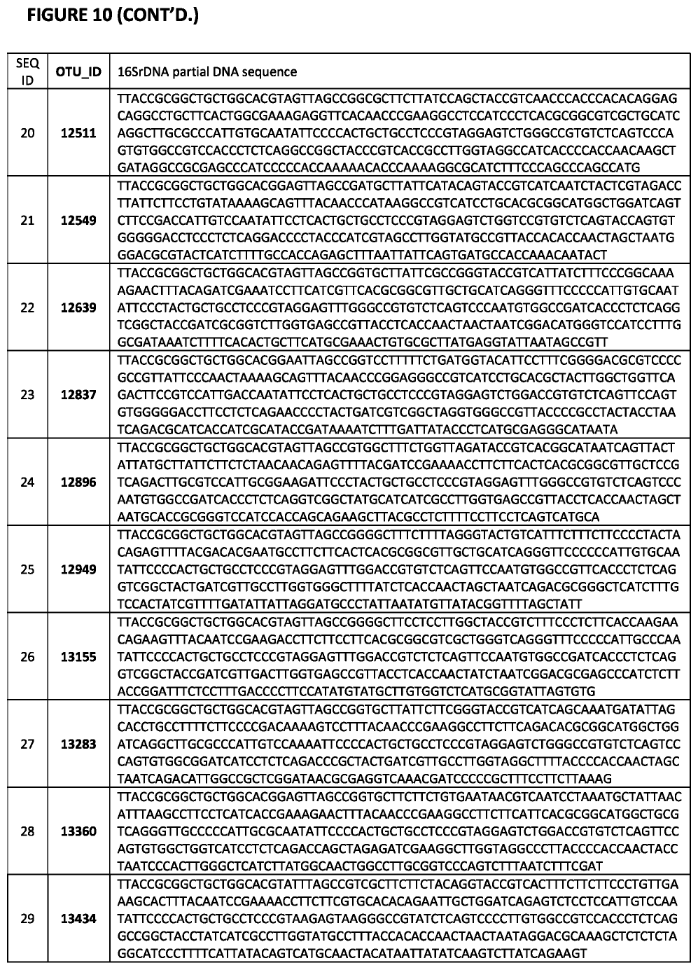

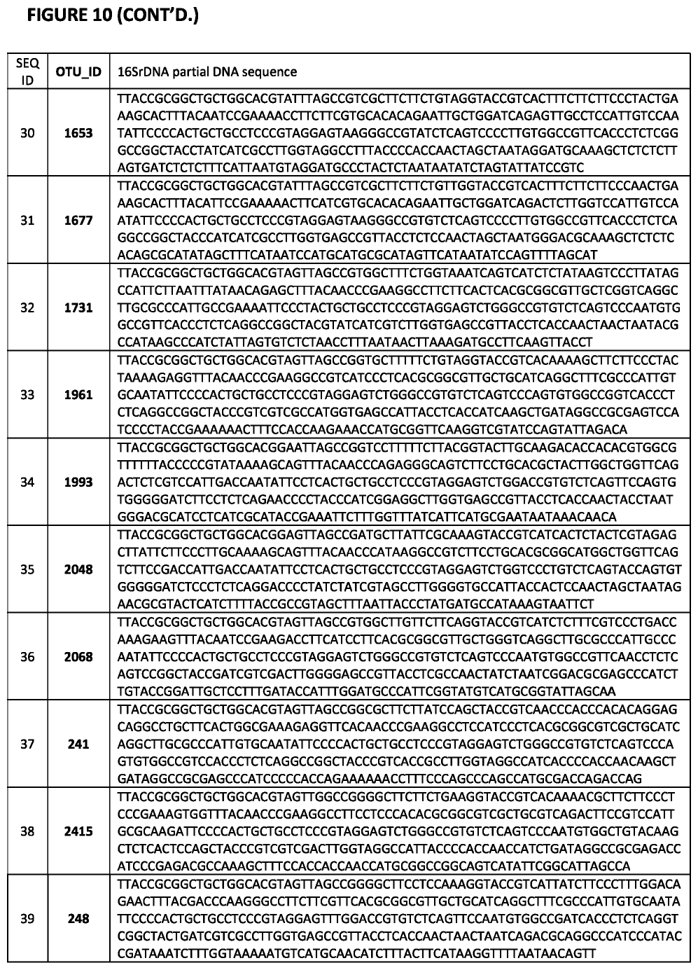

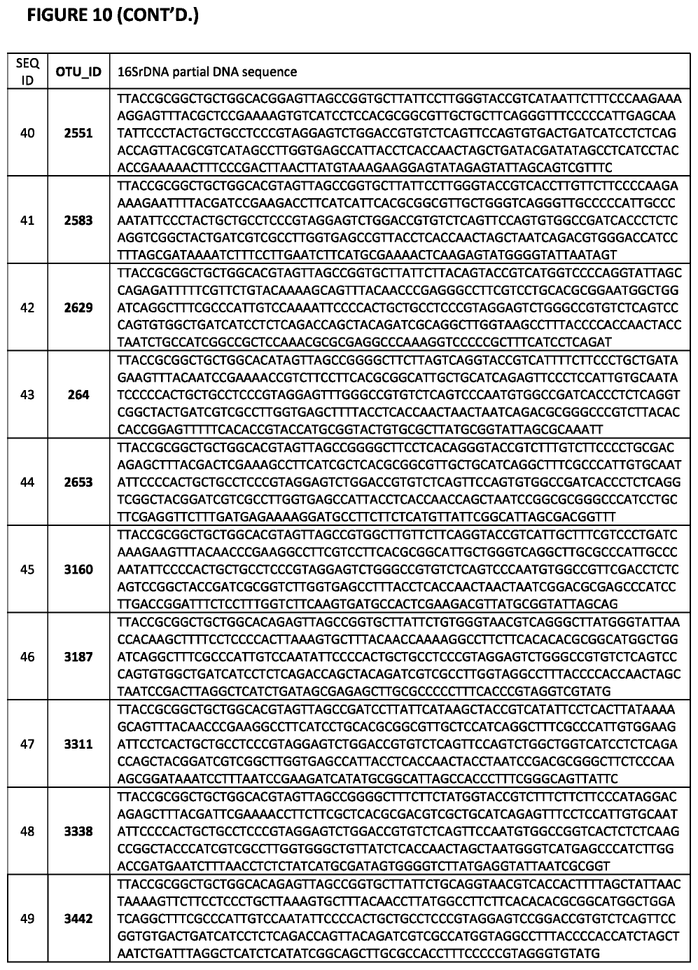

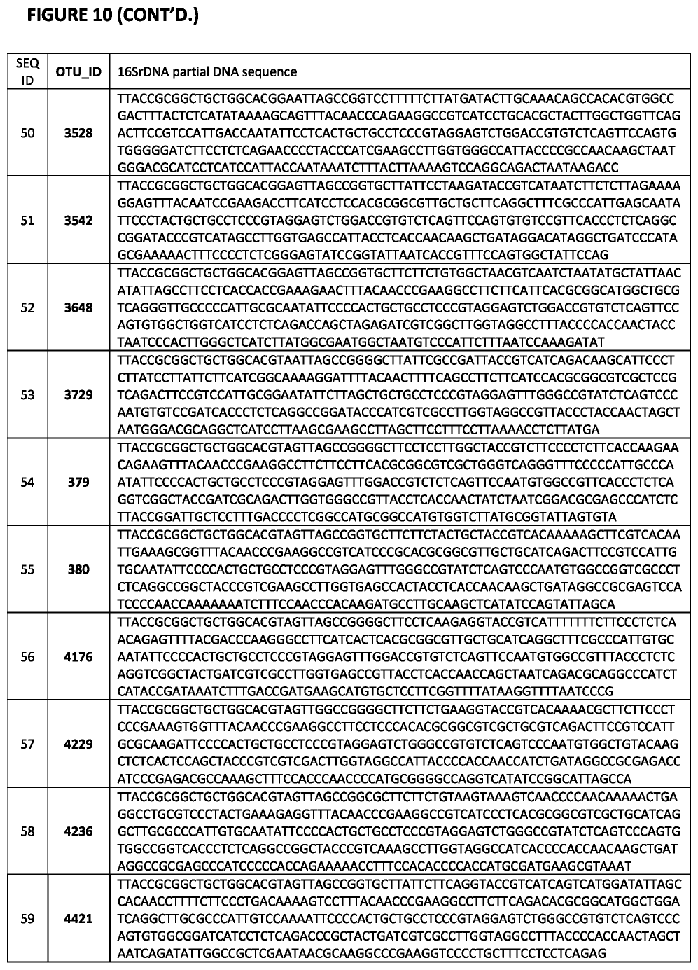

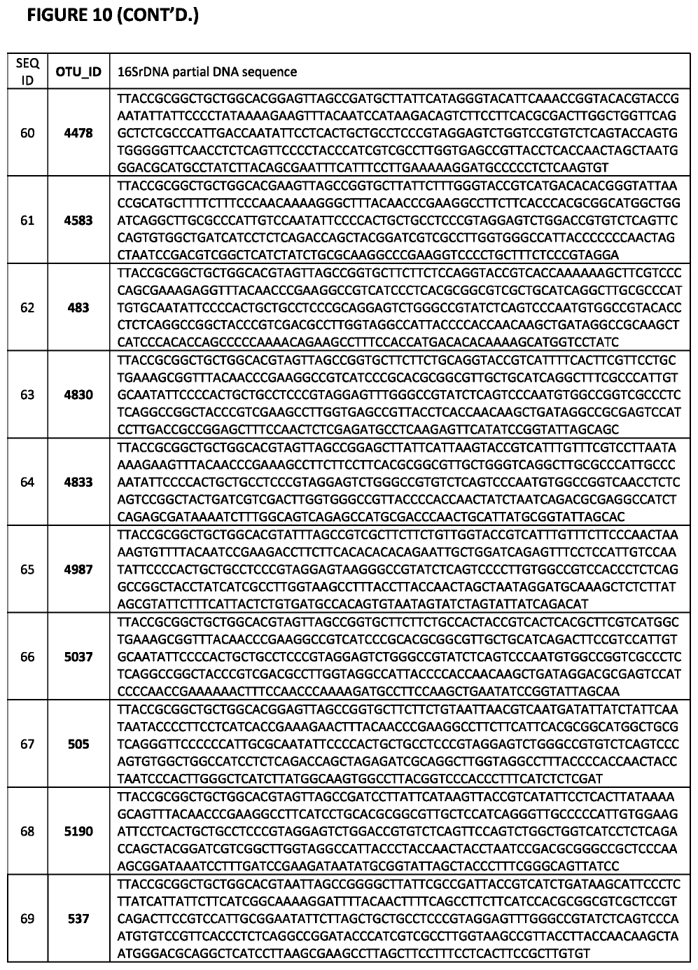

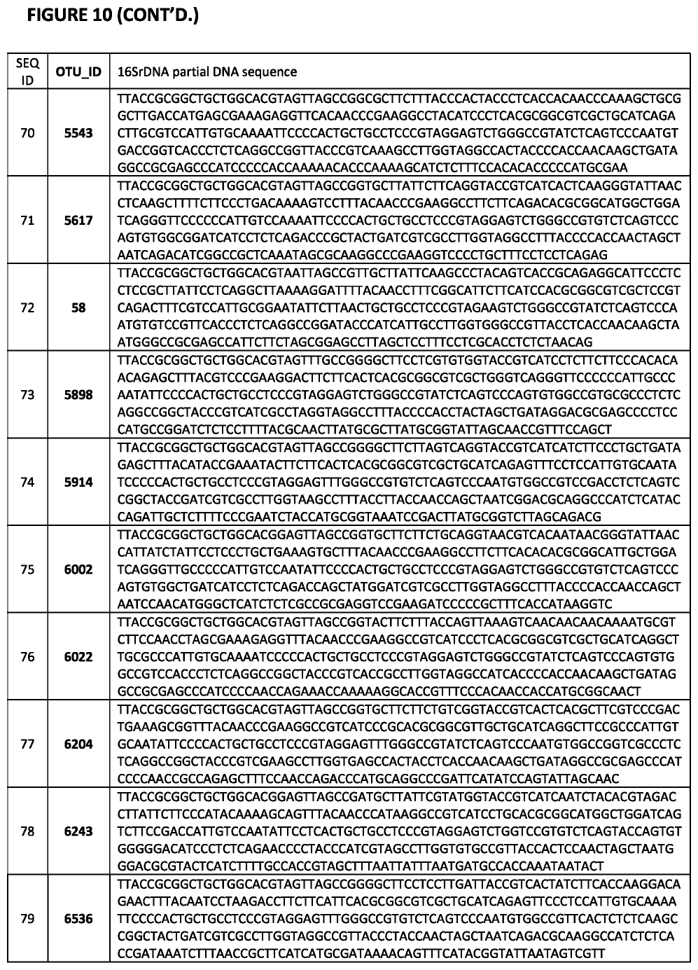

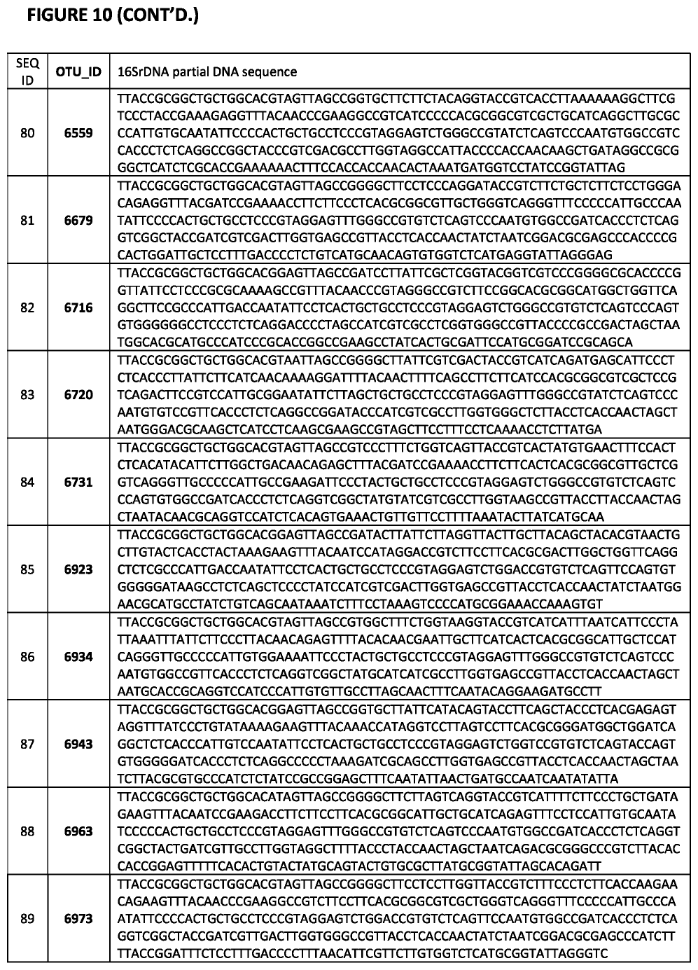

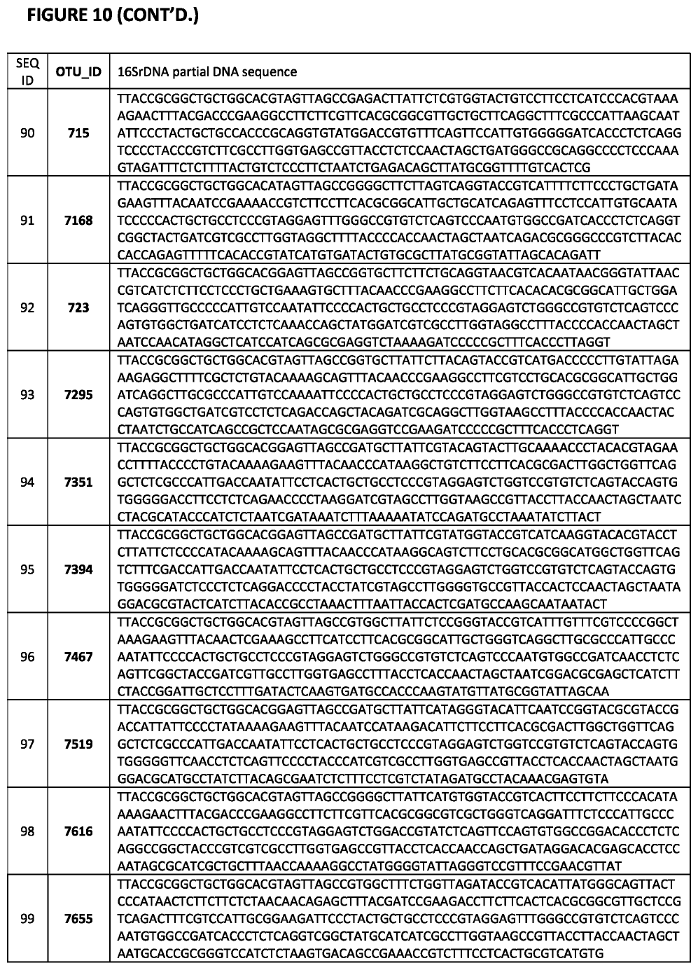

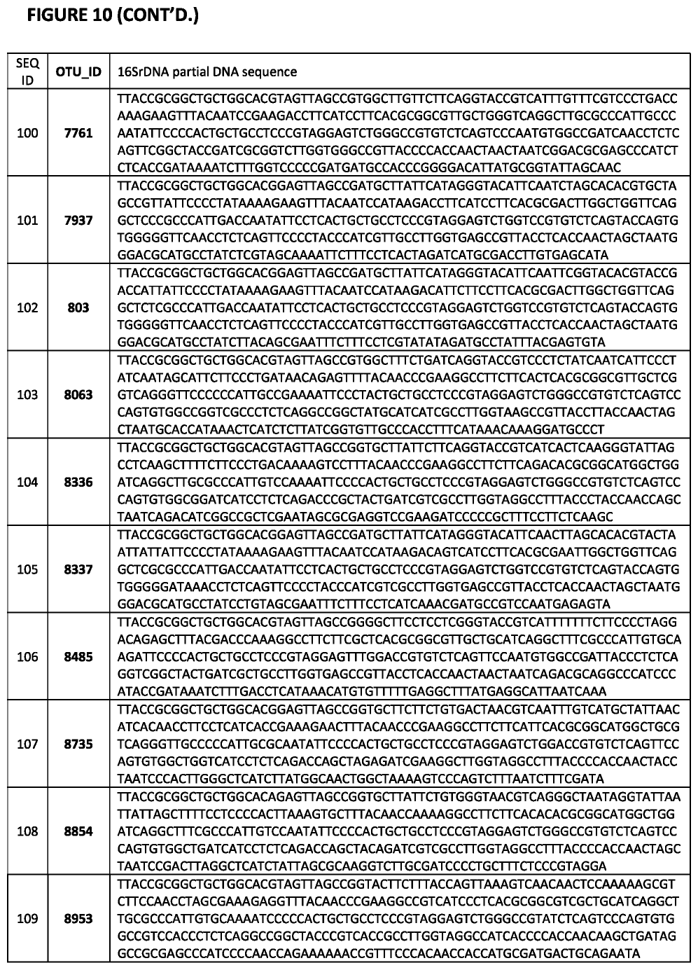

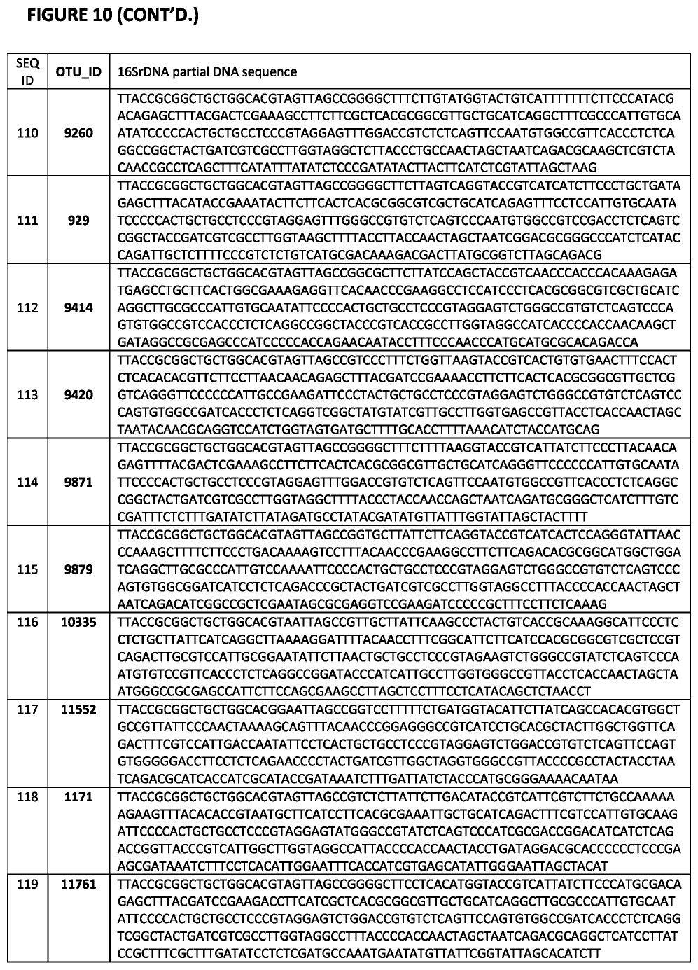

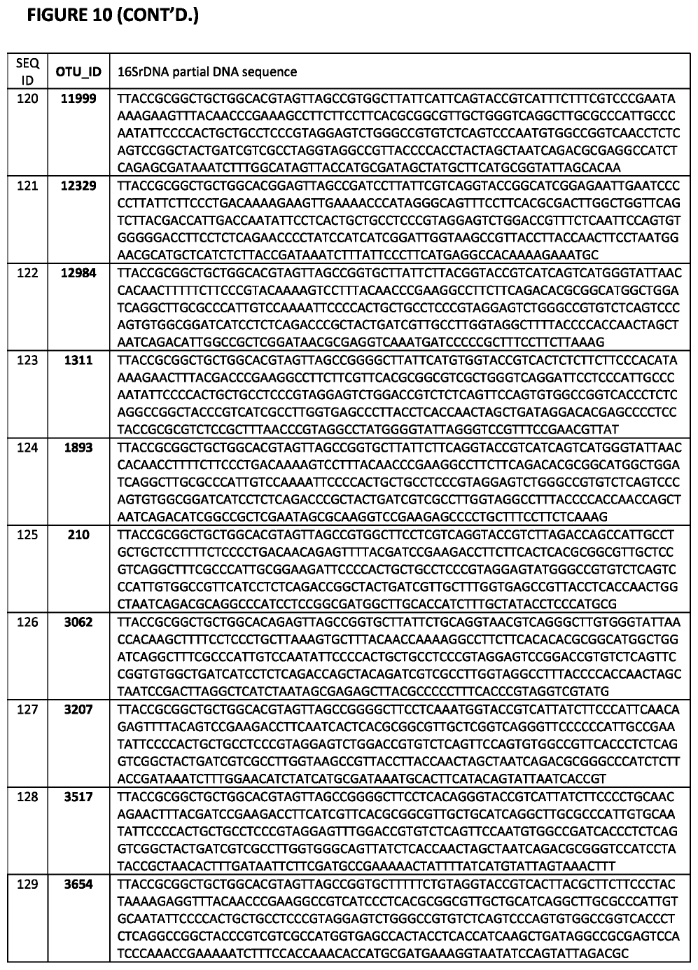

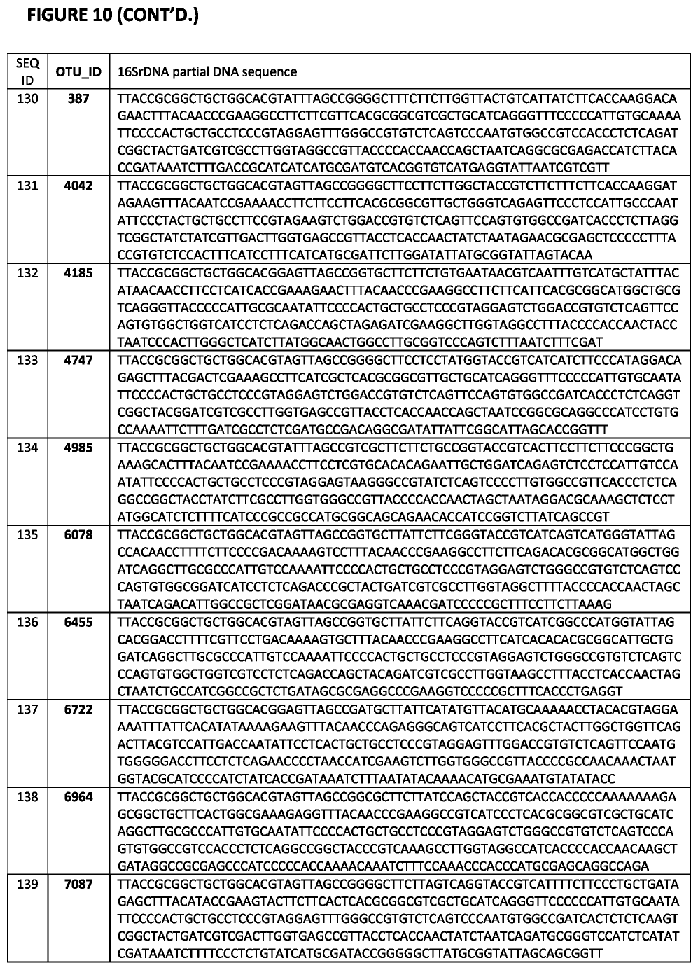

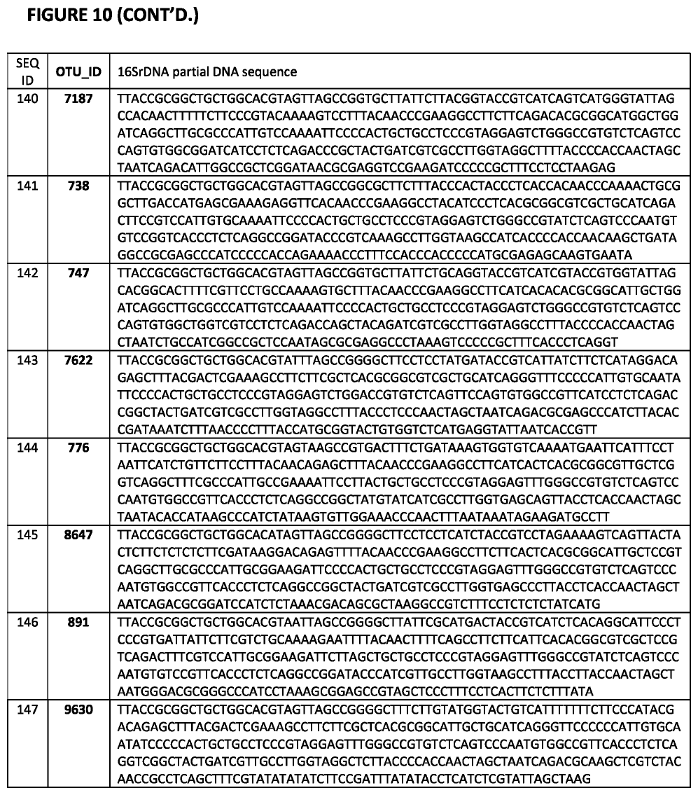

FIG. 7 is an example of bacterial taxa that were shown to have significantly altered relative abundance with age. FIG. 10 is an example of 16S rDNA sequences for the bacterial taxa identified in FIG. 7. The 16S rDNA sequences are DNA sequences descriptive (e.g., up to 98% identity) for a description of the biological age of the oral microbiome in dogs. Any of the bacterial taxa identified in FIG. 7 and FIG. 10 can be used for predicting the age of an animal. For example, one or more of sequences having at least about 95%, about 96%, about 97%, about 98%, about 99%, or about 100% identity to the sequences from bacterial taxa identified in FIG. 7 and FIG. 10 (e.g., SEq ID NOs: 1, 3-6, 8-11, 13-30, 32-60, 62-63, 65-85, 87-88, 90-98, 100-115) can be detected and/or quantified. As another example, one or more of sequences having at least about 95%, about 96%, about 97%, about 98%, about 99%, or about 100% identity to the sequences from bacterial taxa identified FIG. 8 or FIG. 10 (e.g., SEq ID NOs: 116-147) can be detected and/or quantified.

In one embodiment, bacterial taxa can be from a group comprising of the following bacterial families: Actinomycetaceae, Aerococcaceae, Anaerolineaceae, Bacteroidaceae, Campylobacteraceae, Cardiobacteri aceae, Carnobacteriaceae, Christensenellaceae, Clostridiaceae, Comamonadaceae, Corynebacteriaceae, Defluviitaleaceae, Dethiosulfovibrionaceae, Erysipelotrichaceae, Euzebyaceae, Flavobacteriaceae, Fusobacteriaceae, Helicobacteraceae, Lachnospiraceae, Lentimicrobiaceae, Leptotrichiaceae, Microbacteriaceae, Mogibacteriaceae, Moraxellaceae, Neisseriaceae, Paludibacteraceae, Pasteurellaceae, Peptoniphilaceae, Peptostreptococcaceae, Porphyromonadaceae, Prevotellaceae, Propionibacteriaceae, Rikenellaceae, Ruminococcaceae, Spirochaetaceae, Streptococcaceae, Synergistaceae, Tissierellaceae, Weeksellaceae, and Xanthomonadaceae.

In some embodiments, the bacterial taxa can be from a group comprising of the following bacterial families: Actinomycetaceae, Bacteroidaceae, Burkholderiaceae, Christensenellaceae, families belonging to the Clostridiales class, families belonging to the Erysipelotrichaceae class, Lachnospiraceae, Leptotrichiaceae, Marinifilaceae, Microbacteriaceae, Moraxellaceae, Neisseriaceae, Pasteurellaceae, Peptococcaceae, Peptoniphilaceae, Peptostreptococcaceae, Prevotellaceae, Ruminococcaceae, Selenomonadaceae, Spirochaetaceae, and Synergistaceae.

In some embodiments, bacterial taxa can be from a group comprising of the following bacterial genera: Abiotrophia, Actinomyces, Alloprevotella, Anaerovorax, Aquaspirillum, Bacteroides, Bergeyella, Blautia, Campylobacter, Capnocytophaga, Cardiobacterium, Catonella, Clostridium, Comamonas, Conchiformibius, Corynebacterium, Dielma, Enhydrobacter, Erysipelothrix, Eubacterium, Euzebya, Fastidiosipila, Filifactor, Flexilinea, Fretibacterium, Fusibacter, Fusobacterium, Granulicatella, Haemophilus, Hylemonella, Leptotrichia, Leucobacter, Loacibacterium, Luteimonas, Moraxella, Murdochiella, Neisseria, Oceanivirga, Oscillospira, Ottowia, Paludibacter, Pasteurella, Porphyromonas, Prevotella, Propionibacterium, Pseudopropioni-bacterium, Stenotrophomonas, Streptobacillus, Streptococcus, Synergistes, Tammella, Treponema, Weeksellaceae, and Wolinella.

In some embodiments, the bacterial taxa can be from a group comprising of the following bacterial genera: Actinomyces, Alloprevotella, Bacteroides, Conchiformibius, Fretibacterium, Fusibacter, Haemophilus, Helcococcus, Lautropia, Leucobacter, Moraxella, Neisseria, Odoribacter, Oscillospira, Parvimonas, Peptococcus, Peptostreptococcus, Prevotella, Proteocatella, Schwartzia, and Treponema.

In some embodiments, bacterial taxa can be from a group comprising of the following bacterial species: Actinobacteria bacterium COT-406, Actinomyces bowdenii, Actinomyces cardiffensis, Actinomyces coleocanis, Actinomyces hordeovulneris, Actinomyces sp. COT-083, Anaerolineae bacterium FOT-333, Aquaspirillum sp. FOT-079/COT-091, Bacteroides pyogenes, Bergeyella zoohelcum, Blautia sp. COT-337, Campylobacter sp. FOT-100/COT-011/Campylobacter rectus, Capnocytophaga canimorsus, Capnocytophaga cynodegmi, Capnocytophaga sp. COT-329, Cardiobacterium sp. COT-176, Cardiobacterium sp. COT-177, Catonella sp. COT-098/COT-158, Catonella sp. COT-025, Catonella sp. COT-340, Catonella sp. FOT-011, Chloroflexi bacterium COT-408, Chloroflexi bacterium human oral taxon 439, Clostridiales bacterium COT-027, Clostridiales bacterium COT-028, Clostridiales bacterium COT-038, Clostridiales bacterium COT-082, Clostridiales bacterium COT-216, Clostridiales bacterium COT-386, Clostridiales bacterium COT-388, Conchiformibius steedae, Corynebacterium canis, Corynebacterium mustelae, Corynebacterium sp. COT-423, Erysipelotrichaceae bacterium COT-302, Erysipelotrichaceae bacterium COT-381, Erysipelotrichaceae bacterium FOT-121, Filifactor alocis, Filifactor villosus, Fretibacterium COT-178/FOT-215, Fusobacterium sp. COT-189/FOT-120, Lachnospiraceae bacterium COT-106, Lachnospiraceae bacterium COT-161/FOT-003, Lachnospiraceae bacterium FOT-001/COT-073, Leptotrichia sp. COT-345, Leucobacter sp. COT-429, Loacibacterium sp. COT-320, Moraxella sp. COT-018/FOT-087, Moraxella sp. COT-328/Moraxella ovis, Moraxella sp. COT-396/FOT-017, Neisseria canis, Neisseria shayeganii, Neisseria weaveri, Ottowia sp. FOT-161, Pasteurella canis, Pasteurellaceae bacterium COT-272/Haemophilus, Peptostreptococcaceae bacterium COT-019, Peptostreptococcaceae bacterium COT-067/FOT-137, Peptostreptococcaceae bacterium COT-086/FOT-031, Peptostreptococcaceae bacterium COT-168/FOT-067, Peptostreptococcaceae bacterium FOT-028, Peptostreptococcaceae bacterium FOT-064/COT-068, Peptostreptococcaceae bacterium FOT-135/COT-066, Porphyromomas COT-361, Porphyromonadaceae bacterium COT-184, Porphyromonas cangingivalis, Porphyromonas sp. COT-290, Porphyromonas sp. COT-366, Prevotella sp. COT-226, Prevotella sp. COT-282, Prevotella sp. COT-372, Propionibacterium sp. COT-296, Propionibacterium sp. COT-365, Propionibacterium sp. COT-431, Stenotrophomonas sp. FOT-090, Stenotrophomonas sp. FOT-090, Streptobacillus sp. COT-370, Streptococcus constellatus subsp. constellatus/Streptococcus anginosus subsp. Anginosus/Streptococcus intermedius, Streptococcus fryi, Synergistales bacterium COT-138, TM7 phylum sp. COT-308, Treponema sp. COT-170/FOT-205, Treponema sp. COT-359, Treponema sp. FOT-142, and Wolinella sp. FOT-098/Wolinella succinogenes. Bacteria that are indistinguishable in their sequences for the length of the sequence analysed are also be written with a “I” in between the alternative names.

In some embodiments, the bacterial taxa can be selected from a group comprising of the following bacterial species: Actinomyces sp. COT-407, Alloprevotella sp. FOT-167, Bacteroides sp. COT-040, Canibacter oris, Christensenellaceae/Clostridiales bacterium COT-157, Clostridiales bacterium FOT-118, Conchiformibius sp. COT-286, Conchiformibius steedae, Erysipelotrichaceae bacterium COT-255, Fretibacterium sp. FOT-218, Fusibacter/Peptostreptococcaceae bacterium COT-104, Helcococcus sp. COT-069, Lachnospiraceae bacterium FOT-156, Lautropia sp. COT-060, Lautropia sp. COT-175, Neisseria zoodegmatis, Odoribacter denticanis, Peptococcus sp. FOT-012/COT-044, Peptostreptococcaceae bacterium FOT-017, Peptostreptococcaceae bacterium FOT-040, Peptostreptococcus anaerobius, Schwartzia sp. FOT 014/COT-063, SR1 bacterium COT-380, and Treponema sp. COT-397.

Bacterial taxa having an odds ratio when comparing the estimated proportion at two age points (e.g., age 15 and 1) greater than e.g., 1.2, 1.5, 2, 3, 5 are examples of bacterial taxa that can be used. Bacterial taxa having an odds ratio when comparing the estimated proportion at two age points (e.g., age 15 and 1) less than e.g., 0.5, 0.1, 0.05, 0.01, 0.005 are also examples of bacterial taxa that can be used.

Bacterial taxa having an odds ratio greater than 2 when comparing the estimated proportion at age 15 to age 1 are particular examples of bacterial taxa that can be used. By way of example, bacterial taxa selected from the phyla Firmicutes, Actinobacteria, Bacteroidetes, Synergistetetes, TM7, Chloroflexi, and Fusobacteria are examples of bacteria having an inverse correlation with age.

Within the phylum Firmicutes, there were 12 abundant OTUs (>0.3% of the population) that had an odds ratio >2 when comparing the estimated proportion at age 15 to age one: examples included those from the family Peptostreptococcaceae, Erysipelotrichaceae, two belonged to the class Clostridiales and four were species (Blautia sp. COT-337, Granulicatella sp. COT-095, Filifactor villosus, and a novel species belonging to the genus Streptococcus).

Within the phylum Actinobacteria, the four most abundant OTUs (>0.3%) with an odds ratio greater than two were Actinomyces sp. COT-083, Propionibacterium sp. COT-431 and two novel species one from the genus Corynebacterium and the other from the genus Leucobacter.

Of the bacterial taxa OTUs that had a significantly lower proportion at age 15 compared to age 1, the majority belonged to four phyla; Proteobacteria (20 OTUs), Bacteroidetes (19 OTUs), Firmicutes (13 OTUs), and Actinobacteria (11 OTUs). The remaining 11 OTUs belonged to the phyla Fusobacteria and Spirochaetes. With respect to the Proteobacteria phylum, the most abundant members (>0.3%) with the biggest difference between ages 15 and one (odds ratio >2) were three species of Neisseria (N. animolaris, N. shayeganii, and N. weaveri), two species from the genus Moraxella (Moraxella sp. COT-018 and a novel Moraxella species), two novel species from the family Pasteurellaceae, Campylobacter sp. COT-011, and a novel species from the genus Aquaspirillum.

Within the phylum Bacteroidetes, there were two species from the genus Capnocytophaga (C. canimorsus, cynodegmi), a novel species from the genus Bergeyella, Prevotella sp. COT-226, Porphyromonaaceae bacterium COT-184, and two species from the genus Porphyromonas (Porphyromonas sp. COT-290 and a novel Porphyromonas species).

Useful bacterial taxa can include those with a high statistical significance of the Odds Ratio for the difference between 15 years compared to 1 year (e.g., a p-value of <0.05, <0.025, <0.001). Examples of these bacterial taxa include, but are not limited to, Aquaspirillum sp. FOT-079/COT-091., novel Erysipelotrichaceael. (OTU 11710), novel Tissierellaceae/Peptostreptococcacl sp. (OTU11779), Catonella sp. (COT-098/COT-158/FOT-010), and novel Alloprevotella/PrevIlla sp. (OTU 11854). Preferably, the sequence(s) which is/are detected has/have at least about 80%, about 85%, about 90%, about 95%, about 96%, about 97%, about 98%, or about 99% identity to the sequence of SEQ ID NOs: 5, 13, 14, 15 and/or 16. In certain embodiments, the sequence(s) which is/are detected are identical to the sequence set forth in SEQ ID NOs: 5, 13, 14, 15, and/or 16. The accuracy can increase when more species are quantified. For example, sequences having at least about 95%, about 96%, about 97%, about 98%, about 99%, or about 100% identity to at least 2, 3, 4, or all of the sequences of SEQ ID NOs: 5, 13, 14, 15 and 16 are quantified.

In some embodiments, in addition to at least some of the bacterial taxa previously mentioned, at least one of the following bacterial taxa can also be quantified: Blautia sp. (COT-337), Novel Bergeyella/Novel Weeksellaceae/loacibacteriulp. COT-320 (OTU 1233), Capnocytophaga canimorsus, Prevotella sp. COT-226, and Conchiformibius steedae. For example, sequences having at least about 95%, about 96%, about 97%, about 98%, about 99%, or about 100% identity to the sequences of at least 2, 3, 4, or all of SEQ ID NOs: 17, 18, 21, 23 and 27 are quantified.

In some embodiments, at least some of the bacterial taxa shown in FIG. 7 with appropriate sequences as described in FIG. 10 are quantified (e.g., 2-106, 2-100, 3-90, 4-80, 5-70, 6-60, 7-50, 8-40, 9-30, 10-20). In addition, at least some of the bacterial taxa shown in FIG. 8 with appropriate sequences as described in FIG. 10 can be quantified with the bacterial taxa in FIG. 7.

When a sequence is detected which has at least about 80%, about 85%, about 90%, about 95%, about 96%, about 97%, about 98%, or about 99% identity to the 16S rDNA sequences identified herein, the sequence can stem from a bacterium that is either of the same species or a closely related species. For example, where a sequence has 95% identity to the sequence of SEQ ID NO: x, the sequence will preferably stem from a bacterium in the same family, more preferably the same genus as the bacterium from which SEQ ID NO: x was obtained or even preferably be from the same species. For example, in the case of SEQ ID NO: 5, the bacterium would preferably be of the genus Aquaspirillum and most preferably of the species Aquaspirillum sp. FOT-079/COT-091.

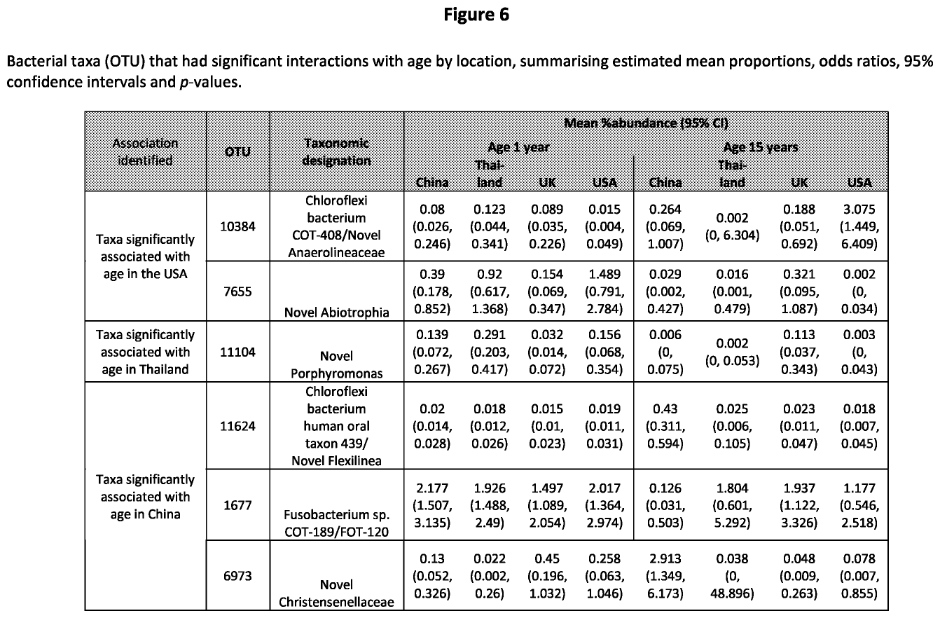

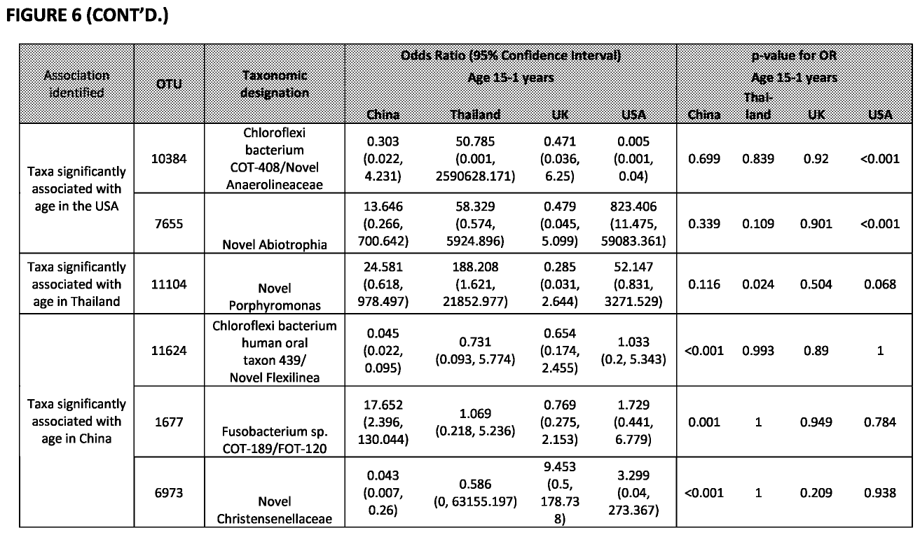

When the canid is from certain geographical locations, further correlations between relative abundance and age have been demonstrated that were not detected in the cohort as a whole. Thus, in certain preferred embodiments, the sample is from the same geographical location as the canids from which the control data set 124 was generated. For example, a sample can be from the USA, and the canids from which the control data set 124 was generated were also from the USA. In certain examples which correlate with the work shown in FIG. 6, the canid from the USA and the control data set 124 is from canids from the USA, and the biological taxa is Chloroflexi bacterium COT-408/Novel Anaerolineaccae, and/or the sequences has at least about 95%, about 96%, about 97%, about 98%, about 99%, or about 100% identity to SEQ ID NO 2. In other embodiments, the sample is from a canid from the USA and the control data set 124 is from canids from the USA, and the biological taxa is a NE Abiotrophia (OTU 7655), and/or the sequences has at least about 95%, about 96%, about 97%, about 98%, about 99%, or about 100% identity to SEQ ID NO 99. In other embodiments, the sample is from a canid from China and the control data set 124 is from canids from China, and the biological taxa is a Chloroflexi bacterium human oral taxon 439/novel Flexilinea (OTU 11624) and/or the sequences has at least about 95%, about 96%, about 97%, about 98%, about 99%, or about 100% identity to SEQ ID NO 12. In other embodiments, the sample is from a canid from China and the control data set 124 is from canids from China, and the biological taxa is a Fusobacterium I COT-189/FOT-120 (OTU 1677) and/or the sequences has at least about 95%, about 96%, about 97%, about 98%, about 99%, or about 100% identity to SEQ ID NO 31. In other embodiments, the sample is from a canid from China and the control data set 124 is from canids from China, and the biological taxa is a novel Christensenellaceae (OTU 6973) and/or the sequences has at least about 95%, about 96%, about 97%, about 98%, about 99% or about 100% identity to SEQ ID NO 89.

In some embodiments, the steps of analyzing or quantifying a bacterial species from the genus Porphyromonas and/or Prevotella, or analyzing or quantifying only bacterial species from the genus Porphyromonas and/or Prevotella can be omitted. In some embodiments, examples of species that cannot be analyzed or quantified include, but are not limited to, Porphyromonas canoris, Porphyromonas salivosa, Porphyromonas cangingivalis, Porphyromonas cansulci, Porphyromonas crevicoricanis, and Prevotella denticola.

The Control Data Set

In some embodiments, determining the age status of a canid's oral microbiome can be performed using a comparison with a control data set 124. To this end, the oral microbiome of one or more (e.g., 2, 3, 4, 5, 10, 15, 20, 25, 30, 35, 40, 45, 50, 55, 60, 65, 70, 75, 80, or more) healthy control canids can be analyzed to determine the abundance or relative abundance components of the oral microbiome. A healthy canid in this context is a canid that does not suffer from an oral cavity disorder. Examples of such disorders include periodontal diseases such as periodontitis or gingivitis. A cohort size for the canids can be chosen to enable an appropriate fold change in relative abundance between age states to be detected for bacterial taxa present at different levels of abundance.

The control canids for generating the control data set 124 can originate from a plurality of geographical locations (e.g., 2, 3, 4, 5, or more) or a single geographical location (e.g., a country, a county, a city). In certain embodiments, the control data set 124 is generated from control canids from the same geographical location as the canid whose oral microbiome status is to be assessed.

The control data set 124 will in general comprise data from two or more canids of different ages, and preferably multiple (e.g., two or more (e.g., 3, 4, 5, 10, 15, 20, 25, 30, 35, 40, 45, 50, 55, 60, 65, 70, 75, 80, or more)) canids from each of a number of different time points, for example, life stages or ages. As an example, there could be two or more (e.g., 3, 4, 5, 10, 15, 20, 25, 30, 35, 40, 45, 50, 55, 60, 65, 70, 75, 80, or more)) puppies, two or more (e.g., 3, 4, 5, 10, 15, 20, 25, 30, 35, 40, 45, 50, 55, 60, 65, 70, 75, 80, or more)) adult canids, two or more (e.g., 3, 4, 5, 10, 15, 20, 25, 30, 35, 40, 45, 50, 55, 60, 65, 70, 75, 80, or more)) senior canids and/or two or more (e.g., 3, 4, 5, 10, 15, 20, 25, 30, 35, 40, 45, 50, 55, 60, 65, 70, 75, 80, or more)) geriatric canids. The time points can be different life stages or ages. The time points can be separated by at least 6 months, 1 year, or any other suitable amount of time.

When the canid is a dog, the control data set 124 can further comprise information from dogs in the same size category (i.e., toy, small, medium, or large) as the dog to be assessed, information from dogs of the same breed size, and/or information from dogs of the same breed as one of the direct ancestors (e.g., parents or grandparents) of the dog.

The control data set 124 can also be from the same canid who has been previously diagnosed or monitored. For example, the oral microbiome age status of the canid can be analyzed and the data can subsequently be used as a control data set 124 to evaluate whether the dog's oral microbiome age status has changed.

Preparing the control data set 124 can comprise analyzing the oral microbiome composition of at least two (e.g., 3, 4, 5, 10, 15, 20, 25, 30, 35, 40, 45, 50, 55, 60, 65, 70, 75, 80, or more) puppies, and/or at least two (e.g., 3, 4, 5, 10, 15, 20, 25, 30, 35, 40, 45, 50, 55, 60, 65, 70, 75, 80, or more) adult canids, and/or at least two (e.g., 3, 4, 5, 10, 15, 20, 25, 30, 35, 40, 45, 50, 55, 60, 65, 70, 75, 80, or more) senior canids and/or at least two (e.g., 3, 4, 5, 10, 15, 20, 25, 30, 35, 40, 45, 50, 55, 60, 65, 70, 75, 80, or more) geriatric canids, determining the abundance or relative abundance of one or more bacterial taxa, and compiling these data into a control data set 124.

It will be understood that the control data set 124 does not need to be prepared every time a diagnosis is performed. Instead, a user can rely on an established control data set 124. An example of a control data set 124 is shown in FIG. 7.

The Sample

In certain non-limiting embodiments, the present disclosure provides methods for obtaining and/or using samples from the oral cavity of the animal. In certain embodiments, the sample from the oral cavity comprises saliva. In certain embodiments, the sample from the oral cavity of the animal comprises oral plaque (e.g., subgingival dental plaque, gingival margin dental plaque, supragingival dental plaque from the cheek, and/or plaque from the tongue). In certain embodiments, the sample can comprise gingival dental plaque, subgingival plaque, and/or supragingival dental plaque. In certain embodiments, the sample comprises gingival dental plaque. In certain embodiments, gingival dental plaque is a gingival marginal plaque. In certain embodiments, the gingival marginal plaque is designated as “GM.” In certain embodiments, the sample comprises subgingival plaque. In certain embodiments, the subgingival plaque is designated as “SB.” In certain embodiments, the sample comprises supragingival plaque.

In certain embodiments, the sample can be collected from an animal undergoing to general anesthesia (e.g., unconscious). In certain embodiments, the sample can be collected from an animal not undergoing to general anesthesia (e.g., conscious). In certain non-limiting embodiments, gingival dental plaque samples (e.g., gingival marginal plaque) can be collected by sweeping a periodontal probe around the entire tooth just above the gingival margins. In certain non-limiting embodiments, subgingival plaque samples can be collected by inserting a periodontal probe just under the gingival margin and sweeping around the base of the crown of the entire tooth. The sample can be fresh, frozen, or stabilized by other means such as the addition to preservation buffers or by dehydration using techniques such as freeze-drying.

After collecting the sample, the sample can be processed to extract DNA from the sample. Any suitable technique for isolation DNA can be employed, for example, as reviewed in reference [8]. Examples of techniques for isolating DNA include, but are not limited to, the Qiagen DNeasy kit™, Qiagen QIAamp Cador Pathogen Mini kit™, the Nucleospin 96 Tissue kit (Macherey-Nagel), and the Epicentre Masterpure Gram Positive DNA Purification Kit as well as Isopropanol DNA Extraction.

Any suitable technique for detecting and quantifying bacterial taxa can be employed. [8]. Examples of techniques for detecting and quantifying bacterial taxa include, but are not limited to, 454 pyrosequencing, polymerase chain reaction (PCR), quantitative PCR (qPCR), 16S rDNA amplicon sequencing, shotgun sequencing, metagenome sequencing, Illumina sequencing, and nanopore sequencing (e.g., MinION and PacBio). For example, the bacterial taxa (e.g., species) can be determined by pPCR amplification and sequencing of the 16S rDNA. Other examples include shotgun sequencing to determine characteristic non-16SrDNA gene sequences or other metabolites and biomarkers for identification of the taxa.

In certain embodiments, the bacterial taxa can be determined by sequencing 16S rDNA. In certain embodiments, the bacterial taxa can be determined by sequencing 16S rRNA. In certain embodiments, the bacterial taxa can be determined by sequence any one or more or any combination of the hypervariable regions V1-V9. In certain embodiments, the bacterial taxa are determined by sequencing the V1-V3 region of the 16S rDNA. In certain embodiments, the bacterial taxa are determined by sequencing the V3-V4 region of the 16S rDNA. In certain embodiments, the bacterial taxa are determined by sequencing the V4 region of the 16S rDNA. In certain embodiments, the bacterial taxa are determined by sequencing one or more of the V1-V3, V3-4, or V4 regions of the 16S rDNA. For example, but without any limitation, sequencing can be performed by pyrosequencing, Sanger sequencing, Illumina sequencing, or nanopore sequencing (e.g., MinION or PacBio).

In certain embodiments, the 16S rRNA is amplified and/or sequenced using a forward and a reverse primer. In certain embodiments, the forward primer comprises or consists of the nucleotide sequence set forth in SEQ ID NO: 148. In certain embodiments, the forward primer comprises or consists of the nucleotide sequence set forth in SEQ ID NO: 149. In certain embodiments, the reverse primer comprises or consists of the nucleotide sequence set forth in SEQ ID NO: 150. SEQ ID Nos: 148-150 are provided below:

(SEQ ID NO: 148)
AGAGTTTGATYMTGGCTCAG
(SEQ ID NO: 149)
AGGGTTCGATTCTGGCTCAG
(SEQ ID NO: 150)
TYACCGCGGCTGCTGG

The bacterial taxa can also be detected by other techniques such as RNA sequencing, protein sequence homology, or other biological markers indicative of the bacterial taxa.

The sequencing data can then be used to determine the abundance or relative abundance of bacterial taxa in the sample. In certain embodiments, the sequences can be clustered at about 80%, about 85%, about 90%, about 92%, about 95%, about 96%, about 97%, about 98%, about 99%, or about 100% identity and abundant taxa can be assessed for their relative proportions. For example, without any limitation, the sequences can be clustered at about 98%, about 99%, or about 100% identity and abundant taxa (e.g., those representing more than about 0.001%, about 0.005%, or about 0.01% of the total sequences) can then be assessed for their relative proportions. In certain embodiments, the sequencing data can then be used to determine the presence or absence of bacterial taxa in the sample. Examples of techniques include, but are not limited to, logistic regression, partial least squares discriminate analysis (PLSDA), random forest analysis, and other multivariate methods.

Changing the Microbiome Age Status

In some embodiments, where there is a discrepancy between the canid's actual age and oral microbiome age, the owner is notified for permission to allow an intervention or treatment to take place. In some embodiments, performing an intervention can comprise changing the composition of the oral microbiome. This can be achieved by administering a change in the oral care regime (such as tooth brushing and/or professional tooth cleaning), a dietary change or a functional food or nutraceutical, composition, pharmaceutical composition, oral chew, and/or oral care solution which is able to change the composition of the oral microbiome. Such as functional foods, nutraceuticals, live biotherapeutic products (LBPs), and pharmaceutical compositions comprising bacteria probiotics [9] and/or prebiotics.

This process can be useful when a canid's oral microbiome age status is found to be incompatible with its actual age. In this case, it can be desirable to make a change in the oral care regime (such as tooth brushing and/or professional tooth cleaning), a dietary change, a change in the administered nutraceutical or pharmaceutical composition, a change in oral chew, and/or a change in an oral care solution to shift the oral microbiome back to the appropriate age status or to an oral microbiome age status that is younger than the canid's actual age. An intervention can also be used to assess the success of a treatment. To this end, a canid can undergo a change in care that is able to change the composition of the oral microbiome. Following the commencement of the treatment (e.g., administration of the pharmaceutical composition), for example after 1 day, 2 days, 5 days, 1 week, 2 weeks, 3 weeks 1 month, 3 months, 6 months, 1 year, etc., the age status of the oral microbiome can be assessed. The age status of the oral microbiome can be determined before and after a change in care.

Dietary changes can include the use of a “dental diet” as the main meal or the administration of particular products which are believed to assist or promote dental health or hygiene, such as Dentastix® or Greenies™, or of chew toys which are known to impede plaque or calculus accumulation. Dental diets can be in the form of kibbles and are available commercially. They can have reduced protein and calcium content which limits mineralization of plaque and tartar. They can also include increased fiber which holds the kibble together for longer which then cleans the surface of the tooth. Furthermore, the size of the kibble can be selected so that it engulfs the tooth before it splits enabling the fibers to exert a gentle abrasive effect to wipe the surface of the tooth clean. The dental diets can include additional ingredients, such as sodium polyphosphate, which binds with calcium in saliva, thus making it unavailable for the formation of tartar, zinc which helps to slow down tartar build-up and has antiseptic properties, therefore, reducing bad breath and green tea polyphenols help to maintain a healthy mouth and gums.

Oral care solutions comprise dental rinses that can be in the form of solutions to add to a dog's water bowl or sprays or gels for application directly into the mouth of the dog. These can contain for example antimicrobial compounds such as chlorhexidine gluconate.

Professional tooth cleaning can comprise a deep cleaning procedure, which can be carried out under anaesthesia. This is a fairly extreme intervention which dog owners cannot utilize themselves, at least not regularly. However, it can be suggested that this is undertaken regularly.

Monitoring Oral Microbiome Health Over Time

The diagnostic system 100 can be configured to perform an intervention one or more times to determine a canid's oral microbiome health. For example, the diagnostic system 100 can perform an intervention two times, three times, four times, five times, six times, seven times, or any other suitable number of times. Performing more than one intervention allows the biological age of the microbiome to be monitored over time. This can be useful for example where a canid is receiving treatment to shift the oral microbiome. The first time an intervention is performed the age status of the microbiome is determined. Following a dietary change or administration of a nutraceutical or pharmaceutical composition, the intervention process can be repeated to assess the influence of the pharmaceutical composition on the age status of the oral microbiome. The age status of the oral microbiome can also be determined for the first time after the canid has received treatment and the intervention can be repeated afterward to assess whether there is a change in the age status of the oral microbiome.

The process can be repeated one week, two weeks, three weeks, one month, two months, three months, four months, five months, six months, 12 months, 18 months, 24 months, 30 months, 36 months, or more than 36 months apart or at least one week, two weeks, three weeks, one month, two months, three months, four months, five months, six months, 12 months, 18 months, 24 months, 30 months, 36 months, or any other suitable number of months apart.

Unless specifically stated, a process or method comprising numerous steps can comprise additional steps at the beginning or end of the method, or can comprise additional intervening steps. Also, steps can be combined, omitted or performed in an alternative order, if appropriate.

Various embodiments of the methods of the present disclosure are described herein. It will be appreciated that the features specified in each embodiment can be combined with other specified features, to provide further embodiments. In particular, embodiments highlighted herein as being suitable, typical or preferred can be combined with each other (except when they are mutually exclusive).

### EXAMPLES

The presently disclosed subject matter will be better understood by reference to the following Example, which is provided as exemplary, and not by way of limitation.

### Example 1: Assessment of Oral Microbiome Characteristics in Dogs

### Background

Periodontal disease is the most common oral disease of dogs worldwide and results from a complex interplay between plaque bacteria, host, and environmental factors. Associations between the canine oral microbiota, geographical location, and age were investigated by determining the composition of subgingival plaque samples from 587 dogs aged between 0.8 and 15 years of age residing in the United Kingdom (UK), United States of America (USA), China, and Thailand using 454-pyrosequencing. The bacterial composition of the subgingival microbiota in the UK dog population has been described previously by Davis et al. [20].

The complex interplay between canine age, health status, and the microbiota was evidenced and described to be independent of geographical location, indicating that oral care monitoring or interventions to maintain health targeted against canine oral bacteria are likely to represent globally relevant means of tracking and maintaining canine oral health.

### Study Cohort

The study cohorts comprised client-owned dogs presenting at pet hospitals in the UK, USA, China, and Thailand. All dogs under general anaesthesia for routine treatment for non-periodontal complications were considered for inclusion in the study. No dogs were anesthetized solely for the collection of plaque samples. Anaesthesia was performed according to best veterinary practices in line with the National guidelines and with Mars Animal care and welfare policies. The study was approved and informed owner consent was obtained for all the dogs that participated in the study.

Dogs over one year of age were included in the study if they had not received corticosteroids, antibiotics, or professional dental cleaning in the preceding three months. Owner surveys were completed for all dogs, including questions on the breed, age, sex, neuter status, and size (small, medium, large) of the dog. Dog breeds predisposed to developing periodontitis (e.g., Greyhounds, Yorkshire terrier, Maltese, and toy/miniature poodles) [21,22,23] and those that had moderate or severe periodontitis (e.g., >25% attachment loss [24]) were excluded from the study due to the potential for confounding based on an assumed genetic predisposition.

### Methods

In this Example, subgingival plaque samples from dogs in the USA were collected from dogs visiting a pet hospital between September 2012 and May 2013. Similar samples from China were collected from dogs visiting a pet hospital between March 2013 and July 2014, while samples from the Thailand canine population were collected from dogs visiting pet hospitals between March 2013 and June 2014. Prior to the start of the study, a sample size calculation was performed using data from a previous UK cross-sectional survey [20]. The calculation assumed that the species diversity and variability in the relative abundance of bacterial species in the other three countries would be similar to that observed in the UK dog population. Based on the power calculation a sample size of 35 dogs per health state (e.g., health, gingivitis, and mild periodontitis) was targeted. This cohort size was indicated to enable at least a 2-fold change in relative abundance between health states to be detected for bacterial species present at high abundance (>2.68% of the total population), a 3-fold change for bacterial species present at medium abundance (>0.37% of the total population) and a 5-fold change for bacterial species present at low abundance (>0.06% of the total population) with a power of at least 80% using an overall significance test level of 5% that incorporates adjustments for multiple testing [32].

### Clinical Assessment

Clinical assessments were performed by three to five veterinary nurses or veterinarians at each of the four collection sites. All received a minimum of two days of training on scoring and recording periodontal disease. The extent of gingivitis and periodontitis was assessed by taking measurements at the gingival margin using a periodontal probe. A probing depth, gingival recession, furcation exposure, and a gingivitis score between 0 and 4 were recorded for every tooth using a modified combination of the gingival index and sulcus bleeding index [24, Tables 2 and 3]. Probing depth was measured from the gingival margin to the bottom of the periodontal pocket. Gingival recession was measured from the cementoenamel junction (CEJ) to the gingival margin using the graduations of a periodontal probe. Total attachment loss was calculated as the sum of the gingival recession and the periodontal probing depth in accordance with established protocols [25,26]. Periodontitis stage 1 (PD1) was classified as being up to 25% attachment loss and periodontitis stage 2 (PD2) as between 25 and 50% attachment loss. A dental chart was completed for each dog where, in addition to recording the clinical status of each individual tooth as described above, missing teeth, crown fractures below the gum line, and foreign bodies were documented.

### Sample Collection

A collection of subgingival plaque samples and clinical assessment were conducted at the time of anaesthesia. A sterile periodontal probe was gently inserted under the gingival margin and swept along the base of the crown. For samples collected in the USA, China, and Thailand plaque was collected from all the teeth in the mouth and placed into a single Eppendorf tube containing 300 μl TE buffer (10 mM Tris-buffer, 1 mM EDTA, pH8). All samples were frozen at −20° C. within 10 minutes of collection. Samples were then stored frozen. For the UK, samples were collected according to the methods described by Davis et al. [20]. Table 2 is an example of a summary of the samples utilized in this example.

TABLE 2
An example of a summary of samples collected

| | Number of | | Storage |
|---|---|---|---|
| Location | samples | Teeth sampled | conditions |
| UK | 209 | Subset of teeth | −20° C. |
| USA | 121 | Whole mouth | −20° C. then −80° C. |
| China | 133 | Whole mouth | −20° C. |
| Thailand | 124 | Whole mouth | −20° C. |

### Microbiologic Analysis

DNA was extracted from the plaque samples and the 16S rDNA gene was amplified according to the method described by Davis et al. [20]. PCR reactions were purified, quantified, and multiplexed 454-pyrosequencing libraries created by pooling PCR amplicons in equimolar amounts. Sequences were generated using the GS FLX Titanium series 454 DNA pyrosequencer (454 Life Sciences). All pre-preparation and sequencing were performed by Eurofins MWG Operon (Ebersberg, Germany). Uni-directional sequencing was initiated from adapter B on the reverse primers. A sequencing depth of 15,000 sequences per sample was targeted which was comparable to that used by Davis et al. [20].

### Sequence Processing

The standard flow gram files (SFF) were initially filtered by selecting reads with at least 360 flows and truncating long reads to 720 flows. Reads were filtered and denoised using the AmpliconNoise software (version V1.21; Quince, 2011 and 2009). For initial filtering, reads were truncated where flow signals dropped below 0.7 indicating poor sequence quality. Subsequently, reads were denoised in three stages: 1) Pyronoise to remove noise from flow grams resulting from 454 sequencing errors (PyronoiseM parameters -s 60, -c 0.01), 2) Seqnoise to remove errors resulting from PCR amplification (SeqNoiseM parameters -s 25, -c 0.08), and 3) Perseus detection and removal of chimeras introduced by PCR recombination. The denoised sequences were then clustered using QIIME v1.7.0. The QIIME script pick_otus.py, which utilizes the Uclust v1.2.22q software program, was used to cluster sequences with ≥98% identity [27]. Uclust was run with modified parameters, with gap opening penalty set to 2.0 and gap extension penalty set to 1.0 and—A flag to ensure optimum alignment [27]. Representative sequences of all OTUs were annotated using BLAST [28] against the Silva SSU database release 119 [29]. If the alignment matched the top BLAST hit with ≥98% sequence identity and ≥98% sequence coverage then a species-level was assigned but if these criteria were not met the next appropriate level of taxonomic assignment was allocated: ≥94% genus, ≥92% family, ≥90% order, ≥85% class, and ≥80% phyla.

### Statistical Analysis

An OTU was classified as rare if none of the locations had an average proportion above 0.05% and/or had a presence in less than two samples [20]. Total sequence depth, age (years), and average gingivitis score were analyzed by generalized least squares linear models with a fixed effect of location and weighting the variance by location. Means were compared using Tukey HSD tests to the 5% level and reported with 95% family-wise confidence intervals. The percentage of rare sequences, healthy teeth, and periodontitis teeth were analyzed by logistic regression analyses (e.g., generalized linear model (GLM) with a quasi-binomial distribution and logit link) for proportions, using the count of rare sequences out of the total sequence depth. The location was investigated as a fixed effect and means were compared using Tukey HSD tests to the 5% level. Contingency tables of breed size, sex, and neuter status by location were analyzed using Chi-square tests for independence using a test level of 5%.

### Analysis of Bacterial Composition by Multivariate Analysis

A multivariate analysis was conducted to assess whether bacterial combinations were associated with cohort parameters. The log 10 relative abundance data describing bacterial profiles in canine plaque samples were analyzed by principal components analyses (PCA). Score plots of the components were investigated for correlations with breed size, age, gender, neuter status, average gingivitis score, and the percentage of healthy and periodontitis teeth in the mouth and the sampled teeth.

Analysis of Individual Bacterial Taxa with Cohort Characteristics

The 280 individual bacterial taxa OTUs were analyzed univariately by logistic regression analyses (e.g., using GLM with a quasi-binomial distribution and logit link) for proportions, using the count for the OTU out of the total number of sequences (i.e., the relative bacterial abundance cf. the total sequence population for each sample). To enable model convergence when an OTU has many zero counts, 2 counts were added to each OTU count and 4 counts were added to the total count (analogous to adding 2 successes and 2 failures) prior to analyses. The models were explored to investigate the correlation of the OTUs with the fixed effects for oral health status, as measured by the percentage of healthy teeth, percentage of periodontitis teeth and the average gingivitis score, and their two-way interactions with location and each other as well as age and breed size as covariates.

Models were built initially to minimize the quasi-Akaike's Information Criterion (qAIC) for binomial distributions with overdispersion [31], with geographical locations fixed to remain in the model. The model with the smallest qAIC was then chosen to be tested for significance. To adjust for multiplicity effects, the p-values of each fixed effect in the minimum qAIC model were adjusted for the 280 OTUs analyzed. Subsequently, effects were removed from the model if found to be non-significant by Benjamini and Hochberg [32] with a false discovery rate of 5% level. Once this minimal model by qAIC and BH adjustment was formed, the data were subjected to a permutation test to assess the sensitivity of the results to possible outliers or deviations from the assumption of the generalized linear model. An effect remained in the model if the proportion of permutations where the significance of the effect was as least as small as the observed effect was less than 5%. Means and odds ratios between levels were then calculated at the covariate averages according to the final model found, with 95% family-wise confidence intervals. P-values for comparisons were calculated using a family-wise error rate of 5%.

### Microbial Diversity

The Shannon Diversity index of each sample was calculated according to the methods of Shannon [33] and the resulting measures of bacterial diversity within subgingival plaque samples were analyzed by linear regression modeling. The model was built using stepwise regression to minimize the AIC, with fixed effects as defined for the univariate analyses and the total number of sequences and location fixed in the model. The data was then subjected to a permutation test according to the minimized model, as for univariate analyses. In this example, a test level of 5% was used.

In this example, statistical analyses were performed in R v3.2.2 statistical software. PCA analysis was performed using the library vegan, comparisons and confidence intervals were calculated using multcomp and the model AICs were calculated using MuMIn. Graphics were generated using ggplot2 [34]. In other examples, any other suitable type of software can be used.

### Cohort Details

Subgingival plaque samples from 587 dogs were included in the study. Table 3 is an example of metadata for the cohort with details per geographical location.

TABLE 3
An example of metadata for each of the four locations

| Factor | UK | USA | China | Thailand |
|---|---|---|---|---|
| Age (years) | | | | |
| Average | 5.78 | 5.61 | 5.02 | 2.67 |
| (95% CI) | (5.26, 6.3)* | (4.93, 6.29)* | (4.17, 5.87)* | (2.16, 3.18)* |
| Range | 1.5 to 15 | 1 to 14.5 | 1 to 14 | 0.8 to 14 |
| Breed size | | | | |
| Small | 36 (17.6%) | 56 (46.3%) | 57 (42.5%) | 22 (17.7%) |
| Medium | 76 (36.9%) | 24 (19.8%) | 43 (32.1%) | 81 (65.3%) |
| Large | 93 (45.1%) | 41 (33.9%) | 33 (24.8%) | 21 (16.9%) |
| Sex | | | | |
| Female | 94 (45.2%) | 54 (44.6%) | 55 (45.1%) | 59 (47.6%) |
| Male | 114 (54.5%) | 67 (54.5%) | 67 (54.5%) | 65 (52.4%) |

*Significance with Tukey HSD homogenous groups at 5% within model

Interactions Between Dog Age and Clinical Oral Health Status with Bacterial Taxa and Sequences Identified in Canine Plaque

After quality filtering, 6,944,757 sequence reads were obtained from the subgingival plaque samples. Clustering of these at ≥98% sequence identity resulted in 280 apparent bacterial taxa OTUs approximating to species-level identification. Following exclusion of rare OTUs present at <0.05% in all four countries, the subgingival plaque from dog populations located in all geographies had a similar community membership although the relative abundance of certain taxa varied significantly across locations.

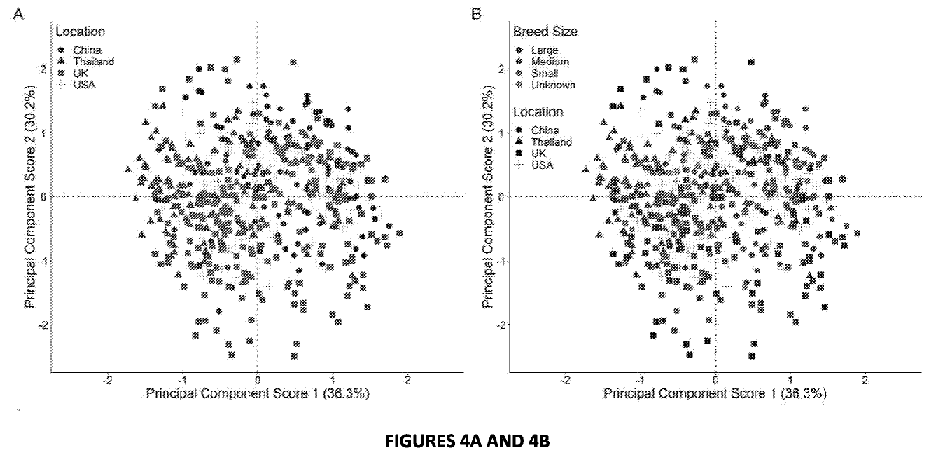

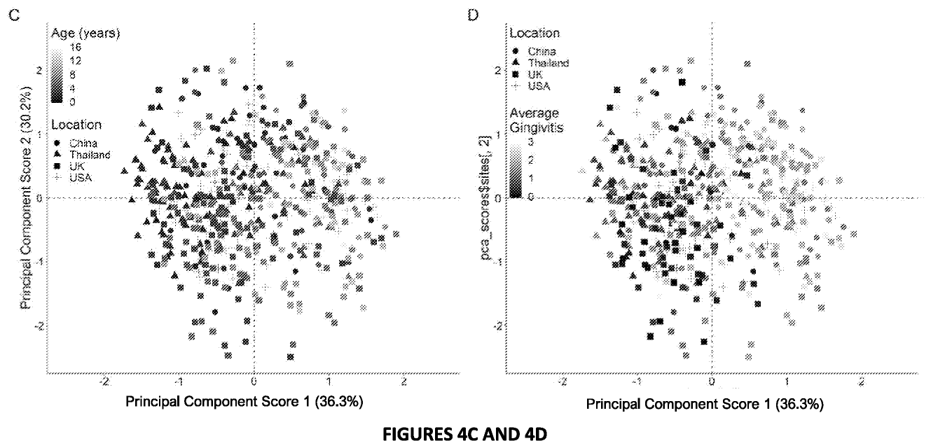

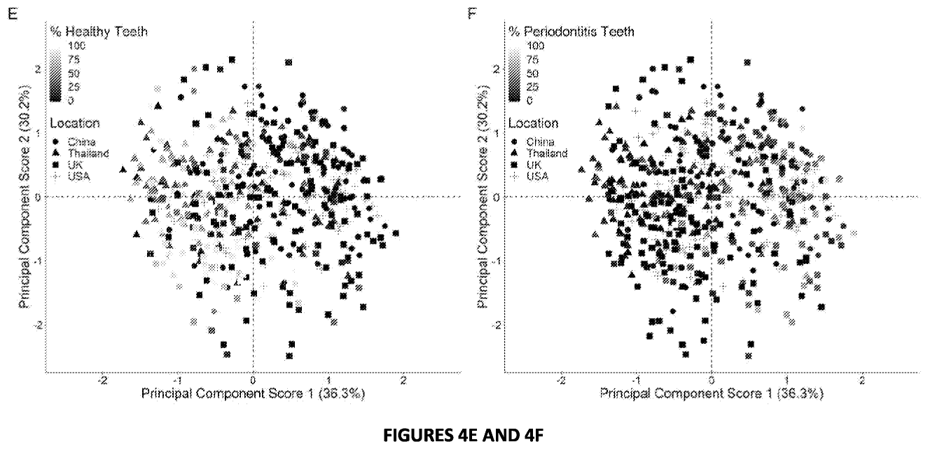

Analysis of the interactions between clinical status and age revealed a marked similarity among the bacteria associated with increasing age in the canine population and those associated with gingivitis. An exploratory PCA did not show discrete clustering of plaque microbiota by location. For example, see FIG. 4A. No discrete clustering was seen by breed size, sex, or neuter status. For example, see FIG. 4B. However, progression was evident by PCA analysis with increasing age suggesting that the variance in the OTUs changes with advancing age. For example, see FIG. 4C. Clinical status was also related to a progression in the OTUs suggesting that the variance in the data was altered with the percentage of healthy teeth identified in the dog and average gingivitis score. For example, see FIGS. 4D and 4E. Progression of the variance in OTU relative abundance data was also evident by PCA in relation to the number of teeth with periodontitis. For example, see FIG. 4F.

These analyses indicate that the composition of the canine plaque microbiota is associated with the extent of gingivitis and periodontitis (i.e., oral health status) and also with the age of the dog. Although no discrete clustering by breed size was observed using multivariate methods that explore patterns based on the total bacterial population, this does not mean that individual OTUs (species) are not associated with breed size. Therefore, OTU associations, with breed size were further explored using a univariate process.

Alterations in the Oral Microbiota with Age

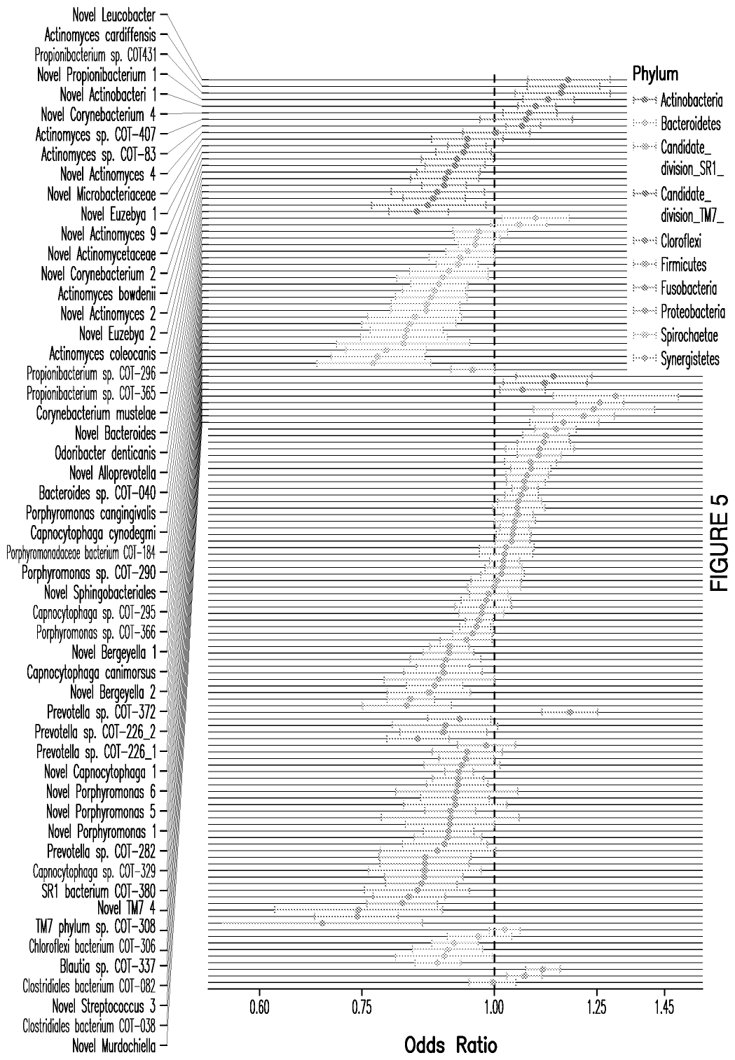

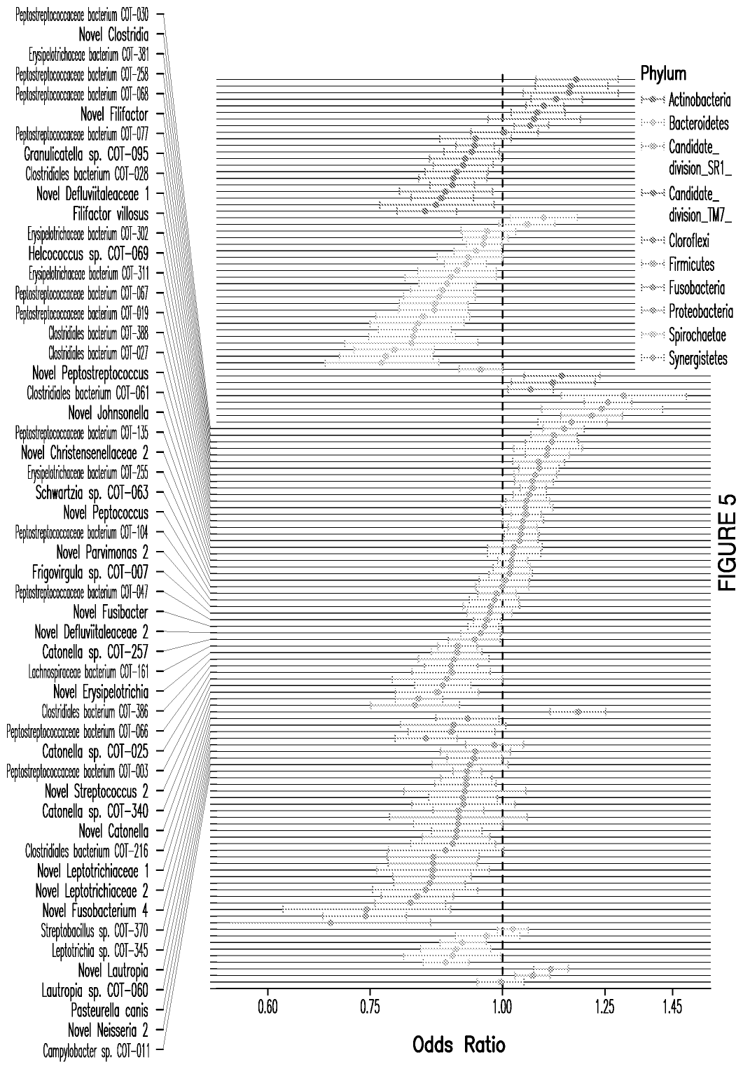

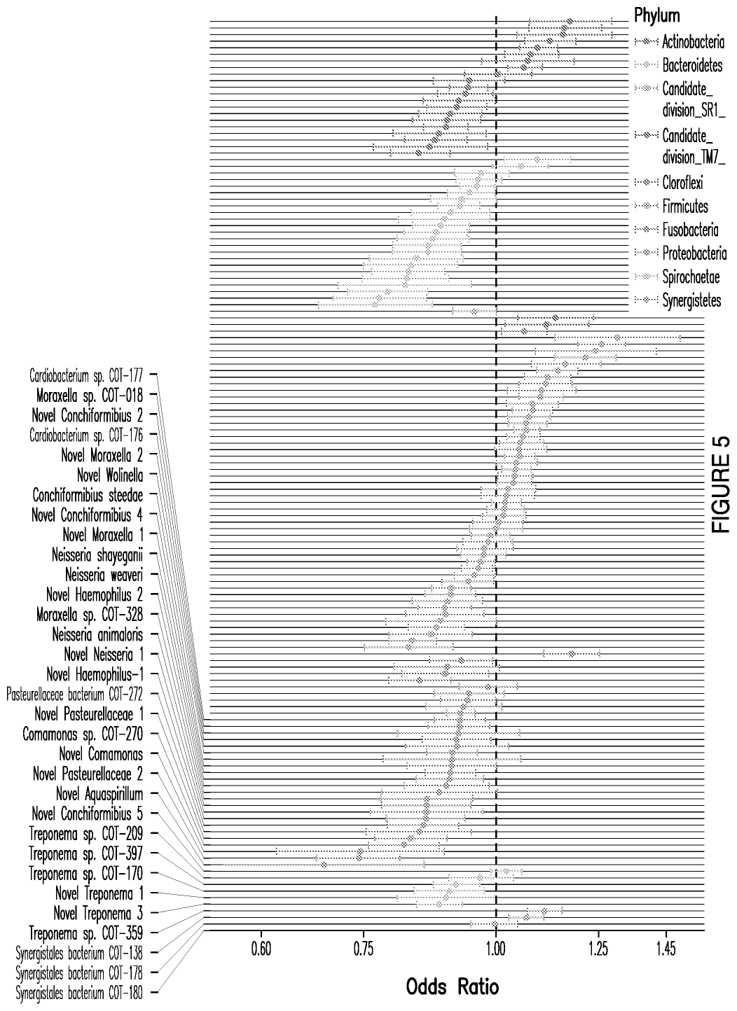

FIG. 6 is an example of bacterial taxa that have significant interaction with age by location. Of the 138 bacterial taxa OTUs without interactions with the clinical status that were found to have significant age effects, 36 OTUs had a significantly higher estimated mean proportion at age 15 than age 1 and 70 OTUs had a significantly lower proportion at age 15 compared to age 1. For example, see FIG. 7. Of those that were significantly more abundant at an older age, the majority belonged to the phyla Firmicutes (22 OTUs) and Actinobacteria (7 OTUs). The remaining seven OTUs were members of the phyla Bacteroidetes, Synergistetes, TM7, Chloroflexi, and Fusobacteria. For example, see FIG. 5.

Within the phylum Firmicutes, there were 12 abundant OTUs (>0.3% of the population) that had an odds ratio >2 when comparing the estimated proportion at age 15 to age one: four belonged to the family Peptostreptococcaceae, two belonged to the family Erysipelotrichaceae, two belonged to the class Clostridiales, and four were species (Blautia sp. COT-337, Granulicatella sp. COT-095, Filifactor villosus, and a novel species belonging to the genus Streptococcus).

With respect to members of the phylum Actinobacteria, the four most abundant OTUs (>0.3%) with an odds ratio greater than two were Actinomyces sp. COT-083, Propionibacterium sp. COT-431 and two novel species one from the genus Corynebacterium and the other from the genus Leucobacter. Of the bacterial taxa OTUs that had a significantly lower proportion at age 15 compared to age 1, the majority belonged to four phyla: Proteobacteria (20 OTUs), Bacteroidetes (19 OTUs), Firmicutes (13 OTUs), and Actinobacteria (11 OTUs). The remaining 11 OTUs belonged to the phyla Fusobacteria and Spirochaetae. With respect to the Proteobacteria phylum, the most abundant members (>0.3%) with the biggest difference between ages 15 and one (odds ratio >2) were three species of Neisseria (N. animolaris, N. shayeganii, and N. weaveri), two species from the genus Moraxella (Moraxella sp. COT-018 and a novel Moraxella species), two novel species from the family Pasteurellaceae, Campylobacter sp. COT-011, and a novel species from the genus Aquaspirillum.

Within the phylum Bacteroidetes, there were two species from the genus Capnocytophaga (C. canimorsus, cynodegmi), a novel species from the genus Bergeyella, Prevotella sp. COT-226, Porphyromonaaceae bacterium COT-184, and two species from the genus Porphyromonas (Porphyromonas sp. COT-290 and a novel Porphyromonas species).

In the phylum Firmicutes, there were two novel species, one from the genus Catonella and one from the genus Streptococcus. With respect to the phylum Actinobacteria there were two species from the genus Corynebacterium (C. mustelae and a novel Corynebacterium species), two novel species from the genus Euzebya, a novel Actinomyces species, and Propionibacterium sp. COT-296.

### Example 2: Breed Size Impacts the Age Status Determination of a Dog

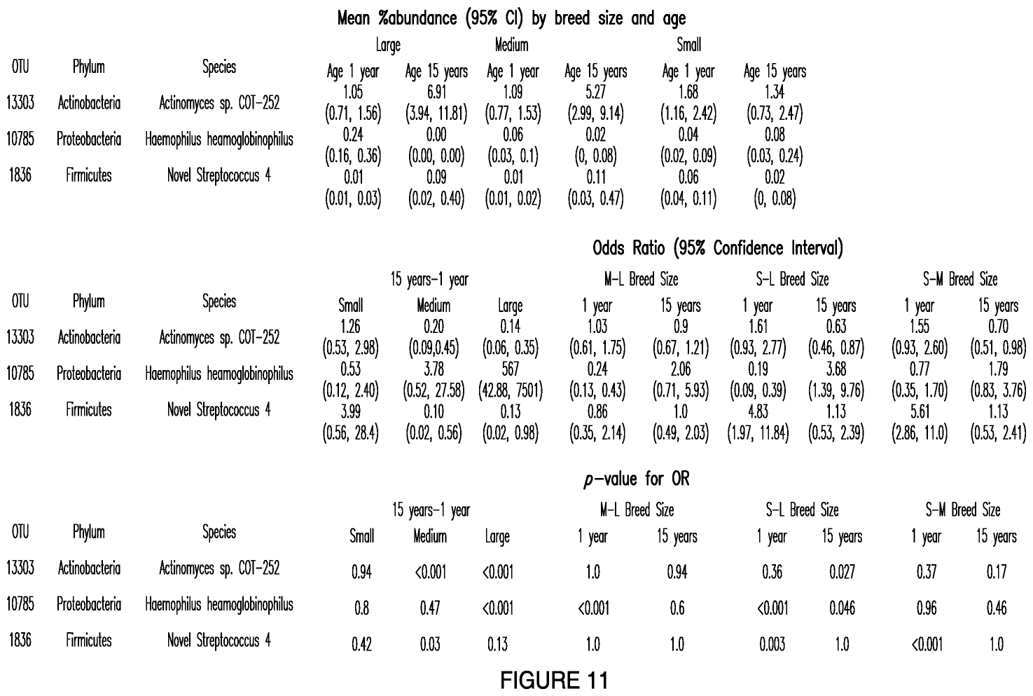

In this Example, the same techniques can be used as described above in Example 1. Despite FIG. 4B showing no discrete clustering in a PCA plot by breed size, when the data is further analyzed to the level of OTUs, it was found that certain OTUs do have significant interactions between age status and breed size. FIG. 11 is an example of a summary of the estimated mean proportions, odds ratios, 95% confidence intervals, and p-values shown in FIG. 4B. FIG. 4B, therefore, only describes the overall bacterial population and at that level, no gross changes in the oral microbiome are observed. FIG. 12 is an example of the 16S rDNA sequences for the bacterial taxa identified in FIG. 4B. Below is an analysis of three OTUs that demonstrate how a breed size impacts the age status determination of a dog via the life stages determination.
OTU 13303 —Actinomyces sp. COT-252

It can be seen that the relative abundance is significantly higher at 15 years of age compared to 1 year of age for medium and large size dogs. At 15 years of age, the relative abundance is significantly higher in large size dogs than small size dogs
OTU 10785—Haemophilus haemoglobinophilus

It can be seen that the relative abundance is significantly higher at 1 year of age compared to 15 years of age for large size dogs. At 1 year of age, the relative abundance in large size dogs is significantly higher than in small size dogs whereas at 15 years of age the opposite is true i.e., more abundant in small size dogs. At 1 year of age, the relative abundance in large size dogs is also significantly higher than in medium size dogs.
OTU 1836—Novel Streptococcus

The relative abundance is significantly higher at 15 years of age compared to 1 year of age for medium size dogs. At 1 year of age, the relative abundance is significantly higher in small size dogs than large and medium size dogs.

### Example 3: Assessment of Oral Microbiome Characteristics in Dogs Using Subgingival and Gingival Margin Plaque Samples

### Background

Periodontal disease represents a significant health issue in canine pet populations across the world. Development of the disease is dependent on the interplay between a number of host and environmental factors. Associations between oral health status, plaque location, canine oral microbiota, and age, and oral health status, plaque location, canine oral microbiota, and breed size were determined by investigating the microbial composition of subgingival (SG) and gingival margin (GM) plaque samples from 381 dogs. The cohort comprised client-owned dogs distributed across subsets in three geographical locations and the plaque samples collected were sequenced using MiSeq Illumina. A comparison of subgingival and gingival margin plaque microbiota for this sample cohort has been previously described in Ruparell et al. (2021) [41].

### Study Cohort

The study comprised client-owned dogs, across three geographically separated cohorts visiting veterinary hospitals in the USA, China and Thailand. Informed owner consent was obtained for all dogs included in the study. The study was approved and complied with all federal regulations regarding clinical investigations in veterinary practice.

### Sample Collection

SG and GM plaque were collected while dogs were under general anesthesia for routine veterinary treatment for non-periodontal disease complications. GM samples were collected by sweeping a periodontal probe around the entire tooth just above the GM. SG plaque samples were collected by inserting a periodontal probe just under the GM and sweeping around the base of the crown of the entire tooth. For both sample types, plaque from every tooth in the mouth was collected, pooled and placed into 300 μLTE buffer (10 mM Tris-buffer, 1 mM EDTA, pH8). Collections in the USA were initially stored at −20° C., then transferred to −80° C., while those in China and Thailand were retained at −20° C. for up to 18 months prior to transit to the UK on dry ice for further processing.

### DNA Extraction

As previously described by Davis et al. [20], the Epicentre Masterpure Gram Positive DNA Purification Kit (Epicentre, USA) was used to extract DNA from the plaque samples, following the manufacturer's instructions with additional overnight lysis.

Amplification of 16S rDNA Gene

A region of approximately 470 bp, spanning the variable V3-V4 regions of the 16S rDNA gene, was amplified from the plaque DNA extractions. Universal bacterial primers designed according to Fadrosh et al. (2014) [37] were used for PCR amplification in conjunction with the Phusion® High-Fidelity PCR Master Mix with HF Buffer (MO531, New England Biolabs, UK). Details of the PCR mixtures and reaction cycling conditions were followed as previously described by Ruparell et al. (2020) [40].

### Library Preparations and Sequencing

Library pool preparation and MiSeq Illumina sequencing were carried out by Eurofins Genomics, Germany. Details on the steps undertaken for quantification, dilution and pooling of amplicons as well as the sequencing itself have been reported in Ruparell et al. (2021) [41].

### Sequence Data Processing

Details on processing steps including assembly of read sequences into contiguous sequences, removal of tags, de-multiplexing, and removal of chimeric sequences are reported in Ruparell et al. (2021) [41].

Sequences were clustered at ≥98% identity to generate operational taxonomic units (OTUs). The most abundant sequences were chosen as cluster representatives were annotated with blastall 2.2.25 [35], which also contained canine and feline oral microbiome sequences. Further details on this are available in Ruparell et al. (2020) [40].

### Statistical Analysis

### Meta-Analysis

A meta-analysis was performed on sample information for the dog ages, average gingivitis score, and the proportions of healthy and periodontitis teeth. Age and gingivitis score were analysed using linear models, and healthy and gingivitis teeth counts were analysed using generalised linear models with binomial error distributions. All models had geographical location as the sole fixed effect. From these models, means values were estimated, with 95% confidence intervals. Tukey tests were also performed comparing geographical locations within each individual measure or factor. Sex was analysed by modelling the ratios of females:males by location. This was done using a generalised linear model with a binomial error distribution and a Tukey test to compare locations.

### Categorisation of OTUs

Prior to analysis, OTU abundance counts were made relative to the total sequence depth. Using these relative abundances, OTUs which were identified as rare/noise were combined into a single pseudo-OTU. OTUs were classified as non-rare if they appeared in at least two samples of any study group at a relative abundance of at least 0.05% [20]. Study groups were defined as sampling location (SG or GM) and health state combinations.

### Multivariate Analyses

Sequence data was analysed using both multivariate and univariate methodology. For multivariate analysis principal components analysis (PCA) was applied to arcsin square root transformed relative abundance data with OTUs mean centred and variance scaled. The arcsin square root transform is an alternative to a log transform for obtaining data which is approaching a normal distribution however has the benefit of being applicable to zero values. OTUs were mean centred and variance scaled to ensure low relative abundance OTUs were represented in the results.

Shannon diversity was calculated using the relative abundance data and modelled using linear mixed effects models. A range of fixed effects were investigated using a backwards rejection stepwise model fitting algorithm with the eventual model having diversity as the response and sampling location as the single fixed effect. Health measures, the interaction between health indicators and sampling location, and age were all excluded. Random effects of geographical location, dog and breed size were included as they were deemed significant by a likelihood ratio test from the stepwise selection routine. Mean diversity was compared between SG and GM at a 5% level.

### Phylum Level Analysis

Phylum level analysis was performed by first labelling each OTU with the assigned phylum and then aggregating the data, on a sample level, to be phylum level counts. These counts and the corresponding sample totals were then modelled using a generalised linear model with a binomial distribution and a logit link. The fixed effects of this model were phylum, sampling location and their interaction. From this model phylum level relative abundances were estimated for each sampling location and contrasts performed comparing sampling location within phyla.

### Oxygen Requirement Status Clustering

Additionally, OTUs were assigned oxygen requirement status and counts were summed within each level. The relative abundance GLMM testing methodology described below was applied to each oxygen requirement level with the same fixed effects aside from health status as this was not of relevance to the oxygen requirement question.

### Univariate Analysis

Prior to univariate modelling the OTUs were split into three data sets; those with fewer than 50% zeroes (182 OTUs) and those with between 50% and 80% zeroes (165 OTUs). This left 280 OTUs unanalyzed due to containing too many zeros. All OTUs were modelled with a series of generalised linear mixed effects models with binomial error distributions and logit link functions. Models applied to OTUs with less than 50% zeroes had relative abundance as the response and those with more than 50% zeroes had a presence/absence indicator. Identification of these groups was done to ensure the distribution assumptions of the parametric models for relative abundance held and to ensure the presence/absence models had sufficient data to accurately fit to the data.

Both sets of models had the same fixed (age) and random (geographical location, dog, breed size) effects. All models also included an observation level random effect to control for overdispersion in the data [38]. For each tooth health measure (proportion of healthy teeth, proportion of periodontitis teeth and average gingivitis score) a series of models were fit with specific fixed effects. These models are detailed in full in Si Appendix. By testing pairs of these models using ANOVA OTUs were grouped into three sets. These were identified as OTUs showing a significant effect with health status which differs by sampling location, showing a significant effect with both health status and sampling location separately, or showing a significant effect with health status only. For each OTU the maximal model, corresponding to the set to which this OTU was assigned, was used to estimate means with 95% confidence intervals.

For the univariate modelling the false discovery rate was controlled to 5%, within each set of model contrasts, using the Benjamini-Hochberg procedure [32]. All statistical analyses were performed using the R statistical programming language [42] with univariate models fit using the 1me4 library [36].

Associations with Age or Breed Size

An initial investigation showed a significant relationship between oral health status and both age and breed size. Therefore, the oral health terms were included in the following analysis to account for these interactions.

Modelling was performed on the OTU data set split by % zeros (see Univariate analysis section above) This was done to ensure the distribution assumptions of the parametric models for relative abundance held and to ensure the presence/absence models had sufficient data to converge on a solution. All OTUs were modelled with a generalised linear mixed effects models with a binomial error distribution and logit link function. Models applied to OTUs with less than 50% zeroes have relative abundance (count out of sample total) as the response and those with more than 50% zeroes have an indicator for a count greater than 0. For each OTU, four models were fit with the following fixed effects:
Age, Gingivitis, Sample Type (SG or GM) and their interaction
Age, PD, Sample Type (SG or GM) and their interaction
Breed Size, Gingivitis, Sample Type (SG or GM) and their interaction
Breed Size, PD, Sample Type (SG or GM) and their interaction
   ‘Gingivitis’ means average (mean) whole mouth gingivitis score. ‘PD’ means proportion of teeth categorised as PD1 or above.

All models had a random structure of Location (USA, China or Thailand) and Sample (Individual ID for each observation). The observation level random effect (OLRE) was included to account for overdispersion in the model. However, where convergence issues were identified in model fitting, the OLRE was not included.

In the Age & Gingivitis models, the significance of the age slope is tested and reported at three levels of Gingivitis (0, 1, 2) in both Sample Types (SB, GM).

In the Age & PD models, the significance of the age slope is tested and reported at three levels of proportion PD (0, 0.2, 0.4) in both Sample Types (SB, GM).

In the Breed Size & Gingivitis models, the significance of the three between breed size comparisons are tested and reported at three levels of Gingivitis (0, 1, 2) in both Sample Types (SB, GM).

In the Breed Size & PD models, the significance of the three between breed size comparisons are tested and reported at three levels of proportion PD (0, 0.2, 0.4) in both Sample Types (SB, GM).

### Results

### Overview/Meta-Data

SG and GM plaque bacterial communities were sampled from a total of 381 client-owned dogs visiting veterinary hospitals in the USA, China and Thailand, as previously described by Wallis et al. (2021) [44]. Per geographical location, the relative study cohorts comprised 120 from USA, 129 from China and 132 from Thailand. Since dog size and age represent putative risk factors for periodontitis, metadata associated with these parameters was collated. Table 4 indicates metadata based on the full cohort, discriminated by geographical location. Tukey analyses revealed the mean age of the dogs was significantly lower for the Thailand sub-cohort compared to both the USA and China sub-populations (p<0.001). Chi-squared analysis showed that there was a significant difference in the distribution of breed size across the dogs sampled in each of the three locations (p<0.001). Dogs of small breed size sampled in Thailand represented a significantly smaller subset compared to those in both the USA and China (p<0.001). In contrast, the number of medium breed size dogs was significantly higher for Thailand than the equivalent subsets in the USA and China cohorts (p<0.0001). A significant difference for breed size was also observed with large dogs, but only between two of the geographical locations where that of the Thailand subset was significantly smaller than that in the USA (p<0.005). No significant differences were observed in the ratio of males to females (p>0.940).

TABLE 4
Metadata based on the full cohort by geographical location

| | USA | China | Thailand |
|---|---|---|---|
| Age (years) | | | |
| Average | 5.61 | 5.00 | 2.78 |
| (Range indicated by 95% | (4.94, 6.29)^{b} | (4.35, 5.66)^{b} | (2.14, 3.43)^{a} |
| confidence intervals) | | | |
| Breed size | | | |
| Small | 56 (46.7%) | 55 (42.6%) | 28 (21.2%) |
| Medium | 24 (20.0%) | 40 (31.0%) | 83 (62.9%) |
| Large | 40 (33.3%) | 34 (26.4%) | 21 (15.9%) |
| Sex | | | |
| Female | 54 (45%) | 57 (44.2%) | 62 (47.0%) |
| Male | 66 (55%) | 70 (54.3%) | 70 (53.0%) |
| Unknown | | 2 (1.6%) | |

### Samples and Sequence Summary

The study generated a total of 772 plaque samples divided into geographical location subsets as follows: USA—240 (120 SG and 120 GM); China—275 (137 SG and 138 GM); and Thailand −257 (132 SG and 125 GM).

Sequencing analysis of the V3-V4 region of the 16S rDNA gene of the 772 plaque samples via MiSeq Illumina generated 51,697,579 assembled reads after bioinformatics processing. Final numbers of sequence reads per sample ranged from 21 to 174,817 with a median of 69,974.5 reads. More specifically, sequence reads for SG plaque ranged from 21 to 174,817 with a median of 63,828 reads and those for GM plaque from 5,410 to 142,161 with a median of 75,776 reads.

A total of 23 samples were removed prior to statistical analysis. This included two SG samples with counts under 1,000 sequence reads. Another 21 samples with missing sample information were also removed, comprising 12 SG plaque samples and 9 GM plaque samples. The total number of sequence reads remaining for the subsequent analysis was 50,370,035.

### Bacterial Composition of Canine Plaque

After assigning the “rare/noise” sequence reads to a separate group, the 50,370,035 assembled sequences were assigned to 627 OTUs. The rare/noise group accounted for 1.17% of the total sequence reads.

Sequence identity comparison of the 627 OTUs against 16S sequences within a combined database containing the Silva database (v132) and the Canine Oral Microbiome Database (COMD) was used to determine taxonomy. Identities ≥98% to 16S sequences within the database were observed for 508 of the 627 OTUs (81.0%). The remaining 119 OTUs (20.0%) aligned to sequences with between 84.9% and 97.9% identity. Of the 627 OTUs, 273 (43.5%) aligned to sequences previously identified as canine oral taxa (COTs) [7]. The remaining 354 OTUs (56.5%) aligned to other taxa within the Silva database. Of these, 92 (14.7%) were designated species level taxonomy.

An assessment of the taxonomic composition of the 627 OTUs was performed at the phylum level. The distribution of 577 OTUs was spread across 13 phyla: Firmicutes (36.4%), Actinobacteria (20.1%), Proteobacteria (16.1%), Bacteroidetes (14.3%), Fusobacteria (2.2%), Spirochaetes (1.6%), Synergistetes (1.3%), Chlorobi (0.28%), Tenericutes (0.24%), Chloroflexi (0.016%), Elusimicrobia (0.013%), Deinococcus-Thermus (0.003%) and Euryarchaeota (0.001%). The remaining 50 OTUs were assigned to six candidate phyla: Saccharibacteria (4.80%), Absconditabacteria (0.67%), WS6 (0.40%), Gracilibacteria (0.35%), WPS-2 (0.008%) and Microgenomates (0.002%).

The 21 most abundant taxa, present at ≥1.5% accounted for approximately 45.7% of the sequences reads (Table 5). Actinomyces sp. COT-404 (OTU #15137) was the most abundant taxa representing 4.51% of the total number of sequence reads. Porphyromonas cangingivalis (OTU #29659) and Moraxella sp. COT-017 (OTU #33608) were the next most abundant representing 3.36% and 3.22% of the sequence reads respectively. A further 7 OTUs represented between 2.63% and 2.04%, and 23 OTUs between 2.00% and 1.00% of the population. The remaining 594 OTUs were below 1.00% and ranged in relative proportion from 0.00003% to 0.93%.

TABLE 5
The 21 most abundant taxa, present at ≥1.5%

| | | | | Percentage |
|---|---|---|---|---|
| | | | Total Number | of total |
| | Assigned Taxonomy | Percentage | of Sequence | sequence |
| OTU | (Family/Genus/Species) | identity | Reads | reads (%) |
| 15137 | Actinomyces sp. COT-404 | 99.76 | 2266818 | 4.50 |
| 29659 | Porphyromonas cangingivalis | 100.0 | 1694765 | 3.36 |
| 33608 | Moraxella sp. COT-017 [1] | 99.76 | 1620361 | 3.22 |
| 24357 | Actinbacteria bacterium COT-406 [2] | 100.0 | 1323304 | 2.63 |
| 3298 | unclassified Actinomyces [novel 37] | 100.0 | 1210253 | 2.40 |
| 24637 | Filifactor villosus | 100.0 | 1169300 | 2.32 |
| 8990 | Peptostreptococcaceae bacterium COT-077 | 99.75 | 1164969 | 2.31 |
| 10101 | Clostridiales bacterium COT-028 | 99.51 | 1150401 | 2.28 |
| 23077 | Saccharibacteria (TM7) sp. COT-305 [2] | 100.0 | 1099958 | 2.18 |
| 15380 | Peptococcus sp. COT-044 | 100.0 | 1028831 | 2.04 |
| 29692 | unclassified Bergeyella sp. [novel 4] | 100.0 | 959988 | 1.91 |
| 27298 | Peptostreptococcaceae bacterium COT-004/005 [2] | 100.0 | 942519 | 1.87 |
| 22852 | Actinomyces sp. COT-252 | 99.76 | 924581 | 1.84 |
| 25947 | Peptostreptococcaceae bacterium COT-047 [2] | 100.0 | 918879 | 1.82 |
| 23691 | Peptostreptococcaceae bacterium COT-019 [3] | 100.0 | 874144 | 1.74 |
| 23739 | Frigovirgula sp. COT-007 [2] | 99.75 | 848892 | 1.69 |
| 20143 | Parvimonas sp. COT-035 | 100.0 | 769673 | 1.53 |
| 12804 | Neisseria canis [6] | 98.60 | 769425 | 1.53 |
| 18962 | Saccharibacteria (TM7) sp. COT-363 [2] | 99.51 | 763570 | 1.52 |
| 2165 | unclassified Capnocytophaga [novel 3] | 100.0 | 763202 | 1.52 |
| 13754 | Granulicatella sp. COT-095 | 100.0 | 756933 | 1.50 |

### Comparison of Subgingival and Gingival Margin Plaque Samples and Geographies

Principal component analysis (PCA) was used to identify the most prominent sources of variability between the samples (FIGS. 13A and 13B). The two plaque locations were found to be similar; however, a number of the SG samples displayed a wider variability in OTU relative abundances not observed with any of the GM samples (FIG. 13A, PC2 above zero). Differentiating the results by geographical location indicated differences between all three locations in the more variable samples with China moving out to the top left, Thailand moving top right and the USA sitting in between (FIG. 13B). A large number of samples, however, were all closely grouped in the centre and bottom left of the figure. From the PCA, the first component explained 7.79% and the second component 5.08% of the variability in the OTU arcsin square root transformed proportions.

The phylogenetic distribution amongst the two plaque sample groups is represented in FIG. 14. There was a consensus in the ordering of the five most abundant phyla: Firmicutes, Actinobacteria, Proteobacteria, Bacteriodetes and Saccharibacteria (TM7, candidate phyla). These accounted for 89.8% and 93.2% of the respective sequence counts for SG and GM plaque. The ordering of the remaining 15 phyla varied between established and candidate phyla for both plaque samples.

### Diversity

The Shannon diversity index indicated a significant difference between the groups of plaque samples (FIG. 15). The Shannon diversity index was significantly lower for GM samples compared to SG samples (p<0.001).

### Oxygen Requirements

For each OTU, oxygen requirements were ascertained using literature searches based on each of the assigned taxonomic identifiers. Generalised linear mixed model (GLMM) analysis was then used to explore for potential differences in aerobes and anaerobes between the plaque locations (FIGS. 16A and 16B). SB plaque samples had a significantly lower proportion of aerobic OTUs (p<0.001) (FIG. 16A) and a significantly higher proportion of anaerobic OTUs than GM plaque samples (p<0.001) (FIG. 16B).

### Health Status Associations

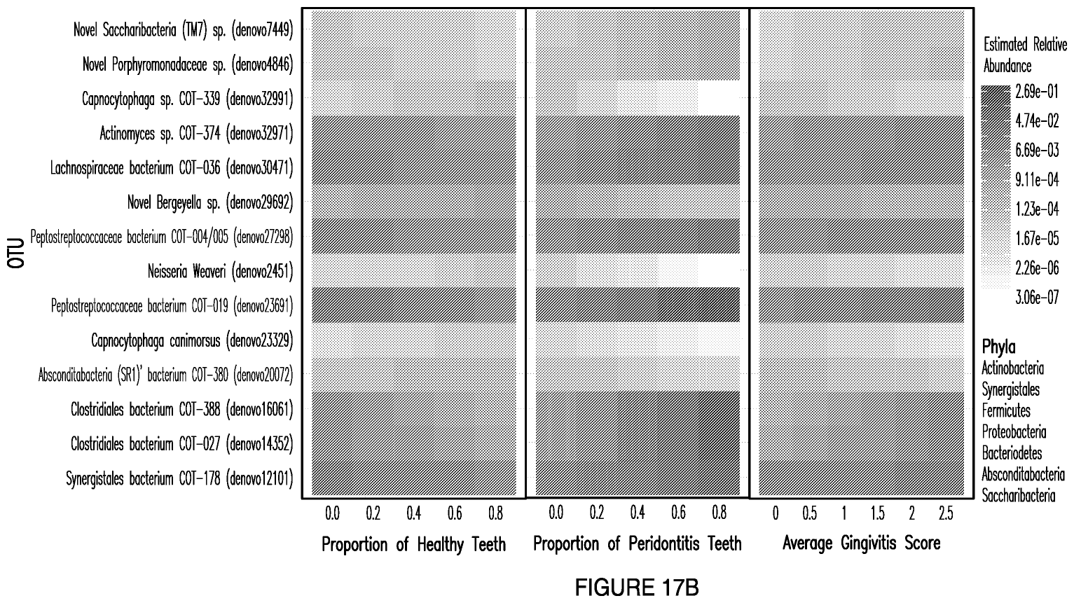

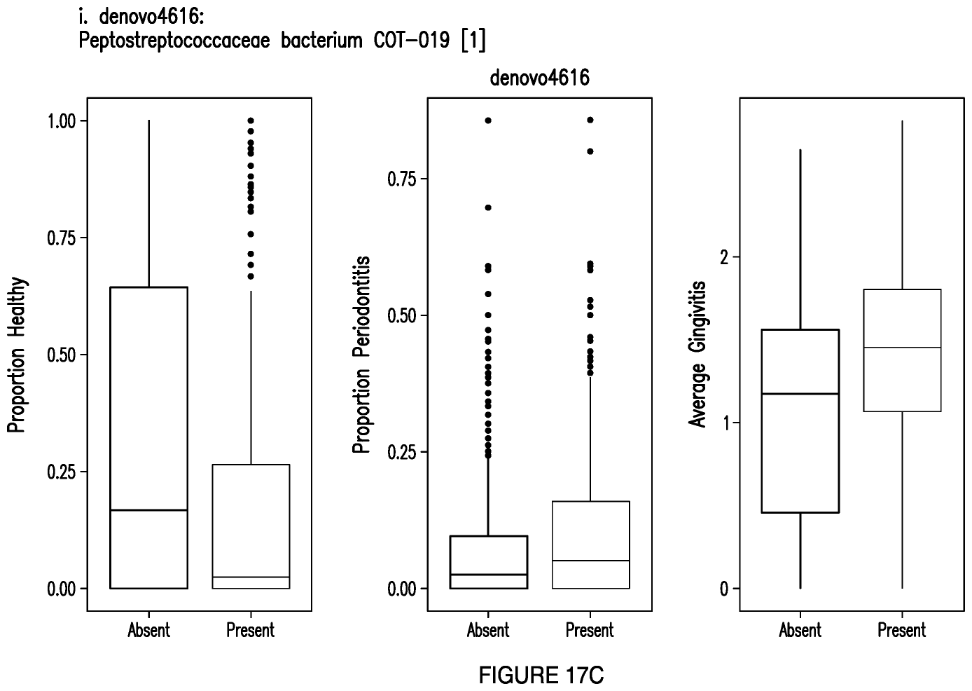

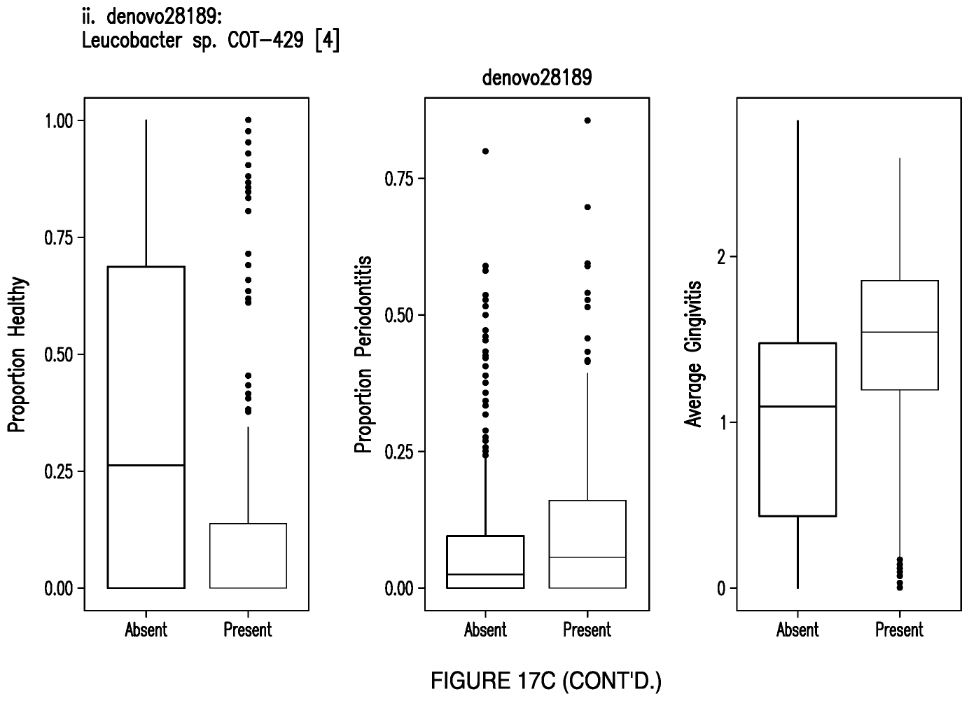

For univariate analyses, 280 of the 627 OTUs were excluded for demonstrating >80% zero sequence counts, as described in the Methods. Of the remaining 347 OTUs, 16 indicated a significant health association (p<0.05) for all three clinical assessment-based measures (proportion of healthy teeth—PHT, proportion of periodontitis teeth—PPT and average gingivitis score—AGS) and no significant effect of the plaque locations (p>0.05) (FIG. 17A). These comprised five taxa previously shown to be associated with health, and nine taxa found to be associated with disease (FIG. 17B). Another two taxa, namely Peptostreptococcaceae bacterium COT-019 (OTU #4616) and Leucobacter sp. COT-429 017 (OTU #28189), were designated solely disease associated; in each instance, the absence of the OTU coincided with a higher score for PHT (FIG. 17C).

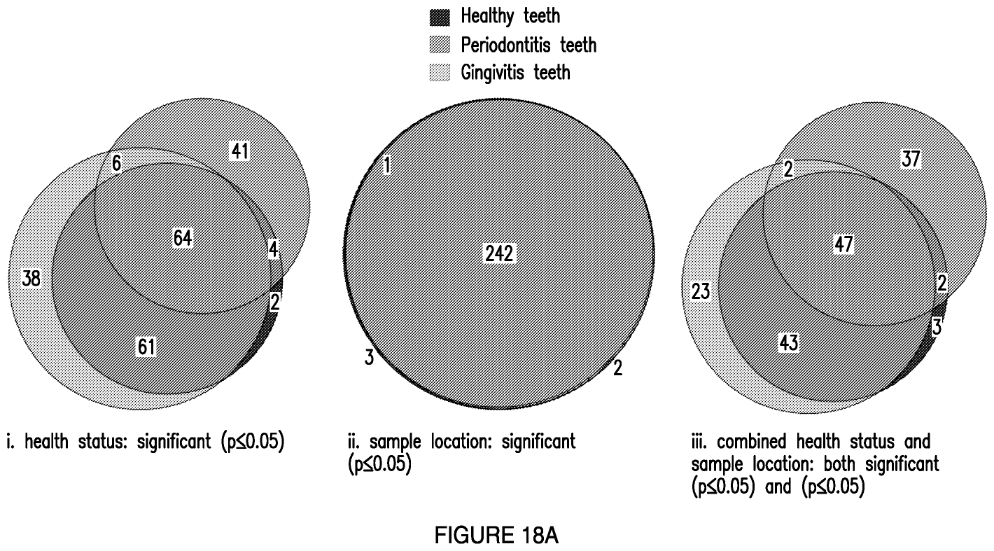

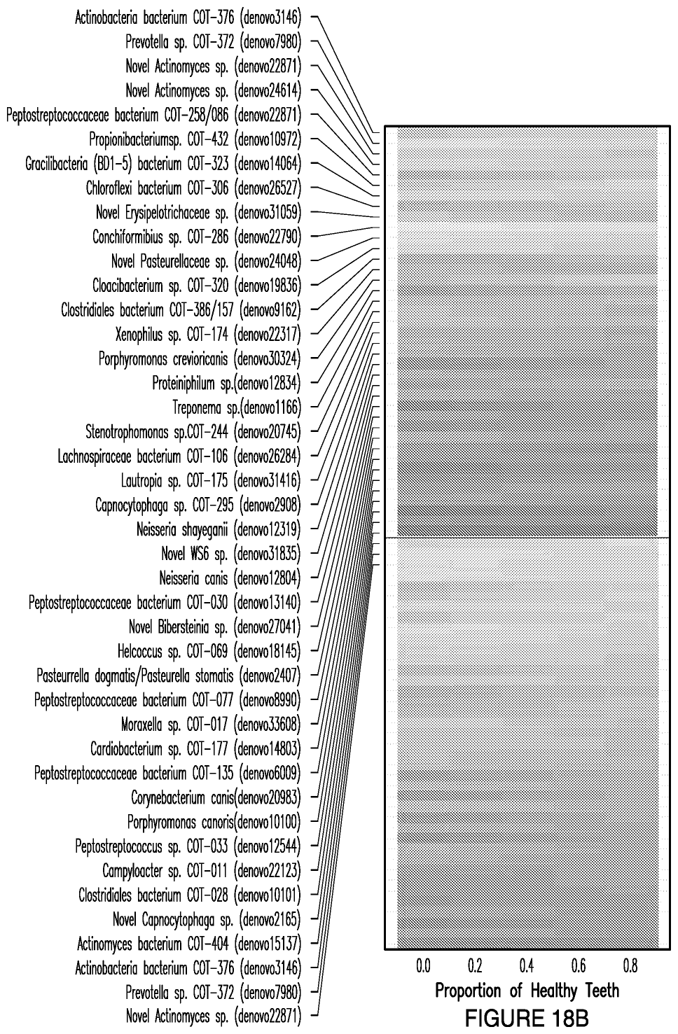

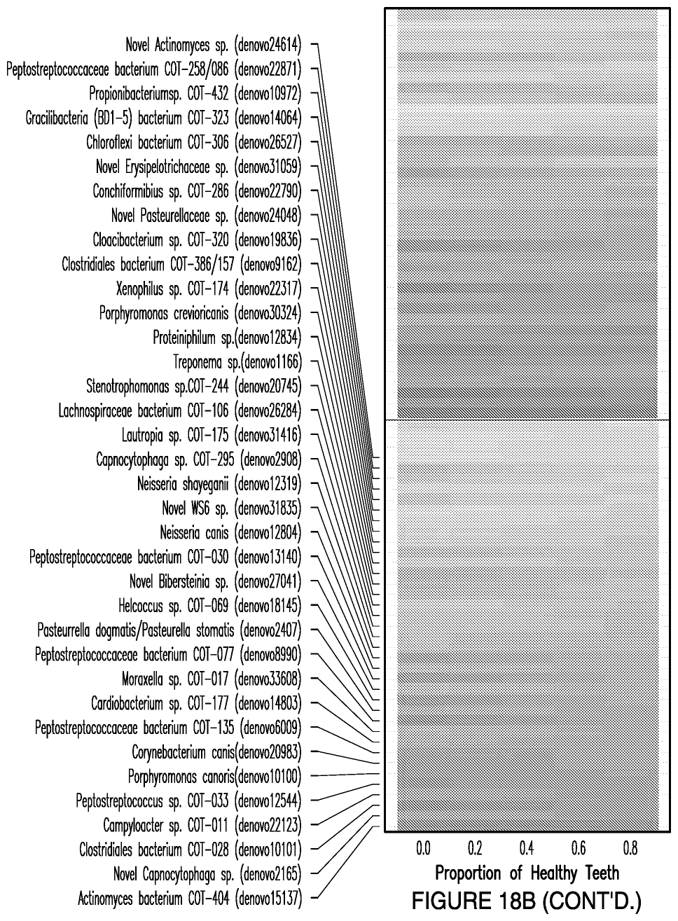

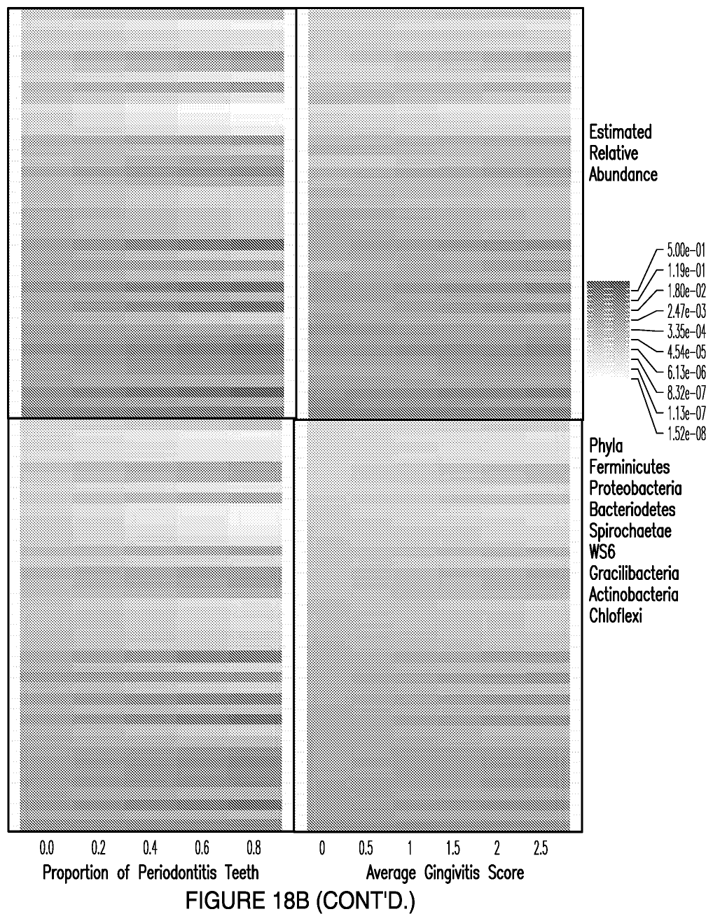

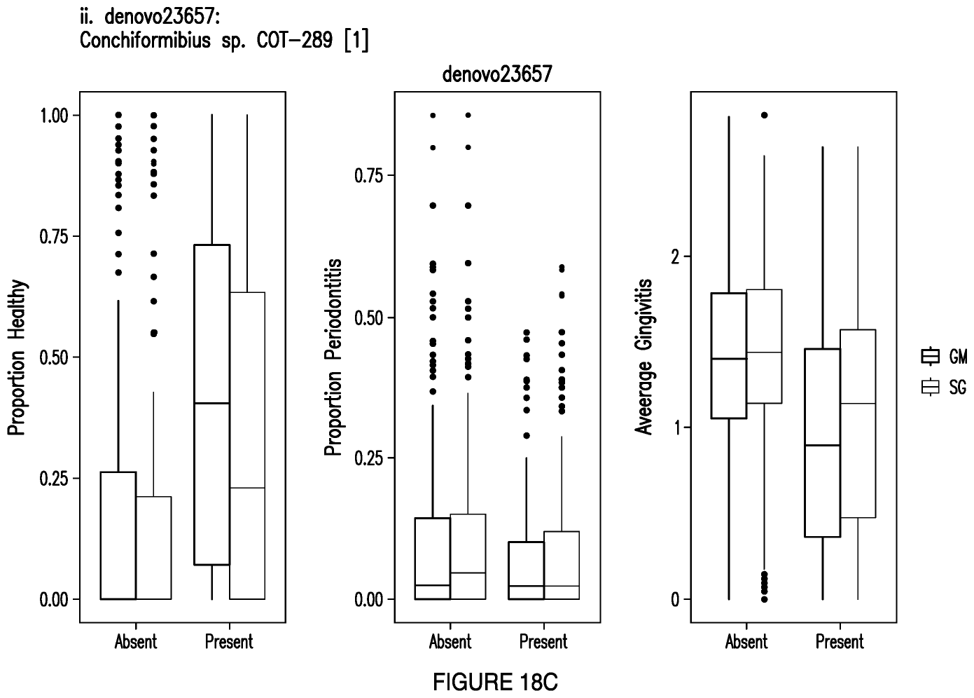

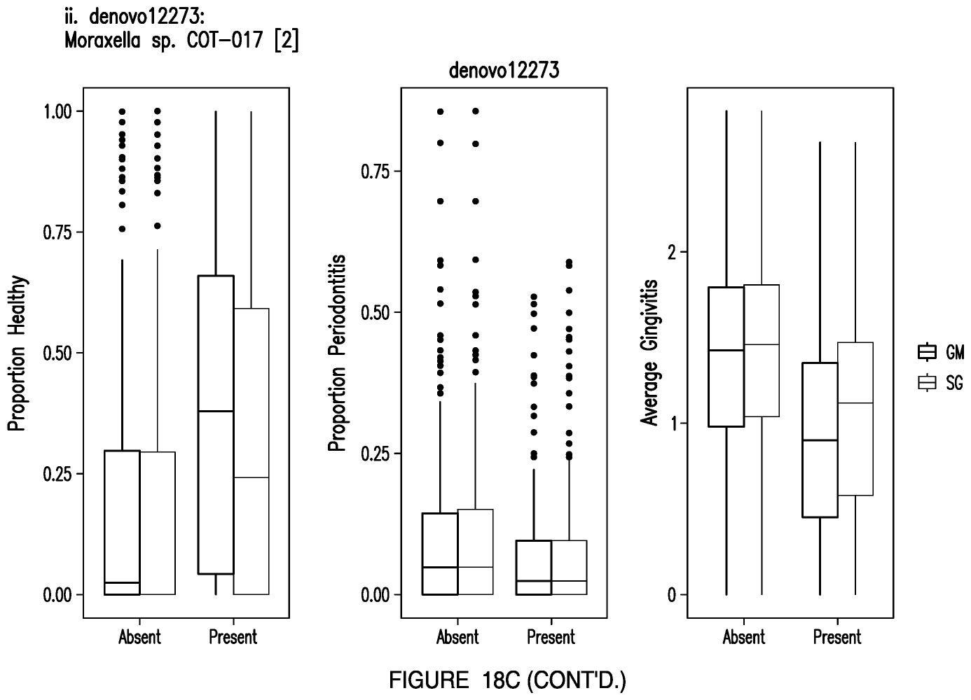

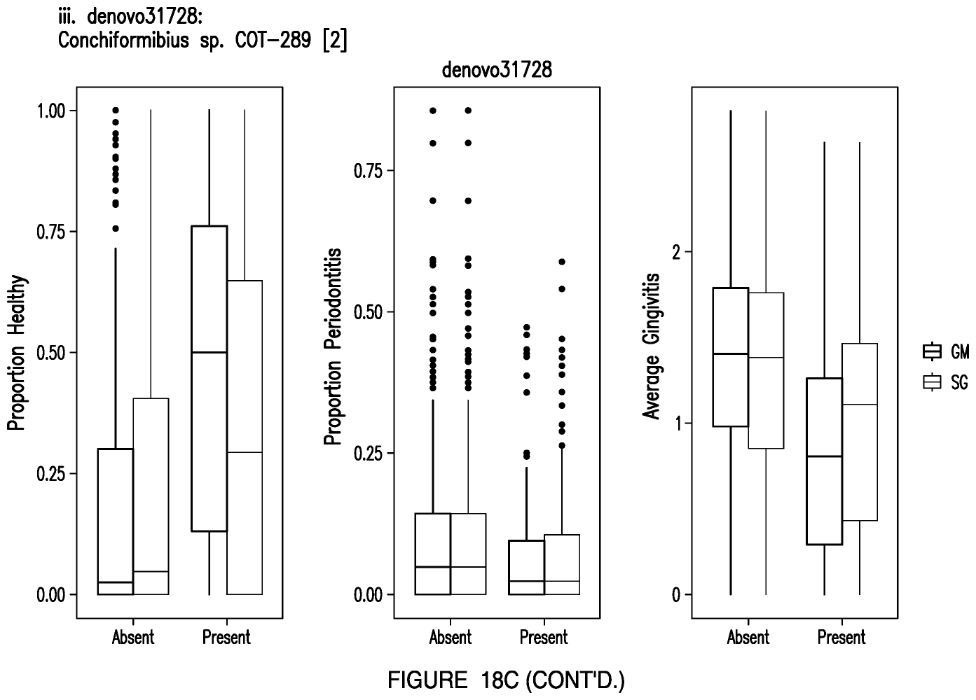

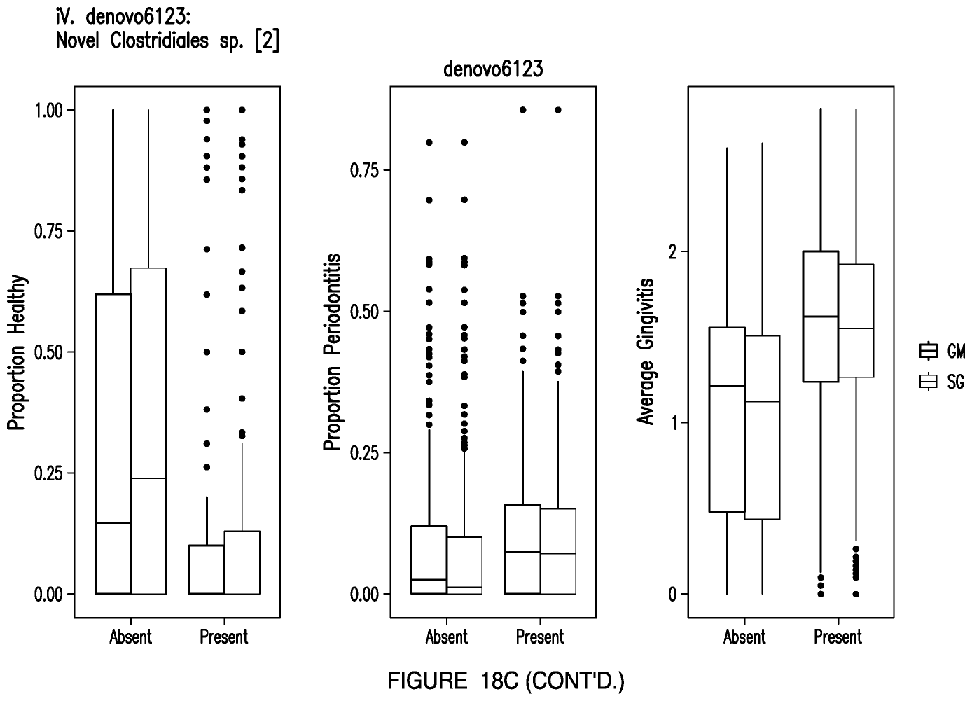

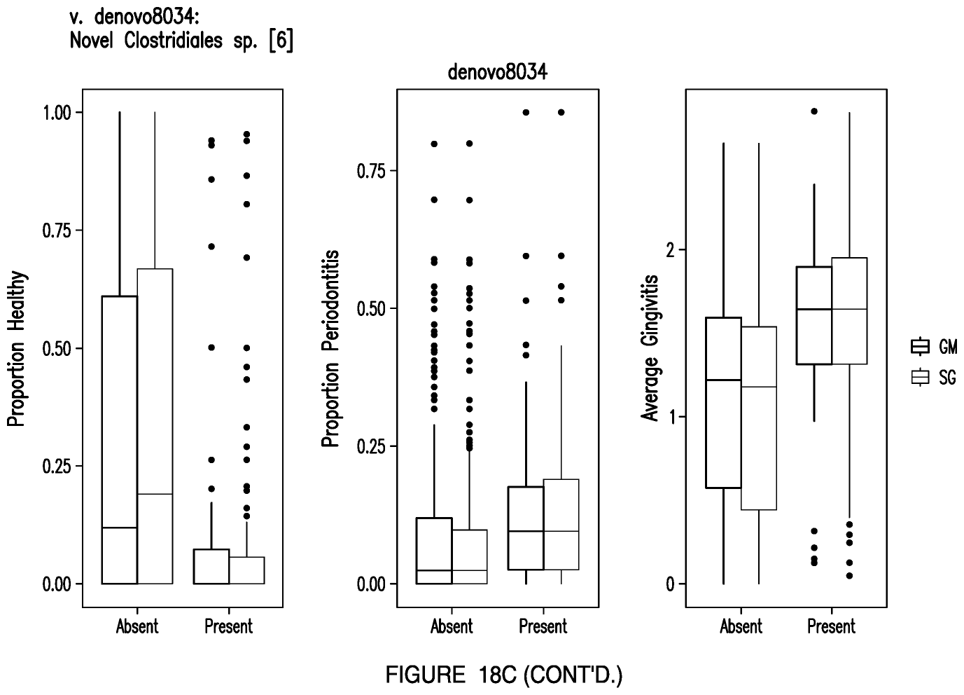

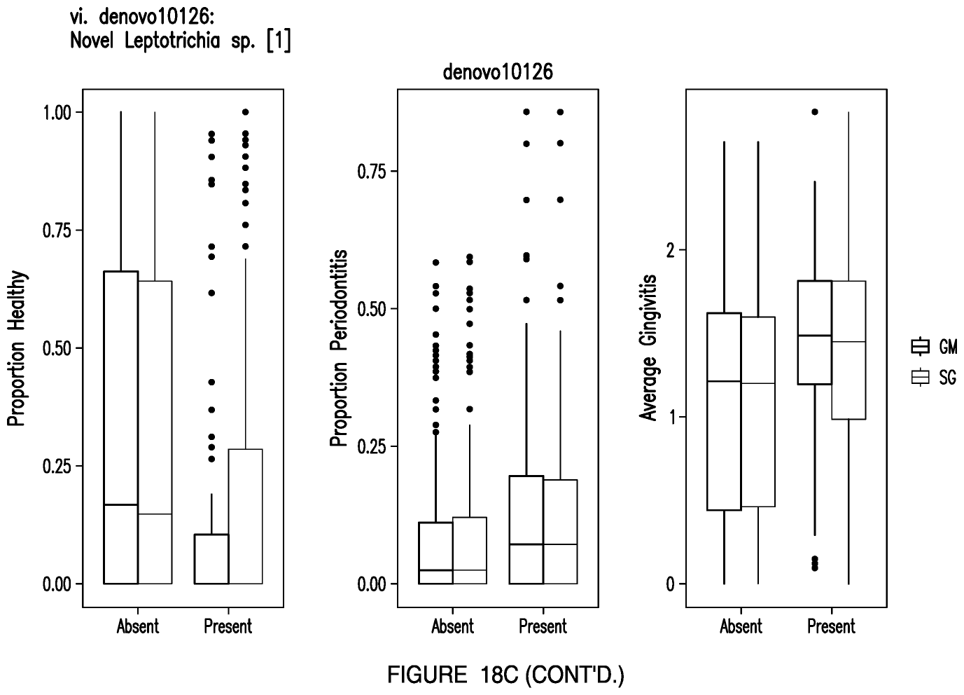

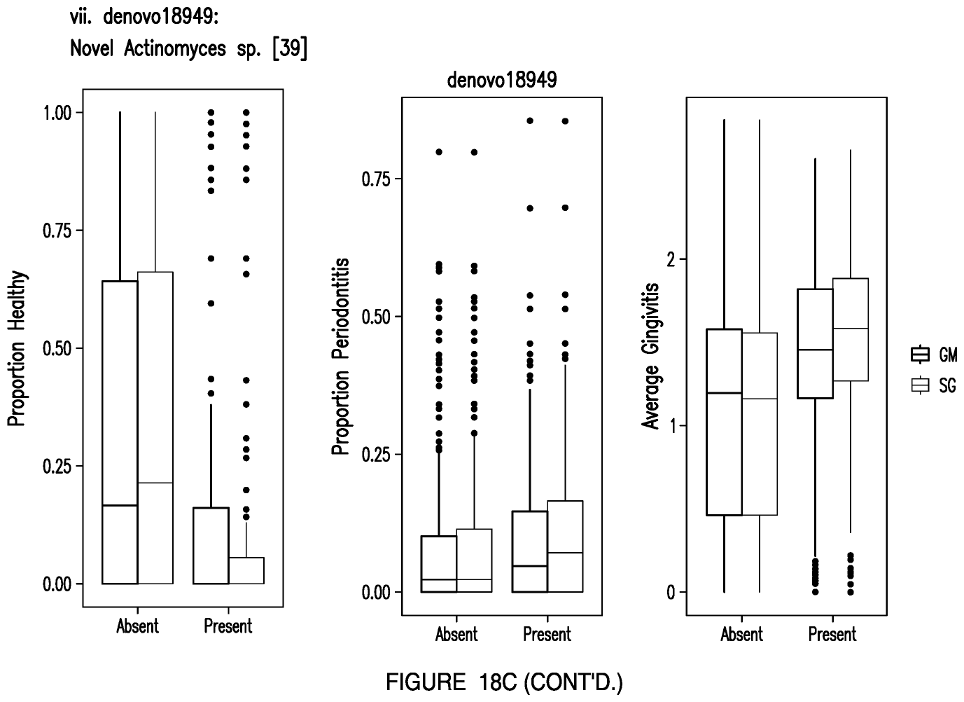

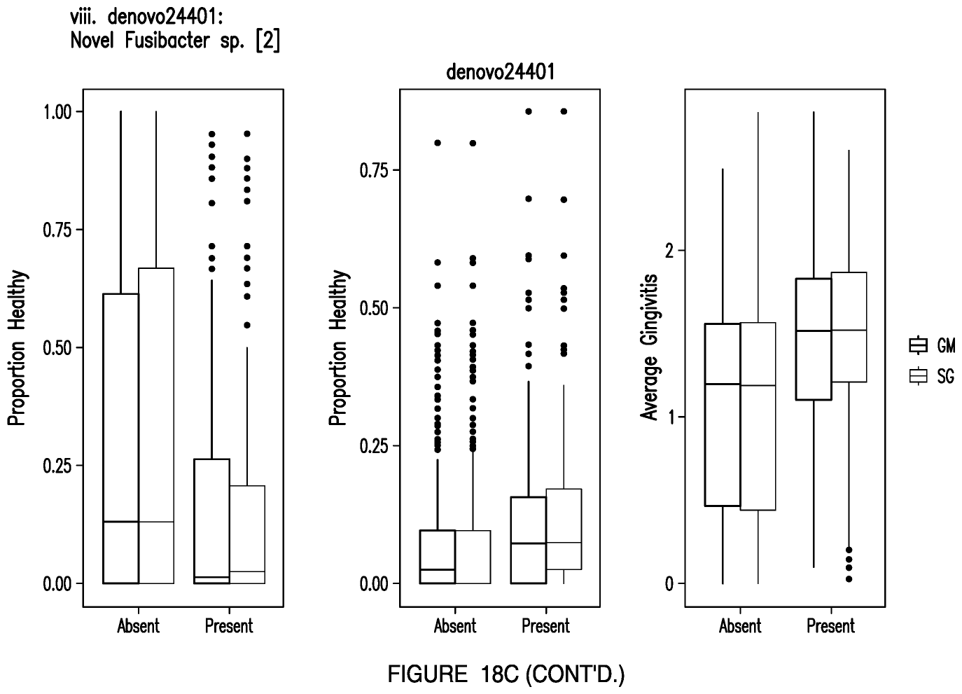

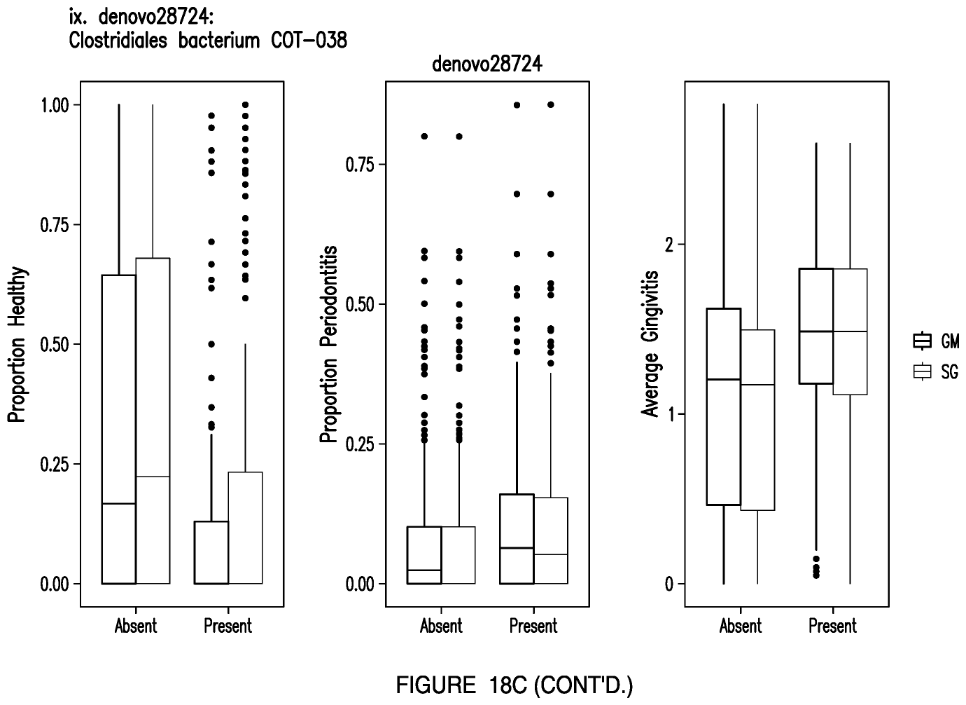

Another 47 OTUs were identified which indicated a significant health status association (p≤0.05) and also a significant effect between SG and GM plaque (p≤0.05) (FIG. 18A). Of these, 24 OTUs were significantly associated with health (FIG. 18B). Thirteen OTUs were more abundant in GM plaque compared to SG plaque; of these, five indicated changes >2-fold. Another 8 OTUs demonstrated higher abundance in SG plaque than GM plaque, of which four were >2-fold (FIG. 18B). The remaining 3 OTUs, namely Moraxella sp. COT-017 (OTU #12273), Conchiformibius sp. COT-289 (OTU #23657) and Conchiformibius sp. COT-289 (OTU #31728), were present more frequently in periodontal health (FIG. 18C, i-iii), a finding suggested by higher PHT and lower AGS when the OTU were present. This was consistent between the two plaque locations, although more pronounced in GM plaque which showed higher PHT and lower AGS. Twenty-three of the remaining 47 OTUs were significantly associated with disease (FIG. 18B). Of these, 10 OTUs showed higher abundance in GM plaque versus SG plaque (three>2-fold changes) and 7 OTUs were more abundant in SG than GM plaque (four>2-fold changes) (FIG. 18B). The 6 remaining OTUs were shown to be absent more frequently in health, present, and associated more often with disease (FIG. 18C, iv.-ix.). These indications were consistently demonstrated by higher median scores when absent compared to present for the PHT, and higher overall median scores for presence with AGS and PPT. These findings were also broadly comparable between SG and GM plaque.

### Age and Breed Size Associations

Of the remaining 347 OTUs, 16 indicated a significant age association (p<0.05) for clinical assessment-based measures on gingivitis. These comprised five taxa which showed a consistent significant relationship with age across both sampling locations and 11 taxa which showed a consistent significant relationship with age within one plaque site but not the other. Another 15 OTUs were shown to have a significant age association (p<0.05) for clinical assessment-based measures on periodontitis. Of these 15, six taxa which showed a consistent significant relationship with age across both sampling locations and 11 taxa which showed a consistent significant relationship with age within one plaque site but not the other.

Similar associations were considered for breed size. Ten OTUs were identified which had a significant age association with gingivitis (p<0.05). Of these, four showed a consistent significant relationship with breed size across both sampling locations, two consistent significant relationship with breed size within both sampling locations but in different directions, and the remaining four a consistent significant relationship with age within one plaque site but not the other. Another 6 showed a significant breed association with periodontitis. These were distributed across the same categories as listed for breed size with gingivitis within the ratio of 2:1:3.

### Discussion

Investigations into the bacterial associations with canine periodontal disease are typically characterized by the sampling of SG plaque. Whilst this represents the perfect candidate from a theoretical perspective, there are a number of drawbacks; these are centered around collector training requirements, the complexity of access, and possible ethics and animal welfare accompanying the use of general anesthesia. The incentives for overcoming such limitations go beyond the rationale discussed here; possibilities to diversify study designs to allow microbiota monitoring over short or regular timescales in the same animal and sample utilization for diagnostic purposes represent just a couple of examples. GM plaque, available supragingivally, but with close proximity to the gum line, offers an alternative plaque source that can potentially be collected from conscious dogs, trained or amenable to mouth handling. To the best of our knowledge, a comparative study of the microbiota between SG and GM plaque in dogs had not previously been performed. The investigation revealed the broad similarity in the microbiota between the two plaque sites sampled. However, a number of differences were also shown, driven by health associations, indicating that while there is good alignment, SG and GM plaque are not identical from a microbial perspective.

The analysis of the SG plaque samples has been reported previously as part of a large-scale cross-sectional study of dogs with healthy gingiva and early periodontal disease across four geographical locations [44]. Remnant samples from the UK-based subset of this investigation were of insufficient volume, hence not considered here. The remaining 772 samples were collected from a broad range of dogs across three geographies. The associated metadata for the study cohort was included in the evaluation given the influence of numerous genetic and environmental factors in the development of periodontal disease. This indicated that the subset sampled in Thailand predominantly comprised medium-sized breeds, and was significantly younger than the subsets in the USA and China, where small breeds were much more frequently sampled. Harvey et al. found many parameters associated with periodontal disease including gingival inflammation and attachment loss, to be more common in smaller and older dogs among a cohort of 350 dogs (Harvey et al. 1994). Analysis of medical records spanning five years across 100 breeds of dog also found risk factors for periodontal disease to be influenced by breed size, weight, and age [43]. Further to that, a study by Marshall et al. highlighted the higher susceptibility and progression rate of periodontal disease in the miniature schnauzer breed, an observation which was also more pronounced in older dogs [39]. Unfortunately, the study presented here was not able to reveal breed-specific insights; this was due to a lack of representation in the numbers of individual breeds. However, the spectrum of breeds achieved spanning the full cohort does strengthen the SG versus GM microbiota investigation, the primary objective of the study. The number of dogs recruited to this study has also enabled the delivery of association insights between specific microbial taxa, health state, and either age or breed size. These are key fundamental insights, not only refining the specificity of microbial associations linked to periodontal health and/or disease but opening new potential opportunities to evolve and optimize approaches to canine periodontal disease in the future.

This study utilized Illumina MiSeq sequencing technology. Many previous clinical investigations have adopted 454-pyrosequencing, and it is important to appreciate those platform variations, such as primers and sequencing chemistry, will influence the output microbes detected. Despite the difference, the overall phyla composition was found to be similar. Some of the historic studies include the cross-sectional studies performed by Wallis et al. [44], who analyzed only the SG sample subset considered here, and Davis et al. [20], who investigated a SG, UK based sample subset. Similar findings were also shown in other canine oral-microbiota focused research publications [49, 50, 51]. The discriminatory phylogenetic analysis between the SG and GM plaque sites in this study showed similarity at the phylum level.

In previous investigations, both phylum and genus level associations with clinical health status have been identified [20]. The data analyzed from SG and GM plaque in this work conform to these earlier findings. For example, Firmicutes, including several species of Actinomyces and Peptostreptococcaceae, were highly abundant amongst the various disease associated taxa identified [20]. Additionally, the health associates, Bacteriodetes were abundant in both the SG and GM plaque sites, and while numerous Proteobacteria were evident, the relationship between these and plaque sample sites was found to significantly differ [20].

Multivariate parameters measuring microbial variability and diversity indicated differences between the two plaque sites. SG plaque samples displayed greater variability and significantly higher diversity compared to the GM sample cohort. This is most likely attributed to a number of environmental factors. Physiologically, the anatomy of the SG site creates a far more anaerobic atmosphere when compared to the GM one [52]; this is undoubtedly a major driver of the difference in the microbiota flourishing between the plaque niches. In addition to the age and breed size aspects of the metadata discussed earlier, differences in feeding behavior would be anticipated to contribute to the variability observed, given the spread of the study cohort across three geographical locations; this theory is consistent with the PCA analysis. This study was unable to explore diet information in-depth, but broad insights were ascertained. While the majority of pet owners in these regions fed commercially available pet foods, with a preference towards dry diets that is consistent with literature-based insights [53, 54, 55], there were regional differences. For example, the Asian countries additionally indicated trends towards the feeding of home-prepared diets, which could represent table scraps as opposed to dedicated offerings.

Statistical modeling combined with the evaluation of three key clinical parameters allowed for specific OTUs to be discriminated by health association across both the SB and GM samples. Many of the taxonomic assignments and associated health statuses defined in this study were found to be comparable with current research findings regarding canine oral microbiota [20], [51]. Several of the health associated bacterial taxa identified have been hypothesized to play a fundamental role in early canine plaque biofilm formation [49]. Suggested primary colonizers identified here include Stenotrophomonas sp. COT-224 (OTU #20745) and three species of the genus Neisseria (OTU #s 2415, 12319 and 12804) as well as potential subsequent joiners such as Moraxella sp. COT-017 (OTU #33608) and Actinomyces species (OTU #s 22817 and 24614). Bacterial OTUs found to have no significant effect on the plaque niches were Capnocytophaga sp. COT-339, and disease associated Actinomyces sp. COT-374; these were amongst the most abundant OTUs reported by Davis et al. [20]. Throughout this study, there was additionally good genus-level alignment for the health associated genera Porphyromonas, Moraxella, and Bergeyella, and the disease associated Peptostreptococcus, Actinomyces, and Peptostreptococcaceae, which Davis et al. [20] concluded to predominate. The consistent identification of certain species associated with clinical health and disease affords opportunities to develop microbial biomarkers as diagnostics for canine gum disease.

This study generated 627 OTUs; statistically significant differences were not identified for the majority of these, broadly highlighting the comparability of microbiota between SG and GM plaque. Furthermore, there was consistency across the three different geographical locations considered for this investigation. Although previously unexplored for the canine model, we believe the insights generated here align closely with the research findings gained in the human field. It is important to clarify that investigations based on the human model have focused on the level of parity between SG and supragingival plaque, rather than the microbiota specifically residing at the GM, considered here. Despite this, and the variation in the explorative technologies, many parallels are still obvious. Several reports comprising healthy and/or disease cohorts analyzed via polymerase chain reaction or checkerboard DNA-DNA hybridization (CKB) technique have observed well correlated microbial profiles between SG and supragingival plaque [56. 57. 58. 59]. 16S rRNA sequencing of biofilms from inflamed peri-implant and periodontal sites in the same seven subjects has also demonstrated no significant differences between the associated microbiomes in SG and supragingival plaque derived biofilms [60], thereby illustrating some level of consistency to the insights gained here. Furthermore, Daniluk et al. [61] have shown the predominance of both anaerobes in SG and aerobes in supragingival plaque. Equivalent insights are evident elsewhere, demonstrating the transition in the abundance of biofilm based-microbes relative to oxygen requirements between the close proximity locations [62, 63]. Such studies have adopted fair sized cohorts (n=185, n=158) and CKB for assessment. In parallel with what has been identified in this study, there are some exceptions to this. For example, He et al. [64] characterized levels of four periodontopathogenic bacteria in 84 Chinese patients using quantitative real-time polymerase chain reaction (qRT-PCR) and found consistency in the frequency of detection across saliva, SG, and supragingival plaque, with the exception being Aggregatibacter actinomycetemcomitans. In CKB-led investigations, differential proportions of certain species have been observed, between the plaque samples including those of Actinomyces [65, 66]. Such variations, however, do not disprove the widely accepted notion that subgingival plaque acts as a reservoir for the resulting SG microbiota [65, 66]. Lastly, Gallimans et al. explored supragingival and tongue dorsum sites as alternatives to SG plaque for bacterial biomarkers of chronic periodontitis [67]. Using 24 subjects and Illumina sequencing, the authors found most OTUs were shared between periodontal health and disease, with a relatively small proportion of OTUs distinct to disease [67]. Similar to the study presented here which additionally identified a handful of OTUs more niche to periodontal health, the consistency in the bulk of the health status associated findings support the use of alternative plaque locations for the bacterial biomarkers for diagnostic monitoring. Evaluations conducted using large cohort sizes in tandem with next generation sequencing approaches could not be identified. We believe such factors add invaluable merit to the study presented here.

The research insights presented have the potential to provide valuable support in the monitoring of canine periodontal disease. The spectrum of periodontal disease risk that aligns with breed and breed size categories has already been discussed [68]. Risk awareness can therefore not only influence but prescribe the frequency of assessments undertaken for a given dog. More regular checks for breeds with higher susceptibility could be undertaken in the veterinary setting with conscious animals. Not only would this prospect reduce the potential number of exposures to anesthesia for a given animal, it can increase the diagnosis of potential underlying disease and also support ongoing conversations with pet owners about the importance of good oral care.

### Example 4—Predicting Age of a Canid from the Relative Abundance of Bacterial Taxa

A dataset of OTU counts, collected from 577 dogs were considered as the inputs for a prediction model. The dogs spanned a range of ages (0.8 to 15 years of age), locations (China, Thailand, US, UK) and breeds (100+ breeds total). The inputs for the prediction model were 106 OTUs, counts of which were collected for each dog (subgingival plaque), that had previously been found to have a significant association with age.

The OTUs were converted to relative abundances by dividing through each observation by the total count of all OTUs found in the animal (from 138 OTU categories plus rares).

### XGBoost Algorithm

An XGBoost algorithm was used to train a prediction model. XGBoost is a well-known implementation of gradient-boosting which typically achieves extremely high accuracy in prediction-based tasks.

⅕ of the samples (or 115 dogs) were randomly selected to be held out from the training period and their ages used to test the predictions of the trained model. The remaining ⅘ (or 462 dogs) were used to train the model. The training period consisted of 10-fold cross-validation being performed on the data under various hyperparameter sets, followed by the training of the model using the hyperparameter set that resulted in the lowest test-set error from the cross-validation stage. The hyperparameters and the tuned values are listed below:
Eta (known as the “learning rate)=0.15
Max.depth (the maximum number of nodes in a constituent decision tree in the model)=2
Nround (the number of rounds the model takes to achieve the best fit)=22

### Prediction Accuracy and Important Features

The prediction accuracy achieved on the test set by the model was:
Root Mean Squared Error (RMSE)=2.83 years
Mean Absolute Error (MAE)=2.24 years
Median Absolute Error (MDAE)=1.86 years

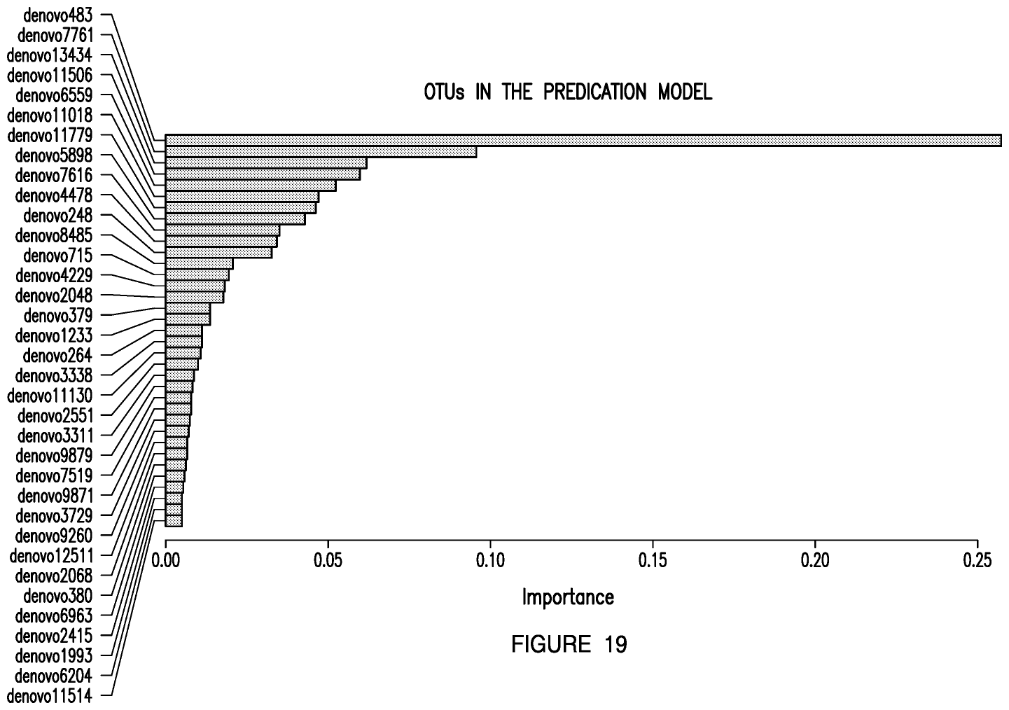

The 10 most important OTUs and their importance are shown below in Table 6. The bar plot in FIG. 19 shows the importance of all OTUs retained by the fitted model.

TABLE 6
Sample of OTUs and their importance

| OTU | Importance |
|---|---|
| denovo483 | 0.257 |
| denovo7761 | 0.095 |
| denovo13434 | 0.061 |
| denovo11506 | 0.059 |
| denovo6559 | 0.052 |
| denovo11018 | 0.046 |
| denovo11779 | 0.045 |
| denovo5898 | 0.042 |
| denovo7616 | 0.035 |
| denovo4478 | 0.034 |

### Including Breed Size in the Model

Further to the above, a second iteration of the model was built including the breed size of the animal (Small, Medium or Large) as an additional potential predictor. The breakdown of the breed sizes in the sample was: 167 Small, 219 Medium, 187 Large, 4 Unknown.

The breed size was encoded in one-hot vectors for each of the three sizes to accommodate the information in the XGBoost model. The method of training for the model (including 10-fold cross validation and hyperparameter tuning) was the same.

To accommodate the inclusion of extra variables, the max.depth of the tree was increased to 5. The tuned hyperparameters were as follows:
Eta=0.15
Max.depth=5
Nround (the number of rounds the model takes to achieve the best fit)=32

### Prediction Accuracy and Important Features

The prediction accuracy achieved on the test set by the model was:
Root Mean Squared Error (RMSE)=2.81 years
Mean Absolute Error (MAE)=2.23 years
Median Absolute Error (MDAE)=1.85 years

The prediction accuracy results of the model with the inclusion of breed size indicate an improvement over the results in the first section.

The 10 most important OTUs are again shown below in Table 7. The most important OTUs include many seen in the previous top 10, but the importances are all reduced, primarily due to the inclusion of more variables in the model as a result of the increased max.depth.

OTU Importance denovo483 0.223 denovo7761 0.049 denovo5898 0.038 denovo13434 0.030 denovo248 0.028 denovo11018 0.026 denovo2415 0.025 denovo11506 0.024 denovo264 0.022 denovo715 0.022

### Table 7—Sample of OTUs and their Importance

### Example 5—Relationship Between the Relative Abundance of OTU #7791 and Age

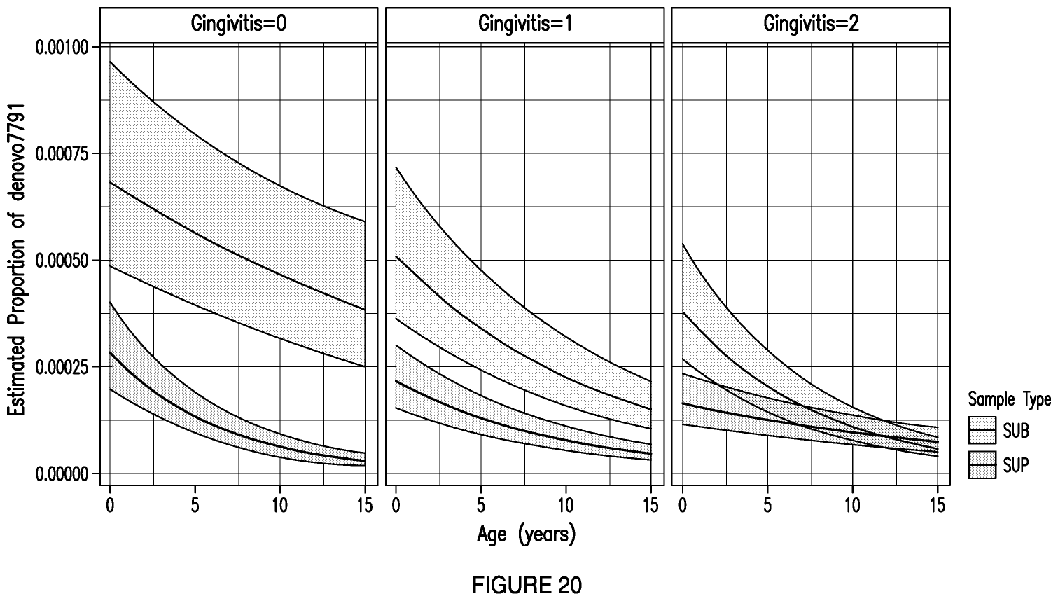

Example 5 describes the relationship between the amount (e.g., relative abundance) of OTU #7791 that is present in the mouth of a canid and its age. FIG. 20 illustrates an example of the relationship between age and the relative abundance of OTU #7791 by location in the mouth (e.g., subgingival or supragingival). Table 8 is an example of the trends between OTU #7791 and age.

TABLE 8
Trends between OTU #7791 and age; GM: gingival margin; SG: subgingival.

| | | | | Mouth | | BH |
|---|---|---|---|---|---|---|
| | Assigned | Assigned | Sample | Gingivitis | Odds | adjusted |
| OTU# | Phylum | Taxonomy | Type | Score(s) | Ratio(s) | p-value(s) |
| denovo7791 | Firmicutes | Lachnospiraceae | GM | 0, 1, 2 | 0.86, | <0.001, |
| | | bacterium COT- | | | 0.903, | <0.001, |
| | | 263 | | | 0.947 [—] | <0.001 |
| denovo7791 | Firmicutes | Lachnospiraceae | SG | 0, 1, 2 | 0.962, | 0.003, |
| | | bacterium COT- | | | 0.922, | <0.001, |
| | | 263 | | | 0.883 [—] | <0.001 |

The relative abundance of OTU #7791 shows a consistent significant relationship with age across both sampling locations (SUP and SUB).

Relative abundance of OTU #7791 significantly decreases with age in gingival margin (SUP) and subgingival (SUB) plaque in dogs with healthy gums/gingiva (G0), very mild (G1) and mild (G2) gingivitis. Relative abundance indicates that OUTUT #7791 is present in quantities of 0 to 0.001 compared with all bacteria present in the canid (i.e., maximum of 1.0). The data reflect that the SG and GM follow the same trend in G0, G1 and G2.

Generally, OTU abundance is higher in supragingival (SUB) compared to gingival margin (SUP) plaque.

### Example 6—Relationship Between the Relative Abundance of OTU #28682 and Breed Size

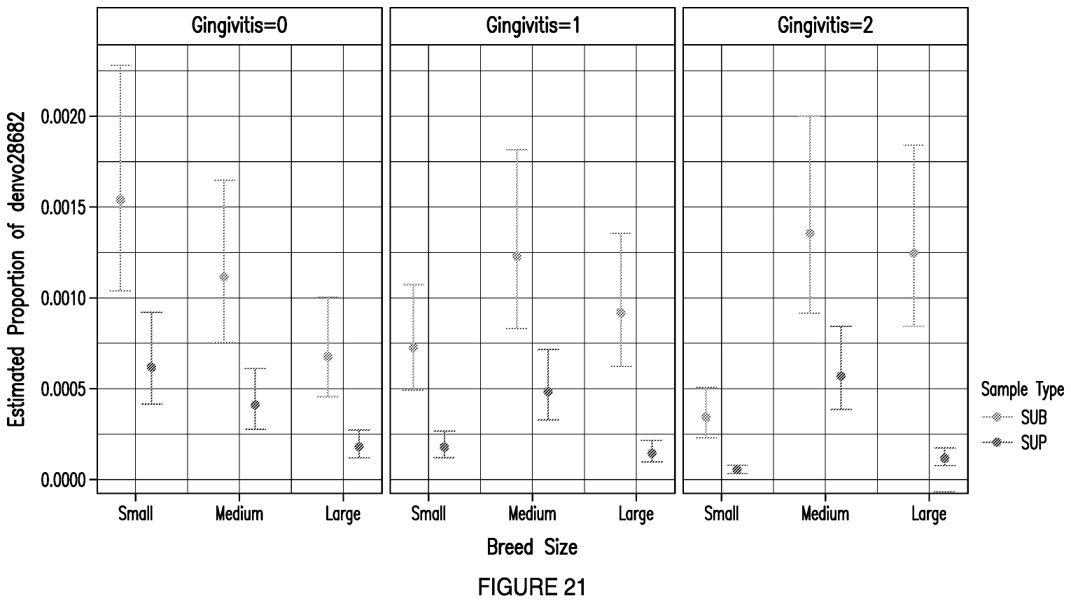

Example 6 describes the relationship between the amount (e.g., relative abundance) of OTU #28682 that is present in the mouth of a canid and its breed size. FIG. 21 illustrates an example of the relationship between breed size and the relative abundance of OTU #28682 by location in the mouth (e.g., subgingival or supragingival). Table 9 is an example of trends between OTU #28682 and breed size.

TABLE 9
Trends between OTU #28682 and breed size

| | | | | | | | BH |
|---|---|---|---|---|---|---|---|
| | | | | Mouth | | | adjusted |
| | Assigned | Assigned | Sample | Gingivitis | Breed | Odds | p- |
| OTU# | Phylum | Taxonomy | Type | Score(s) | Size | Ratio(s) | value(s) |
| denovo28682 | Spirochaetae | Treponema | GM | 0, 1, 2 | Med- | 0.666, | <0.001, |
| | | sp. COT- | | | Small | 2.69, | <0.001, |
| | | 359 | | | | 10.9 | <0.001 |
| denovo28682 | Spirochaetae | Treponema | GM | 0, 1, 2 | Large- | 0.295, | <0.001, |
| | | sp. COT- | | | Small | 0.811, | <0.001, |
| | | 359 | | | | 2.23 | <0.001 |
| denovo28682 | Spirochaetae | Treponema | GM | 0, 1, 2 | Large- | 0.442, | <0.001, |
| | | sp. COT- | | | Med | 0.301, | <0.001, |
| | | 359 | | | | 0.205 | <0.001 |
| denovo28682 | Spirochaetae | Treponema | SG | 0, 1, 2 | Med- | 0.704, | <0.001, |
| | | sp. COT- | | | Small | 1.69, | <0.001, |
| | | 359 | | | | 3.95 | <0.001 |
| denovo28682 | Spirochaetae | Treponema | SG | 0, 1, 2 | Large- | 0.439, | <0.001, |
| | | sp. COT- | | | Small | 1.26, | <0.001, |
| | | 359 | | | | 3.63 | <0.001 |
| denovo28682 | Spirochaetae | Treponema | SG | 0, 1, 2 | Large- | 0.607, | <0.001, |
| | | sp. COT- | | | Med | 0.747, | <0.001, |
| | | 359 | | | | 0.919 | 0.008 |

The relative abundance of OTU #28682 shows a consistent significant relationship with breed size across both sampling locations (SUP and SUB).

The relative abundance of OTU #28682 is significantly lower in large breed dogs with healthy gingiva (G0) compared to small and medium breed dogs with healthy gingiva (G0) in both plaque locations (SUP and SUB).

In mild gingivitis (G2), the relative abundance of OTU #28682 is significantly higher in large breeds than small breeds in both plaque locations (SUP and SUB).

### Example 7—Relationship Between the Relative Abundance of OTU #23212 and Age

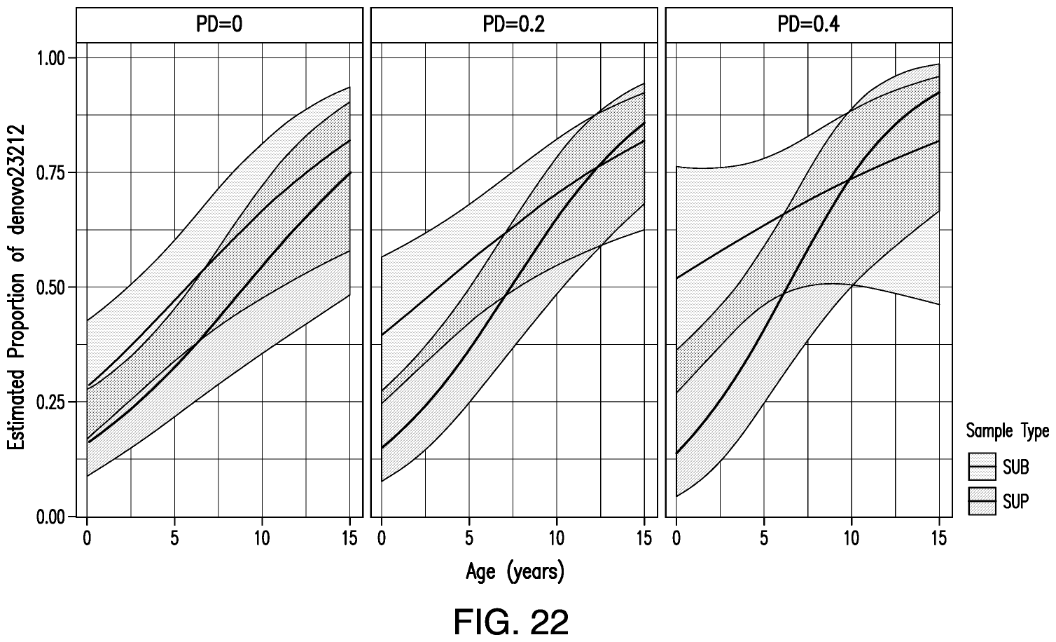

Example 7 describes the relationship between the amount (e.g., relative abundance) of OTU #23212 that is present in the mouth of a canid and its age. FIG. 22 illustrates an example of the relationship between age and the relative abundance of OTU #23212 by location in the mouth (e.g., subgingival or supragingival). Table 10 is an example of the trends between OTU #23212 and age.

TABLE 10
Trends between periodontitis and age for OTU #23212

| | | | | | | BH |
|---|---|---|---|---|---|---|
| | | | | | | adjusted |
| | Assigned | Assigned | Sample | Proportion | Odds | p- |
| OTU# | Phylum | Taxonomy | Type | of PD | Ratio(s) | value(s) |
| denovo23212 | Synergistetes | Novel | GM | 0, 0.2, 0.4 | 1.2, 1.27, | 0.006, <0.001, |
| | | Synergistetes | | | 1.33 | 0.021, |
| | | bacterium | | | | |
| denovo23212 | Synergistetes | Novel | SG | 0, 0.2, 0.4 | 1.18, 1.14, | 0.014, 0.027, |
| | | Synergistetes | | | 1.1 | 0.430 |
| | | bacterium | | | | |

The presence of OTU #23212 significantly increases with age in healthy dogs (PD=0) and those with 20% (PD=0.2) and 40% (PD=0.4) of the teeth with periodontitis in the mouth in both plaque locations (SUP and SUB).

The presence of OTU #23212 is higher in subgingival (SUB) plaque in younger dogs, but in the instance of dogs with periodontitis (PD=0.2, PD=0.4) the levels of OTU #23212 become higher in gingival margin (SUP) plaque in older dogs (e.g., on PD=0.4 plot, this occurs at approximately 10 years).

### Example 8—Relationship Between Different Bacterial Taxa OTUS and Breed Size

Table 11 is an example of the relationship between different types of bacterial taxa OTUs and the breed size of a canid. More specifically, Table 11 shows the trends between breed size and different types of bacterial OTUs based on their locations (e.g., gingival or subgingival) in the mouth of the candid. The first column identifies a bacterial taxa OTU type. The second column identifies the relationship type that is being analyzed. The third column identifies the location in the mouth where the bacterial taxa OTU is present. The fourth column identifies trends between a bacterial taxa OTU and breed size. The fifth, sixth, seventh, and eighth columns provide scoring information for the relationship between a bacterial taxa OTU and breed size. The fifth column gives a score that indicates how well a bacterial taxa OTU correlates with breed size. A higher numeric score indicates a higher correlation between a bacterial taxa OTU and breed size. The sixth column gives a score as a secondary indicator for how well a bacterial taxa OTU correlated with breed size. Once again, a higher numeric score Indicates a higher correlation between a bacterial taxa OTU and breed size.

TABLE 11
Relationship between different bacterial taxa OTUs and breed size

| | | | | | | Highest | Sum of |
|---|---|---|---|---|---|---|---|
| | | | | | | Fold | Fold |
| GM vs. SB | | | | Highest | Sum of | Change | Change |
| Insights | What | Where | Trend | Score | Scores | Scores | Scores |
| Novel | Gingivitis/ | Both | N/A | 3 | 20 | 1 | 5 |
| Flavobacterium sp. | Breed | locations | (no consistent direction | | | | |
| denovo5982 | Size | | of the trend across G0, | | | | |
| | | | 1, 2) | | | | |
| Novel | Gingivitis/ | Both | [↑] | 1 | 1 | N/A | N/A |
| Saccharibacteria | Breed | locations | (with G0, 1, 2) | | | | |
| (TM7) sp. | Size | | | | | | |
| denovo7449 | | | | | | | |
| Treponema sp. | Gingivitis/ | Both | N/A | 3 | 16 | 3 | 7 |
| COT-359 | Breed | locations | (no consistent direction | | | | |
| denovo28682 | Size | | of the trend across G0, | | | | |
| | | | 1, 2) | | | | |
| Novel WS6 sp. | Gingivitis/ | Both | [↑] | 1 | 3 | N/A | N/A |
| denovo31835 | Breed | locations | (with G0, 1, 2) | | | | |
| | Size | | | | | | |
| Parvimonas sp. | PD/ | Both | [↓] | N/A | N/A | N/A | N/A |
| COT-035 | Breed | locations | (with G0, 1, 2) | | | | |
| denovo20143 | Size | | | | | | |
| Peptostreptococcaceae | PD/ | Both | [↓] | 3 | 25 | 1 | 7 |
| bacterium COT-104 | Breed | locations | (with G0, 1, 2) | | | | |
| denovo20699 | Size | | | | | | |
| Corynebacterium | Gingivitis/ | Both | N/A | 3 | 15 | 1 | 5 |
| sp. COT-423 | Breed | locations | (no consistent direction | | | | |
| denovo5665 | Size | | of the trend across G0, | | | | |
| | | | 1, 2) | | | | |
| Novel | Gingivitis/ | Both | [↑] | 3 | 10 | 1 | 3 |
| Saccharibacteria | Breed | locations | (with G0, 1, 2) | | | | |
| (TM7) sp. | Size | | | | | | |
| denovo24699 | | | | | | | |
| Lachnospiraceae | Gingivitis/ | GM | N/A | 3 (2 GM | 10 (4 | 1 (SG | 1 (SG |
| bacterium COT-062 | Breed | | (no consistent direction | count | GM | count) | count) |
| denovo12563 | Size | | of the trend across G0, | only) | count | | |
| | | | 1, 2) | | only) | | |
| Gracilibacteria | Gingivitis/ | SG | N/A | 3 (2 SB | 17 (7 SB | 2 (1 SG | 4 (2 SG |
| bacterium COT-323 | Breed | | (no consistent direction | count | count | count | count |
| denovo14064 | Size | | of the trend across G0, | only) | only) | only) | only) |
| | | | 1, 2) | | | | |
| Peptostreptococcaceae | Gingivitis/ | SG/GM | [↑] | 3 | 20 | 3 | 8 |
| bacterium COT-096 | Breed | | [as gingivitis] | | | | |
| denovo30311 | Size | | | | | | |
| Saccharibacteria | Gingivitis/ | GM | [↓] | 2 | 11 | 1 | |
| (TM7) sp. COT-237 | Breed | | [as gingivitis] | | | | |
| denovo31301 | Size | | | | | | |
| Xenophilus sp. | PD/ | Both | N/A | 3 | 13 | 1 | 3 |
| COT-174 | Breed | locations | (no consistent direction | | | | |
| denovo22317 | Size | | of the trend across G0, | | | | |
| | | | 1, 2) | | | | |
| Peptostreptococcaceae | PD/ | GM | [↓] | 3 (2 GM | 24 (12 | 1 | 7 (4 GM |
| bacterium COT-104 | Breed | | (with G0, 1, 2) | score | GM | | count |
| denovo20699 | Size | | | only) | score | | only) |
| | | | | | only) | | |
| Treponema sp. | PD/ | GM | N/A | 3 (2 GM | 16 (5 | 2 (1 GM | 5 (1 GM |
| COT-359 | Breed | | (no consistent direction | score | GM | count | count |
| denovo28682 | Size | | of the trend across G0, | only) | score | only) | only) |
| | | | 1, 2) | | only) | | |
| Porphyromonas | PD/ | GM | N/A | 2 (1 GM | 10 (1 | 1 (0 GM | 1 (0 GM |
| sp. COT-239 | Breed | | (no consistent direction | score | GM | count | count |
| denovo33199 | Size | | of the trend across G0, | only) | score | only) | only) |
| | | | 1, 2) | | only) | | |

### Example 9—Relationship Between Different Bacterial Taxa OTUs and Age

Table 12 is an example of the relationship between different types of bacterial taxa OTUs and the age of a canid. More specifically, Table 12 shows the trends between age and different types of bacterial OTUs based on their locations (e.g., gingival or subgingival) in the mouth of the candid.

The first column identifies a bacterial taxa OTU type. The second column identifies the relationship type that is being analyzed. The third column identifies the location in the mouth where the bacterial taxa OTU is present. The fourth column identifies trends between a bacterial taxa OTU and age. The fifth and sixth columns provide scoring information for the relationship between a bacterial taxa OTU and age. The fifth column gives a score that indicates how well a bacterial taxa OTU correlates with age. A higher numeric score indicates a higher correlation between a bacterial taxa OTU and age. The sixth column gives a score as a secondary indicator for how well a bacterial taxa OTU correlated with age. Once again, a higher numeric score indicates a higher correlation between a bacterial taxa OTU and age.

TABLE 12
Relationship between different types of bacterial taxa OTUs and age

| | | | | Highest | Sum of |
|---|---|---|---|---|---|
| | | | | Prevalence | Prevalence |
| GM vs. SB Insights | What | Where | Trend | Score | Scores |
| Catonella sp. COT-257 | Gingivitis/Age | Both | [↓] | N/A | N/A |
| denovo2148 | | locations | (with age for G0, 1, 2) | | |
| Lachnospiraceae | Gingivitis/Age | Both | [↓] | N/A | N/A |
| bacterium COT-263 | | locations | (with age for G0, 1, 2) | | |
| denovo7791 | | | | | |
| Catonella sp. COT-025 | Gingivitis/Age | Both | [↓] | N/A | N/A |
| denovo9179 | | locations | (with age for G0, 1, 2) | | |
| Peptostreptococcaceae | Gingivitis/Age | Both | [↑] | 1 | 6 |
| bacterium COT-030 | | locations | (with age for G0, 1, 2) | | |
| denovo13140 | | | | | |
| Helcococcus sp. | Gingivitis/Age | Both | [↑] | 1 | 6 |
| COT-069 | | locations | (with age for G0, 1, 2) | | |
| denovo18145 | | | | | |
| Propionibacterium sp. | PD/Age | Both | [↓] | N/A | N/A |
| COT-296 | | locations | (with age for G0, 1, 2) | | |
| denovo5120 | | | | | |
| Peptostreptococcaceae | PD/Age | Both | [↑] | 1 | 6 |
| bacterium COT-068 | | locations | (with age for G0, 1, 2) | | |
| denovo17789 | | | | | |
| Stenotrophomonas sp. | PD/Age | Both | [↓] | N/A | N/A |
| COT-224 | | locations | (with age for G0, 1, 2) | | |
| denovo20745 | | | | | |
| Novel Actinomyces sp. | PD/Age | Both | [↓] | N/A | N/A |
| denovo24614 | | locations | (with age for G0, 1, 2) | | |
| Porphyromonas sp. | PD/Age | Both | [↓] | N/A | N/A |
| COT-366 | | locations | (with age for G0, 1, 2) | | |
| denovo29921 | | | | | |
| Lautropia sp. COT-175 | PD/Age | Both | [↓] | N/A | N/A |
| denovo31416 | | locations | (with age for G0, 1, 2) | | |
| Novel Saccharibacteria | Gingivitis/Age | SG | [↑] | 1 | 2 |
| (TM7) sp. | | | (with age for G0, 1, 2) | | |
| denovo7449 | | | | | |
| Prevotella sp. COT-372 | Gingivitis/Age | GM | [↓] | N/A | N/A |
| denovo7980 | | | (with age for G0, 1, 2) | | |
| Peptostreptococcaceae | Gingivitis/Age | SG | [↑] | 1 | 5 (3) |
| bacterium COT-077 | | | (with age for G0, 1, 2) | | |
| denovo8990 | | | | | |
| Peptostreptococcaceae | Gingivitis/Age | SG | [↓] | N/A | N/A |
| bacterium COT-003 | | | (with age for G0, 1, 2) | | |
| denovo9908 | | | | | |
| Clostridiales bacterium | Gingivitis/Age | SG | [↑] | 1 | 5 (3) |
| COT-028 | | | (with age for G0, 1, 2) | | |
| denovo10101 | | | | | |
| Proteiniphilum sp. | Gingivitis/Age | GM | [↑] | 1 | 5 (3) |
| COT-385 | | | (with age for G0, 1, 2) | | |
| denovo12834 | | | | | |
| Corynebacterium | Gingivitis/Age | SG | [↓] | N/A | N/A |
| mustelae | | | (with age for G0, 1, 2) | | |
| denovo13076 | | | | | |
| Streptobacillus sp. | Gingivitis/Age | GM | [↓] | N/A | N/A |
| COT-370 | | | (with age for G0, 1, 2) | | |
| denovo13638 | | | | | |
| Spirochaeta sp. COT-314 | Gingivitis/Age | GM | [↓] | 1 | 1 |
| denovo22492 | | | (with age for G0, 1, 2) | | |
| Erysipelotrichaceae | Gingivitis/Age | SG | [↑] | 2 | 6 (4) |
| bacterium COT-302 | | | | | |
| denovo29017 | | | | | |
| Porphyromonas sp. | Gingivitis/Age | SG | [↓] | N/A | N/A |
| COT-366 | | | (with age for G0, 1, 2) | | |
| denovo29921 | | | | | |
| Moraxella sp. COT-018 | PD/Age | GM | [↓] | N/A | N/A |
| denovo4589 | | | (with age for G0, 1, 2) | | |
| Novel Flavobacterium sp. | PD/Age | GM | [↓] | N/A | N/A |
| denovo5982 | | | (with age for G0, 1, 2) | | |
| Ottowia sp. COT-014 | PD/Age | GM | [↓] | N/A | N/A |
| denovo7130 | | | (with age for G0, 1, 2) | | |
| Prevotella sp. COT-284 | PD/Age | GM | [↓] | N/A | N/A |
| denovo7755 | | | (with age for G0, 1, 2) | | |
| Lachnospiraceae | PD/Age | SG | [↓] | N/A | N/A |
| bacterium COT-263 | | | (with age for G0, 1, 2) | | |
| denovo7791 | | | | | |
| Pseudoclavibacter sp. | PD/Age | GM | [↓] | N/A | N/A |
| COT-392 | | | (with age for G0, 1, 2) | | |
| denovo13919 | | | | | |
| Peptostreptococcaceae | PD/Age | GM | [↓] | N/A | N/A |
| bacterium COT-057 | | | (with age for G0, 1, 2) | | |
| denovo15059 | | | | | |
| Actinobacteria bacterium | PD/Age | SG | [↓] | N/A | N/A |
| COT-406 | | | (with age for G0, 1, 2) | | |
| denovo24357 | | | | | |
| Novel Bibersteinia sp. | PD/Age | SG | [↓] | N/A | N/A |
| denovo27041 | | | (with age for G0, 1, 2) | | |
| Treponema sp. COT-207 | PD/Age | SG | [↓] | N/A | N/A |
| denovo29045 | | | (with age for G0, 1, 2) | | |
| Novel Rikenellaceae sp. | PD/Age | GM | [↑] | 1 | 3 |
| denovo29611 | | | (with age for G0, 1, 2) | | |
| Saccharibacteria (TM7- | PD/Age | SG | [↑] | 1 | 4 (3 SG |
| sp. COT-308 | (Based on | | (with age for G0, 1, 2) | | count only) |
| denovo12286 | presence/absence | | | | |
| | data) | | | | |
| Novel Synergistetes | PD/Age | GM | [↑] | N/A | N/A |
| bacterium | (Based on | | (with age) | | |
| denovo23212 | presence/absence | | | | |
| | data) | | | | |

### REFERENCE LIST

[1] Frank et al. (2007) Proc. Natl. Acad. Sci. USA 104, 13780-13785.
[2] Gevers et al. (2014) Cell Host Microbe 15, 382-392.
[3] Ni et al. (2017) Sci. Transl. Med. 9, eaah6888.
[4] Kostic et al. (2013) Cell Host Microbe 14, 207-215.
[5] Johnson and Foster (2018) Nature Reviews Microbiology, Oct; 16(10):647-655
[6] Kirchoff et al. (2018) PeerJ Preprints 6: e26990v1
[7] Dewhirst F E, Klein E A, Thompson E C, Blanton J M, Chen T, L. Milella, C. M. Buckley, I. J. Davis, M. L. Bennett and Z. V. Marshall-Jones (2012) The canine oral microbiome. PLOS ONE 7: e36067.
[8] Hart et al. (2015) PLoS One. Nov 24; 10(11): e0143334
[9] WO2018/006080
[10] Gennaro (2000) Remington: The Science and Practice of Pharmacy. 20th edition, ISBN: 0683306472.
[11] Molecular Biology Techniques: An Intensive Laboratory Course, (Ream et al., eds., 1998, Academic Press).
[12] Methods In Enzymology (S. Colowick and N. Kaplan, eds., Academic Press, Inc.)
[13] Handbook of Experimental Immunology, Vols. I IV (D. M. Weir and C. C. Blackwell, eds, 1986, Blackwell Scientific Publications)
[14] Sambrook et al. (2001) Molecular Cloning: A Laboratory Manual, 3rd edition (Cold Spring Harbor Laboratory Press).
[15] Handbook of Surface and Colloidal Chemistry (Birdi, K. S. ed., CRC Press, 1997)
[16] Ausubel et al. (eds) (2002) Short protocols in molecular biology, 5th edition (Current Protocols).
[17] PCR (Introduction to Biotechniques Series), 2nd ed. (Newton & Graham eds., 1997, Springer Verlag)
[18] Current Protocols in Molecular Biology (F. M. Ausubel et al., eds., 1987) Supplement 30
[19] Smith & Waterman (1981) Adv. Appl. Math. 2: 482-489.
[20] Davis, I. J., et al., A cross-sectional survey of bacterial species in plaque from client owned dogs with healthy gingiva, gingivitis or mild periodontitis. PLoS One, 2013. 8(12): p. e83158.
[21] Hamp S-E, et al., A macroscopic and radiological investigation of dental diseases of the dog. Veterinary Radiology, 1984. 25(2): p. 86-92.
[22] Kyllar, M. and K. Witter, Prevalence of dental disorders in pet dogs. Veterinarni Medicina, 2005. 50(11): p. 496-505.
[23] Hoffman, T. and P. Gaengler, Epidemiology of periodontal disease in poodles. Journal of Small Animal Practice, 1996. 37: p. 309-316.
[24] Wiggs R and Lobprise H, Chapter 8—Periodontology. In Veterinary Dentistry: Principles and Practice. Raven: Lippencott, 1997.
[25] Gorrel, Chapter 9—periodontal disease. In Veterinary Dentistry for the General Practitioner. Oxford: W. B. Saunders, 2004.
[26] Harvey, C. E., Management of periodontal disease: understanding the options. Vet Clin North Am Small Anim Pract, 2005. 35(4): p. 819-36, vi.
[27] Caporaso, J. G., et al., QIIME allows analysis of high-throughput community sequencing data. Nat Methods, 2010. 7(5): p. 335-6.
[28] Altschul S F, Madden T L, Schäffer AA, Zhang J, Zhang Z, Miller W, Lipman D J. Gapped BLAST and PSI-BLAST: a new generation of protein database search programs. Nucleic Acids Res. 1997 Sep. 1; 25(17):3389-402.
[29] Pruesse, E., et al., SILVA: a comprehensive online resource for quality checked and aligned ribosomal RNA sequence data compatible with ARB. Nucleic Acids Res, 2007. 35(21): p. 7188-96.
[30] Agresti A, Coull, and B. A, Approximate is better than ‘exact’ for interval estimation of binomial proportions. The American Statistician 1998. 52: p. 119-126.
[31] Burnham and Anderson, Model Selection and Multimodel Inference. A Practical Information-Theoretic Approach. 2002: p. 67.
[32] Benjamini Y and Hochberg Y, Controlling the false discovery rate: a practical and powerful approach to multiple testing. Journal of the Royal Statistical Society Series B, 1995. 57: p. 289-300.
[33] Shannon, C. E., The mathematical theory of communication. MD Comput, 1997. 14(4): p. 306-17.
[34] Wickham, H., ggplot2: Elegant Graphics for Data Analysis. Springer-Verlag New York, 2009.
[35] Altschul, S. F., W. Gish, W. Miller, E. W. Myers and D. J. Lipman (1990). “Basic local alignment search tool.” J Mol Biol 215(3): 403-410.
[36] Bates, D., M. Mächler, B. Bolker and S. Walker (2015). “Fitting Linear Mixed-Effects Models Using 1me4.” 2015 67(1): 48.
[37] Fadrosh, D. W., et al. An improved dual-indexing approach for multiplexed 16S rRNA gene sequencing on the Illumina MiSeq platform. Microbiome, 2014. 2(1): 6.
[38] Harrison, X. A. (2014). “Using observation-level random effects to model overdispersion in count data in ecology and evolution.” PeerJ 2: e616.
[39] Marshall, M. D., C. V. Wallis, L. Milella, A. Colyer, A. D. Tweedie and S. Harris (2014). “A longitudinal assessment of periodontal disease in 52 Miniature Schnauzers.” BMC Vet Res 10:

166.
[40] Ruparell, A., et al., The canine oral microbiome: Variation in bacterial populations across different niches. BMC Microbiol., 2020
[41] Ruparell, A., et al., Comparison of subgingival and gingival margin plaque microbiota from dogs with healthy gingiva and early periodontal disease. Res Vet Sci. 2021. 136:396-407
[42] Team, R. C. (2017). “R: A language and environment for statistical computing.”.
[43] Wallis, C., et al. (a), Association of periodontal disease with breed size, breed, weight, and age in pure-bred client. Vet J. 2021. 275:105717
[44] Wallis, C., et al. (b), Subgingival microbiota of dogs with healthy gingiva or early periodontal disease from different geographical locations. BMC Vet Res. 2021. 17:7
[45] Debowes, L J, Mosier, D, Logan, E, Harvey, CE, Lowry, S, Richardson D C. 1996. Association of periodontal disease and histologic lesions in multiple organs from 45 dogs. J Vet Dent, 13, 57-60
[46] Pavlica, Z, Petelin, M, Junes, P, Erzen, D, Crossley, DA, Skaleric, U. 2008. Periodontal disease burden and pathological changes in organs of dogs. J Vet Dent, 25, 97-105
[47] Glickman, LT, Glickman, NW, Moore, GE, Goldstein, GS, Lewis, H B. 2009. Evaluation of the risk of endocarditis and other cardiovascular events on the basis of the severity of periodontal disease in dogs. J Am Vet Med Assoc, 234, 486-94
[48] Pereira dos Santos, JD, Cunha, E, Nunes, T, Tavares, L, Oliveira, M. 2019. Relation between periodontal disease and systemic diseases in dogs. Res Vet Sci, 125, 136-140
[49] Holcombe et al., (2014) PLoS ONE, 9(12), p. e113744
[50] Sturgeon et al., (2013) Veterinary Microbiology, 162(2-4), pp. 891-898
[51] Wallis et al., (2015) Veterinary Microbiology, 181(3-4), pp. 271-282
[52] Wilkins, E. (2009). Clinical Practice of the Dental Hygienist, Philadelphia: Wolters Kluwer Health/Lippincott Williams & Wilkins
[53] Laflamme et al., (2008) Journal of the American Veterinary Medical Association 232: 687-694
[54] Malmsten, (2018) Daxue Consulting https.//daxueconsuiting.com/pet-food-market-in-china/[55]
[55] Prateepsawangwong, N. (2018). How Can Pet Food Brands Break into Thailand's Ecommerce? EcommerceIQ
[56] Sakellari, et al., (2001) Oral Microbiol Immunol 16(6): 376-382
[57] Mayanagi et al., (2004) Oral Microbiol Immunol 19(6): 379-385
[58] Haffajee et al., (2008) Oral Microbiol Immunol 23(3): 196-205
[59] Papaioannou et al., (2009) Oral Microbiol Immunol 24(3): 183-189
[60] Schaumann et al., (2014) BMC Oral Health 14: 157
[61] Daniluk et al., (2006) Adv Med Sci 51 Suppl 1: 81-85
[62] Socransky et al., (1998) J Clin Periodontol 25(2): 134-144
[63] Haffajee et al., (2008) Oral Microbiol Immunol 23(3): 196-205
[64] He et al., (2012) Clin Oral Investig 16(6): 1579-1588
[65] Ximenez-Fyvie et al., (2000) J Clin Periodontol 27(9): 648-657
[66] Ximenez-Fyvie et al., (2000) J Clin Periodontol 27(10): 722-732
[67] Galimanas et al., (2014) Microbiome 2: 32
[68] Wallis and Holcombe, (2020) J Small Anim Pract, 61(9), pp. 529-540

While several embodiments have been provided in the present disclosure, it should be understood that the disclosed systems and methods might be embodied in many other specific forms without departing from the spirit or scope of the present disclosure. The present examples are to be considered as illustrative and not restrictive, and the intention is not to be limited to the details given herein. For example, the various elements or components can be combined or integrated in another system or certain features can be omitted, or not implemented.

In addition, techniques, systems, subsystems, and methods described and illustrated in the various embodiments as discrete or separate can be combined or integrated with other systems, modules, techniques, or methods without departing from the scope of the present disclosure. Other items shown or discussed as coupled or directly coupled or communicating with each other can be indirectly coupled or communicating through some interface, device, or intermediate component whether electrically, mechanically, or otherwise. Other examples of changes, substitutions, and alterations are ascertainable by one skilled in the art and could be made without departing from the spirit and scope disclosed herein.

1. A device, comprising:
a memory operable to store health information associated with a plurality of animals; and
a processor operably coupled to the memory, configured to;
obtain input data for an animal, wherein:
the animal is a member of the canid family;
the input data comprises a first array comprising a first plurality of entries; and
each entry within the first plurality of entries comprises a numerical value that indicates an amount of a type of bacteria that is present within a sample from the animal;
input the input data for the animal into a machine learning model, wherein the machine learning model is configured to:
receive the input data for the animal; and
output an animal age value based at least in part on the input data for the animal, wherein the animal age value identifies a predicted age for the animal;
obtain the animal age value from the machine learning model; and
output the animal age value.

2. The device of claim 1, wherein:
the input data for the animal further comprises an animal size classification value; and
the machine learning model is further configured to output the animal age value based at least in part on the animal size classification.

3. The device of claim 1, wherein:
the input data for the animal further comprises an animal breed identifier that identifies a breed of the animal; and
the machine learning model is further configured to output the animal age value based at least in part on the animal breed identifier.

4. The device of claim 1, wherein:
the input data for the animal further comprises a weight value that identifies a weight for the animal; and
the machine learning model is further configured to output the animal age value based at least in part on the weight value.

5. The device of claim 1, wherein:
the input data for the animal further comprises a gingivitis value for the animal;
the gingivitis value is associated with a time to bleeding when probing a mouth of the animal; and
the machine learning model is further configured to output the animal age value based at least in part on the gingivitis value.

6. The device of claim 1, wherein:
the input data for the animal further comprises a periodontitis value for the animal;
the periodontitis value is associated with an amount of periodontitis that is present in a mouth of the animal; and
the machine learning model is further configured to output the animal age value based at least in part on the periodontitis value.

7. The device of claim 1, wherein:
the input data for the animal further comprises geographic location information for a physical location associated with the animal; and
the machine learning model is further configured to output the animal age value based at least in part on the geographical information.

8. The device of claim 1, wherein the sample is collected while the animal is conscious.

9. The device of claim 8, wherein the sample comprises bacteria from a gingival area in a mouth of the animal.

10. The device of claim 8, wherein the sample comprises bacteria from a supragingival area in a mouth of the animal.

11. The device of claim 1, wherein the sample is collected while the animal is unconscious.

12. The device of claim 11, wherein the sample comprises bacteria from a gingival area in a mouth of the animal.

13. The device of claim 11, wherein the sample comprises bacteria from a subgingival area in a mouth of the animal.

14. The device of claim 11, wherein the sample comprises bacteria from a supragingival area in a mouth of the animal.

15. The device of claim 1, wherein the processor is further configured to:
obtain training data for a second plurality of animals, wherein the training data indicates an amount of a type of bacteria that is present within a sample for each animal from among the second plurality of animals;
associate the training data with animal age values, wherein associating the training data with the animal age values comprises associating each animal from among the second plurality of animals with an animal age value; and
train the machine learning model using the training data that is associated with the animal age values.

16. The device of claim 15, wherein the processor is further configured to:
associate the training data with animal size classification values before training the machine learning model, wherein associating the training data with the animal size classification values comprises associating each animal from among the second plurality of animals with an animal size classification value.

17. The device of claim 15, wherein the processor is further configured to:
associate the training data with animal breed identifiers before training the machine learning model, wherein associating the training data with the animal breed identifiers comprises associating each animal from among the second plurality of animals with an animal breed identifier.

18. The device of claim 15, wherein the processor is further configured to:
associate the training data with weight values before training the machine learning model, wherein associating the training data with the weight values comprises associating each animal from among the second plurality of animals with a weight value.

19. The device of claim 15, wherein the processor is further configured to:
associate the training data with gingivitis values before training the machine learning model, wherein associating the training data with the gingivitis values comprises associating each animal from among the second plurality of animals with a gingivitis value.

20. The device of claim 15, wherein the processor is further configured to:
associate the training data with periodontitis values before training the machine learning model, wherein associating the training data with the periodontitis values comprises associating each animal from among the second plurality of animals with a periodontitis value.

21. The device of claim 15, wherein the processor is further configured to:
associate the training data with geographic location information before training the machine learning model, wherein associating the training data with the geographic location information comprises associating each animal from among the second plurality of animals with a physical location.

22. The device of claim 1, wherein the sample comprises:
a) one or more bacteria selected from a group comprising denovo483, denovo7761, denovo13434, denovo11506, denovo6559, denovo11018, denovo11779, denovo5898, denovo7616, and denovo4478; and/or
b) one or more bacteria selected from a group comprising denovo483, denovo7761, denovo5898, denovo13434, denovo248, denovo11018, denovo2415, denovo11506, denovo264, and denovo715.

23. (canceled)

24. An age determination method, comprising:
obtaining input data for an animal, wherein:
the animal is a member of the canid family;
the input data comprises a first array comprising a first plurality of entries; and
each entry within the first plurality of entries comprises a numerical value that indicates an amount of a type of bacteria that is present within a sample from the animal;
inputting the input data for the animal into a machine learning model, wherein the machine learning model is configured to:
receive the input data for the animal; and
output an animal age value based at least in part on the input data for the animal, wherein the animal age value identifies a predicted age for the animal;
obtaining the animal age value from the machine learning model; and
outputting the animal age value.

25. The method of claim 24, wherein:
a) the input data for the animal further comprises
i) an animal size classification value,
ii) an animal breed identifier that identifies a breed of the animal,
iii) a weight value that identifies a weight for the animal,
iv) a gingivitis value for the animal, wherein the gingivitis value is associated with a time to bleeding when probing a mouth of the animal,
v) a periodontitis value for the animal, wherein the periodontitis value is associated with an amount of periodontitis that is present in a mouth of the animal, and/or
vi) geographic location information for a physical location associated with the animal; and
b) the machine learning model is further configured to
i) output the animal age value based at least in part on the animal size classification,
ii) output the animal age value based at least in part on the animal breed identifier,
iii) output the animal age value based at least in part on the weight value,
iv) output the animal age value based at least in part on the gingivitis value,
v) output the animal age value based at least in part on the periodontitis value, and/or
vi) output the animal age value based at least in part on the geographical information.

26.-37. (canceled)

38. The method of claim 24, further comprising:
obtaining training data for a second plurality of animals, wherein the training data indicates an amount of a type of bacteria that is present within a sample for each animal from among the second plurality of animals;
associating the training data with animal age values, wherein associating the training data with the animal age values comprises associating each animal from among the second plurality of animals with an animal age value; and
training the machine learning model using the training data that is associated with the animal age values.

39. The method of claim 38, further comprising:
a) associating the training data with animal size classification values before training the machine learning model, wherein associating the training data with the animal size classification values comprises associating each animal from among the second plurality of animals with an animal size classification value;
b) associating the training data with animal breed identifiers before training the machine learning model, wherein associating the training data with the animal breed identifiers comprises associating each animal from among the second plurality of animals with an animal breed identifier;
c) associating the training data with weight values before training the machine learning model, wherein associating the training data with the weight values comprises associating each animal from among the second plurality of animals with a weight value;
d) associating the training data with gingivitis values before training the machine learning model, wherein associating the training data with the gingivitis values comprises associating each animal from among the second plurality of animals with a gingivitis value;
e) associating the training data with periodontitis values before training the machine learning model, wherein associating the training data with the periodontitis values comprises associating each animal from among the second plurality of animals with a periodontitis value;
f) associating the training data with geographic location information before training the machine learning model, wherein associating the training data with the geographic location information comprises associating each animal from among the second plurality of animals with a physical location.

40.-44. (canceled)

45. The method of claim 24, wherein the sample comprises a) one or more bacteria selected from a group comprising denovo483, denovo7761, denovo13434, denovo11506, denovo6559, denovo11018, denovo11779, denovo5898, denovo7616, and denovo4478; or b) one or more bacteria selected from a group comprising denovo483, denovo7761, denovo5898, denovo13434, denovo248, denovo11018, denovo2415, denovo11506, denovo264, and denovo715.

46. (canceled)

47. A computer program comprising executable instructions stored in a non-transitory computer-readable medium that when executed by a processor causes the processor to perform the method of claim 2.

48.-69. (canceled)

70. A method of determining the oral microbiome age status of a canid, comprising
quantifying one or more bacterial taxa in a sample obtained from an oral cavity of the canid to determine the abundance or relative abundance of the bacterial taxa;
comparing the abundance or relative abundance of said bacterial taxa in the sample to the abundance or relative abundance of the bacterial taxa in a control data set; and
determining the oral microbiome age status.

71. The method of claim 70, wherein
a) the control data set comprises oral microbiome data from at least two, preferably three, preferably all four life stages of a canid selected from the list consisting of a puppy, an adult canid, a senior canid and a geriatric canid;
b) the control data set comprises i) oral microbiome data taken from canids from a plurality of geographical locations, or ii) oral microbiome data taken from canids from a single geographical location, wherein optionally the canid is also from the same geographical location; and/or
c) the control data set consists of oral microbiome data taken from canids of one breed size and the canid to be assessed is of the same breed size.

72.-73. (canceled)

74. The method of claim 70, wherein
the method includes quantifying one or more bacterial taxa selected from the group consisting of the taxa specified in FIG. 7 and/or FIG. 8, and optionally,
the bacterial taxa has a 16S rDNA with at least 95%, at least 96%, at least 97%, at least 98%, at least 99% or 100% identity to the sequence of any one of SEQ ID Nos: 1, 3-6, 8-11, 13-30, 32-60, 62-63, 65-85, 87-88, 90-98, 100-147.

75. The method of claim 74, wherein
the method includes quantifying one or more bacterial taxa selected from the group consisting of Aquaspirillum sp FOT-079/COT-091, novel Erysipelotrichaceae sp (OTU 11710), novel Tissierellaceae/Peptostreptococcaceae sp (OTU 11779), Catonella sp. (COT-098/COT-158/FOT-010) and novel Alloprevotella/Prevotella sp. (OTU 11854) and optionally
the bacterial taxa has a 16S rDNA with at least 95%, at least 96%, at least 97%, at least 98%, at least 99% or 100% identity to the sequence of any one of SEQ ID NOs 5, 13, 14, 15 and/or 16.

76. The method of claim 75, further comprising
quantifying one or more bacterial taxa selected from the group Blautia sp. (COT-337), Novel Bergeyella/Novel Weeksellaceae/loacibacterium sp. COT-320 (OTU 1233), Capnocytophaga canimorsus, Prevotella sp. COT-226 and Conchiformibius steedae and optionally
the bacterial taxa has a 16S rDNA with at least 95%, 95%, 96%, 97%, 98%, 99% or 100% identity to the sequences of at least 2, 3, 4 or all of SEQ ID NOs: 17, 18, 21, 23 and 27.

77. The method of claim 76, wherein at least 2, 3, 4, 5 bacterial taxa are quantified.

78.-81. (canceled)

82. The method of claim 70, wherein the method comprises quantifying one or more bacterial taxa selected from the group Peptostreptococcaceae bacterium COT-030, Helcococcus sp. COT-069, Peptostreptococcaceae bacterium COT-068, Novel Saccharibacteria (TM7) sp., Peptostreptococcaceae bacterium COT-077, Clostridiales bacterium COT-028, Proteiniphilum sp. COT-385, Spirochaeta sp. COT-314, Erysipelotrichaceae bacterium COT-302, Novel Rikenellaceae sp., and Saccharibacteria (TM7) sp. COT-308.

83. A method of monitoring a canid, comprising a step of determining the oral microbiome age status of the canid by the method of claim 70 on at least two time points.

84.-85. (canceled)

86. A method of monitoring the oral microbiome age status in a canid that has undergone a dietary change, and/or who has received a supplement, a functional food, a nutraceutical composition, a pharmaceutical composition or a preparation, which is able to change the oral microbiome composition, comprising determining the oral microbiome age status by the method of claim 83.

87.-88. (canceled)

89. A method of assessing the oral microbiome age status of a canid to determine whether an intervention is required, comprising:
quantifying one or more bacterial taxa in a sample obtained from the oral cavity of the canid;
determining the abundance or relative abundance of said bacterial taxa;
comparing the abundance or relative abundance determined in step (b) to that of a control data set;
wherein if the comparing of step (c) indicates a difference in oral microbiome age status to actual age of the canid, an intervention is recommended.

90.-97. (canceled)

98. A machine learning model training method, comprising:
obtaining training data for a plurality of animals, wherein:
the training data indicates an amount of a type of bacteria that is present within a sample for each animal from among the plurality of animals; and
the plurality of animals are members of the canid family;
associating the training data with animal age values, wherein associating the training data with the animal age values comprises associating each animal from among the second plurality of animals with an animal age value; and
training a machine learning model using the training data that is associated with the animal age values, wherein the machine learning model is configured to:
receive input data for an animal; and
output an animal age value based at least in part on the input data for the animal, wherein the animal age value identifies a predicted age for the animal.

99. The method of claim 98, further comprising:
a) associating the training data with animal size classification values before training the machine learning model, wherein associating the training data with the animal size classification values comprises associating each animal from among the plurality of animals with an animal size classification value;
b) associating the training data with animal breed identifiers before training the machine learning model, wherein associating the training data with the animal breed identifiers comprises associating each animal from among the plurality of animals with an animal breed identifier;
c) associating the training data with weight values before training the machine learning model, wherein associating the training data with the weight values comprises associating each animal from among the plurality of animals with a weight value;
d) associating the training data with gingivitis values before training the machine learning model, wherein associating the training data with the gingivitis values comprises associating each animal from among the plurality of animals with a gingivitis value;
e) associating the training data with periodontitis values before training the machine learning model, wherein associating the training data with the periodontitis values comprises associating each animal from among the plurality of animals with a periodontitis value; and/or
f) associating the training data with geographic location information before training the machine learning model, wherein associating the training data with the geographic location information comprises associating each animal from among the plurality of animals with a physical location.

100.-104. (canceled)


SEQUENCE LISTING (153 sequences)

SEQ ID NO: 1 - DNA, 350 bp, Unknown
  source: /note=“Description of Unknown Clostridiales bacterium COT-386 sequence”
ttaccgcggctgctggcacgtagttagccggggcttccttcttggctaccgtcattatcttcaccaaggatagaagtttacaatccgaaaaccttcttccttcacgcggcgttgctgggtcagagtttcctccattgcccaatattccctactgctgccttccgtagaagtctggaccgtgtctcagttccagtgtggccgatcaccctcttaggtcggctatctatcgtagacttggtgagcctttacctcaccaactatctaatagaacgcgagctccccctttaccgtgtttccactttcctccaaaaaccatgcggtccccagaaattatgcggtattagtacaag

SEQ ID NO: 2 - DNA, 350 bp, Unknown
  source: /note=“Description of Unknown Chloroflexi bacterium COT-408 sequence”
ttaccgcggctgctggcacgtagttagccgagacttattctcgtggtactgtccttcctcatcccacgtaaaagaactttacgacccgaaggccttcttcgttcacgcggcgttgctgcttcaggctttcgcccattaagcaatattccctactgctgccacccgcaggtgtatggaccgtgtttcagttccattgtgggggatcaccctctcaggtcccctacccgtcttcgccttggtgagccgttacctctccaactagctgatgggccgcaggcccctcctaaagtagatttctcttttactatgagccttctaatctctcatagcttatgcggttttgtcactcg

SEQ ID NO: 3 - DNA, 350 bp, Pasteurellaceae sp.
ttaccgcggctgctggcacggagttagccggtgcttcttctgtggctaacgtcaatcttaagtactattagcacttaagccttcctcaccaccgaaagaactttacaacccgaaggccttcttcattcacgcggcatggctgcgtcagggttgcccccattgcgcaatattccccactgctgcctcccgtaggagtctggaccgtgtctcagttccagtgtggctggtcatcctctcagaccagctagagatcgtcggcttggtaggcctttaccccaccaactacctaatcccacttgggctcatcttatggcgaatggctaatgtcccattctttaatccgtagatgt

SEQ ID NO: 4 - DNA, 350 bp, Unknown
  source: /note=“Description of Unknown Novel Oceanivirga/Novel Leptotrichaceae sequence”
ttaccgcggctgctggcacgtatttagccgtcgcttcttctgttggtaccgtcatttctttctccccaactaaaagtgctttacaatccgaagaccttcttcgcacacacagaattgctggatcagagttacctccattgtccaatattccccactgctgcctcccgtaggagtaagggccgtatctcagtccccttgtggccgttcaccctctcaggccggctacctatcatcgccttggtaggcctttaccccaccaactagctaataggacgcaaagctctccactagtaatctctttttcatttaaagtagatgcctactttcaacttatccggttttatcagaaa

SEQ ID NO: 5 - DNA, 350 bp, Aquaspirillum sp.
ttaccgcggctgctggcacgtagttagccggtgcttattctttgggtactctcataatctccagctcttaaccagatacctttgctccccaacaaaagtcctttacaacccgaaggccttcttcagacacgcggcatggctggatcagggttccccccattgtccaaaattccccactgctgcctcccgtaggagtctgggccgtctctcagtcccagtgtggcggatcatcctctcagacccgctactgatcgtcgccttggtaagcctttacctcaccaactagctaatcagacattggccgctcttatatcgccaggtcaaacgatccccagctttccccctcaggg

SEQ ID NO: 6 - DNA, 350 bp, Bacteroides pyogenes
ttaccgcggctgctggcacggagttagccgatgcttattcataacggtacatgcaataaggtacccgtacctcatgttattcccgtataaaagaagtttacaacccagagggcagtcatccttcacgctacttggctggttcagacttccgtccattgaccaatattcctcactgctgcctcccgtaggagtttggaccgtgtctcagttccaatgtgggggaccttcctctcagaacccctatccatcgaaggtttggtgggccgttaccccgccaactgcctaatggaacgcatccccatcggttaccggaatcctttggcgtatcaatcaggagaaagatacacaac

SEQ ID NO: 7 - DNA, 350 bp, Porphyromonas sp.
ttaccgcggctgctggcacggagttagccgatgcttattcatagtatacattcaactttggaacgaatccaatcctttattctactataaaagaagtttacaattcataaaacagtcttccttcacgcgacttggctggttcaggctctcgcccattgaccaatattcctcactgctgcctcccgtaggagtctggtccgtgtctcagtaccagtgtgggggttcaacctctcagttcccctacccatcgtcgccttggtgagccgttacctcaccaactagctaatgggacgcatgcctatcttatagcaattaatctttccttatctaatgatgccactaaataagaa

SEQ ID NO: 8 - DNA, 350 bp, Catonella sp.
ttaccgcggctgctggcacgtagttagccggggcttcttagtcaggtaccgtcatcatcttccctgctgatagagctttacataccgaaatacttcttcactcacgcggcgtcgctgcatcagagtttcctccattgtgcaatatcccccactgctgcctcccgtaggagtttgggccgtgtctcagtcccaatgtggccgtccgacctctcagtccggctaccgatcgtcgccttggtgagccgttacctcaccaaccagctaatcggacgcgggcccatctcataccagattgctcttttcccctccgcttcatgcgaagcaaaggacttatgcggtcttatcagaca

SEQ ID NO: 9 - DNA, 350 bp, Corynebacterium sp.
ttaccgcggctgctggcacgtagttagccggtgcttcttctcaaactaccgtcaccacccccaaaaaaaagggcagcttcgtcataagcgaaaggagtttacaacccgaaggccgtcatcccccacgcggcgtcgctgcatcaggcttgcgcccattgtgcaatattccccactgctgcctcccgtaagagtctgggccgtatctcagtcccaatgtggccgtccaccctctcaggccggctacccgtcgacgccttggtaggccattaccccaccaacaagctgataggccgcgagctcatccccaaccaaaaaacatctttccaccacacacaccactgcatggtcct

SEQ ID NO: 10 - DNA, 350 bp, Unknown
  source: /note=“Description of Unknown Erysipelotrichaceae bacterium COT-302 sequence”
ttaccgcggctgctggcacgtagttagccgtggctttctaataaagtaccgtcactctgagattctttcccagtcgttcttcctttacaacagagctttacaacccgaaggccgtcttcactcacgcggcgttgctcggtcaggctttcgcccattgccgaaaattccctactgctgcctcccgtaggagtttgggccgtgtctcagtcccaatgtggccgatcaccctctcaggtcggctatgtatcatcgccatggtgagccgttacctcaccatctagctaatacaccataagcccatccatttgtgtctctaacctttaacaagatggggatgccccctccttgcc

SEQ ID NO: 11 - DNA, 350 bp, Capnocytophaga canimorsus
ttaccgcggctgctggcacggagttagccgatgcttattcatacagtaccgtcatcaatctactcgtagaccttattcttcctgtataaaagcagtttacaacccataaggccgtcatcctgcacgcggcatggctggatcagtcttccgaccattgtccaatattcctcactgctgcctcccgtaggagtctggtccgtgtctcagtaccagtgtgggggacatccctctcagaacccctacccatcgtagccttggtgtgccgttaccactccaactagctaatgggacgcgtactcatcttttaccaccgtagctttaattatttaatgatgccataaaataatact

SEQ ID NO: 12 - DNA, 350 bp, Unknown
  source: /note=“Description of Unknown Chloroflexi bacterium human oral taxon 439 sequence”
ttaccgcggctgctggcacgtagttagccgagacttattcccgtggtactgtccttcctcttcccacgtaaaagaactttacgacccgaaggccttcttcgttcacgcggcgttgctgcttcaggctttcgcccatttagcaatattccctactgctgccacccgtaggtgtatggaccgtgtttcagttccattgtgggggatcaccctctcaggtcccctacccgtcttcgccttggtgagccgttacctctccaactagctgatgggccgcaggcccctcccaaagtagattcctcttttactatcggctctctgactcccgatagcttatgcggttttgtcgctcg

SEQ ID NO: 13 - DNA, 350 bp, Unknown
  source: /note=“Description of Unknown Novel Erysipelotrichaceae sequence”
ttaccgcggctgctggcacgtagttagccgtggctttctggtaaggtaccgtcatcaaagtgccatttcctacactccttattcttcccttacaacagagctttacacaacgaattgcgtcttcactcacgcggcattgctccatcagggttgcccccattgtggaaaattccctactgctgcctcccgtaggagtttgggccgtgtctcagtcccaatgtggccgttcaccctctcaggtcggctatgcatcatcgccttggggagccgttacctctccaactagctaatgcaccgcaggtccatctcagtgcgttgcatccgcaactttcaatgactctagatgccta

SEQ ID NO: 14 - DNA, 350 bp, Unknown
  source: /note=“Description of Unknown Novel Tissierellaceae/Novel Peptostreptococcaceae sequence”
ttaccgcggctgctggcacgtagttagccggggcttcctcctatggtaccgtcattatctttccataggacagagctttacgaaacgaattccttcttcgctcacgcggcgtcgctgcatcagggttccccccattgtgcaaaattccccactgctgcctcccgtaggagtctggaccgtctctcagttccagtgtggccgttcactctctcaagccggctaccgatcattgccttggtgagctgttatctctccaaccagctaatcggacgcgagcccatcttccaccgatttctctttagttaccttatcatgcgattcgttaacttcatatggtattaattaccgtt

SEQ ID NO: 15 - DNA, 350 bp, Unknown
  source: /note=“Description of Unknown Catonella COT-098/COT-158/FOT-010 sequence”
ttaccgcggctgctggcacgtagttagccggggcttcttagtcaggtaccgtcatcatcttccctgctgatagagctttacataccgaaatacttcttcactcacgcggcgtcgctgcatcagagtttcctccattgtgcaatatcccccactgctgcctcccgtaggagtttgggccgtgtctcagtcccaatgtggccgtccgacctctcagtccggctactgatcgttgccttggtaagccgttaccttaccaactagctaatcagacgcgggcccatctcataccagattgctcttttcccttctcagtcatgcgaccaaaaagacgtatgcggtcttagcagacg

SEQ ID NO: 16 - DNA, 350 bp, Unknown
  source: /note=“Description of Unknown Novel Alloprevotella/Novel Prevotella sequence”
ttaccgcggctgctggcacggaattagccggtcctttttctgatggtacattcctttcggggacgcgtccccgccgttattcccaactaaaagcagtttacaacccggagggccgtcatcctgcacgctacttggctggttcagactttcgtccattgaccaatattcctcactgctgcctcccgtaggagtctggaccgtgtctcagttccagtgtgggggaccttcctctcagaacccctactgatcgtcggctaggtgggccgttaccccgcctactacctaatcagacgcatcaccatcgcataccgataaaatctttgattatctccccatgcggggaaacaata

SEQ ID NO: 17 - DNA, 350 bp, Blautia sp.
ttaccgcggctgctggcacgtagttagccggggcttcttagtcaggtaccgtcattttcttccctgctgatagagctttacataccgaaatacttcatcactcacgcggcgtcgctgcatcagggtttcccccattgtgcaatattccccactgctgcctcccgtaggagtttgggccgtgtctcagtcccaatgtggccggtcaccctctcaggtcggctactgatcgtcgccttggtaagccgttaccttaccaactagctaatcagacgcgggtccatctcataccaccggagtttttcccactgagtcatgcgactctgtggtcttatgcggtattagcagctatt

SEQ ID NO: 18 - DNA, 350 bp, Unknown
  source: /note=“Description of Unknown Novel Bergeyella/Novel Weeksellaceae/loacibacterium sp. COT-320 sequence”
ttaccgcggctgctggcacggagttagccggtgcttattcgtatagtaccttcagctaccctcacgagagtaggtttatccctatacaaaagaagtttacaacccatagggcagtcgtccttcacgcgggatggctggatcaggcttccacccattgtccaatattcctcactgctgcctcccgtaggagtctgggccgtgtctcagtcccagtgtgggggatcaccctctcaggccccctaaagatcgtcgccttggtgagccgttacctcaccaactagctaatcttgcgcgtgcccatccttatccaccgtagtttttaatataaagtgatgccactctatatgtta

SEQ ID NO: 19 - DNA, 350 bp, Propionibacterium sp.
tcaccgcggctgctggcacgtagttagccggtgcttcttctgttggtaccgtcactcacgcttcgtcccaactgaaagcggtttacaacccgaaggccgtcatcccgcacgcggcgttgctgcatcaggcttccgcccattgtgcaatattccccactgctgcctcccgtaggagtttgggccgtatctcagtcccaatgtggccggtcgccctctcaggccggctacccgtcgaagccttggtgagccactacctcaccaacaagctgataggccgcgagcccatccccaaccgcaaaagctttccaaccagacccatgcaggcccagctcatatccagtattagcaac

SEQ ID NO: 20 - DNA, 350 bp, Actinomyces bowdenii
ttaccgcggctgctggcacgtagttagccggcgcttcttatccagctaccgtcaacccacccacacaggagcaggcctgcttcactggcgaaagaggttcacaacccgaaggcctccatccctcacgcggcgtcgctgcatcaggcttgcgcccattgtgcaatattccccactgctgcctcccgtaggagtctgggccgtgtctcagtcccagtgtggccgtccaccctctcaggccggctacccgtcaccgccttggtaggccatcaccccaccaacaagctgataggccgcgagcccatcccccaccaaaaacacccaaaaggcgcatctttcccagcccagccatg

SEQ ID NO: 21 - DNA, 350 bp, Capnocytophaga canimorsus
ttaccgcggctgctggcacggagttagccgatgcttattcatacagtaccgtcatcaatctactcgtagaccttattcttcctgtataaaagcagtttacaacccataaggccgtcatcctgcacgcggcatggctggatcagtcttccgaccattgtccaatattcctcactgctgcctcccgtaggagtctggtccgtgtctcagtaccagtgtgggggacctccctctcaggacccctacccatcgtagccttggtatgccgttaccacaccaactagctaatgggacgcgtactcatcttttgccaccagagctttaattattcagtgatgccaccaaacaatact

SEQ ID NO: 22 - DNA, 350 bp, Unknown
  source: /note=“Description of Unknown Peptostreptococcaceae bacterium COT-168/FOT-067/Novel Fusibacter sequence”
ttaccgcggctgctggcacgtagttagccggtgcttattcgccgggtaccgtcattatctttcccggcaaaagaactttacagatcgaaatccttcatcgttcacgcggcgttgctgcatcagggtttcccccattgtgcaatattccctactgctgcctcccgtaggagtttgggccgtgtctcagtcccaatgtggccgatcaccctctcaggtcggctaccgatcgcggtcttggtgagccgttacctcaccaactaactaatcggacatgggtccatcctttggcgataaatcttttcacactgcttcatgcgaaactgtgcgcttatgaggtattaatagccgtt

SEQ ID NO: 23 - DNA, 350 bp, Prevotella sp.
ttaccgcggctgctggcacggaattagccggtcctttttctgatggtacattcctttcggggacgcgtccccgccgttattcccaactaaaagcagtttacaacccggagggccgtcatcctgcacgctacttggctggttcagacttccgtccattgaccaatattcctcactgctgcctcccgtaggagtctggaccgtgtctcagttccagtgtgggggaccttcctctcagaacccctactgatcgtcggctaggtgggccgttaccccgcctactacctaatcagacgcatcaccatcgcataccgataaaatctttgattataccctcatgcgagggcataata

SEQ ID NO: 24 - DNA, 350 bp, Granulicatella sp.
ttaccgcggctgctggcacgtagttagccgtggctttctggttagataccgtcacggcataatcagttactattatgcttattcttctctaacaacagagttttacgatccgaaaaccttcttcactcacgcggcgttgctccgtcagacttgcgtccattgcggaagattccctactgctgcctcccgtaggagtttgggccgtgtctcagtcccaatgtggccgatcaccctctcaggtcggctatgcatcatcgccttggtgagccgttacctcaccaactagctaatgcaccgcgggtccatccaccagcagaagcttacgcctcttttccttcctcagtcatgca

SEQ ID NO: 25 - DNA, 350 bp, Filifactor alocis
ttaccgcggctgctggcacgtagttagccggggctttcttttagggtactgtcatttctttcttcccctactacagagttttacgacacgaatgccttcttcactcacgcggcgttgctgcatcagggttccccccattgtgcaatattccccactgctgcctcccgtaggagtttggaccgtgtctcagttccaatgtggccgttcaccctctcaggtcggctactgatcgttgccttggtgggcttttatctcaccaactagctaatcagacgcgggctcatctttgtccactatcgttttgatattattaggatgccctattaatatgttatacggttttagctatt

SEQ ID NO: 26 - DNA, 350 bp, Unknown
  source: /note=“Description of Unknown Clostridiales bacterium COT-028 sequence”
ttaccgcggctgctggcacgtagttagccggggcttcctccttggctaccgtctttccctcttcaccaagaacagaagtttacaatccgaagaccttcttccttcacgcggcgtcgctgggtcagggtttcccccattgcccaatattccccactgctgcctcccgtaggagtttggaccgtctctcagttccaatgtggccgatcaccctctcaggtcggctaccgatcgttgacttggtgagccgttacctcaccaactatctaatcggacgcgagcccatctcttaccggatttctcctttgaccccttccatatgtatgcttgtggtctcatgcggtattagtgtg

SEQ ID NO: 27 - DNA, 350 bp, Conchiformibius steedae
ttaccgcggctgctggcacgtagttagccggtgcttattcttcgggtaccgtcatcagcaaatgatattagcacctgccttttcttccccgacaaaagtcctttacaacccgaaggccttcttcagacacgcggcatggctggatcaggcttgcgcccattgtccaaaattccccactgctgcctcccgtaggagtctgggccgtgtctcagtcccagtgtggcggatcatcctctcagacccgctactgatcgttgccttggtaggcttttaccccaccaactagctaatcagacattggccgctcggataacgcgaggtcaaacgatcccccgctttccttcttaaag

SEQ ID NO: 28 - DNA, 350 bp, Unknown
  source: /note=“Description of Unknown Pasteurellaceae bacterium COT-272/ Haemophilus sequence”
ttaccgcggctgctggcacggagttagccggtgcttcttctgtgaataacgtcaatcctaaatgctattaacatttaagccttcctcatcaccgaaagaactttacaacccgaaggccttcttcattcacgcggcatggctgcgtcagggttgcccccattgcgcaatattccccactgctgcctcccgtaggagtctggaccgtgtctcagttccagtgtggctggtcatcctctcagaccagctagagatcgaaggcttggtaggcccttaccccaccaactacctaatcccacttgggctcatcttatggcaactggccttgcggtcccagtctttaatctttcgat

SEQ ID NO: 29 - DNA, 350 bp, Leptotrichia sp.
ttaccgcggctgctggcacgtatttagccgtcgcttcttctacaggtaccgtcactttcttcttccctgttgaaagcactttacaatccgaaaaccttcttcgtgcacacagaattgctggatcagagtctcctccattgtccaatattccccactgctgcctcccgtaagagtaagggccgtatctcagtccccttgtggccgtccaccctctcaggccggctacctatcatcgccttggtatgcctttaccacaccaactaactaataggacgcaaagctctctctaggcatcccttttcattatacagtcatgcaactacataattatatcaagtcttatcagaagt

SEQ ID NO: 30 - DNA, 350 bp, Streptobacillus sp.
ttaccgcggctgctggcacgtatttagccgtcgcttcttctgtaggtaccgtcactttcttcttccctactgaaagcactttacaatccgaaaaccttcttcgtgcacacagaattgctggatcagagttgcctccattgtccaatattccccactgctgcctcccgtaggagtaagggccgtatctcagtccccttgtggccgttcaccctctcgggccggctacctatcatcgccttggtaggcctttaccccaccaactagctaataggatgcaaagctctctcttagtgatctctctttcattaatgtaggatgccctactctaataatatctagtattatccgtc

SEQ ID NO: 31 - DNA, 350 bp, Fusobacterium sp.
ttaccgcggctgctggcacgtatttagccgtcgcttcttctgttggtaccgtcactttcttcttcccaactgaaagcactttacattccgaaaaacttcatcgtgcacacagaattgctggatcagactcttggtccattgtccaatattccccactgctgcctcccgtaggagtaagggccgtgtctcagtccccttgtggccgttcaccctctcaggccggctacccatcatcgccttggtgagccgttacctctccaactagctaatgggacgcaaagctctctcacagcgcatatagctttcataatccatgcatgcgcatagttcataatatccagttttagcat

SEQ ID NO: 32 - DNA, 350 bp, Unknown
  source: /note=“Description of Unknown Erysipelotrichaceae bacterium FOT-121 sequence”
ttaccgcggctgctggcacgtagttagccgtggctttctggtaaatcagtcatctctataagtcccttatagccattcttaatttataacagagctttacaacccgaaggccttcttcactcacgcggcgttgctcggtcaggcttgcgcccattgccgaaaattccctactgctgcctcccgtaggagtctgggccgtgtctcagtcccaatgtggccgttcaccctctcaggccggctacgtatcatcgtcttggtgagccgttacctcaccaactaactaatacgccataagcccatctattagtgtctctaacctttaataacttaaagatgccttcaagttacct

SEQ ID NO: 33 - DNA, 350 bp, Leucobacter sp.
ttaccgcggctgctggcacgtagttagccggtgctttttctgtaggtaccgtcacaaaagcttcttccctactaaaagaggtttacaacccgaaggccgtcatccctcacgcggcgttgctgcatcaggctttcgcccattgtgcaatattccccactgctgcctcccgtaggagtctgggccgtgtctcagtcccagtgtggccggtcaccctctcaggccggctacccgtcgtcgccatggtgagccattacctcaccatcaagctgataggccgcgagtccatcccctaccgaaaaaactttccaccaagaaaccatgcggttcaaggtcgtatccagtattagaca

SEQ ID NO: 34 - DNA, 350 bp, Prevotella sp.
ttaccgcggctgctggcacggaattagccggtcctttttcttacggtacttgcaagacaccacacgtggcgttttttacccccgtataaaagcagtttacaacccagagggcagtcttcctgcacgctacttggctggttcagactctcgtccattgaccaatattcctcactgctgcctcccgtaggagtctggaccgtgtctcagttccagtgtgggggatcttcctctcagaacccctacccatcggaggcttggtgagccgttacctcaccaactacctaatgggacgcatcctcatcgcataccgaaattctttggtttatcattcatgcgaataataaacaaca

SEQ ID NO: 35 - DNA, 350 bp, Capnocytophaga sp.
ttaccgcggctgctggcacggagttagccgatgcttattcgcaaagtaccgtcatcactctactcgtagagcttattcttcccttgcaaaagcagtttacaacccataaggccgtcttcctgcacgcggcatggctggttcagtcttccgaccattgaccaatattcctcactgctgcctcccgtaggagtctggtccctgtctcagtaccagtgtgggggatctccctctcaggacccctatctatcgtagccttggggtgccattaccactccaactagctaatagaacgcgtactcatcttttaccgccgtagctttaattaccctatgatgccataaagtaattct

SEQ ID NO: 36 - DNA, 350 bp, Unknown
  source: /note=“Description of Unknown Clostridiales bacterium COT-388/Novel Clostridium sequence”
ttaccgcggctgctggcacgtagttagccgtggcttgttcttcaggtaccgtcatctctttcgtccctgaccaaagaagtttacaatccgaagaccttcatccttcacgcggcgttgctgggtcaggcttgcgcccattgcccaatattccccactgctgcctcccgtaggagtctgggccgtgtctcagtcccaatgtggccgttcaacctctcagtccggctaccgatcgtcgacttggggagccgttacctcgccaactatctaatcggacgcgagcccatcttgtaccggattgctcctttgataccatttggatgcccattcggtatgtcatgcggtattagcaa

SEQ ID NO: 37 - DNA, 350 bp, Actinomyces sp.
ttaccgcggctgctggcacgtagttagccggcgcttcttatccagctaccgtcaacccacccacacaggagcaggcctgcttcactggcgaaagaggttcacaacccgaaggcctccatccctcacgcggcgtcgctgcatcaggcttgcgcccattgtgcaatattccccactgctgcctcccgtaggagtctgggccgtgtctcagtcccagtgtggccgtccaccctctcaggccggctacccgtcaccgccttggtaggccatcaccccaccaacaagctgataggccgcgagcccatcccccaccagaaaaaacctttcccagcccagccatgcgaccagaccag

SEQ ID NO: 38 - DNA, 350 bp, Unknown
  source: /note=“Description of Unknown Actinobacteria bacterium COT-406/Novel Euzebya sequence”
ttaccgcggctgctggcacgtagttggccggggcttcttctgaaggtaccgtcacaaaacgcttcttccctcccgaaagtggtttacaacccgaaggccttcctcccacacgcggcgtcgctgcgtcagacttccgtccattgcgcaagattccccactgctgcctcccgtaggagtctgggccgtgtctcagtcccaatgtggctgtacaagctctcactccagctacccgtcgtcgacttggtaggccattaccccaccaaccatctgataggccgcgagaccatcccgagacgccaaagctttccaccaccaaccatgcggccggcagtcatattcggcattagcca

SEQ ID NO: 39 - DNA, 350 bp, Unknown
  source: /note=“Description of Unknown Peptostreptococcaceae bacterium FOT-064/COT-068/Novel Mogibacteriaceae sequence”
ttaccgcggctgctggcacgtagttagccggggcttcctccaaaggtaccgtcattatcttccctttggacagaactttacgacccaagggccttcttcgttcacgcggcgttgctgcatcaggctttcgcccattgtgcaatattccccactgctgcctcccgtaggagtttggaccgtgtctcagttccaatgtggccgatcaccctctcaggtcggctactgatcgtcgccttggtgagccgttacctcaccaactaactaatcagacgcaggcccatcccataccgataaatctttggtaaaaatgtcatgcaacatctttacttcataaggttttaataacagtt

SEQ ID NO: 40 - DNA, 350 bp, Unknown
  source: /note=“Description of Unknown Campylobacter sp. FOT-100/COT-011/Campylobacter rectus sequence”
ttaccgcggctgctggcacggagttagccggtgcttattccttgggtaccgtcataattctttcccaagaaaaggagtttacgctccgaaaagtgtcatcctccacgcggcgttgctgcttcagggtttcccccattgagcaatattccctactgctgcctcccgtaggagtctggaccgtgtctcagttccagtgtgactgatcatcctctcagaccagttacgcgtcatagccttggtgagccattacctcaccaactagctgatacgatatagcctcatcctacaccgaaaaactttcccgacttaacttatgtaaagaaggagtatagagtattagcagtcgtttc

SEQ ID NO: 41 - DNA, 350 bp, Unknown
  source: /note=“Description of Unknown Peptostreptococcaceae bacterium COT-019/Novel Fusibacter sequence”
ttaccgcggctgctggcacgtagttagccggtgcttattccttgggtaccgtcaccttgttcttccccaagaaaagaattttacgatccgaagaccttcatcattcacgcggcgttgctgggtcagggttgcccccattgcccaatattccctactgctgcctcccgtaggagtctggaccgtgtctcagttccagtgtggccgatcaccctctcaggtcggctactgatcgtcgccttggtgagccgttacctcaccaactagctaatcagacgtgggaccatcctttagcgataaaatctttccttgaatcttcatgcgaaaactcaagagtatggggtattaatagt

SEQ ID NO: 42 - DNA, 350 bp, Unknown
  source: /note=“Description of Unknown Ottowia sp. FOT-161/Novel Hylemonella/Novel Comamonas sequence”
ttaccgcggctgctggcacgtagttagccggtgcttattcttacagtaccgtcatggtccccaggtattagccagagatttttcgttctgtacaaaagcagtttacaacccgagggccttcgtcctgcacgcggaatggctggatcaggctttcgcccattgtccaaaattccccactgctgcctcccgtaggagtctgggccgtgtctcagtcccagtgtggctgatcatcctctcagaccagctacagatcgcaggcttggtaagcctttaccccaccaactacctaatctgccatcggccgctccaaacgcgcgaggcccaaaggtcccccgctttcatcctcagat

SEQ ID NO: 43 - DNA, 350 bp, Unknown
  source: /note=“Description of Unknown Lachnospiraceae bacterium FOT-001/COT-073 sequence”
ttaccgcggctgctggcacatagttagccggggcttcttagtcaggtaccgtcattttcttccctgctgatagaagtttacaatccgaaaaccgtcttccttcacgcggcattgctgcatcagagttccctccattgtgcaatatcccccactgctgcctcccgtaggagtttgggccgtgtctcagtcccaatgtggccgatcaccctctcaggtcggctactgatcgtcgccttggtgagcttttacctcaccaactaactaatcagacgcgggcccgtcttacaccaccggagtttttcacaccgtaccatgcggtactgtgcgcttatgcggtattagcgcaaatt

SEQ ID NO: 44 - DNA, 350 bp, Unknown
  source: /note=“Description of Unknown Peptostreptococcaceae bacterium COT-086/FOT-031 sequence”
ttaccgcggctgctggcacgtagttagccggggcttcctcacagggtaccgtctttgtcttcccctgcgacagagctttacgactcgaaagccttcatcgctcacgcggcgttgctgcatcaggctttcgcccattgtgcaatattccccactgctgcctcccgtaggagtctggaccgtgtctcagttccagtgtggccgatcaccctctcaggtcggctacggatcgtcgccttggtgagccattacctcaccaaccagctaatccggcgcgggcccatcctgcttcgaggttctttgatgagaaaaggatgccttcttctcatgttattcggcattagcgacggttt

SEQ ID NO: 45 - DNA, 350 bp, Unknown
  source: /note=“Description of Unknown Clostridiales bacterium COT-082/Novel Clostridium sequence”
ttaccgcggctgctggcacgtagttagccgtggcttgttcttcaggtaccgtcattgctttcgtccctgatcaaagaagtttacaacccgaaggccttcgtccttcacgcggcattgctgggtcaggcttgcgcccattgcccaatattccccactgctgcctcccgtaggagtctgggccgtgtctcagtcccaatgtggccgttcgacctctcagtccggctaccgatcgcggtcttggtgagcctttacctcaccaactaactaatcggacgcgagcccatccttgaccggatttctcctttggtcttcaagtgatgccactcgaagacgttatgcggtattagcag

SEQ ID NO: 46 - DNA, 350 bp, Unknown
  source: /note=“Description of Unknown Moraxella sp. COT-328/Moraxella ovis sequence”
ttaccgcggctgctggcacagagttagccggtgcttattctgtgggtaacgtcagggcttatgggtattaaccacaagcttttcctccccacttaaagtgctttacaaccaaaaggccttcttcacacacgcggcatggctggatcaggctttcgcccattgtccaatattccccactgctgcctcccgtaggagtctgggccgtgtctcagtcccagtgtggctgatcatcctctcagaccagctacagatcgtcgccttggtaggcctttaccccaccaactagctaatccgacttaggctcatctgatagcgagagcttgcgccccctttcacccgtaggtcgtatg

SEQ ID NO: 47 - DNA, 350 bp, Unknown
  source: /note=“Description of Unknown TM7 sequence”
ttaccgcggctgctggcacgtagttagccgatccttattcataagctaccgtcatattcctcacttataaaagcagtttacaacccgaaggccttcatcctgcacgcggcgttgctccatcaggctttcgcccattgtggaagattcctcactgctgcctcccgtaggagtctggaccgtgtctcagttccagtctggctggtcatcctctcagaccagctacggatcgtcggcttggtgagccattacctcaccaactacctaatccgacgcgggcttctcccaaagcggataaatcctttaatccgaagatcatatgcggcattagccaccctttcgggcagttattc

SEQ ID NO: 48 - DNA, 350 bp, Unknown
  source: /note=“Description of Unknown Novel Tissierellaceae/Novel Peptostreptococcaceae sequence”
ttaccgcggctgctggcacgtagttagccggggctttcttctatggtaccgtctttcttcttcccataggacagagctttacgattcgaaaaccttcttcgctcacgcgacgtcgctgcatcagagtttcctccattgtgcaatattccccactgctgcctcccgtaggagtctggaccgtgtctcagttccaatgtggccggtcactctctcaagccggctacccatcgtcgccttggtgggctgttatctcaccaactagctaatgggtcatgagcccatcttggaccgatgaatctttaacctctctatcatgcgatagtggggtcttatgaggtattaatcgcggt

SEQ ID NO: 49 - DNA, 350 bp, Unknown
  source: /note=“Description of Unknown Moraxella sp. COT-018/FOT-087/Novel Enhydrobacter sequence”
ttaccgcggctgctggcacagagttagccggtgcttattctgcaggtaacgtcaccacttttagctattaactaaaagttcttcctccctgcttaaagtgctttacaaccttatggccttcttcacacacgcggcatggctggatcaggctttcgcccattgtccaatattccccactgctgcctcccgtaggagtccggaccgtgtctcagttccggtgtgactgatcatcctctcagaccagttacagatcgtcgccatggtaggcctttaccccaccatctagctaatctgatttaggctcatctcatatcggcagcttgcgccacctttcccccgtagggtgtatg

SEQ ID NO: 50 - DNA, 350 bp, Prevotella sp.
ttaccgcggctgctggcacggaattagccggtcctttttcttatgatacttgcaaacagccacacgtggccgactttactctcatataaaagcagtttacaacccagaaggccgtcatcctgcacgctacttggctggttcagacttccgtccattgaccaatattcctcactgctgcctcccgtaggagtctggaccgtgtctcagttccagtgtgggggatcttcctctcagaacccctacccatcgaagccttggtgggccattaccccgccaacaagctaatgggacgcatcctcatccattaccaataaatctttacttaaaagtccaggcagactaataagacc

SEQ ID NO: 51 - DNA, 350 bp, Unknown
  source: /note=“Description of Unknown Wolinella sp. FOT-098/Wolinella succinogenes sequence”
ttaccgcggctgctggcacggagttagccggtgcttattcctaagataccgtcataatcttctcttagaaaaggagtttacaatccgaagaccttcatcctccacgcggcgttgctgcttcaggctttcgcccattgagcaatattccctactgctgcctcccgtaggagtctggaccgtgtctcagttccagtgtgtccgttcaccctctcaggccggatacccgtcatagccttggtgagccattacctcaccaacaagctgataggacataggctgatcccatagcgaaaaactttcccctctcgggagtatccggtattaatcaccgtttccagtggctattccag

SEQ ID NO: 52 - DNA, 350 bp, Unknown
  source: /note=“Description of Unknown Novel Pasteurellaceae sequence”
ttaccgcggctgctggcacggagttagccggtgcttcttctgtggctaacgtcaatctaatatgctattaacatattagccttcctcaccaccgaaagaactttacaacccgaaggccttcttcattcacgcggcatggctgcgtcagggttgcccccattgcgcaatattccccactgctgcctcccgtaggagtctggaccgtgtctcagttccagtgtggctggtcatcctctcagaccagctagagatcgtcggcttggtaggcctttaccccaccaactacctaatcccacttgggctcatcttatggcgaatggctaatgtcccattctttaatccaaagatat

SEQ ID NO: 53 - DNA, 350 bp, Treponema sp.
ttaccgcggctgctggcacgtaattagccggggcttattcgccgattaccgtcatcagacaagcattccctcttatccttattcttcatcggcaaaaggattttacaacttttcagccttcttcatccacgcggcgtcgctccgtcagacttccgtccattgcggaatattcttagctgctgcctcccgtaggagtttgggccgtatctcagtcccaatgtgtccgatcaccctctcaggccggatacccatcgtcgccttggtaggccgttaccctaccaactagctaatgggacgcaggctcatccttaagcgaagccttagcttcctttccttaaaacctcttatga

SEQ ID NO: 54 - DNA, 350 bp, Unknown
  source: /note=“Description of Unknown Clostridiales bacterium COT-038 sequence”
ttaccgcggctgctggcacgtagttagccggggcttcctccttggctaccgtcttcccctcttcaccaagaacagaagtttacaacccgaaggccttcttccttcacgcggcgtcgctgggtcagggtttcccccattgcccaatattccccactgctgcctcccgtaggagtttggaccgtctctcagttccaatgtggccgttcaccctctcaggtcggctaccgatcgcagacttggtgggccgttacctcaccaactatctaatcggacgcgagcccatctcttaccggattgctcctttgacccctcggccatgcggccatgtggtcttatgcggtattagtgta

SEQ ID NO: 55 - DNA, 350 bp, Unknown
  source: /note=“Description of Unknown Pseudopropionibacterium propionicum/Novel Corynebacterium sequence”
ttaccgcggctgctggcacgtagttagccggtgcttcttctactgctaccgtcacaaaaagcttcgtcacaattgaaagcggtttacaacccgaaggccgtcatcccgcacgcggcgttgctgcatcagacttccgtccattgtgcaatattccccactgctgcctcccgtaggagtttgggccgtatctcagtcccaatgtggccggtcgccctctcaggccggctacccgtcgaagccttggtgagccactacctcaccaacaagctgataggccgcgagtccatccccaaccaaaaaaatctttccaacccacaagatgccttgcaagctcatatccagtattagca

SEQ ID NO: 56 - DNA, 350 bp, Unknown
  source: /note=“Description of Unknown Peptostreptococcaceae bacterium FOT-135/COT-066/Novel  Anaerovorax sequence”
ttaccgcggctgctggcacgtagttagccggggcttcctcaagaggtaccgtcatttttttcttccctctcaacagagttttacgacccaagggccttcatcactcacgcggcgttgctgcatcaggctttcgcccattgtgcaatattccccactgctgcctcccgtaggagtttggaccgtgtctcagttccaatgtggccgtttaccctctcaggtcggctactgatcgtcgccttggtgagccgttacctcaccaaccagctaatcagacgcaggcccatctcataccgataaatctttgaccgatgaagcatgtgctccttcggttttataaggttttaatcccg

SEQ ID NO: 57 - DNA, 350 bp, Unknown
  source: /note=“Description of Unknown Actinobacteria bacterium COT-406/Novel Euzebya sequence”
ttaccgcggctgctggcacgtagttggccggggcttcttctgaaggtaccgtcacaaaacgcttcttccctcccgaaagtggtttacaacccgaaggccttcctcccacacgcggcgtcgctgcgtcagacttccgtccattgcgcaagattccccactgctgcctcccgtaggagtctgggccgtgtctcagtcccaatgtggctgtacaagctctcactccagctacccgtcgtcgacttggtaggccattaccccaccaaccatctgataggccgcgagaccatcccgagacgccaaagctttccacccaaccccatgcggggccaggtcatatccggcattagcca

SEQ ID NO: 58 - DNA, 350 bp, Actinomyces hordeovulneris
ttaccgcggctgctggcacgtagttagccggcgcttcttctgtaagtaaagtcaaccccaacaaaaactgaggcctgcgtccctactgaaagaggtttacaacccgaaggccgtcatccctcacgcggcgtcgctgcatcaggcttgcgcccattgtgcaatattccccactgctgcctcccgtaggagtctgggccgtatctcagtcccagtgtggccggtcaccctctcaggccggctacccgtcaaagccttggtaggccatcaccccaccaacaagctgataggccgcgagcccatcccccaccagaaaaacctttccacaccccaccatgcgatgaagcgtaaat

SEQ ID NO: 59 - DNA, 350 bp, Neisseria weaveri
ttaccgcggctgctggcacgtagttagccggtgcttattcttcaggtaccgtcatcagtcatggatattagccacaaccttttcttccctgacaaaagtcctttacaacccgaaggccttcttcagacacgcggcatggctggatcaggcttgcgcccattgtccaaaattccccactgctgcctcccgtaggagtctgggccgtgtctcagtcccagtgtggcggatcatcctctcagacccgctactgatcgtcgccttggtaggcctttaccccaccaactagctaatcagatattggccgctcgaataacgcaaggcccgaaggtcccctgctttcctcctcagag

SEQ ID NO: 60 - DNA, 350 bp, Porphyromonas sp.
ttaccgcggctgctggcacggagttagccgatgcttattcatagggtacattcaaaccggtacacgtaccgaatattattcccctataaaagaagtttacaatccataagacagtcttccttcacgcgacttggctggttcaggctctcgcccattgaccaatattcctcactgctgcctcccgtaggagtctggtccgtgtctcagtaccagtgtgggggttcaacctctcagttcccctacccatcgtcgccttggtgagccgttacctcaccaactagctaatgggacgcatgcctatcttacagcgaatttcatttccttgaaaaaggatgccccctctcaagtgt

SEQ ID NO: 61 - DNA, 350 bp, Stenotrophomonas sp.
ttaccgcggctgctggcacgaagttagccggtgcttattctttgggtaccgtcatgacacacgggtattaaccgcatgcttttctttcccaacaaaagggctttacaacccgaaggccttcttcacccacgcggcatggctggatcaggcttgcgcccattgtccaatattccccactgctgcctcccgtaggagtctggaccgtgtctcagttccagtgtggctgatcatcctctcagaccagctacggatcgtcgccttggtgggccattacccccccaactagctaatccgacgtcggctcatctatctgcgcaaggcccgaaggtcccctgctttctcccgtagga

SEQ ID NO: 62 - DNA, 350 bp, Corynebacterium canis
ttaccgcggctgctggcacgtagttagccggtgcttcttctccaggtaccgtcaccaaaaaagcttcgtccccagcgaaagaggtttacaacccgaaggccgtcatccctcacgcggcgtcgctgcatcaggcttgcgcccattgtgcaatattccccactgctgcctcccgcaggagtctgggccgtatctcagtcccaatgtggccgtacaccctctcaggccggctacccgtcgacgccttggtaggccattaccccaccaacaagctgataggccgcaagctcatcccacaccagcccccaaaacagaagcctttccaccatgacacacaaaagcatggtcctatc

SEQ ID NO: 63 - DNA, 350 bp, Unknown
  source: /note=“Description of Unknown Novel Propionibacteriaceae sequence”
ttaccgcggctgctggcacgtagttagccggtgcttcttctgcaggtaccgtcattttcacttcgttcctgctgaaagcggtttacaacccgaaggccgtcatcccgcacgcggcgttgctgcatcaggctttcgcccattgtgcaatattccccactgctgcctcccgtaggagtttgggccgtatctcagtcccaatgtggccggtcgccctctcaggccggctacccgtcgaagccttggtgagccgttacctcaccaacaagctgataggccgcgagtccatccttgaccgccggagctttccaactctcgagatgcctcaagagttcatatccggtattagcagc

SEQ ID NO: 64 - DNA, 350 bp, Oscillospira sp.
ttaccgcggctgctggcacgtagttagccggagcttattcattaagtaccgtcatttgtttcgtccttaataaaagaagtttacaacccgaaagccttcttccttcacgcggcgttgctgggtcaggcttgcgcccattgcccaatattccccactgctgcctcccgtaggagtctgggccgtgtctcagtcccaatgtggccggtcaacctctcagtccggctactgatcgtcgacttggtgggccgttaccccaccaactatctaatcagacgcgaggccatctcagagcgataaaatctttggcagtcagagccatgcgacccaactgcattatgcggtattagcac

SEQ ID NO: 65 - DNA, 350 bp, Unknown
  source: /note=“Description of Unknown Novel Leptotrichiaceae sequence”
ttaccgcggctgctggcacgtatttagccgtcgcttcttctgttggtaccgtcatttgtttcttcccaactaaaagtgttttacaatccgaagaccttcttcacacacacagaattgctggatcagagtttcctccattgtccaatattccccactgctgcctcccgtaggagtaagggccgtatctcagtccccttgtggccgtccaccctctcaggccggctacctatcatcgccttggtaagcctttaccttaccaactagctaataggatgcaaagctctcttatagcgtattctttcattactctgtgatgccacagtgtaatagtatctagtattatcagacat

SEQ ID NO: 66 - DNA, 350 bp, Propionibacterium sp.
ttaccgcggctgctggcacgtagttagccggtgcttcttctgccactaccgtcactcacgcttcgtcatggctgaaagcggtttacaacccgaaggccgtcatcccgcacgcggcgttgctgcatcagacttccgtccattgtgcaatattccccactgctgcctcccgtaggagtctgggccgtatctcagtcccaatgtggccggtcgccctctcaggccggctacccgtcgacgccttggtaggccattaccccaccaacaagctgataggacgcgagtccatccccaaccgaaaaaactttccaacccaaaagatgccttccaagctgaatatccggtattagcaa

SEQ ID NO: 67 - DNA, 350 bp, Pasteurella canis
ttaccgcggctgctggcacggagttagccggtgcttcttctgtaattaacgtcaatgatattatctattcaataataccccttcctcatcaccgaaagaactttacaacccgaaggccttcttcattcacgcggcatggctgcgtcagggttccccccattgcgcaatattccccactgctgcctcccgtaggagtctgggccgtgtctcagtcccagtgtggctggccatcctctcagaccagctagagatcgcaggcttggtaggcctttaccccaccaactacctaatcccacttgggctcatcttatggcaagtggccttacggtcccaccctttcatctctcgat

SEQ ID NO: 68 - DNA, 350 bp, Unknown
  source: /note=“Description of Unknown TM7 phylum sp. COT-308 sequence”
ttaccgcggctgctggcacgtagttagccgatccttattcataagttaccgtcatattcctcacttataaaagcagtttacaacccgaaggccttcatcctgcacgcggcgttgctccatcagggttgcccccattgtggaagattcctcactgctgcctcccgtaggagtctggaccgtgtctcagttccagtctggctggtcatcctctcagaccagctacggatcgtcggcttggtaggccattaccctaccaactacctaatccgacgcgggccgctcccaaagcggataaatcctttgatccgaagataatatgcggtattagctaccctttcgggcagttatcc

SEQ ID NO: 69 - DNA, 350 bp, Treponema sp.
ttaccgcggctgctggcacgtaattagccggggcttattcgccgattaccgtcatctgataagcattccctcttatcattattcttcatcggcaaaaggattttacaacttttcagccttcttcatccacgcggcgtcgctccgtcagacttccgtccattgcggaatattcttagctgctgcctcccgtaggagtttgggccgtatctcagtcccaatgtgtccgttcaccctctcaggccggatacccatcgtcgccttggtaagccgttaccttaccaacaagctaatgggacgcaggctcatccttaagcgaagccttagcttcctttcctcacttccgcttgtgt

SEQ ID NO: 70 - DNA, 350 bp, Actinomyces cardiffensis
ttaccgcggctgctggcacgtagttagccggcgcttctttacccactaccctcaccacaacccaaagctgcggcttgaccatgagcgaaagaggttcacaacccgaaggcctacatccctcacgcggcgtcgctgcatcagacttgcgtccattgtgcaaaattccccactgctgcctcccgtaggagtctgggccgtatctcagtcccaatgtgaccggtcaccctctcaggccggttacccgtcaaagccttggtaggccactaccccaccaacaagctgataggccgcgagcccatcccccaccaaaaacacccaaaagcatctctttccacacacccccatgcgaa

SEQ ID NO: 71 - DNA, 350 bp, Neisseria canis
ttaccgcggctgctggcacgtagttagccggtgcttattcttcaggtaccgtcatcactcaagggtattaacctcaagcttttcttccctgacaaaagtcctttacaacccgaaggccttcttcagacacgcggcatggctggatcagggttccccccattgtccaaaattccccactgctgcctcccgtaggagtctgggccgtgtctcagtcccagtgtggcggatcatcctctcagacccgctactgatcgtcgccttggtaggcctttaccccaccaactagctaatcagacatcggccgctcaaatagcgcaaggcccgaaggtcccctgctttcctcctcagag

SEQ ID NO: 72 - DNA, 350 bp, Treponema sp.
ttaccgcggctgctggcacgtaattagccgttgcttattcaagccctacagtcaccgcagaggcattccctcctccgcttattcctcaggcttaaaaggattttacaacctttcggcattcttcatccacgcggcgtcgctccgtcagactttcgtccattgcggaatattcttaactgctgcctcccgtagaagtctgggccgtatctcagtcccaatgtgtccgttcaccctctcaggccggatacccatcattgccttggtgggccgttacctcaccaacaagctaatgggccgcgagccattcttctagcggagccttagctcctttcctcgcacctctctaacag

SEQ ID NO: 73 - DNA, 350 bp, Unknown
  source: /note=“Description of Unknown Synergistales bacterium COT-138/Novel Tammella sequence”
ttaccgcggctgctggcacgtagtttgccggggcttcctcgtgtggtaccgtcatcctcttcttcccacacaacagagctttacgtcccgaaggacttcttcactcacgcggcgtcgctgggtcagggttccccccattgcccaatattccccactgctgcctcccgtaggagtctgggccgtatctcagtcccagtgtggccgtgcgccctctcaggccggctacccgtcatcgcctaggtaggcctttaccccacctactagctgataggacgcgagcccctcccatgccggatctctccttttacgcaacttatgcgcttatgcggtattagcaaccgtttccagct

SEQ ID NO: 74 - DNA, 350 bp, Catonella sp.
ttaccgcggctgctggcacgtagttagccggggcttcttagtcaggtaccgtcatcatcttccctgctgatagagctttacataccgaaatacttcttcactcacgcggcgtcgctgcatcagagtttcctccattgtgcaatatcccccactgctgcctcccgtaggagtttgggccgtgtctcagtcccaatgtggccgtccgacctctcagtccggctaccgatcgtcgccttggtaagcctttaccttaccaaccagctaatcggacgcaggcccatctcataccagattgctcttttcccgaatctaccatgcggtaaatccgacttatgcggtcttagcagacg

SEQ ID NO: 75 - DNA, 350 bp, Cardiobacterium sp.
ttaccgcggctgctggcacggagttagccggtgcttcttctgcaggtaacgtcacaataacgggtattaaccattatctattcctccctgctgaaagtgctttacaacccgaaggccttcttcacacacgcggcattgctggatcagggttgcccccattgtccaatattccccactgctgcctcccgtaggagtctgggccgtgtctcagtcccagtgtggctgatcatcctctcagaccagctatggatcgtcgccttggtaggcctttaccccaccaaccagctaatccaacatgggctcatctctcgccgcgaggtccgaagatcccccgctttcaccataaggtc

SEQ ID NO: 76 - DNA, 350 bp, Actinomyces coleocanis
ttaccgcggctgctggcacgtagttagccggtacttctttaccagttaaagtcaacaacaacaaaatgcgtcttccaacctagcgaaagaggtttacaacccgaaggccgtcatccctcacgcggcgtcgctgcatcaggcttgcgcccattgtgcaaaatcccccactgctgcctcccgtaggagtctgggccgtatctcagtcccagtgtggccgtccaccctctcaggccggctacccgtcaccgccttggtaggccatcaccccaccaacaagctgataggccgcgagcccatccccaaccagaaaccaaaaaggcaccgtttcccacaaccaccatgcggcaact

SEQ ID NO: 77 - DNA, 350 bp, Propionibacterium sp.
ttaccgcggctgctggcacgtagttagccggtgcttcttctgtcggtaccgtcactcacgcttcgtcccgactgaaagcggtttacaacccgaaggccgtcatcccgcacgcggcgttgctgcatcaggcttccgcccattgtgcaatattccccactgctgcctcccgtaggagtttgggccgtatctcagtcccaatgtggccggtcgccctctcaggccggctacccgtcgaagccttggtgagccactacctcaccaacaagctgataggccgcgagcccatccccaaccgccagagctttccaaccagacccatgcaggcccgattcatatccagtattagcaac

SEQ ID NO: 78 - DNA, 350 bp, Capnocytophaga cynodegmi
ttaccgcggctgctggcacggagttagccgatgcttattcgtatggtaccgtcatcaatctacacgtagaccttattcttcccatacaaaagcagtttacaacccataaggccgtcatcctgcacgcggcatggctggatcagtcttccgaccattgtccaatattcctcactgctgcctcccgtaggagtctggtccgtgtctcagtaccagtgtgggggacatccctctcagaacccctacccatcgtagccttggtgtgccgttaccactccaactagctaatgggacgcgtactcatcttttgccaccgtagctttaattatttaatgatgccaccaaataatact

SEQ ID NO: 79 - DNA, 350 bp, Unknown
  source: /note=“Description of Unknown Novel Murdochiella/Novel Tissierellaceae sequence”
ttaccgcggctgctggcacgtagttagccggggcttcctccttgattaccgtcactatcttcaccaaggacagaactttacaatcctaagaccttcttcattcacgcggcgtcgctgcatcagagttccctccattgtgcaaaattccccactgctgcctcccgtaggagtttgggccgtgtctcagtcccaatgtggccgttcactctctcaagccggctactgatcgtcgccttggtaggccgttaccctaccaactagctaatcagacgcaaggccatctctcaccgataaatctttaaccgcttcatcatgcgataaaacagtttcatacggtattaatagtcgtt

SEQ ID NO: 80 - DNA, 350 bp, Corynebacterium mustelae
ttaccgcggctgctggcacgtagttagccggtgcttcttctacaggtaccgtcaccttaaaaaaggcttcgtccctaccgaaagaggtttacaacccgaaggccgtcatcccccacgcggcgtcgctgcatcaggcttgcgcccattgtgcaatattccccactgctgcctcccgtaggagtctgggccgtatctcagtcccaatgtggccgtccaccctctcaggccggctacccgtcgacgccttggtaggccattaccccaccaacaagctgataggccgcgggctcatctcgcaccgaaaaaactttccaccaccaacactaaatgatggtcctatccggtattag

SEQ ID NO: 81 - DNA, 350 bp, Unknown
  source: /note=“Description of Unknown Clostridiales bacterium COT-027 sequence”
ttaccgcggctgctggcacgtagttagccggggcttcctcccaggataccgtcttctgctcttctcctgggacagaggtttacgatccgaaaaccttcttccctcacgcggcgttgctgggtcagggtttcccccattgcccaatattccccactgctgcctcccgtaggagtttgggccgtgtctcagtcccaatgtggccgatcaccctctcaggtcggctaccgatcgtcgacttggtgagccgttacctcaccaactatctaatcggacgcgagcccaccccgcactggattgctcctttgacccctctgtcatgcaacagtgtggtctcatgaggtattagggag

SEQ ID NO: 82 - DNA, 350 bp, Unknown
  source: /note=“Description of Unknown Novel Lentimicrobiaceae/Novel Rikenellaceae sequence”
ttaccgcggctgctggcacggagttagccgatccttattcgctcggtacggtcgtcccggggcgcaccccggttattcctcccgcgcaaaagccgtttacaacccgtagggccgtcttccggcacgcggcatggctggttcaggcttccgcccattgaccaatattcctcactgctgcctcccgtaggagtctgggccgtgtctcagtcccagtgtggggggcctccctctcaggacccctagccatcgtcgcctcggtgggccgttaccccgccgactagctaatggcacgcatgcccatcccgcaccggccgaagcctatcactgcgattccatgcggatccgcagca

SEQ ID NO: 83 - DNA, 350 bp, Treponema sp.
ttaccgcggctgctggcacgtaattagccggggcttattcgtcgactaccgtcatcagatgagcattccctctcacccttattcttcatcaacaaaaggattttacaacttttcagccttcttcatccacgcggcgtcgctccgtcagacttccgtccattgcggaatattcttagctgctgcctcccgtaggagtttgggccgtatctcagtcccaatgtgtccgttcaccctctcaggccggatacccatcgtcgccttggtgggctcttacctcaccaactagctaatgggacgcaagctcatcctcaagcgaagccgtagcttcctttcctcaaaacctcttatga

SEQ ID NO: 84 - DNA, 350 bp, Streptococcus fryi
ttaccgcggctgctggcacgtagttagccgtccctttctggtcagttaccgtcactatgtgaactttccactctcacatacattcttggctgacaacagagctttacgatccgaaaaccttcttcactcacgcggcgttgctcggtcagggttgcccccattgccgaagattccctactgctgcctcccgtaggagtctgggccgtgtctcagtcccagtgtggccgatcaccctctcaggtcggctatgtatcgtcgccttggtaagccgttaccttaccaactagctaatacaacgcaggtccatctcacagtgaaactgttgttccttttaaatacttatcatgcaa

SEQ ID NO: 85 - DNA, 350 bp, Porphyromonas cangingivalis
ttaccgcggctgctggcacggagttagccgatacttattcttaggttacttgcttacagctacacgtaactgcttgtactcacctactaaagaagtttacaatccataggaccgtcttccttcacgcgacttggctggttcaggctctcgcccattgaccaatattcctcactgctgcctcccgtaggagtctggaccgtgtctcagttccagtgtgggggataagcctctcagctcccctatccatcgtcgacttggtgagccgttacctcaccaactatctaatggaacgcatgcctatctgtcagcaataaatctttcctaaagtccccatgcggaaaccaaagtgt

SEQ ID NO: 86 - DNA, 350 bp, Dielma sp.
ttaccgcggctgctggcacgtagttagccgtggctttctggtaaggtaccgtcatcatttaatcattccctattaaatttattcttcccttacaacagagttttacacaacgaattgcttcatcactcacgcggcattgctccatcagggttgcccccattgtggaaaattccctactgctgcctcccgtaggagtttgggccgtgtctcagtcccaatgtggccgttcaccctctcaggtcggctatgcatcatcgccttggtgagccgttacctcaccaactagctaatgcaccgcaggtccatcccattgtgttgccttagcaactttcaatacaggaagatgcctt

SEQ ID NO: 87 - DNA, 350 bp, Unknown
  source: /note=“Description of Unknown Bergeyella zoohelcum/Novel Weeksellaceae sequence”
ttaccgcggctgctggcacggagttagccggtgcttattcatacagtaccttcagctaccctcacgagagtaggtttatccctgtataaaagaagtttacaaaccataggtccttagtccttcacgcgggatggctggatcaggctctcacccattgtccaatattcctcactgctgcctcccgtaggagtctggtccgtgtctcagtaccagtgtgggggatcaccctctcaggccccctaaagatcgcagccttggtgagccgttacctcaccaactagctaatcttacgcgtgcccatctctatccgccggagctttcaatattaactgatgccaatcaatatatta

SEQ ID NO: 88 - DNA, 350 bp, Unknown
  source: /note=“Description of Unknown Lachnospiraceae bacterium COT-106 sequence”
ttaccgcggctgctggcacatagttagccggggcttcttagtcaggtaccgtcattttcttccctgctgatagaagtttacaatccgaagaccttcttccttcacgcggcattgctgcatcagagtttcctccattgtgcaatatcccccactgctgcctcccgtaggagtttgggccgtgtctcagtcccaatgtggccgatcaccctctcaggtcggctactgatcgttgccttggtaggcttttaccctaccaactagctaatcagacgcgggcccgtcttacaccaccggagtttttcacactgtactatgcagtactgtgcgcttatgcggtattagcacagatt

SEQ ID NO: 89 - DNA, 350 bp, Unknown
  source: /note=“Description of Unknown Christensenellaceae family sequence”
ttaccgcggctgctggcacgtagttagccggggcttcctccttggttaccgtctttccctcttcaccaagaacagaagtttacaacccgaaggccgtcttccttcacgcggcgtcgctgggtcagggtttcccccattgcccaatattccccactgctgcctcccgtaggagtctggaccgtgtctcagttccaatgtggccgatcaccctctcaggtcggctaccgatcgttgacttggtgggccgttacctcaccaactatctaatcggacgcgagcccatcttttaccggatttctcctttgacccctttaacattcgttcttgtggtctcatgcggtattagggtc

SEQ ID NO: 90 - DNA, 350 bp, Unknown
  source: /note=“Description of Unknown Anaerolineae bacterium FOT-333 sequence”
ttaccgcggctgctggcacgtagttagccgagacttattctcgtggtactgtccttcctcatcccacgtaaaagaactttacgacccgaaggccttcttcgttcacgcggcgttgctgcttcaggctttcgcccattaagcaatattccctactgctgccacccgcaggtgtatggaccgtgtttcagttccattgtgggggatcaccctctcaggtcccctacccgtcttcgccttggtgagccgttacctctccaactagctgatgggccgcaggcccctcccaaagtagatttctcttttactgtctcccttctaatctgagacagcttatgcggttttgtcactcg

SEQ ID NO: 91 - DNA, 350 bp, Unknown
  source: /note=“Description of Unknown Lachnospiraceae bacterium COT-161/FOT-003 sequence”
ttaccgcggctgctggcacatagttagccggggcttcttagtcaggtaccgtcattttcttccctgctgatagaagtttacaatccgaaaaccgtcttccttcacgcggcattgctgcatcagagtttcctccattgtgcaatatcccccactgctgcctcccgtaggagtttgggccgtgtctcagtcccaatgtggccgatcaccctctcaggtcggctactgatcgtcgccttggtaggcttttaccccaccaactagctaatcagacgcgggcccgtcttacaccaccagagtttttcacaccgtatcatgtgatactgtgcgcttatgcggtattagcacagatt

SEQ ID NO: 92 - DNA, 350 bp, Cardiobacterium sp.
ttaccgcggctgctggcacggagttagccggtgcttcttctgcaggtaacgtcacaataacgggtattaaccgtcatctcttcctccctgctgaaagtgctttacaacccgaaggccttcttcacacacgcggcattgctggatcagggttgcccccattgtccaatattccccactgctgcctcccgtaggagtctgggccgtgtctcagtcccagtgtggctgatcatcctctcaaaccagctatggatcgtcgccttggtaggcctttaccccaccaactagctaatccaacataggctcatccatcagcgcgaggtctaaaagatcccccgctttcacccttaggt

SEQ ID NO: 93 - DNA, 350 bp, Comamonas sp.
ttaccgcggctgctggcacgtagttagccggtgcttattcttacagtaccgtcatgacccccttgtattagaaagaggcttttcgctctgtacaaaagcagtttacaacccgaaggccttcgtcctgcacgcggcattgctggatcaggcttgcgcccattgtccaaaattccccactgctgcctcccgtaggagtctgggccgtgtctcagtcccagtgtggctgatcgtcctctcagaccagctacagatcgcaggcttggtaagcctttaccccaccaactacctaatctgccatcagccgctccaatagcgcgaggtccgaagatcccccgctttcaccctcaggt

SEQ ID NO: 94 - DNA, 350 bp, Unknown
  source: /note=“Description of Unknown Porphyromonadaceae bacterium COT-184/Novel Paludibacter sequence”
ttaccgcggctgctggcacggagttagccgatgcttattcgtacagtacttgcaaaaccctacacgtagaaccttttacccctgtacaaaagaagtttacaacccataaggctgtcttccttcacgcgacttggctggttcaggctctcgcccattgaccaatattcctcactgctgcctcccgtaggagtctggtccgtgtctcagtaccagtgtgggggaccttcctctcagaacccctaaggatcgtagccttggtaagccgttaccttaccaactagctaatcctacgcatacccatctctaatcgataaatctttaaaaatatccagatgcctaaatatcttact

SEQ ID NO: 95 - DNA, 350 bp, Capnocytophaga sp.
ttaccgcggctgctggcacggagttagccgatgcttattcgtatggtaccgtcatcaaggtacacgtacctcttattctccccatacaaaagcagtttacaacccataaggcagtcttcctgcacgcggcatggctggttcagtctttcgaccattgaccaatattcctcactgctgcctcccgtaggagtctggtccgtgtctcagtaccagtgtgggggatctccctctcaggacccctacctatcgtagccttggggtgccgttaccactccaactagctaataggacgcgtactcatcttacaccgcctaaactttaattaccactcgatgccaagcaataatact

SEQ ID NO: 96 - DNA, 350 bp, Unknown
  source: /note=“Description of Unknown Clostridiales bacterium COT-216/Novel Clostridium sequence”
ttaccgcggctgctggcacgtagttagccgtggcttattctccgggtaccgtcatttgtttcgtccccggctaaagaagtttacaactcgaaagccttcatccttcacgcggcattgctgggtcaggcttgcgcccattgcccaatattccccactgctgcctcccgtaggagtctgggccgtgtctcagtcccaatgtggccgatcaacctctcagttcggctaccgatcgttgccttggtgagcctttacctcaccaactagctaatcggacgcgagctcatcttctaccggattgctcctttgatactcaagtgatgccacccaagtatgttatgcggtattagcaa

SEQ ID NO: 97 - DNA, 350 bp, Porphyromonas sp.
ttaccgcggctgctggcacggagttagccgatgcttattcatagggtacattcaatccggtacgcgtaccgaccattattcccctataaaagaagtttacaatccataagacattcttccttcacgcgacttggctggttcaggctctcgcccattgaccaatattcctcactgctgcctcccgtaggagtctggtccgtgtctcagtaccagtgtgggggttcaacctctcagttcccctacccatcgtcgccttggtgagccgttacctcaccaactagctaatgggacgcatgcctatcttacagcgaatctctttcctcgtctatagatgcctacaaacgagtgta

SEQ ID NO: 98 - DNA, 350 bp, Unknown
  source: /note=“Description of Unknown Fretibacterium COT-178/FOT-215/ Novel Synergistes/ Novel Dethiosulfovi brionaceae sequence”
ttaccgcggctgctggcacgtagttagccggggcttattcatgtggtaccgtcacttccttcttcccacataaaagaactttacgacccgaaggccttcttcgttcacgcggcgtcgctgggtcaggatttctcccattgcccaatattccccactgctgcctcccgtaggagtctggaccgtatctcagttccagtgtggccggacaccctctcaggccggctacccgtcgtcgccttggtgagccgttacctcaccaaccagctgataggacacgagcacctccaatagcgcatcgctgctttaaccaaaaggcctatggggtattagggtccgtttccgaacgttat

SEQ ID NO: 99 - DNA, 350 bp, Abiotrophia sp.
ttaccgcggctgctggcacgtagttagccgtggctttctggttagataccgtcacattatgggcagttactcccataactcttcttctctaacaacagagctttacgatccgaagaccttcttcactcacgcggcgttgctccgtcagactttcgtccattgcggaagattccctactgctgcctcccgtaggagtttgggccgtgtctcagtcccaatgtggccgatcaccctctcaggtcggctatgcatcatcgccttggtaagccgttaccttaccaactagctaatgcaccgcgggtccatctctaagtgacagccgaaaccgtctttcctcactgcgtcatgtg

SEQ ID NO: 100 - DNA, 350 bp, Unknown
  source: /note=“Description of Unknown Novel Fastidiosipila/Novel Clostridium sequence”
ttaccgcggctgctggcacgtagttagccgtggcttgttcttcaggtaccgtcatttgtttcgtccctgaccaaagaagtttacaatccgaagaccttcatccttcacgcggcgttgctgggtcaggcttgcgcccattgcccaatattccccactgctgcctcccgtaggagtctgggccgtgtctcagtcccaatgtggccgatcaacctctcagttcggctaccgatcgcggtcttggtgggccgttaccccaccaactaactaatcggacgcgagcccatctctcaccgataaaatctttggtcccccgatgatgccacccggggacattatgcggtattagcaac

SEQ ID NO: 101 - DNA, 350 bp, Porphyromonas sp.
ttaccgcggctgctggcacggagttagccgatgcttattcatagggtacattcaatctagcacacgtgctagccgttattcccctataaaagaagtttacaatccataagaccttcatccttcacgcgacttggctggttcaggctcccgcccattgaccaatattcctcactgctgcctcccgtaggagtctggtccgtgtctcagtaccagtgtgggggttcaacctctcagttcccctacccatcgttgccttggtgagccgttacctcaccaactagctaatgggacgcatgcctatctcgtagcaaaattctttcctcactagatcatgcgaccttgtgagcata

SEQ ID NO: 102 - DNA, 350 bp, Porphyromonas sp.
ttaccgcggctgctggcacggagttagccgatgcttattcatagggtacattcaattcggtacacgtaccgaccattattcccctataaaagaagtttacaatccataagacattcttccttcacgcgacttggctggttcaggctctcgcccattgaccaatattcctcactgctgcctcccgtaggagtctggtccgtgtctcagtaccagtgtgggggttcaacctctcagttcccctacccatcgttgccttggtgagccgttacctcaccaactagctaatgggacgcatgcctatcttacagcgaatttctttcctcgtatatagatgcctatttacgagtgta

SEQ ID NO: 103 - DNA, 350 bp, Unknown
  source: /note=“Description of Unknown Erysipelotrichaceae bacterium COT-381 sequence”
ttaccgcggctgctggcacgtagttagccgtggctttctgatcaggtaccgtccctctatcaatcattccctatcaatagcattcttccctgataacagagttttacaacccgaaggccttcttcactcacgcggcgttgctcggtcagggttccccccattgccgaaaattccctactgctgcctcccgtaggagtctgggccgtgtctcagtcccagtgtggccggtcgccctctcaggccggctatgcatcatcgccttggtaagccgttaccttaccaactagctaatgcaccataaactcatctcttatcggtgttgcccacctttcataaacaaaggatgccct

SEQ ID NO: 104 - DNA, 350 bp, Neisseria sp.
ttaccgcggctgctggcacgtagttagccggtgcttattcttcaggtaccgtcatcactcaagggtattagcctcaagcttttcttccctgacaaaagtcctttacaacccgaaggccttcttcagacacgcggcatggctggatcaggcttgcgcccattgtccaaaattccccactgctgcctcccgtaggagtctgggccgtgtctcagtcccagtgtggcggatcatcctctcagacccgctactgatcgtcgccttggtaggcctttaccctaccaaccagctaatcagacatcggccgctcgaatagcgcgaggtccgaagatcccccgctttccttctcaagc

SEQ ID NO: 105 - DNA, 350 bp, Porphyromonas sp.
ttaccgcggctgctggcacggagttagccgatgcttattcatagggtacattcaacttagcacacgtactaattattattcccctataaaagaagtttacaatccataagacagtcatccttcacgcgaattggctggttcaggctcgcgcccattgaccaatattcctcactgctgcctcccgtaggagtctggtccgtgtctcagtaccagtgtgggggataaacctctcagttcccctacccatcgtcgccttggtgagccgttacctcaccaactagctaatgggacgcatgcctatcctgtagcgaatttctttcctcatcaaacgatgccgtccaatgagagta

SEQ ID NO: 106 - DNA, 350 bp, Unknown
  source: /note=“Description of Unknown Peptostreptococcaceae bacterium COT-067/FOT-137/Novel Mogibacteriaceae sequence”
ttaccgcggctgctggcacgtagttagccggggcttcctcctcgggtaccgtcatttttttcttcccctaggacagagctttacgacccaaaggccttcttcgctcacgcggcgttgctgcatcaggctttcgcccattgtgcaagattccccactgctgcctcccgtaggagtttggaccgtgtctcagttccaatgtggccgattaccctctcaggtcggctactgatcgctgccttggtgagccgttacctcaccaactaactaatcagacgcaggcccatcccataccgataaatctttgacctcataaacatgtgtttttgaggctttatgaggcattaatcaaa

SEQ ID NO: 107 - DNA, 350 bp, Haemophilus sp.
ttaccgcggctgctggcacggagttagccggtgcttcttctgtgactaacgtcaatttgtcatgctattaacatcacaaccttcctcatcaccgaaagaactttacaacccgaaggccttcttcattcacgcggcatggctgcgtcagggttgcccccattgcgcaatattccccactgctgcctcccgtaggagtctggaccgtgtctcagttccagtgtggctggtcatcctctcagaccagctagagatcgaaggcttggtaggcctttaccccaccaactacctaatcccacttgggctcatcttatggcaactggctaaaagtcccagtctttaatctttcgata

SEQ ID NO: 108 - DNA, 350 bp, Moraxella sp.
ttaccgcggctgctggcacagagttagccggtgcttattctgtgggtaacgtcagggctaataggtattaattattagcttttcctccccacttaaagtgctttacaaccaaaaggccttcttcacacacgcggcatggctggatcaggctttcgcccattgtccaatattccccactgctgcctcccgtaggagtctgggccgtgtctcagtcccagtgtggctgatcatcctctcagaccagctacagatcgtcgccttggtaggcctttaccccaccaactagctaatccgacttaggctcatctattagcgcaaggtcttgcgatcccctgctttctcccgtagga

SEQ ID NO: 109 - DNA, 350 bp, Actinomyces sp.
ttaccgcggctgctggcacgtagttagccggtacttctttaccagttaaagtcaacaactccaaaaagcgtcttccaacctagcgaaagaggtttacaacccgaaggccgtcatccctcacgcggcgtcgctgcatcaggcttgcgcccattgtgcaaaatcccccactgctgcctcccgtaggagtctgggccgtatctcagtcccagtgtggccgtccaccctctcaggccggctacccgtcaccgccttggtaggccatcaccccaccaacaagctgataggccgcgagcccatccccaaccagaaaaaaccgtttcccacaaccaccatgcgatgactgcagaata

SEQ ID NO: 110 - DNA, 350 bp, Unknown
  source: /note=“Description of Unknown Novel Peptostreptococcaceae/Novel Eubacterium sequence”
ttaccgcggctgctggcacgtagttagccggggctttcttgtatggtactgtcatttttttcttcccatacgacagagctttacgactcgaaagccttcttcgctcacgcggcgttgctgcatcaggctttcgcccattgtgcaatatcccccactgctgcctcccgtaggagtttggaccgtctctcagttccaatgtggccgttcaccctctcaggccggctactgatcgtcgccttggtaggctcttaccctgccaactagctaatcagacgcaagctcgtctacaaccgcctcagctttcatatttatatctcccgatatacttacttcatctcgtattagctaag

SEQ ID NO: 111 - DNA, 350 bp, Catonella sp.
ttaccgcggctgctggcacgtagttagccggggcttcttagtcaggtaccgtcatcatcttccctgctgatagagctttacataccgaaatacttcttcactcacgcggcgtcgctgcatcagagtttcctccattgtgcaatatcccccactgctgcctcccgtaggagtttgggccgtgtctcagtcccaatgtggccgtccgacctctcagtccggctaccgatcgtcgccttggtaagcttttaccttaccaactagctaatcggacgcgggcccatctcataccagattgctcttttcccgtctctgtcatgcgacaaagacgacttatgcggtcttagcagacg

SEQ ID NO: 112 - DNA, 350 bp, Actinomyces sp.
ttaccgcggctgctggcacgtagttagccggcgcttcttatccagctaccgtcaacccacccacaaagagatgagcctgcttcactggcgaaagaggttcacaacccgaaggcctccatccctcacgcggcgtcgctgcatcaggcttgcgcccattgtgcaatattccccactgctgcctcccgtaggagtctgggccgtgtctcagtcccagtgtggccgtccaccctctcaggccggctacccgtcaccgccttggtaggccatcaccccaccaacaagctgataggccgcgagcccatcccccaccagaacaatacctttcccaacccatgcatgcgcacagacca

SEQ ID NO: 113 - DNA, 350 bp, Unknown
  source: /note=“Description of Unknown Streptococcus constellatus subsp. constellatus/Streptococcus anginosus subsp. Anginosus/Streptococcus intermedius sequence”
ttaccgcggctgctggcacgtagttagccgtccctttctggttaagtaccgtcactgtgtgaactttccactctcacacacgttcttccttaacaacagagctttacgatccgaaaaccttcttcactcacgcggcgttgctcggtcagggttccccccattgccgaagattccctactgctgcctcccgtaggagtctgggccgtgtctcagtcccagtgtggccgatcaccctctcaggtcggctatgtatcgttgccttggtgagccgttacctcaccaactagctaatacaacgcaggtccatctggtagtgatgcttttgcaccttttaaacatctaccatgcag

SEQ ID NO: 114 - DNA, 350 bp, Filifactor villosus
ttaccgcggctgctggcacgtagttagccggggctttcttttaaggtaccgtcattatcttcccttacaacagagttttacgactcgaaagccttcttcactcacgcggcgttgctgcatcagggttccccccattgtgcaatattccccactgctgcctcccgtaggagtttggaccgtgtctcagttccaatgtggccgttcaccctctcaggccggctactgatcgtcgccttggtaggcttttaccctaccaaccagctaatcagatgcgggctcatctttgtccgatttctctttgatatcttatagatgcctatacgatatgttatttggtattagctactttt

SEQ ID NO: 115 - DNA, 350 bp, Neisseria shayeganii
ttaccgcggctgctggcacgtagttagccggtgcttattcttcaggtaccgtcatcactccagggtattaacccaaagcttttcttccctgacaaaagtcctttacaacccgaaggccttcttcagacacgcggcatggctggatcaggcttgcgcccattgtccaaaattccccactgctgcctcccgtaggagtctgggccgtgtctcagtcccagtgtggcggatcatcctctcagacccgctactgatcgtcgccttggtaggcctttaccccaccaactagctaatcagacatcggccgctcgaatagcgcgaggtccgaagatcccccgctttccttctcaaag

SEQ ID NO: 116 - DNA, 350 bp, Treponema sp.
ttaccgcggctgctggcacgtaattagccgttgcttattcaagccctactgtcaccgcaaaggcattccctcctctgcttattcatcaggcttaaaaggattttacaacctttcggcattcttcatccacgcggcgtcgctccgtcagacttgcgtccattgcggaatattcttaactgctgcctcccgtagaagtctgggccgtatctcagtcccaatgtgtccgttcaccctctcaggccggatacccatcattgccttggtgggccgttacctcaccaactagctaatgggccgcgagccattcttccagcgaagccttagctcctttcctcatacagctctaacct

SEQ ID NO: 117 - DNA, 350 bp, Alloprevotella sp.
ttaccgcggctgctggcacggaattagccggtcctttttctgatggtacattcttatcagccacacgtggctgccgttattcccaactaaaagcagtttacaacccggagggccgtcatcctgcacgctacttggctggttcagactttcgtccattgaccaatattcctcactgctgcctcccgtaggagtctggaccgtgtctcagttccagtgtgggggaccttcctctcagaacccctactgatcgttggctaggtgggccgttaccccgcctactacctaatcagacgcatcaccatcgcataccgataaatctttgattatctacccatgcgggaaaacaataa

SEQ ID NO: 118 - DNA, 350 bp, Unknown
  source: /note=“Description of Unknown SR1 bacterium COT-380 sequence”
ttaccgcggctgctggcacgtagttagccgtctcttattcttgacataccgtcattcgtcttctgccaaaaaagaagtttacacaccgtaatgcttcatccttcacgcgaaattgctgcatcagactttcgtccattgtgcaagattccccactgctgcctcccgtaggagtatgggccgtatctcagtcccatcgcgaccggacatcatctcagaccggttacccgtcattggcttggtaggccattaccccaccaactacctgataggacgcacccccctcccgaagcgataaatctttcctcacattggaatttcaccatcgtgagcatattgggaattagctacat

SEQ ID NO: 119 - DNA, 350 bp, Proteocatella sp.
ttaccgcggctgctggcacgtagttagccggggcttcctcacatggtaccgtcattatcttcccatgcgacagagctttacgatccgaagaccttcatcgctcacgcggcgttgctgcatcaggcttgcgcccattgtgcaatattccccactgctgcctcccgtaggagtctggaccgtgtctcagttccagtgtggccgatcaccctctcaggtcggctactgatcgtcgccttggtaggcctttaccccaccaactagctaatcagacgcaggctcatccttatccgctttcgctttgatatcctctcgatgccaaatgaatatgttattcggtattagcacatctt

SEQ ID NO: 120 - DNA, 350 bp, Unknown
  source: /note=“Description of Unknown Clostridiales bacterium FOT-118 sequence”
ttaccgcggctgctggcacgtagttagccgtggcttattcattcagtaccgtcatttctttcgtcccgaataaaagaagtttacaacccgaaagccttcttccttcacgcggcgttgctgggtcaggcttgcgcccattgcccaatattccccactgctgcctcccgtaggagtctgggccgtgtctcagtcccaatgtggccggtcaacctctcagtccggctactgatcgtcgcctaggtaggccgttaccccacctactagctaatcagacgcgaggccatctcagagcgataaatctttggcatagttaccatgcgatagctatgcttcatgcggtattagcacaa

SEQ ID NO: 121 - DNA, 350 bp, Odoribacter denticanis
ttaccgcggctgctggcacggagttagccgatccttattcgtcaggtaccggcatcggagaattgaatcccccttattcttccctgacaaaagaagttgaaaacccatagggcagtttccttcacgcgacttggctggttcagtcttacgaccattgaccaatattcctcactgctgcctcccgtaggagtctggaccgtttctcaattccagtgtgggggaccttcctctcagaacccctatccatcatcggattggtaagccgttaccttaccaacttcctaatggaacgcatgctcatctcttaccgataaatctttattcccttcatgaggccacaaaagaaatgc

SEQ ID NO: 122 - DNA, 350 bp, Conchiformibius steedae
ttaccgcggctgctggcacgtagttagccggtgcttattcttacggtaccgtcatcagtcatgggtattaaccacaactttttcttcccgtacaaaagtcctttacaacccgaaggccttcttcagacacgcggcatggctggatcaggcttgcgcccattgtccaaaattccccactgctgcctcccgtaggagtctgggccgtgtctcagtcccagtgtggcggatcatcctctcagacccgctactgatcgttgccttggtaggcttttaccccaccaactagctaatcagacattggccgctcggataacgcgaggtcaaatgatcccccgctttccttcttaaag

SEQ ID NO: 123 - DNA, 350 bp, Fretibacterium sp.
ttaccgcggctgctggcacgtagttagccggggcttattcatgtggtaccgtcactctcttcttcccacataaaagaactttacgacccgaaggccttcttcgttcacgcggcgtcgctgggtcaggattcctcccattgcccaatattccccactgctgcctcccgtaggagtctggaccgtctctcagttccagtgtggccggtcaccctctcaggccggctacccgtcatcgccttggtgagcccttacctcaccaactagctgataggacacgagcccctcctaccgcgcgtctccgctttaacccgtaggcctatggggtattagggtccgtttccgaacgttat

SEQ ID NO: 124 - DNA, 350 bp, Neisseria zoodegmatis
ttaccgcggctgctggcacgtagttagccggtgcttattcttcaggtaccgtcatcagtcatgggtattaaccacaaccttttcttccctgacaaaagtcctttacaacccgaaggccttcttcagacacgcggcatggctggatcaggcttgcgcccattgtccaaaattccccactgctgcctcccgtaggagtctgggccgtgtctcagtcccagtgtggcggatcatcctctcagacccgctactgatcgtcgccttggtaggcctttaccccaccaaccagctaatcagacatcggccgctcgaatagcgcaaggtccgaagagcccctgctttccttctcaaag

SEQ ID NO: 125 - DNA, 350 bp, Schwartzia sp.
ttaccgcggctgctggcacgtagttagccgtggcttcctcgtcaggtaccgtcttagaccagccattgcctgctgctccttttctcccctgacaacagagttttacgatccgaagaccttcttcactcacgcggcgttgctccgtcaggctttcgcccattgcggaagattccccactgctgcctcccgtaggagtatgggccgtgtctcagtcccattgtggccgttcatcctctcagaccggctactgatcgttgctttggtgagccgttacctcaccaactggctaatcagacgcaggcccatcctccggcgatggcttgcaccatctttgctatacctcccatgcg

SEQ ID NO: 126 - DNA, 350 bp, Moraxella sp.
ttaccgcggctgctggcacagagttagccggtgcttattctgcaggtaacgtcagggcttgtgggtattaaccacaagcttttcctccctgcttaaagtgctttacaaccaaaaggccttcttcacacacgcggcatggctggatcaggctttcgcccattgtccaatattccccactgctgcctcccgtaggagtccggaccgtgtctcagttccggtgtggctgatcatcctctcagaccagctacagatcgtcgccttggtaggcctttaccccaccaactagctaatccgacttaggctcatctaatagcgagagcttacgccccctttcacccgtaggtcgtatg

SEQ ID NO: 127 - DNA, 350 bp, Unknown
  source: /note=“Description of Unknown Fusibacter/ Peptostreptococcaceae bacterium COT-104 sequence”
ttaccgcggctgctggcacgtagttagccggggcttcctcaaatggtaccgtcattatcttcccattcaacagagttttacagtccgaagaccttcaatcactcacgcggcgttgctcggtcagggttccccccattgccgaatattccccactgctgcctcccgtaggagtctggaccgtgtctcagttccagtgtggccgttcaccctctcaggtcggctactgatcgtcgccttggtaagccgttaccttaccaactagctaatcagacgcgggcccatctcttaccgataaatctttggaacatctatcatgcgataaatgcacttcatacagtattaatcaccgt

SEQ ID NO: 128 - DNA, 350 bp, Peptostreptococcus anaerobius
ttaccgcggctgctggcacgtagttagccggggcttcctcacagggtaccgtcattatcttcccctgcaacagaactttacgatccgaagaccttcatcgttcacgcggcgttgctgcatcaggcttgcgcccattgtgcaatattccccactgctgcctcccgtaggagtttggaccgtgtctcagttccaatgtggccgatcaccctctcaggtcggctactgatcgtcgccttggtgggcagttatctcaccaactagctaatcagacgcgggtccatcctataccgctaacactttgataattcttcgatgccgaaaaactattttatcatgtattagtaaacttt

SEQ ID NO: 129 - DNA, 350 bp, Canibacter oris
ttaccgcggctgctggcacgtagttagccggtgctttttctgtaggtaccgtcacttacgcttcttccctactaaaagaggtttacaacccgaaggccgtcatccctcacgcggcgttgctgcatcaggcttgcgcccattgtgcaatattccccactgctgcctcccgtaggagtctgggccgtgtctcagtcccagtgtggccggtcaccctctcaggccggctacccgtcgtcgccatggtgagccactacctcaccatcaagctgataggccgcgagtccatcccaaaccgaaaaatctttccaccaaacaccatgcgatgaaaggtaatatccagtattagacgc

SEQ ID NO: 130 - DNA, 350 bp, Helcococcus sp.
ttaccgcggctgctggcacgtatttagccggggctttcttcttggttactgtcattatcttcaccaaggacagaactttacaacccgaaggccttcttcgttcacgcggcgtcgctgcatcagggtttcccccattgtgcaaaattccccactgctgcctcccgtaggagtttgggccgtgtctcagtcccaatgtggccgtccaccctctcagatcggctactgatcgtcgccttggtaggccgttaccccaccaaccagctaatcaggcgcgagaccatcttacaccgataaatctttgaccgcatcatcatgcgatgtcacggtgtcatgaggtattaatcgtcgtt

SEQ ID NO: 131 - DNA, 350 bp, Unknown
  source: /note=“Description of Unknown Christensenellaceae/Clostridiales bacterium COT-157 sequence”
ttaccgcggctgctggcacgtagttagccggggcttccttcttggctaccgtcttctttcttcaccaaggatagaagtttacaatccgaaaaccttcttccttcacgcggcgttgctgggtcagagttccctccattgcccaatattccctactgctgccttccgtagaagtctggaccgtgtctcagttccagtgtggccgatcaccctcttaggtcggctatctatcgttgacttggtgagccgttacctcaccaactatctaatagaacgcgagctccccctttaccgtgtctccactttcatcctttcatcatgcgattcttggatattatgcggtattagtacaa

SEQ ID NO: 132 - DNA, 350 bp, Haemophilus sp.
ttaccgcggctgctggcacggagttagccggtgcttcttctgtgaataacgtcaatttgtcatgctatttacataacaaccttcctcatcaccgaaagaactttacaacccgaaggccttcttcattcacgcggcatggctgcgtcagggttacccccattgcgcaatattccccactgctgcctcccgtaggagtctggaccgtgtctcagttccagtgtggctggtcatcctctcagaccagctagagatcgaaggcttggtaggcctttaccccaccaactacctaatcccacttgggctcatcttatggcaactggccttgcggtcccagtctttaatctttcgat

SEQ ID NO: 133 - DNA, 350 bp, Unknown
  source: /note=“Description of Unknown Peptostreptococcaceae bacterium FOT-040 sequence”
ttaccgcggctgctggcacgtagttagccggggcttcctcctatggtaccgtcatcatcttcccataggacagagctttacgactcgaaagccttcatcgctcacgcggcgttgctgcatcagggtttcccccattgtgcaatattccccactgctgcctcccgtaggagtctggaccgtgtctcagttccagtgtggccgatcaccctctcaggtcggctacggatcgtcgccttggtgagccgttacctcaccaaccagctaatccggcgcaggcccatcctgtgccaaaattctttgatcgcctctcgatgccgacaggcgatattattcggcattagcaccggttt

SEQ ID NO: 134 - DNA, 350 bp, Unknown
  source: /note=“Description of Unknown Leptotrichiaceae family sequence”
ttaccgcggctgctggcacgtatttagccgtcgcttcttctgccggtaccgtcacttccttcttcccggctgaaagcactttacaatccgaaaaccttcctcgtgcacacagaattgctggatcagagtctcctccattgtccaatattccccactgctgcctcccgtaggagtaagggccgtatctcagtccccttgtggccgttcaccctctcaggccggctacctatcttcgccttggtgggccgttaccccaccaactagctaataggacgcaaagctctcctatggcatctcttttcatcccgccgccatgcggcagcagaacaccatccggtcttatcagccgt

SEQ ID NO: 135 - DNA, 350 bp, Conchiformibius steedae
ttaccgcggctgctggcacgtagttagccggtgcttattcttcgggtaccgtcatcagtcatgggtattagccacaaccttttcttccccgacaaaagtcctttacaacccgaaggccttcttcagacacgcggcatggctggatcaggcttgcgcccattgtccaaaattccccactgctgcctcccgtaggagtctgggccgtgtctcagtcccagtgtggcggatcatcctctcagacccgctactgatcgtcgccttggtaggcttttaccccaccaactagctaatcagacattggccgctcggataacgcgaggtcaaacgatcccccgctttccttcttaaag

SEQ ID NO: 136 - DNA, 350 bp, Lautropia sp.
ttaccgcggctgctggcacgtagttagccggtgcttattcttcaggtaccgtcatcggcccatggtattagcacggaccttttcgttcctgacaaaagtgctttacaacccgaaggccttcatcacacacgcggcattgctggatcaggcttgcgcccattgtccaaaattccccactgctgcctcccgtaggagtctgggccgtgtctcagtcccagtgtggctggtcgtcctctcagaccagctacagatcgtcgccttggtaagcctttacctcaccaactagctaatctgccatcggccgctctgatagcgcgaggcccgaaggtcccccgctttcaccctgaggt

SEQ ID NO: 137 - DNA, 350 bp, Bacteroides sp.
ttaccgcggctgctggcacggagttagccgatgcttattcatatgttacatgcaaaaacctacacgtaggaaaatttattcacatataaaagaagtttacaacccagagggcagtcatccttcacgctacttggctggttcagacttacgtccattgaccaatattcctcactgctgcctcccgtaggagtttggaccgtgtctcagttccaatgtgggggaccttcctctcagaacccctaaccatcgaagtcttggtgggccgttaccccgccaacaaactaatggtacgcatccccatctatcaccgataaatctttaatatacaaaacatgcgaaatgtatatacc

SEQ ID NO: 138 - DNA, 350 bp, Actinomyces sp.
ttaccgcggctgctggcacgtagttagccggcgcttcttatccagctaccgtcaccacccccaaaaaaagagcggctgcttcactggcgaaagaggtttacaacccgaaggccgtcatccctcacgcggcgtcgctgcatcaggcttgcgcccattgtgcaatattccccactgctgcctcccgtaggagtctgggccgtgtctcagtcccagtgtggccgtccaccctctcaggccggctacccgtcaaagccttggtaggccatcaccccaccaacaagctgataggccgcgagcccatcccccaccaaaacaaatctttccaaacccacccatgcgagcaggccaga

SEQ ID NO: 139 - DNA, 350 bp, Unknown
  source: /note=“Description of Unknown Lachnospiraceae bacterium FOT-156 sequence”
ttaccgcggctgctggcacgtagttagccggggcttcttagtcaggtaccgtcattttcttccctgctgatagagctttacataccgaagtacttcttcactcacgcggcgtcgctgcatcagggttccccccattgtgcaatattccccactgctgcctcccgtaggagtttgggccgtgtctcagtcccaatgtggccgatcactctctcaagtcggctactgatcgtcgacttggtgagccgttacctcaccaactatctaatcagatgcgggtccatctcatatcgataaatcttttccctctgtatcatgcgataccgggggcttatgcggtattagcagcggtt

SEQ ID NO: 140 - DNA, 350 bp, Conchiformibius sp.
ttaccgcggctgctggcacgtagttagccggtgcttattcttacggtaccgtcatcagtcatgggtattagccacaactttttcttcccgtacaaaagtcctttacaacccgaaggccttcttcagacacgcggcatggctggatcaggcttgcgcccattgtccaaaattccccactgctgcctcccgtaggagtctgggccgtgtctcagtcccagtgtggcggatcatcctctcagacccgctactgatcgtcgccttggtaggcttttaccccaccaactagctaatcagacattggccgctcggataacgcgaggtccgaagatcccccgctttcctcctaagag

SEQ ID NO: 141 - DNA, 350 bp, Actinomyces sp.
ttaccgcggctgctggcacgtagttagccggcgcttctttacccactaccctcaccacaacccaaaactgcggcttgaccatgagcgaaagaggttcacaacccgaaggcctacatccctcacgcggcgtcgctgcatcagacttccgtccattgtgcaaaattccccactgctgcctcccgtaggagtctgggccgtatctcagtcccaatgtgtccggtcaccctctcaggccggatacccgtcaaagccttggtaagccatcaccccaccaacaagctgataggccgcgagcccatcccccaccagaaaaccctttccacccacccccatgcgagagcaagtgaata

SEQ ID NO: 142 - DNA, 350 bp, Lautropia sp.
ttaccgcggctgctggcacgtagttagccggtgcttattctgcaggtaccgtcatcgtaccgtggtattagcacggcacttttcgttcctgccaaaagtgctttacaacccgaaggccttcatcacacacgcggcattgctggatcaggcttgcgcccattgtccaaaattccccactgctgcctcccgtaggagtctgggccgtgtctcagtcccagtgtggctggtcgtcctctcagaccagctacagatcgtcgccttggtaggcctttaccccaccaactagctaatctgccatcggccgctccaatagcgcgaggccctaaagtcccccgctttcaccctcaggt

SEQ ID NO: 143 - DNA, 350 bp, Parvimonas sp.
ttaccgcggctgctggcacgtatttagccggggcttcctcctatgataccgtcattatcttctcataggacagagctttacgactcgaaagccttcttcgctcacgcggcgtcgctgcatcagggtttcccccattgtgcaatattccccactgctgcctcccgtaggagtctggaccgtgtctcagttccagtgtggccgttcatcctctcagaccggctactgatcgtcgccttggtaggcctttaccctcccaactagctaatcagacgcgagcccatcttacaccgataaatctttaacccctttaccatgcggtactgtggtctcatgaggtattaatcaccgtt

SEQ ID NO: 144 - DNA, 350 bp, Unknown
  source: /note=“Description of Unknown Erysipelotrichaceae bacterium COT-255 sequence”
ttaccgcggctgctggcacgtagtaagccgtgactttctgataaagtggtgtcaaaatgaattcatttcctaattcatctgttcttcctttacaacagagctttacaacccgaaggccttcatcactcacgcggcgttgctcggtcaggctttcgcccattgccgaaaattccttactgctgcctcccgtaggagtttgggccgtgtctcagtcccaatgtggccgttcaccctctcaggccggctatgtatcatcgccttggtgagcagttacctcaccaactagctaatacaccataagcccatctataagtgttggaaacccaactttaataaatagaagatgcctt

SEQ ID NO: 145 - DNA, 350 bp, Peptococcus sp.
ttaccgcggctgctggcacatagttagccggggcttcctcctcatctaccgtcctagaaaagtcagttactactcttctctctcttcgataaggacagagttttacaacccgaaggccttcttcactcacgcggcattgctccgtcaggcttgcgcccattgcggaagattccccactgctgcctcccgtaggagtttgggccgtgtctcagtcccaatgtggccgttcaccctctcaggccggctactgatcgtcgccttggtgagcccttacctcaccaactagctaatcagacgcggatccatctctaaacgacagcgctaaggccgtctttcctctctctatcatg

SEQ ID NO: 146 - DNA, 350 bp, Treponema sp.
ttaccgcggctgctggcacgtaattagccggggcttattcgcatgactaccgtcatctcacaggcattccctcccgtgattattcttcgtctgcaaaagaattttacaacttttcagccttcttcattcacacggcgtcgctccgtcagactttcgtccattgcggaagattcttagctgctgcctcccgtaggagtttgggccgtatctcagtcccaatgtgtccgttcaccctctcaggccggatacccatcgttgccttggtaagcctttaccttaccaactagctaatgggacgcgggcccatcctaaagcggagccgtagctccctttcctcacttctctttata

SEQ ID NO: 147 - DNA, 350 bp, Unknown
  source: /note=“Description of Unknown Peptostreptococcaceae bacterium FOT-017 sequence”
ttaccgcggctgctggcacgtagttagccggggctttcttgtatggtactgtcatttttttcttcccatacgacagagctttacgactcgaaagccttcttcgctcacgcggcattgctgcatcagggttccccccattgtgcaatatcccccactgctgcctcccgtaggagtttgggccgtgtctcagtcccaatgtggccgttcaccctctcaggtcggctactgatcgttgccttggtaggctcttaccccaccaactagctaatcagacgcaagctcgtctacaaccgcctcagctttcgtatatatatcttccgatttatatacctcatctcgtattagctaag

SEQ ID NO: 148 - DNA, 20 bp, Artificial Sequence
  source: /note=“Description of Artificial Sequence Synthetic primer”
agagtttgatymtggctcag

SEQ ID NO: 149 - DNA, 20 bp, Artificial Sequence
  source: /note=“Description of Artificial Sequence Synthetic primer”
agggttcgattctggctcag

SEQ ID NO: 150 - DNA, 16 bp, Artificial Sequence
  source: /note=“Description of Artificial Sequence Synthetic primer”
tyaccgcggctgctgg

SEQ ID NO: 151 - DNA, 350 bp, Actinomyces sp.
ttaccgcggctgctggcacgtagttagccggcgcttcttctgtaagtaaagtcaacccaaacaaaagcctaggcctgcgtccttactgaaagaggtttacaacccgaaggccgtcatccctcacgcggcgtcgctgcatcaggctttcgcccattgtgcaatattccccactgctgcctcccgtaggagtctgggccgtatctcagtcccagtgtgaccggtcaccctctcaggccggctacccgtcaaagccttggtaggccatcaccccaccaacaagctgataggccgcgagcccatcccccaccagaaaaacctttccacacaccaccatgcgacaatgcgtgaat

SEQ ID NO: 152 - DNA, 350 bp, Haemophilus haemoglobinophilus
ttaccgcggctgctggcacggagttagccggtgcttcttctgtgactaacgtcaatcctaaatgctattaacattcaagccttcctcatcaccgaaagaactttacaacccgaaggccttcttcattcacgcggcatggctgcgtcagggttgcccccattgcgcaatattccccactgctgcctcccgtaggagtctggaccgtgtctcagttccagtgtggctggtcatcctctcagaccagctagagatcgaaggcttggtaggcctttaccccaccaactacctaatcccacttgggctcatcttatggcaactggctaaaagtcccagtctttaatcttccgata

SEQ ID NO: 153 - DNA, 350 bp, Streptococcus sp.
ttaccgcggctgctggcacgtagttagccgtccctttctggtaagataccgtcacagtgtgaactttccactctcacactcgttcttctcttacaacagagctttacgatccgaaaaccttcttcactcacgcggcgttgctcggtcagacttccgtccattgccgaagattccctactgctgcctcccgtaggagtctgggccgtgtctcagtcccagtgtggccgatcaccctctcaggtcggctatgtatcgtagccttggtgagccgttaccccaccaactagctaatacaacgcaggtccatctggtagtggtgcaattgcacctttcaattgactatcatgcaa